SEQUENCE LISTING (29 sequences)

SEQ ID NO: 1 - protein, 413 aa, Homo sapiens
MGQPGNGSAFLLAPNRSHAPDHDVTQQRDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPFKYQSLLTKNKARVIILMVWIVSGLTSFLPIQMHWYRATHQEAINCYANETCCDFFTNQAYAIASSIVSFYVPLVIMVFVYSRVFQEAKRQLQKIDKSEGRFHVQNLSQVEQDGRTGHGLRRSSKFCLKEHKALKTLGIIMGTFTLCWLPFFIVNIVHVIQDNLIRKEVYILLNWIGYVNSGFNPLIYCRSPDFRIAFQELLCLRRSSLKAYGNGYSSNGNTGEQSGYHVEQEKENKLLCEDLPGTEDFVGHQGTVPSDNIDSQGRNCSTNDSLL

SEQ ID NO: 2 - protein, 465 aa, Homo sapiens
MFRQEQPLAEGSFAPMGSLQPDAGNASWNGTEAPGGGARATPYSLQVTLTLVCLAGLLMLLTVFGNVLVIIAVFTSRALKAPQNLFLVSLASADILVATLVIPFSLANEVMGYWYFGKAWCEIYLALDVLFCTSSIVHLCAISLDRYWSITQAIEYNLKRTPRRIKAIIITVWVISAVISFPPLISIEKKGGGGGPQPAEPRCEINDQKWYVISSCIGSFFAPCLIMILVYVRIYQIAKRRTRVPPSRRGPDAVAAPPGGTERRPNGLGPERSAGPGGAEAEPLPTQLNGAPGEPAPAGPRDTDALDLEESSSSDHAERPPGPRRPERGPRGKGKARASQVKPGDSLPRRGPGATGIGTPAAGPGEERVGAAKASRWRGRQNREKRFTFVLAVVIGVFVVCWFPFFFTYTLTAVGCSVPRTLFKFFFWFGYCNSSLNPVIYTIFNHDFRRAFKKILCRGDRKRIV

SEQ ID NO: 3 - protein, 590 aa, Homo sapiens
MTLHNNSTTSPLFPNISSSWIHSPSDAGLPPGTVTHFGSYNVSRAAGNFSSPDGTTDDPLGGHTVWQVVFIAFLTGILALVTIIGNILVIVSFKVNKQLKTVNNYFLLSLACADLIIGVISMNLFTTYIIMNRWALGNLACDLWLAIDYVASNASVMNLLVISFDRYFSITRPLTYRAKRTTKRAGVMIGLAWVISFVLWAPAILFWQYFVGKRTVPPGECFIQFLSEPTITFGTAIAAFYMPVTIMTILYWRIYKETEKRTKELAGLQASGTEAETENFVHPTGSSRSCSSYELQQQSMKRSNRRKYGRCHFWFTTKSWKPSSEQMDQDHSSSDSWNNNDAAASLENSASSDEEDIGSETRAIYSIVLKLPGHSTILNSTKLPSSDNLQVPEEELGMVDLERKADKLQAQKSVDDGGSFPKSFSKLPIQLESAVDTAKTSDVNSSVGKSTATLPLSFKEATLAKRFALKTRSQITKRKRMSLVKEKKAAQTLSAILLAFIITWTPYNIMVLVNTFCDSCIPKTFWNLGYWLCYINSTVNPVCYALCNKTFRTTFKMLLLCQCDKKKRRKQQYQQRQSVIFHKRAPEQAL

SEQ ID NO: 4 - protein, 458 aa, Homo sapiens
MVNLRNAVHSFLVHLIGLLVWQCDISVSPVAAIVTDIFNTSDGGRFKFPDGVQNWPALSIVIIIIMTIGGNILVIMAVSMEKKLHNATNYFLMSLAIADMLVGLLVMPLSLLAILYDYVWPLPRYLCPVWISLDVLFSTASIMHLCAISLDRYVAIRNPIEHSRFNSRTKAIMKIAIVWAISIGVSVPIPVIGLRDEEKVFVNNTTCVLNDPNFVLIGSFVAFFIPLTIMVITYCLTIYVLRRQALMLLHGHTEEPPGLSLDFLKCCKRNTAEEENSANPNQDQNARRRKKKERRPRGTMQAINNERKASKVLGIVFFVFLIMWCPFFITNILSVLCEKSCNQKLMEKLLNVFVWIGYVCSGINPLVYTLFNKIYRRAFSNYLRCNYKVEKKPPVRQIPRVAATALSGRELNVNIYRHTNEPVIEKASDNEPGIEMQVENLELPVNPSSVVSERISSV

SEQ ID NO: 5 - protein, 443 aa, Homo sapiens
MDPLNLSWYDDDLERQNWSRPFNGSDGKADRPHYNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMISIVWVLSFTISCPLLFGLNNADQNECIIANPAFVVYSSIVSFYVPFIVTLLVYIKIYIVLRRRRKRVNTKRSSRAFRAHLRAPLKGNCTHPEDMKLCTVIMKSNGSFPVNRRRVEAARRAQELEMEMLSSTSPPERTRYSPIPPSHHQLTLPDPSHHGLHSTPDSPAKPEKNGHAKDHPKIAKIFEIQTMPNGKTRTSLKTMSRRKLSQQKEKKATQMLAIVLGVFIICWLPFFITHILNIHCDCNIPPVLYSAFTWLGYVNSAVNPIIYTTFNIEFRKAFLKILHC

SEQ ID NO: 6 - protein, 20 aa, Artificial sequence
  Peptide fragment of Ga protein
VFAAVKDTILQLNLKEYNLV

SEQ ID NO: 7 - protein, 20 aa, Artificial sequence
  Peptide fragment of Ga protein
VFNDCRDIIQRMHLRQYELL

SEQ ID NO: 8 - protein, 20 aa, Artificial sequence
  Peptide fragment of Ga protein
VFDAVTDVIIKNNLKDCGLF

SEQ ID NO: 9 - protein, 481 aa, Homo sapiens
MALSYRVSELQSTIPEHILQSTFVHVISSNWSGLQTESIPEEMKQIVEEQGNKLHWAALLILMVIIPTIGGNTLVILAVSLEKKLQYATNYFLMSLAVADLLVGLFVMPIALLTIMFEAMWPLPLVLCPAWLFLDVLFSTASIMHLCAISVDRYIAIKKPIQANQYNSRATAFIKITVVWLISIGIAIPVPIKGIETDVDNPNNITCVLTKERFGDFMLFGSLAAFFTPLAIMIVTYFLTIHALQKKAYLVKNKPPQRLTWLTVSTVFQRDETPCSSPEKVAMLDGSRKDKALPNSGDETLMRRTSTIGKKSVQTISNEQRASKVLGIVFFLFLLMWCPFFITNITLVLCDSCNQTTLQMLLEIFVWIGYVSSGVNPLVYTLFNKTFRDAFGRYITCNYRATKSVKTLRKRSSKIYFRNPMAENSKFFKKHGIRNGINPAMYQSPMRLRSSTIQSSSIILLDTLLLTENEGDKTEERVSYV

SEQ ID NO: 10 - protein, 440 aa, Homo sapiens
MVPEPGPTANSTPAWGAGPPSAPGGSGWVAAALCVVIALTAAANSLLIALICTQPALRNTSNFFLVSLFTSDLMVGLVVMPPAMLNALYGRWVLARGLCLLWTAFDVMCCSASILNLCLISLDRYLLILSPLRYKLRMTPLRALALVLGAWSLAALASFLPLLLGWHELGHARPPVPGQCRLLASLPFVLVASGLTFFLPSGAICFTYCRILLAARKQAVQVASLTTGMASQASETLQVPRTPRPGVESADSRRLATKHSRKALKASLTLGILLGMFFVTWLPFFVANIVQAVCDCISPGLFDVLTWLGYCNSTMNPIIYPLFMRDFKRALGRFLPCPRCPRERQASLASPSLRTSHSGPRPGLSLQQVLPLPLPPDSDSDSDAGSGGSSGLRLTAQLLLPGEATQDPPLPTRAAAAVNFFNIDPAEPELRPHPLGIPTN

SEQ ID NO: 11 - protein, 241 aa, Artificial sequence
  cpEGFP
NVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYN

SEQ ID NO: 12 - protein, 246 aa, Artificial sequence
  CpmApple
PVVSERMYPEDGALKSEIKKGLRLKDGGHYAAEVKTTYKAKKPVQLPGAYIVDIKLDIVSHNEDYTIVEQCERAEGRHSTGGMDELYKGGTGGSLVSKGEEDNMAIIKEFMRFKVHMEGSVNGHEFEIEGEGEGRPYEAFQTAKLKVTKGGPLPFAWDILSPQFMYGSKAYIKHPADIPDYFKLSFPEGFRWERVMNFEDGGIIHVNQDSSLQDGVFIYKVKLRGTNFPPDGPVMQKKTMGWEATR

SEQ ID NO: 13 - protein, 697 aa, Artificial sequence
  GRAB-EPI 1.0
METDTLLLWVLLLWVPGSTGDYPYDVPDYAGAQPARSGQPGNGSAFLLAPNGSHAPDHDVTQERDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPFKYQSLLTKNKARVIILMVWIVSGLTSFLPIQMHWYRATHQEAINCYANETCCDFFTNQAYAIASSIVSFYVPLVIMVFVYSRVFQEAKRQLQKIDKSEGRFHVQNLSQVEQGGNVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNGGAAADGRTGHGLRRSSKFCLKEHKALKTLGIIMGTFTLCWLPFFIVNIVHVIQDNLIRKEVYILLNWIGYVNSGFNPLIYCRSPDFRIAFQELLCLRRSSLKAYGNGYSSNGNTGEQSGYHVEQEKENKLLCEDLPGTEDFVGHQGTVPSDNIDSQGRNCSTNDSLL

SEQ ID NO: 14 - protein, 697 aa, Artificial sequence
  GRAB-EPI 2.0
METDTLLLWVLLLWVPGSTGDYPYDVPDYAGAQPARSGQPGNGSAFLLAPNGSHAPDHDVTQERDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPFKYQSLLTKNKARVIILMVWIVSGLTSFLPIQMHWYRATHQEAINCYANETCCDFFTNQAYAIASSIVSFYVPLVIMVFVYSRVFQEAKRQLQKIDKSEGRFHVQNLSQVEQGGNVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVRKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNAPSVADGRTGHGLRRSSKFCLKEHKALKTLGIIMGTFTLCWLPFFIVNIVHVIQDNLIRKEVYILLNWIGYVNSGFNPLIYCRSPDFRIAFQELLCLRRSSLKAYGNGYSSNGNTGEQSGYHVEQEKENKLLCEDLPGTEDFVGHQGTVPSDNIDSQGRNCSTNDSLL

SEQ ID NO: 15 - protein, 682 aa, Artificial sequence
  GRAB-ACh1.0
METDTLLLWVLLLWVPGSTGDTSLYKKVGTMTLHNNSTTSPLFPNISSSWIHSPSDAGLPPGTVTHFGSYNVSRAAGNFSSPDGTTDDPLGGHTVWQVVFIAFLTGILALVTIIGNILVIVSFKVNKQLKTVNNYFLLSLACADLIIGVISMNLFTTYIIMNRWALGNLACDLWLAIDYVASNASVMNLLVISFDRYFSITRPLTYRAKRTTKRAGVMIGLAWVISFVLWAPAILFWQYFVGKRTVPPGECFIQFLSEPTITFGTAIAAFYMPVTIMTILYWRVFQEAKRQLQKIDKSEGRFHVQNLSQVEQGGNVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNGGAAADGRTGHGLRRSSKFCLKEHKALKTLSAILLAFIITWTPYNIMVLVNTFCDSCIPKTFWNLGYWLCYINSTVNPVCYALCNKTFRTTFKMLLLCQCDKKKRRKQQYQQRQSVIFHKRAPEQAL

SEQ ID NO: 16 - protein, 682 aa, Artificial sequence
  GRAB-ACh2.0
METDTLLLWVLLLWVPGSTGDTSLYKKVGTMTLHNNSTTSPLFPNISSSWIHSPSDAGLPPGTVTHFGSYNVSRAAGNFSSPDGTTDDPLGGHTVWQVVFIAFLTGILALVTIIGNILVIVSFKVNKQLKTVNNYFLLSLACADLIIGVISMNLFTTYIIMNRWALGNLACDLWLAIDYVASNASVMNLLVISFDRYFSITRPLTYRAKRTTKRAGVMIGLAWVISFVLWAPAILFWQYFVGKRTVPPGECFIQFLSEPTITFGTAIAAFYMPVTIMTILYWRVFQEAKRQLQKIDKSEGRFHVQNLSQVEQGGNVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVRKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNAPSVADGRTGHGLRRSSKFCLKEHKALKTLSAILLAFIITWTPYNIMVLVNTFCDSCIPKTFWNLGYWLCYINSTVNPVCYALCNKTFRTTFKMLLLCQCDKKKRRKQQYQQRQSVIFHKRAPEQAL

SEQ ID NO: 17 - protein, 733 aa, Artificial sequence
  GRAB-ACh-Cter580
METDTLLLWVLLLWVPGSTGDTSLYKKVGTTLHNNSTTSPLFPNISSSWIHSPSDAGLPPGTVTHFGSYNVSRAAGNFSSPDGTTDDPLGGHTVWQVVFIAFLTGILALVTIIGNILVIVSFKVNKQLKTVNNYFLLSLACADLIIGVISMNLFTTYIIMNRWALGNLACDLWLAIDYVASNASVMNLLVISFDRYFSITRPLTYRAKRTTKRAGVMIGLAWVISFVLWAPAILFWQYFVGKRTVPPGECFIQFLSEPTITFGTAIAAFYMPVTIMTILYWRVFQEAKRQLQKIDKSEGRFHVQNLSQVEQDGRTGHGLRRSSKFCLKEHKALKTLSAILLAFIITWTPYNIMVLVNTFCDSCIPKTFWNLGYWLCYINSTVNPVCYALCNKTFRTTFKMLLLCQCDKKKRRKQQYQQRQSVIGGNVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVRKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNGGAAAFHKRAPEQALVFAAVKDTILQLNLKEYNLVKSRITSEGEYIPLDQIDINVNSFCYENEVALT

SEQ ID NO: 18 - protein, 793 aa, Artificial sequence
  GRAB-DA-Cter580
METDTLLLWVLLLWVPGSTGDTSLYKKVGTDPLNLSWYDDDLERQNWSRPFNGSDGKADRPHYNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMISIVWVLSFTISCPLLFGLNNADQNECIIANPAFVVYSSIVSFYVPFIVTLLVYIKIYIVLRRRRKRVNTKRSSRAFRAHLRAPLKGNCTHPEDMKLCTVIMKSNGSFPVNRRRVEAARRAQELEMEMLSSTSPPERTRYSPIPPSHHQLTLPDPSHHGLHSTPDSPAKPEKNGHAKDHPKIAKIFEIQTMPNGKTRTSLKTMSRRKLSQQKEKKATQMLAIVLGVFIICWLPFFITHILNIHCDCNIPPVLYSAFTWLGYVNSAVNPIIYTTFNIEFRKAFLKILRRKQQYQQRQSVIGGNVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVRKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNGGAAAFHKRAPEQALVFAAVKDTILQLNLKEYNLVKSRITSEGEYIPLDQIDINVNSFCYENEVALT

SEQ ID NO: 19 - protein, 836 aa, Artificial sequence
  GRAB-His-Cter580
METDTLLLWVLLLWVPGSTGDTSLYKKVGTSLPNSSCLLEDKMCEGNKTTMASPQLMPLVVVLSTICLVTVGLNLLVLYAVRSERKLHTVGNLYIVSLSVADLIVGAVVMPMNILYLLMSKWSLGRPLCLFWLSMDYVASTASIFSVFILCIDRYRSVQQPLRYLKYRTKTRASATILGAWFLSFLWVIPILGWNHFMQQTSVRREDKCETDFYDVTWFKVMTAIINFYLPTLLMLWFYAKIYKAVRQHCQHRELINRSLPSFSEIKLRPENPKGDAKKPGKESPWEVLKRKPKDAGGGSVLKSPSQTPKEMKSPVVFSQEDDREVDKLYCFPLDIEHMQAAAEGSSRDYVAVNRSHGQLKTDEQGLNTHGASEISEDQMLGDSQSFSRTDSDTTTETAPGKGKLRSGSNTGLDYIKFTWKRLRSHSRQYVSGLHMNRERKAAKQLGFIMAAFILCWIPYFIFFMVIAFCKNCCNEHLHMFTIWLGYINSTLNPLIYPLCNENFKKTFKRILKRRKQQYQQRQSVIGGNVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVRKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNGGAAAFHKRAPEQALVFAAVKDTILQLNLKEYNLVKSRITSEGEYIPLDQIDINVNSFCYENEVALT

SEQ ID NO: 20 - protein, 685 aa, Artificial sequence
  GRAB-5HT-Cter580
METDTLLLWVLLLWVPGSTGDTSLYKKVGTDKLDANVSSNEGFRSVEKVVLLTFLAVVILMAILGNLLVMVAVCRDRQLRKIKTNYFIVSLAFADLLVSVLVMPFGAIELVQDIWAYGEMFCLVRTSLDVLLTTASIFHLCCISLDRYYAICCQPLVYRNKMTPLRIALMLGGCWVLPMFISFLPIMQGWNNIGIVDVIEKRKFSHNSNSTWCVFMVNKPYAITCSVVAFYIPFLLMVLAYYRIYVTAKEHAQQIQMLQRAGATSESRPQPADQHSTHRMRTETKAAKTLCVIMGCFCFCWAPFFVTNIVDPFIDYTVPEQVWTAFLWLGYINSGLNPFLYAFLNKSFRRAFLIILCCDDERYRKQQYQQRQSVIGGNVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVRKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNGGAAAFHKRAPEQALVFAAVKDTILQLNLKEYNLVKSRITSEGEYIPLDQIDINVNSFCYENEVALT

SEQ ID NO: 21 - protein, 706 aa, Artificial sequence
  GRAB-Oxt-Cter580
METDTLLLWVLLLWVPGSTGDTSLYKKVGTEGALAANWSAEAANASAAPPGAEGNRTAGPPRRNEALARVEVAVLCLILLLALSGNACVLLALRTTRQKHSRLFFFMKHLSIADLVVAVFQVLPQLLWDITFRFYGPDLLCRLVKYLQVVGMFASTYLLLLMSLDRCLAICQPLRSLRRRTDRLAVLATWLGCLVASAPQVHIFSLREVADGVFDCWAVFIQPWGPKAYITWITLAVYIVPVIVLAACYGLISFKIWQNLRLKTAAAAAAEAPEGAAAGDGGRVALARVSSVKLISKAKIRTVKMTFIIVLAFIVCWTPFFFVQMWSVWDANAPKEASAFIIVMLLASLNSCCNPWIYMLFTGHLFHELVQRFLCCSASYLKKRRKQQYQQRQSVIGGNVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVRKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNGGAAAFHKRAPEQALVFAAVKDTILQLNLKEYNLVKSRITSEGEYIPLDQIDINVNSFCYENEVALT

SEQ ID NO: 22 - protein, 698 aa, Artificial sequence
  GRAB-Epi-Cter580
METDTLLLWVLLLWVPGSTGDTSLYKKVGTGQPGNGSAFLLAPNGSHAPDHDVTQERDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPFKYQSLLTKNKARVIILMVWIVSGLTSFLPIQMHWYRATHQEAINCYANETCCDFFTNQAYAIASSIVSFYVPLVIMVFVYSRVFQEAKRQLQKIDKSEGRFHVQNLSQVEQDGRTGHGLRRSSKFCLKEHKALKTLGIIMGTFTLCWLPFFIVNIVHVIQDNLIRKEVYILLNWIGYVNSGFNPLIYCRSPDFRIAFQELLCLRRSSRRKQQYQQRQSVIGGNVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVRKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNGGAAAFHKRAPEQALVFAAVKDTILQLNLKEYNLVKSRITSEGEYIPLDQIDINVNSFCYENEVALT

SEQ ID NO: 23 - protein, 665 aa, Artificial sequence
  GRAB-NE2.1
METDTLLLWVLLLWVPGSTGDTSLYKKVGTTGGSLQPDAGNASWNGTEAPGGGARATPYSLQVTLTLVCLAGLLMLLTVFGNVLVIIAVFTSRALKAPQNLFLVSLASADILVATLVIPFSLANEVMGYWYFGKAWCEIYLALDVLFCTSSIVHLCAISLDRYWSITQAIEYNLKRTPRRIKAIIITVWVISAVISFPPLISIEKKGGGGGPQPAEPRCEINDQKWYVISSCIGSFFAPCLIMILVYVRIYQIAKRRTRVPPSRRGPDAVAAPPGGTERRPNGLGPERSAGPGGAEAEPLPTQLNGAPGEPAPAGPRDTDALDLEEGGNVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVRKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNTGAAARWRGRQNREKRFTFVLAVVIGVFVVCWFPFFFTYTLTAVGCSVPRTLFKFFFWFGYCNSSLNPVIYTIFNHDFRRAFKKILCRGDRKRIVL

SEQ ID NO: 24 - protein, 665 aa, Artificial sequence
  GRAB-NE2.2
METDTLLLWVLLLWVPGSTGDTSLYKKVGTTGGSLQPDAGNASWNGTEAPGGGARATPYSLQVTLTLVCLAGLLMLLTVFGNVLVIIAVFTSRALKAPQNLFLVSLASADILVATLVIPFSLANEVMGYWYFGKAWCEIYLALDVLFCTSSIVHLCAISLDRYWSITQAIEYNLKRTPRRIKAIIITVWVISAVISFPPLISIEKKGGGGGPQPAEPRCEINDQKWYVISSCIGSFFAPCLIMILVYVRIYQIAKRRTRVPPSRRGPDAVAAPPGGTERRPNGLGPERSAGPGGAEAEPLPTQLNGAPGEPAPAGPRDTDALDLEEGGNVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVRKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNTGAAARWRGRQNREKRFKFVLAVVIGVFVVCWFPFFFTYTLTAVGCSVPRTLFKFFFWFGYCNSSLNPVIYTIFNHDFRRAFKKILCRGDRKRIVL

SEQ ID NO: 25 - protein, 682 aa, Artificial sequence
  GRAB-5-HT
METDTLLLWVLLLWVPGSTGDTSLYKKVGTMVNLRNAVHSFLVHLIGLLVWQCDISVSPVAAIVTDIFNTSDGGRFKFPDGVQNWPALSIVIIIIMTIGGNILVIMAVSMEKKLHNATNYFLMSLAIADMLVGLLVMPLSLLAILYDYVWPLPRYLCPVWISLDVLFSTASIMHLCAISLDRYVAIRNPIEHSRFNSRTKAIMKIAIVWAISIGVSVPIPVIGLRDEEKVFVNNTTCVLNDPNFVLIGSFVAFFIPLTIMVITYCLTIYVLRRQALMLLNGNVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVRKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNGFAAANNERKASKVLGIVFFVFLIMWCPFFITNILSVLCEKSCNQKLMEKLLNVFVWIGYVCSGINPLVYTLFNKIYRRAFSNYLRCNYKVEKKPPVRQIPRVAATALSGRELNVNIYRHTNEPVIEKASDNEPGIEMQVENLELPVNPSSVVSERISSV

SEQ ID NO: 26 - protein, 616 aa, Artificial sequence
  GRAB-GDA3.0
METDTLLLWVLLLWVPGSTGDTSLYKKVGTMDPLNLSWYDDDLERQNWSRPFNGSDGKADRPHYNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPMLYNTRYSSKRRVTVMISIVWVLSFTISCPLLFGLNNADQNECIIANPAFVVYSSIVSFYVPFIVTLLVYIKIYIVLRRRRKRVNTKRSSRAFRAHLRAPLKGNCTHPEDMKLGGNVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVRKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNGGAAAMSRRKLSQQKEKKATQMLAIVLGVFIICWLPFFITHILNIHCDCNIPPVLYSAFTWLGYVNSAVNPIIYTTFNIEFRKAFLKILHC

SEQ ID NO: 27 - protein, 487 aa, Homo sapiens
MSLPNSSCLLEDKMCEGNKTTMASPQLMPLVVVLSTICLVTVGLNLLVLYAVRSERKLHTVGNLYIVSLSVADLIVGAVVMPMNILYLLMSKWSLGRPLCLFWLSMDYVASTASIFSVFILCIDRYRSVQQPLRYLKYRTKTRASATILGAWFLSFLWVIPILGWNHFMQQTSVRREDKCETDFYDVTWFKVMTAIINFYLPTLLMLWFYAKIYKAVRQHCQHRELINRSLPSFSEIKLRPENPKGDAKKPGKESPWEVLKRKPKDAGGGSVLKSPSQTPKEMKSPVVFSQEDDREVDKLYCFPLDIEHMQAAAEGSSRDYVAVNRSHGQLKTDEQGLNTHGASEISEDQMLGDSQSFSRTDSDTTTETAPGKGKLRSGSNTGLDYIKFTWKRLRSHSRQYVSGLHMNRERKAAKQLGFIMAAFILCWIPYFIFFMVIAFCKNCCNEHLHMFTIWLGYINSTLNPLIYPLCNENFKKTFKRILHIRS

SEQ ID NO: 28 - protein, 388 aa, Homo sapiens
MDKLDANVSSEEGFGSVEKVVLLTFLSTVILMAILGNLLVMVAVCWDRQLRKIKTNYFIVSLAFADLLVSVLVMPFGAIELVQDIWIYGEVFCLVRTSLDVLLTTASIFHLCCISLDRYYAICCQPLVYRNKMTPLRIALMLGGCWVIPTFISFLPIMQGWNNIGIIDLIEKRKFNQNSNSTYCVFMVNKPYAITCSVVAFYIPFLLMVLAYYRIYVTAKEHAHQIQMLQRAGASSESRPQSADQHSTHRMRTETKAAKTLCIIMGCFCLCWAPFFVTNIVDPFIDYTVPGQVWTAFLWLGYINSGLNPFLYAFLNKSFRRAFLIILCCDDERYRRPSILGQTVPCSTTTINGSTHVLRDAVECGGQWESQCHPPATSPLVAAQPSDT

SEQ ID NO: 29 - protein, 389 aa, Homo sapiens
MEGALAANWSAEAANASAAPPGAEGNRTAGPPRRNEALARVEVAVLCLILLLALSGNACVLLALRTTRQKHSRLFFFMKHLSIADLVVAVFQVLPQLLWDITFRFYGPDLLCRLVKYLQVVGMFASTYLLLLMSLDRCLAICQPLRSLRRRTDRLAVLATWLGCLVASAPQVHIFSLREVADGVFDCWAVFIQPWGPKAYITWITLAVYIVPVIVLAACYGLISFKIWQNLRLKTAAAAAAEAPEGAAAGDGGRVALARVSSVKLISKAKIRTVKMTFIIVLAFIVCWTPFFFVQMWSVWDANAPKEASAFIIVMLLASLNSCCNPWIYMLFTGHLFHELVQRFLCCSASYLKGRRLGETSASKKSNSSSFVLSHRSSSQRSCSQPSTA

(19) United States
(12) Patent Application Publication
(10) Pub. No.: US 2020/0400567 A1
(43) Pub. Date: Dec. 24, 2020

(54) FUSION POLYPEPTIDE

(71) Applicant: PEKING UNIVERSITY, Beijing (CN)

(72) Inventors: Yulong LI, Beijing (CN); Miao JING, Beijing (CN); Jiesi FENG, Beijing (CN); Huan WANG, Beijing (CN); Jinxia WAN, Beijing (CN); Fangmiao SUN, Beijing (CN); Jianzhi ZENG, Beijing (CN)

(73) Assignee: PEKING UNIVERSITY, Beijing (CN)

(21) Appl. No.: 16/651,288

(22) PCT Filed: Sep. 26, 2018
(86) PCT No.: PCT/CN2018/107533

§ 371 Date: Jul. 21, 2020

(30) Foreign Application Priority Data
Sep. 27, 2017 (CN) 201710892931.0
Apr. 9, 2018 (CN) 201810345711.0

(51) Int. Cl. G01N 21/64; C07K 14/705; C07K 14/72; G01N 33/74

(52) U.S. Cl. CPC G01N 21/6428; C07K 14/705; C07K 14/723; C07K 2319/73; G01N 33/74; C07K 2319/60; C07K 2319/61; C07K 14/70571

(57) ABSTRACT

Using the characteristics of G-protein coupled receptors (GPCR) for sensing specific ligands and undergoing conformational change, inserting a signal molecule in an intracellular region of the G-protein coupled receptors, converting the conformational change of the G-protein coupled receptors into an optical signal change, and detecting the presence and/or concentration of a specific ligand by means of detecting the change in the optical signal; a GPCR activated fluorescent probe (GRAB probe) is constructed according to this principle. A method for using the GRAB probe to detect a specific ligand.

### CROSS-REFERENCE OF RELATED APPLICATIONS

The present application claims the priorities of Chinese Patent Application No. 201710892931.0 filed with the China Patent Office on Sep. 27, 2017, titled “Fluorescent Sensor Constructed Based on G Protein-Coupled Receptors”, and Chinese Patent Application No. 201810345711.0 filed with the China Patent Office on Apr. 9, 2018, titled “Fusion Polypeptide”, each of which is incorporated by reference in its entirety.

### FIELD

The present disclosure relates to a fusion polypeptide, and in particular, to a fusion polypeptide constructed based on G protein-coupled receptors.

### BACKGROUND

G Protein-Coupled Receptor (GPCR) is an important protein in cell signaling and an important drug target, with about 40% of clinical prescription drugs directly or indirectly targeting GPCR according to statistics. The signal transduction mechanism and drug screening of GPCR are research hotspots. These receptors are relatively conservative in structure, and all have seven transmembrane alpha helices. GPCR is capable of interacting with various G proteins, causing a series of cellular effects such as the generation of intracellular second messenger. G protein-coupled receptors can recognize a variety of ligands and stimuli, including hormones and neurotransmitters, chemokines, prostaglandins, proteases, biogenic amines, nucleosides, lipids, growth factors, odorant molecules, and light. These receptors act as intracellular mediators to regulate complex pathway network. One important type ligand is neurotransmitter. Due to the important role of neurotransmitters in the nervous system, scientists have carried out various researches on the properties, synthesis, storage, release and functions of neurotransmitters in the nearly 100 years since the identification of the first neurotransmitter acetylcholine (Valenstein, E. S. The discovery of chemical neurotransmitters. Brain and cognition 49, 73-95 (2002)). However, compared with the rapid development in the field of cognitive neurobiology nowadays, the technique of detecting neurotransmitters is still limited by spatial and temporal resolution and cell specificity, making it difficult to characterize the release and function of the transmitter.

Microdialysis coupled with biochemical analysis is one of the classic methods for studying neurotransmitter release. This method was first developed by Bito L in 1966 and used to detect the content and dynamic changes of various amino acids in the brain (Justice, J. B. Quantitative microdialysis of neurotransmitters, Journal of Neuroscience Methods 48, 263-276 (1993)). Understedt and Pycock, as pioneers in this field, improved the microdialysis technique and applied it to detect a variety of important neurotransmitters such as dopamine in the neural circuit of the brain (Watson, C. J., Venton, B. J. & Kennedy, R. T. In vivo measurements of neurotransmitters by microdialysis sampling. Analytical chemistry 78, 1391-1399 (2006)). Although achieving the purpose of detecting neurotransmitters, this method requires obtaining neurotransmitters through the dialysis membrane and isolating and identifying specific molecules via biochemical methods, and therefore greatly loses the spatiotemporal information about transmitter release; and due to the complicated operation, it is difficult to guarantee a complete reflection of the physiological state. The more-refined nano-LC-microdialysis developed nowadays allows us to precisely separate and profile a very small amount of neurotransmitters in the tissue, with a sample volume as small as 4 nL and a time resolution of a few seconds. However, the poor spatial resolution of this method results in the defect of lacking cell-specific detection (Olive, M. F., Mehmert, K. K. & Hodge, C. W. Microdialysis in the mouse nucleus accumbens: A method for detection of monoamine and amino acid neurotransmitters with simultaneous assessment of locomotor activity. Brain Research Protocols 5, 16-24 (2000); Lee, G. J., Park, J. H. & Park, H. K. Microdialysis applications in neuroscience. Neurological research 30, 661-668 (2008)).

In addition to the biochemical methods, the electrochemical techniques developed by chemical redox methods have been used to detect monoamine neurotransmitters such as dopamine, serotonin, etc. and became one of the most widely used methods to detect neurotransmitter release currently. In this method, changes can be coupled with electrical signals, so it has good sensitivity and time resolution, and thus plays an important role in the study of the release of monoamine neurotransmitters such as dopamine and serotonin, and also the regulatory mechanisms. (Zhou, Z. & Misler, S. Amperometric detection of stimulus-induced quantal release of catecholamines from cultured superior cervical ganglion neurons, Proceedings of the National Academy of Sciences of the United States of America 92, 6938-6942 (1995); Bruns, D. Detection of transmitter release with carbon fiber electrodes, Methods (San Diego, Calif.) 33, 312-321 (2004)). However, since it is difficult to accurately target a specific synapse position using an electrode, this method is actually difficult to achieve precise cell and subcellular specificity. And due to factors such as the insertion of electrode, this method causes certain damage to tissues and cells so that it is not suitable for parallel detection of multiple areas. On the other hand, since this method is developed based on the principle of chemical redox, it is only applicable for monoamine neurotransmitters, and cannot be used for other important neurotransmitters such as acetylcholine. Although this method plays a key role in studying the function and release of neurotransmitters, it has limited role in today's neurobiology research that requires cell-specific and spatial-temporal specificity.

The optical imaging methods for detecting neurotransmitters have been rapidly developed in recent years due to its high sensitivity and real-time observation. The neurotransmitter detection method CNiFERs, which is based on the detection cell line, was developed by the David Kleinfeld laboratory of the University of San Diego, San Francisco, USA. This method can detect the neurotransmitter release in the brain region by implanting the modified human HEK293 cells into a specific brain region (Muller, A., Joseph, V, Slesinger, P. A. & Kleinfeld, D. Cell-based reporters reveal in vivo dynamics of dopamine and norepinephrine release in murine cortex. Nature methods 11, 1245-1252 (2014); Nguyen, Q. T. et al. An in vivo biosensor for neurotransmitter release and in situ receptor activity. Nature neuroscience 13, 127-132 (2010)). The cells express a G protein-coupled receptor corresponding to a specific neurotransmitter, and the receptor is coupled to a downstream fluorescent calcium indicator, so the binding of the neurotransmitter is transferred into an intracellular calcium signal. This method can be used for specific neurotransmitter detection and plays an important role in the detection of epinephrine, dopamine and acetylcholine. In addition, the detection signal is not the neurotransmitter binding itself, but the secondary calcium which is amplified by the downstream cascade reaction, giving the method higher sensitivity and second-level time resolution. However, because the need to transplant exogenous cells into a specific location in the brain, it is still difficult to detect the functions of neurotransmitters on endogenous neurons, subcellular axons, dendrites and even single synapse. On the other hand, the complicated operation process and potential immune rejection also limit the wide application of this method in the field of neurobiology.

Therefore, there is an urgent need for a method capable of detecting the binding of G protein-coupled receptors and ligands, especially for the spatiotemporal specific detection of neurotransmitters or drug candidates.

### SUMMARY

The inventors unexpectedly found that by inserting a signal molecule capable of responding to a conformational change into GPCR at the position where a conformational change occurs, the inserted signal molecule can respond to the conformational change caused by the binding of the ligand to the GPCR and generate a signal intensity change. The inventors successfully develop a fusion polypeptide sensor capable of indicating GPCR activity in vivo for the first time and complete the present disclosure. Herein, the fusion polypeptide of the present disclosure is referred to GPCR Activation Based (GRAB) sensor.

Since the receptor of most classical neurotransmitters is G protein-coupled receptor (GPCR), the GRAB sensor of the present disclosure and the detection method using the sensor are particularly suitable for detecting neurotransmitters. Drugs that directly/indirectly target GPCR account for a considerable proportion in the clinic. By constructing specific G protein-coupled receptors capable of binding with neurotransmitters/candidate drugs (as ligands), the conformation changes of these receptors initiated by ligands are directly coupled to the output of signaling molecules to reflect the binding status and dynamic changes of neurotransmitter/candidate drug and GPCR.

The present disclosure provides the following technical solutions:

1. A fusion polypeptide comprising a G protein-coupled receptor (GPCR) part and a signal molecule part, wherein the G protein-coupled receptor is capable of specifically binding to ligand thereof, and the signal molecule is capable of directly or indirectly generating a detectable signal, such as an optical signal or a chemical signal, in response to the binding.

2. The fusion polypeptide according to embodiment 1, wherein the signal molecule is connected to an intracellular region of the G protein-coupled receptor; particularly, the signal molecule is connected to an intracellular loop or the C-terminus of the GPCR, for example the first intracellular loop, the second intracellular loop, the third intracellular loop or the C-terminus of the GPCR, preferably the third intracellular loop or C-terminus of the GPCR, and more preferably the third intracellular loop of the GPCR.

3. The fusion polypeptide according to embodiment 2, wherein the signal molecule is connected to the third intracellular loop or C-terminus of the GPCR, and the third intracellular loop or C-terminus is a truncated third intracellular loop or C-terminus, preferably, the third intracellular loop or the C-terminus is truncated 10-200 amino acids, such as 10, 20, 30, 40, 50, 60, 70, 80, 90, 100, 110, 120, 130, 140, 150, 160, 170, 180, 190, 200 amino acids, or a range between any two of the values thereof.

4. The fusion polypeptide according to embodiment 2 or 3, wherein the signal molecule is connected to the GPCR through a peptide linker, for example, the signal molecule is connected to the third intracellular loop of the GPCR through a linker peptide; preferably, the peptide linker comprises a flexible amino acid; more preferably, the flexible amino acid comprises glycine and/or alanine; even more preferably, the peptide linker consists of glycine and alanine; and most preferably, the peptide linker at the N-terminus of the signal molecule is GG, and/or the peptide linker at the C-terminus of the signal molecule is GGAAA.

5. The fusion polypeptide according to any one of embodiments 1 to 4, wherein the detectable signal is an optical signal; preferably, the signal molecule is a fluorescent protein or luciferase; more preferably, the signal molecule is a circular permutated fluorescent protein or a circular permutated luciferase.

6. The fusion polypeptide according to embodiment 5, wherein the signal molecule is a circular permutated fluorescent protein,

for example, the circular permutated fluorescent protein is selected from the group consisting of circular permutated green fluorescent protein (cpGFP), circular permutated yellow fluorescent protein (cpYFP), circular permutated red fluorescent protein (cpRFP), circular permutated blue fluorescent protein (cpBFP), circular permutated enhanced green fluorescent protein (cpEGFP), circular permutated enhanced yellow fluorescence protein (cpEYFP) and circular permutated infrared fluorescent protein (cpiRFP);

for example, the circular permutated enhanced green fluorescent protein is from GCaMP6s, GCaMP6m or G-GECO;

for example, the circular permutated red fluorescent protein is selected from the group consisting of cpmApple, cpmCherry, cpmRuby2, cpmKate2, and cpFushionRed,
particularly, the cpmApple is from R-GECO1;

for example, the circular permutated yellow fluorescent protein is selected from circular permutated Venus (cpVenus) and circular permutated Citrin (cpCitrine).

7. The fusion polypeptide according to any one of embodiments 1 to 6, wherein the GPCR is capable of specifically binding to ligand thereof, wherein the ligand is selected from the group consisting of a neurotransmitter, hormone, metabolic molecule, nutrition molecule, and an artificially synthesized small molecule or drug candidate capable of activating a specific receptor; and the GPCR is capable of specifically binding to the neurotransmitter, hormone, metabolic molecule, nutrition molecule, or the artificially synthesized small molecule or drug candidate;

for example, the neurotransmitter is epinephrine, norepinephrine, acetylcholine, serotonin and/or dopamine;

for example, the artificially synthesized small molecule or drug candidate capable of activating a specific receptor is isoproterenol (ISO);

for example, the G protein-coupled receptor is derived from human or mammalian G protein-coupled receptor;

for example, the fusion polypeptide is a fluorescent sensor for detecting epinephrine, and the GPCR is capable of specifically binding to epinephrine; particularly, the GPCR capable of specifically binding to epinephrine is a human β2 adrenergic receptor, and the fusion polypeptide is a fluorescent sensor constructed based on the human β2 adrenergic receptor.

8. The fusion polypeptide according to embodiment 7, wherein the signal molecule is the circular permutated fluorescent protein and the circular permutated fluorescent protein is inserted into the third intracellular loop of the human β2 adrenergic receptor through peptide linkers at the N-terminus and the C-terminus;

preferably, the lengths of the peptide linkers are 1 or 2 amino acids at the N-terminus and/or 1, 2, 3, 4 or 5 amino acids at the C-terminus of the circular permutated fluorescent protein, respectively;

more preferably, the lengths of the peptide linkers are 2 amino acids at the N-terminus and 5 amino acids at the C-terminus of the circular permutated fluorescent protein, respectively; and
preferably, the peptide linkers are GG at the N-terminus and GGAAA at the C-terminus of the circular permutated fluorescent protein, respectively, or
the peptide linkers are GG at the N-terminus and SPSVA at the C-terminus of the circular permutated fluorescent protein, respectively, or
the peptide linkers are GG at the N-terminus and APSVA at the C-terminus of the circular permutated fluorescent protein, respectively;

or

more preferably, the lengths of the peptide linkers are 1 amino acid at the N-terminus and 1 amino acid at the C-terminus of the circular permutated fluorescent protein, respectively; particularly preferably, the peptide linkers are G at the N-terminus and G at the C-terminus of the circular permutated fluorescent protein, respectively;

further preferably, the circular permutated fluorescent protein inserted into the human β2 adrenergic receptor is cpEGFP; preferably, the cpEGFP is cpEGFP from GCaMP6s, GCaMP6m or GECO1.2,

particularly preferably, the amino acid sequence of the human β2 adrenergic receptor is:

(SEQ ID NO: 1)
MGQPGNGSAFLLAPNRSHAPDHDVTQQRDEVWVVGMGIVMSLIVLAIVFG
NVLVITAIAKFERLQTVTNYFITSLACADLVMGLAVVPFGAAHILMKMWT
FGNFWCEFWTSIDVLCVTASIETLCVIAVDRYFAITSPFKYQSLLTKNKA
RVIILMVWIVSGLTSFLPIQMHWYRATHQEAINCYANETCCDFFTNQAYA
IASSIVSFYVPLVEVIVFVYSRVFQEAKRQLQKIDKSEGRFHVQNLSQVE
QDGRTGHGLRRSSKFCLKEHKALKTLGIEVIGTFTLCWLPFFIVNIVHVI
QDNLIRKEVYILLNWIGYVNSGFNPLIYCRSPDFRIAFQELLCLRRSSLK
AYGNGYSSNGNTGEQSGYHVEQEKENKLLCEDLPGTEDFVGHQGTVPSDN
IDSQGRNCSTNDSLL,

wherein the underlined part is the third intracellular loop;

preferably, the circular permutated fluorescent protein is inserted into the human β2 adrenergic receptor between amino acid position 240 and amino acid position 241, or between amino acid position 250 and amino acid position 251.

9. The fusion polypeptide according to any one of embodiments 1 to 7, wherein the fusion polypeptide is a fluorescent sensor for detecting epinephrine and/or norepinephrine, and the GPCR is capable of specifically binding to adrenaline and/or norepinephrine;

preferably, the GPCR capable of specifically binding to adrenaline and/or norepinephrine is a human ADRA2A receptor, and the fusion polypeptide is a fluorescent sensor constructed based on the human ADRA2A receptor;

further preferably, the third intracellular loop of the human ADRA2A receptor is truncated and a circular permutated fluorescent protein is inserted at the truncated position;

further preferably, the circular permutated fluorescent protein is inserted into the third intracellular loop of the human ADRA2A receptor through peptide linkers at the N-terminus and the C-terminus, and the lengths of the peptide linkers are 2 amino acids at the N-terminus and 5 amino acids at the C-terminus of the circular permutated fluorescent protein, respectively;
preferably, the peptide linkers are GG at the N-terminus and GGAAA at the C-terminus of the circular permutated fluorescent protein, respectively, or the peptide linkers are GG at the N-terminus and TGAAA at the C-terminus of the circular permutated fluorescent protein, respectively;

further preferably, the circular permutated fluorescent protein inserted into the human ADRA2A receptor is cpEGFP; preferably, the cpEGFP is cpEGFP from GCaMP6s, GCaMP6m or GECO1.2;

more preferably, the amino acid sequence of the human ADRA2A receptor is:

(SEQ ID NO: 2)
MFRQEQPLAEGSFAPMGSLQPDAGNASWNGTEAPGGGARATPYSLQVTL
TLVCLAGLLMLLTVFGNVLVIIAVFTSRALKAPQNLFLVSLASADILVA
TLVIPFSLANEVMGYWYFGKAWCEIYLALDVLFCTSSIVHLCAISLDRY
WSITQAIEYNLKRTPRRIKAIIITVWVISAVISFPPLISIEKKGGGGGP
QPAEPRCEINDQKWYVISSCIGSFEAPCLEVIILVYVRIYQIAKRRTRV
PPSRRGPDAVAAPPGGTERRPNGLGPERSAGPGGAEAEPLPTQLNGAPG
EPAPAGPRDTDALDLEESSSSDHAERPPGPRRPERGPRGKGKARASQVK
PGDSLPRRGPGATGIGTPAAGPGEERVGAAKASRWRGRQNREKRFTFVL
AVVIGVFVVCWFPFFFTYTLTAVGCSVPRTLFKFFFWFGYCNSSLNPVI
YTIFNHDFRRAFKKILCRGDRKRIV,

wherein the underlined part is the third intracellular loop;

preferably, amino acids 71-130 of the third intracellular loop of the human ADRA2A receptor are truncated, and the circular permutated fluorescent protein is inserted at the truncated positions; or amino acids 71-135 of the third intracellular loop of the human ADRA2A receptor are truncated, and the circular permutated fluorescent protein is inserted at the truncated positions.

10. The fusion polypeptide according to any one of embodiments 1 to 7, wherein the fusion polypeptide is a fluorescent sensor constructed based on a G protein-coupled receptor for detecting acetylcholine, and the G protein-coupled receptor is capable of specifically binding to acetylcholine;

preferably, the GPCR capable of specifically binding to adrenaline is a human acetylcholine receptor M3R subtype, and the fluorescent protein constructed based on the G protein-coupled receptor is a fluorescent sensor constructed based on the human acetylcholine receptor M3R subtype;

further preferably, the third intracellular loop of the human acetylcholine receptor M3R subtype is truncated and a circular permutated fluorescent protein is inserted at the truncated positions;

further preferably, the circular permutated fluorescent protein is inserted into the third intracellular loop of the human acetylcholine receptor M3R subtype through peptide linkers at the N-terminus and the C-terminus; preferably, the lengths of the peptide linker are 2 amino acids at the N-terminus and 5 amino acids at the C-terminus of the circular permutated fluorescent protein, respectively;

preferably, the peptide linkers are GG at the N-terminus and GGAAA at the C-terminus of the circular permutated fluorescent protein, respectively, or the peptide linkers are GG at the N-terminus and HGAAA at the C-terminus of the circular permutated fluorescent protein, respectively, or the peptide linkers are GG at the N-terminus and HNAAA at the C-terminus of the circular permutated fluorescent protein, respectively, or the peptide linkers are GG at the N-terminus and HNAK at the C-terminus of the circular permutated fluorescent protein, respectively;

more preferably, the circular permutated fluorescent protein inserted into the human acetylcholine receptor M3R subtype is cpEGFP; preferably, the cpEGFP is cpEGFP from GCaMP6s, GCaMP6m, or GECO1.2;

more preferably, the amino acid sequence of the human acetylcholine receptor M3R subtype is:

(SEQ ID NO: 3)
MTLHNNSTTSPLFPNISSSWIHSPSDAGLPPGTVTHFGSYNVSRAAGNF
SSPDGTTDDPLGGHTVWQVVFIAFLTGILALVTIIGNILVIVSFKVNKQ
LKTVNNYFLLSLACADLIIGVISMNLFTTYIIMNRWALGNLACDLWLAI
DYVASNASVMNLLVISFDRYFSITRPLTYRAKRTTKRAGVMIGLAWVIS
FVLWAPAILFWQYFVGKRTVPPGECFIQFLSEPTITFGTAIAAFYMPVT
IMTILYWRIYKETEKRTKELAGLQASGTEAETENFVHPTGSSRSCSSYE
LQQQSMKRSNRRKYGRCHFWFTTKSWKPSSEQMDQDHSSSDSWNNNDAA
ASLENSASSDEEDIGSETRAIYSIVLKLPGHSTILNSTKLPSSDNLQVP
EEELGMVDLERKADKLQAQKSVDDGGSFPKSFSKLPIQLESAVDTAKTS
DVNSSVGKSTATLPLSFKEATLAKRFALKTRSQITKRKRMSLVKEKKAA
QTLSAILLAFIITWTPYNIMVLVNTFCDSCIPKTFWNLGYWLCYINSTV
NPVCYALCNKTFRTTFKMLLLCQCDKKKRRKQQYQQRQSVIFHKRAPEQ
AL,

wherein the underlined part is the third intracellular loop;

preferably, amino acids 260-490 of the third intracellular loop of the human acetylcholine receptor M3R subtype are truncated, and the circular permutated fluorescent protein is inserted at the truncated positions; or amino acids 260-491 of the third intracellular loop of the human acetylcholine receptor M3R subtype are truncated, and the circular permutated fluorescent protein is inserted at the truncated positions.

11. The fusion polypeptide according to any one of embodiments 1 to 7, wherein the fusion polypeptide is a fluorescent sensor for detecting serotonin, and the GPCR is capable of specifically binding to serotonin;

preferably, the GPCR capable of specifically binding to serotonin is a human HTR2C receptor, and the fusion polypeptide is a fluorescent sensor constructed based on the human HTR2C receptor;

further preferably, the third intracellular loop of the human HTR2C receptor is truncated and a circular permutated fluorescent protein is inserted at the truncated position;

further preferably, the circular permutated fluorescent protein is connected to the third intracellular loop of the human HTR2C receptor through peptide linkers at the N-terminus and the C-terminus, and the lengths of the peptide linkers are 2 amino acids at the N-terminus and 5 amino acids at the C-terminus of the circular permutated fluorescent protein, respectively;
preferably, the peptide linkers are GG at the N-terminus and GGAAA at the C-terminus of the circular permutated fluorescent protein, respectively, or the peptide linkers are NG at the N-terminus and GFAAA at the C-terminus of the circular permutated fluorescent protein, respectively;

more preferably, the circular permutated fluorescent protein inserted into the human HTR2C receptor is cpEGFP; preferably, the cpEGFP is cpEGFP from GCaMP6s, GCaMP6m or GECO1.2;

particularly preferably, the amino acid sequence of the human HTR2C receptor is:

(SEQ ID NO: 4)
MVNLRNAVHSFLVHLIGLLVWQCDISVSPVAAIVTDIFNTSDGGRFKFP
DGVQNWPALSIVIIIIMTIGGNILVIMAVSMEKKLHNATNYFLMSLAIA
DMLVGLLVMPLSLLAILYDYVWPLPRYLCPVWISLDVLFSTASIMHLCA
ISLDRYVAIRNPIEHSRFNSRTKAIMKIAIVWAISIGVSVPIPVIGLRD
EEKVFVNNTTCVLNDPNFVLIGSFVAFFIPLTIMVITYCLTIYVLRRQA
LMLLHGHTEEPPGLSLDFLKCCKRNTAEEENSANPNQDQNARRRKKKER
RPRGTMQAINNERKASKVLGIVFFVFLIMWCPFFITNILSVLCEKSCNQ
KLMEKLLNVFVWIGYVCSGINPLVYTLFNKIYRRAFSNYLRCNYKVEKK
PPVRQIPRVAATALSGRELNVNIYRHTNEPVIEKASDNEPGIEMQVENL
ELPVNPSSVVSERISSV,

wherein the underlined part is the third intracellular loop;

preferably, amino acids 16-55 of the third intracellular loop of the human HTR2C receptor are truncated, and a circular permutated fluorescent protein is inserted at the truncated positions; or amino acids 11-60 of the third intracellular loop of the human HTR2C receptor are truncated, and a circular permutated fluorescent protein is inserted at the truncated positions; or amino acids 16-70 of the third intracellular loop of the human HTR2C receptor are truncated, and a circular permutated fluorescent protein is inserted at the truncated positions; or amino acids 15-68 of the third intracellular loop of the human HTR2C receptor are truncated, and a circular permutated fluorescent protein is inserted at the truncated positions; or amino acids 15-68 of the third intracellular loop of the human HTR2C receptor are truncated, and a circular permutated fluorescent protein is inserted at the truncated position, and the leucine L at position 13 of the third intracellular loop is replaced with phenylalanine F.

12. The fusion polypeptide according to embodiment 11, wherein the circular permutated fluorescent protein is inserted into the third intracellular loop of the human HTR2C receptor through peptide linkers at the N-terminus and the C-terminus; preferably, the lengths of the peptide linkers are 5 amino acids at the N-terminus and 3 amino acids at the C-terminus of the circular permutated fluorescent protein, respectively; more preferably, the peptide linkers are PVVSE at the N-terminus and ATR at the C-terminus of the circular permutated fluorescent protein, respectively;

preferably, the circular permutated fluorescent protein inserted into the human HTR2C receptor is cpmApple; preferably, the cpmApple is cpmApple from R-GECO1;

further preferably, the amino acid sequence of the human HTR2C receptor is:

(SEQ ID NO: 4)
MDPLNLSWYDDDLERQNWSRPFNGSDGKADRPHYNYYATLLTLLIAVIVF
GNVLVCMAVSREKALQTTTNYLIVSLAVADLLVATLVMPWVVYLEVVGEW
KFSRIHCDIFVTLDVMIVICTASILNLCAISIDRYTAVAMPMLYNTRYSS
KRRVTVMISIVWVLSFTISCPLLFGLNNADQNECIIANPAFVVYSSIVSF
YVPFIVTLLVYIKIYIVLRRRRKRVNTKRSSRAFRAHLRAPLKGNCTHPE
DMKLCTVIMKSNGSFPVNRRRVEAARRAQELEMEMLSSTSPPERTRYSPI
PPSHHQLTLPDPSHHGLHSTPDSPAKPEKNGHAKDHPKIAKIFEIQTMPN
GKTRTSLKTMSRRKLSQQKEKKATQMLAIVLGVFIICWLPFFITHILNIH
CDCNIPPVLYSAFTWLGYVNSAVNPIIYTTFNIEFRKAFLKILHC,

wherein the underlined part is the third intracellular loop;

preferably, amino acids 241-306 of the third intracellular loop of the human HTR2C receptor are truncated, and the circular permutated fluorescent protein is inserted at the truncated positions; or amino acids 240-309 of the third intracellular loop of the human HTR2C receptor are truncated, and the circular permutated fluorescent protein is inserted at the truncated positions.

13. The fusion polypeptide according to any one of embodiments 1 to 7, wherein the fusion polypeptide is a fluorescent sensor for detecting dopamine, and the GPCR is capable of specifically binding to dopamine;

preferably, the GPCR capable of specifically binding to dopamine is a human DRD2 receptor, and the fusion polypeptide is a fluorescent sensor constructed based on the human DRD2 receptor;

further preferably, the third intracellular loop of the human DRD2 receptor is truncated and a circular permutated fluorescent protein is inserted at the truncated position;

further preferably, the circular permutated fluorescent protein is inserted into the third intracellular loop of the human DRD2 receptor through peptide linkers at the N-terminus and the C-terminus; preferably, the lengths of the peptide linkers are 2 amino acids at the N-terminus and 5 amino acids at the C-terminus of the circular permutated fluorescent protein, respectively; further preferably, the peptide linkers are GG at the N-terminus and GGAAA at the C-terminus of the circular permutated fluorescent protein, respectively;

further preferably, the circular permutated fluorescent protein inserted into the human DRD2 receptor is cpEGFP; preferably, the cpEGFP is cpEGFP from GCaMP6s, GCaMP6m or GECO1.2;

particularly preferably, the amino acid sequence of the human DRD2 receptor is:

(SEQ ID NO: 5)
MDPLNLSWYDDDLERQNWSRPFNGSDGKADRPHYNYYATLLTLLIAVIV
FGNVLVCMAVSREKALQTTTNYLIVSLAVADLLVATLVMPWVVYLEVVG
EWKFSRIHCDIFVTLDVMMCTASILNLCAISIDRYTAVAMPMLYNTRYS
SKRRVTVMISIVWVLSFTISCPLLFGLNNADQNECIIANPAFVVYSSIV
SFYVPFIVTLLVYIKIYIVLRRRRKRVNTKRSSRAFRAHLRAPLKGNCT
HPEDMKLCTVIMKSNGSFPVNRRRVEAARRAQELEMEMLSSTSPPERTR
YSPIPPSHHQLTLPDPSHHGLHSTPDSPAKPEKNGHAKDHPKIAKIFEI
QTMPNGKTRTSLKTMSRRKLSQQKEKKATQMLAIVLGVFIICWLPFFIT
HILNIHCDCNIPPVLYSAFTWLGYVNSAVNPIIYTTFNIEFRKAFLKIL
HC,

wherein the underlined part is the third intracellular loop;

preferably, amino acids 253-357 of the third intracellular loop of the human DRD2 receptor are truncated, and the circular permutated fluorescent protein is inserted at the truncated positions; or amino acids 254-360 of the third intracellular loop of the human DRD2 receptor are truncated, and the circular permutated fluorescent protein is inserted at the truncated positions.

14. The fusion polypeptide according to embodiment 13, wherein the circular permutated fluorescent protein is inserted into the third intracellular loop of the human DRD2 receptor through peptide linkers at the N-terminus and the C-terminus; preferably, the lengths of the peptide linkers are 5 amino acids at the N-terminus and 3 amino acids at the C-terminus of the circular permutated fluorescent protein, respectively; more preferably, the peptide linkers are PVVSE at the N-terminus and ATR at the C-terminus of the circular permutated fluorescent protein, respectively;

preferably, the circular permutated fluorescent protein inserted into the human DRD2 receptor is cpmApple; preferably, the cpmApple is cpmApple from R-GECO1;

further preferably, the amino acid sequence of the human DRD2 receptor is:

(SEQ ID NO: 5)
MDPLNLSWYDDDLERQNWSRPFNGSDGKADRPHYNYYATLLTLLIAVIV
FGNVLVCMAVSREKALQTTTNYLIVSLAVADLLVATLVMPWVVYLEVVG
EWKFSRIHCDIFVTLDVMMCTASILNLCAISIDRYTAVAMPMLYNTRYS
SKRRVTVMISIVWVLSFTISCPLLFGLNNADQNECIIANPAFVVYSSIV
SFYVPFIVTLLVYIKIYIVLRRRRKRVNTKRSSRAFRAHLRAPLKGNCT
HPEDMKLCTVIMKSNGSFPVNRRRVEAARRAQELEMEMLSSTSPPERTR
YSPIPPSHHQLTLPDPSHHGLHSTPDSPAKPEKNGHAKDHPKIAKIFEI
QTMPNGKTRTSLKTMSRRKLSQQKEKKATQMLAIVLGVFIICWLPFFIT
HILNIHCDCNIPPVLYSAFTWLGYVNSAVNPIIYTTFNIEFRKAFLKIL
HC,

wherein the underlined part is the third intracellular loop;

preferably, amino acids 223-349 of the third intracellular loop of the human DRD2 receptor are truncated, and the circular permutated fluorescent protein is inserted at the truncated positions; or amino acids 268-364 of the third intracellular loop of the human DRD2 receptor are truncated, and the circular permutated fluorescent protein is inserted at the truncated positions; or amino acids 224-365 of the third intracellular loop of the human DRD2 receptor are truncated, and the circular permutated fluorescent protein is inserted at the truncated positions.

15. The fusion polypeptide according to any one of embodiments 1-14, wherein the fusion polypeptide further comprises a Gα peptide segment connected to the C-terminus of the GPCR, for example, the Gα peptide segment is 20 amino acids at the C-terminus of the Gα protein; preferably, the Gα peptide segment is connected to the last amino acid at the C-terminus of the GPCR; more preferably, the sequence of the Gα peptide segment is selected from the group consisting of VFAAVKDTILQLNLKEYNLV (SEQ ID NO: 6), VFNDCRDIIQRMHLRQYELL (SEQ ID NO: 7) and VFDAVTDVIIKNNLKDCGLF (SEQ ID NO: 8).

16. The fusion polypeptide according to any one of embodiments 1 to 15, wherein the fusion polypeptide further comprises a luciferase connected to the C-terminus of the GPCR, and the light emitted by the luciferase-catalyzed chemical reaction excites the circular permutated fluorescent protein; preferably, the luciferase is Nanoluc, Fluc (firefly luciferase) or Rluc (renilla luciferase);

for example, the fusion polypeptide is a fluorescent sensor constructed based on a human HTR2C receptor, the luciferase is inserted into the C-terminus of the fusion polypeptide, and the luciferase is inserted into the C-terminus of the fusion polypeptide through peptide linkers at the N-terminus and the C-terminus of the luciferase, and the peptide linkers at the N-terminus and the C-terminus of the luciferase both are GSG;

for example, the luciferase is inserted between amino acid positions 582 and 583 of the fluorescent sensor GRAB-5-HT2.0, and the luciferase are connected to the fluorescent sensor GRAB-5-HT2.0 through peptide linkers at the N-terminus and the C-terminus, wherein the peptide linkers at the N-terminus and the C-terminus of the luciferase both are GSG;

wherein fluorescent sensor GRAB-5-HT2.0 is a fluorescent sensor obtained by deleting the amino acid residues at the positions 15-68 of the third intracellular loop of the human HTR2C receptor, and inserting cpEGFP at the deleted position, wherein the N-terminus of cpEGFP is connected to the human HTR2C receptor through the N-terminal peptide linker NG, and the C-terminus of cpEGFP is connected to the human HTR2C receptor through the C-terminal peptide linker GFAAA;

wherein the amino acid sequence of the human HTR2C receptor is

(SEQ ID NO: 4)
MVNLRNAVHSFLVHLIGLLVWQCDISVSPVAAIVTDIFNTSDGGRFKFP
DGVQNWPALSIVIIIIMTIGGNILVIMAVSMEKKLHNATNYFLMSLAIA
DMLVGLLVMPLSLLAILYDYVWPLPRYLCPVWISLDVLFSTASIMHLCA
ISLDRYVAIRNPIEHSRFNSRTKAIMKIAIVWAISIGVSVPIPVIGLRD
EEKVFVNNTTCVLNDPNFVLIGSFVAFFIPLTIMVITYCLTIYVLRRQA
LMLLHGHTEEPPGLSLDFLKCCKRNTAEEENSANPNQDQNARRRKKKER
RPRGTMQAINNERKASKVLGIVFFVFLIMWCPFFITNILSVLCEKSCNQ
KLMEKLLNVFVWIGYVCSGINPLVYTLFNKIYRRAFSNYLRCNYKVEKK
PPVRQIPRVAATALSGRELNVNIYRHTNEPVIEKASDNEPGIEMQVENL
ELPVNPSSVVSERISSV,

wherein the underlined part is the third intracellular loop;

preferably, the cpEGFP is cpEGFP from GCaMP6s.

17. A composition comprising:

a ligand recognition polypeptide comprising 1) the extracellular region of the G protein-coupled receptor (GPCR) in the fusion polypeptide of any one of embodiments 1 to 16, and 2) a first protein interaction segment; and

a signal generating polypeptide comprising 1) a second protein interaction segment capable of specifically binding to the first protein interaction segment, and 2) the transmembrane and intracellular regions of the G protein-coupled receptor (GPCR) and the signal molecule in the fusion polypeptide of any one of embodiments 1 to 16;

preferably, the extracellular region of the G protein-coupled receptor (GPCR) in the ligand recognition polypeptide and the transmembrane region and intracellular regions of the G protein-coupled receptor (GPCR) in the signal generating polypeptide are derived from different G protein-coupled receptors.

18. The composition according to embodiment 17, wherein the protein interaction segment is a leucine zipper domain; preferably, one of the protein interaction segments is BZip (RR), and the other protein interaction segment is AZip (EE).

19. The composition of embodiment 17, wherein the first and second protein interaction segments are selected from the group consisting of:

1) PSD95-Dlgl-zo-1 (PDZ) domain;

2) Streptavidin and streptavidin binding protein (SBP);

3) FTORP binding domain (FRB) and FK506 binding protein (FKBP) of mTOR;

4) Cyclophilin-Fas fusion protein (CyP-Fas) and FK506 binding protein (FKBP);

5) Calcineurin A (CNA) and FK506 binding protein (FKBP);

6) SNAP tags and Halo tags; and

7) PYL and ABI.

20. A ligand recognition polypeptide comprising 1) the extracellular region of the G protein-coupled receptor (GPCR) in the fusion polypeptide of any one of embodiments 1 to 16, and 2) a protein interaction segment, wherein the protein interaction segment can interact with another protein interaction segment, preferably, the protein interaction segment is selected from:

leucine zipper domain, PSD95-Dlgl-zo-1 (PDZ) domain, streptavidin, streptavidin binding protein (SBP), FKBP binding domain (FRB) of mTOR, Cyclophilin-Fas fusion protein (CyP-Fas), Calcineurin A (CNA) and FK506 binding protein (FKBP), Snap tag, Halo tag, PYL and ABI.

21. A signal generating polypeptide comprising 1) a protein interaction segment, and 2) the transmembrane and intracellular regions of the G protein-coupled receptor (GPCR) and the signal molecule in the fusion polypeptide of any one of embodiments 1 to 16; wherein the protein interaction segment can interact with another protein interaction segment, preferably, the protein interaction segment is selected from:

leucine zipper domain, PSD95-Dlgl-zo-1 (PDZ) domain, streptavidin, streptavidin binding protein (SBP), FKBP binding domain (FRB) of mTOR, Cyclophilin-Fas fusion protein (CyP-Fas), Calcineurin A (CNA) and FK506 binding protein (FKBP), Snap tag, Halo tag, PYL and ABI.

22. A polynucleotide encoding the fusion polypeptide of any one of embodiments 1 to 16, the composition of any one of embodiments 17 to 19, the ligand recognition polypeptide of embodiment 20, or the signal generating polypeptide of embodiment 21.

23. An expression vector comprising the polynucleotide of embodiment 22.

24. A cell, such as a neuronal cell, comprising the fusion polypeptide of any one of embodiments 1 to 16, the composition of any one of embodiments 17 to 19, the ligand recognition polypeptide of embodiment 20, the signal generating polypeptide of embodiment 21, the polynucleotide of embodiment 22, and/or the expression vector of embodiment 23.

25. A transgenic animal comprising the fusion polypeptide of any one of embodiments 1 to 16, the composition of any one of embodiments 17 to 19, the ligand recognition polypeptide of embodiment 20, the signal generating polypeptide of embodiment 21, the polynucleotide of embodiment 22, the expression vector of embodiment 23, and/or the cell of embodiment 24.

26. A method for detecting a GPCR ligand in an object, comprising

exposing the object to the fusion polypeptide of any one of embodiments 1 to 16, the composition of any one of embodiments 17 to 19 and/or the cell of embodiment 24, wherein the GPCR in the fusion polypeptide or the extracellular region of the GPCR in the ligand recognizing polypeptide of the composition is capable of specifically binding to the ligand,

comparing the detectable signal caused by the exposure with one or more references containing a predetermined amount of the ligand, and analyzing the presence, content, or time and/or spatial change of the ligand in the analysis object;

for example, the one or more references containing a predetermined amount of the ligand include at least a reference not containing the ligand, and preferably also include at least one reference containing a non-zero amount of the ligand.

27. The method of embodiment 26, wherein the detectable signal is an optical signal; preferably the fusion polypeptide is a fluorescent sensor that responds to the specific binding of the GPCR to its ligand, wherein the specific binding causes a change in the fluorescent signal, such as a change in the intensity of the fluorescent signal, for example an increase or decrease in the intensity of the fluorescent signal.

28. The method according to embodiment 26 or 27, wherein the detection is performed in an ex vivo cell or in a living body; for example, the detection is to detect the distribution of the ligand in a living body; or for example, the ligand is selected from a neurotransmitter, hormone, metabolite and nutrient.

29. A method for identifying a substance targeting a GPCR, comprising

exposing a test substance to the fusion polypeptide of any one of embodiments 1 to 16, the composition of any one of embodiments 17 to 19 and/or the cell of embodiment 24, wherein the GPCR in the fusion polypeptide or the extracellular region of the GPCR in the ligand recognizing polypeptide of the composition is capable of specifically binding to its ligand,

comparing the detectable signal caused by the exposure with one or more references containing a predetermined amount of the ligand, and analyzing the binding of the test substance to the GPCR or the extracellular region of the GPCR, which indicates that the test substance is a candidate active substance targeting the GPCR;

for example, the one or more references containing a predetermined amount of the ligand include at least a reference not containing the ligand, and preferably also include at least one reference containing a non-zero amount of the ligand.

30. The method according to embodiment 29, wherein the detectable signal is an optical signal; preferably the fusion polypeptide is a fluorescent sensor that responds to the specific binding of the GPCR to its ligand, wherein the specific binding causes a change in the fluorescent signal, such as a change in the intensity of the fluorescent signal, for example an increase or decrease in the intensity of the fluorescent signal.

31. A method for identifying a substance targeting a GPCR, comprising

contacting a test substance and/or a determined ligand for the GPCR with the fusion polypeptide of any one of embodiments 1 to 16, the composition of any one of embodiments 17 to 19 and/or the cell of embodiment 24, wherein the GPCR in the fusion polypeptide or the extracellular region of the GPCR in the ligand recognizing polypeptide of the composition is capable of specifically binding to the determined ligand,

comparing the detectable signal caused by the system comprising the test substance, the determined ligand and the fusion polypeptide of any one of embodiments 1 to 16, the composition of any one of embodiments 17 to 19 and/or the cell of embodiment 24, and the detectable signal caused by the system comprising the determined ligand but not the test substance, and the fusion polypeptide of any one of embodiments 1 to 16, the composition of any one of embodiments 17 to 19 and/or the cell of embodiment 24, and the difference indicates that the test substance interferes with the binding between the determined ligand and the fusion polypeptide or the composition, and further indicates that the test substance is a candidate active substance targeting the GPCR.

32. The method according to embodiment 31, wherein the detectable signal is an optical signal; preferably the fusion polypeptide is a fluorescent sensor that responds to the specific binding of the GPCR to its ligand, wherein the specific binding causes a change in the fluorescent signal, such as a change in the intensity of the fluorescent signal, for example an increase or decrease in the intensity of the fluorescent signal.

In some embodiments, in the fusion polypeptide constructed based on the human β2 adrenergic receptor, the circular permutated fluorescent protein is connected to the third intracellular loop of the human β2 adrenergic receptor through peptide linkers at the N-terminus and the C-terminus. In some preferred embodiments, the lengths of the peptide linkers of the circular permutated fluorescent protein are 1 or 2 amino acids at the N-terminus, and/or 1, 2, 3, 4 or 5 amino acids at the C-terminus, respectively. In some more preferred embodiments, the lengths of the peptide linkers of the circular permutated fluorescent protein are 2 amino acids at the N-terminus and 5 amino acids at the C-terminus, respectively. In other preferred embodiments, the lengths of the peptide linkers of the circular permutated fluorescent protein are 1 amino acid at the N-terminus and 1 amino acid at the C-terminus, respectively. In some preferred embodiments, the peptide linkers of the circular permutated fluorescent protein are GG at the N-terminus and GGAAA at the C-terminus, respectively. In other preferred embodiments, the peptide linkers of the circular permutated fluorescent protein are GG at the N-terminus and SPSVA at the C-terminus, respectively. In other preferred embodiments, the peptide linkers of the circular permutated fluorescent protein are GG at the N-terminus and APSVA at the C-terminus, respectively. In other preferred embodiments, the peptide linkers of the circular permutated fluorescent protein are G at the N-terminus and G at the C-terminus, respectively.

In some embodiments, the circular permutated fluorescent protein inserted into the human β2 adrenergic receptor is cpEGFP. In some embodiments, the cpEGFP is cpEGFP from GCaMP6s. In other embodiments, the cpEGFP is cpEGFP from GCaMP6m or GECO1.2.

In a preferred embodiment, the amino acid sequence of the human β2 adrenergic receptor is:

(SEQ ID NO: 1)
MGQPGNGSAFLLAPNRSHAPDHDVTQQRDEVWVVGMGIVMSLIVLAIVF
GNVLVITAIAKFERLQTVTNYFITSLACADLVMGLAVVPFGAAHILMKM
WTFGNFWCEFWTSIDVLCVTASIETLCVIAVDRYFAITSPFKYQSLLTK
NKARVIILMVWIVSGLTSFLPIQMHWYRATHQEAINCYANETCCDFFTN
QAYAIASSIVSFYVPLVIMVFVYSRVFQEAKRQLQKIDKSEGRFHVQNL
SQVEQDGRTGHGLRRSSKFCLKEHKALKTLGIIMGTFTLCWLPFFIVNI
VHVIQDNLIRKEVYILLNWIGYVNSGFNPLIYCRSPDFRIAFQELLCLR
RSSLKAYGNGYSSNGNTGEQSGYHVEQEKENKLLCEDLPGTEDFVGHQG
TVPSDNIDSQGRNCSTNDSLL;

wherein the underlined part is the third intracellular loop.

In some embodiments, the circular permutated fluorescent protein is inserted into the human β2 adrenergic receptor between amino acid position 240 and amino acid position 241. In some embodiments, the circular permutated fluorescent protein is inserted into the human β2 adrenergic receptor between amino acid position 250 and amino acid position 251.

In some embodiments, in the fusion polypeptide constructed based on the human ADRA2A receptor, the third intracellular loop of the human ADRA2A receptor is truncated and a circular permutated fluorescent protein is inserted at the truncated position.

In some preferred embodiments, in the fusion polypeptide constructed based on the human ADRA2A receptor, the circular permutated fluorescent protein is connected to the third intracellular loop of the human ADRA2A receptor through peptide linkers at the N-terminus and the C-terminus. In some preferred embodiments, the lengths of the peptide linkers of the circular permutated fluorescent protein are 2 amino acids at the N-terminus and 5 amino acids at the C-terminus, respectively. In some preferred embodiments, the peptide linkers of the circular permutated fluorescent protein are GG at the N-terminus and GGAAA at the C-terminus, respectively. In some preferred embodiments, the peptide linkers of the circular permutated fluorescent protein are GG at the N-terminus and TGAAA at the C-terminus, respectively.

In some embodiments, the circular permutated fluorescent protein inserted into the human ADRA2A receptor is cpEGFP. In some embodiments, the cpEGFP is cpEGFP from GCaMP6s. In other embodiments, the cpEGFP is cpEGFP from GCaMP6m or GECO1.2.

In some preferred embodiments, the amino acid sequence of the human ADRA2A receptor is:

(SEQ ID NO: 2)
MFRQEQPLAEGSFAPMGSLQPDAGNASWNGTEAPGGGARATPYSLQVTL
TLVCLAGLLMLLTVFGNVLVIIAVFTSRALKAPQNLFLVSLASADILVA
TLVIPFSLANEVMGYWYFGKAWCEIYLALDVLFCTSSIVHLCAISLDRY
WSITQAIEYNLKRTPRRIKAIIITVWVISAVISFPPLISIEKKGGGGGP
QPAEPRCEINDQKWYVISSCIGSFEAPCLEVIILVYVRIYQIAKRRTRV
PPSRRGPDAVAAPPGGTERRPNGLGPERSAGPGGAEAEPLPTQLNGAPG
EPAPAGPRDTDALDLEESSSSDHAERPPGPRRPERGPRGKGKARASQVK
PGDSLPRRGPGATGIGTPAAGPGEERVGAAKASRWRGRQNREKRFTFVL
AVVIGVFVVCWFPFFFTYTLTAVGCSVPRTLFKFFFWFGYCNSSLNPVI
YTIFNHDFRRAFKKILCRGDRKRIV;

wherein the underlined part is the third intracellular loop.

In some preferred embodiments, amino acids 79-138 of the third intracellular loop of the human ADRA2A receptor are deleted, and a circular permutated fluorescent protein is inserted at the deleted position. In other preferred embodiments, amino acids 79-143 of the third intracellular loop of the human ADRA2A receptor are deleted, and a circular permutated fluorescent protein is inserted at the deleted position.

In some embodiments, in the fusion polypeptide constructed based on the human acetylcholine receptor M3R subtype, the third intracellular loop of the human acetylcholine receptor M3R subtype is truncated and a circular permutated fluorescent protein is inserted at the truncated position.

In some embodiments, in the fusion polypeptide constructed based on the human acetylcholine receptor M3R subtype, the circular permutated fluorescent protein is connected to the third intracellular loop of the human acetylcholine receptor M3R subtype through peptide linkers at the N-terminus and the C-terminus. In some embodiments, the lengths of the peptide linkers of the circular permutated fluorescent protein are 2 amino acids at the N-terminus and 5 amino acids at the C-terminus, respectively. In some preferred embodiments, the peptide linkers of the circular permutated fluorescent protein are GG at the N-terminus and GGAAA at the C-terminus, respectively. In other preferred embodiments, the peptide linkers of the circular permutated fluorescent protein are GG at the N-terminus and HGAAA at the C-terminus, respectively. In other preferred embodiments, the peptide linkers of the circular permutated fluorescent protein are GG at the N-terminus and HNAAA at the C-terminus, respectively. In other preferred embodiments, the peptide linkers of the circular permutated fluorescent protein are GG at the N-terminus and HNAK at the C-terminus, respectively.

In some embodiments, the circular permutated fluorescent protein inserted into the human acetylcholine receptor M3R subtype is cpEGFP. In some embodiments, the cpEGFP is cpEGFP from GCaMP6s. In other embodiments, the cpEGFP is cpEGFP from GCaMP6m or GECO1.2.

In a preferred embodiment, the amino acid sequence of the human acetylcholine receptor M3R subtype is:

(SEQ ID NO: 3)
MTLHNNSTTSPLFPNISSSWIHSPSDAGLPPGTVTHFGSYNVSRAAGNF
SSPDGTTDDPLGGHTVWQVVFIAFLTGILALVTIIGNILVIVSFKVNKQ
LKTVNNYFLLSLACADLIIGVISMNLFTTYIIMNRWALGNLACDLWLAI
DYVASNASVMNLLVISFDRYFSITRPLTYRAKRTTKRAGVMIGLAWVIS
FVLWAPAILFWQYFVGKRTVPPGECFIQFLSEPTITFGTAIAAFYMPVT
IMTILYWRIYKETEKRTKELAGLQASGTEAETENFVHPTGSSRSCSSYE
LQQQSMKRSNRRKYGRCHFWFTTKSWKPSSEQMDQDHSSSDSWNNNDAA
ASLENSASSDEEDIGSETRAIYSIVLKLPGHSTILNSTKLPSSDNLQVP
EEELGMVDLERKADKLQAQKSVDDGGSFPKSFSKLPIQLESAVDTAKTS
DVNSSVGKSTATLPLSFKEATLAKRFALKTRSQITKRKRMSLVKEKKAA
QTLSAILLAFIITWTPYNIMVLVNTFCDSCIPKTFWNLGYWLCYINSTV
NPVCYALCNKTFRTTFKMLLLCQCDKKKRRKQQYQQRQSVIFHKRAPEQ
AL;

wherein the underlined part is the third intracellular loop (ICL3), which is the amino acids 253-491.

In some embodiments, amino acids 260-490 of the third intracellular loop of the human acetylcholine receptor M3R subtype are deleted, and a circular permutated fluorescent protein is inserted at the deleted position. In some embodiments, amino acids 260-491 of the third intracellular loop of the human acetylcholine receptor M3R subtype are deleted, and a circular permutated fluorescent protein is inserted at the deleted position.

In some embodiments, in the fusion polypeptide constructed based on the human HTR2C receptor, the third intracellular loop of the human HTR2C receptor is truncated and a circular permutated fluorescent protein is inserted at the truncated position.

In some preferred embodiments, in the fluorescent sensor constructed based on the human HTR2C receptor, the circular permutated fluorescent protein is connected to the third intracellular loop of the human HTR2C receptor through peptide linkers at the N-terminus and the C-terminus. In some preferred embodiments, the lengths of the peptide linkers of the circular permutated fluorescent protein are 2 amino acids at the N-terminus and 5 amino acids at the C-terminus, respectively. In some preferred embodiments, the peptide linkers of the circular permutated fluorescent protein are GG at the N-terminus and GGAAA at the C-terminus, respectively. In some preferred embodiments, the peptide linkers of the circular permutated fluorescent protein are NG at the N-terminus and GFAAA at the C-terminus, respectively.

In some embodiments, the circular permutated fluorescent protein inserted into the human HTR2C receptor is cpEGFP. In some embodiments, the cpEGFP is cpEGFP from GCaMP6s. In other embodiments, the cpEGFP is cpEGFP from GCaMP6m or GECO1.2.

In some preferred embodiments, the amino acid sequence of the human HTR2C receptor is:

(SEQ ID NO: 4)
MVNLRNAVHSFLVHLIGLLVWQCDISVSPVAAIVTDIFNTSDGGRFKFP
DGVQNWPALSIVIIIIMTIGGNILVIMAVSMEKKLHNATNYFLMSLAIA
DMLVGLLVMPLSLLAILYDYVWPLPRYLCPVWISLDVLFSTASIMHLCA
ISLDRYVAIRNPIEHSRFNSRTKAIMKIAIVWAISIGVSVPIPVIGLRD
EEKVFVNNTTCVLNDPNFVLIGSFVAFFIPLTIMVITYCLTIYVLRRQA
LMLLHGHTEEPPGLSLDFLKCCKRNTAEEENSANPNQDQNARRRKKKER
RPRGTMQAINNERKASKVLGIVFFVFLIMWCPFFITNILSVLCEKSCNQ
KLMEKLLNVFVWIGYVCSGINPLVYTLFNKIYRRAFSNYLRCNYKVEKK
PPVRQIPRVAATALSGRELNVNIYRHTNEPVIEKASDNEPGIEMQVENL
ELPVNPSSVVSERISSV;

wherein the underlined part is the third intracellular loop.

In some preferred embodiments, amino acids 16-55 of the third intracellular loop of the human HTR2C receptor are deleted, and a circular permutated fluorescent protein is inserted at the deleted position. In some preferred embodiments, amino acids 11-60 of the third intracellular loop of the human HTR2C receptor are deleted, and a circular permutated fluorescent protein is inserted at the deleted position. In some preferred embodiments, amino acids 16-70 of the third intracellular loop of the human HTR2C receptor are deleted, and a circular permutated fluorescent protein is inserted at the deleted position. In other preferred embodiments, amino acids 15-68 of the third intracellular loop of the human HTR2C receptor are deleted, and a circular permutated fluorescent protein is inserted at the deleted position.

In some more preferred embodiments, amino acids 15-68 of the third intracellular loop of the human HTR2C receptor are deleted, and a circular permutated fluorescent protein is inserted at the deleted position, and the leucine (L) at position 13 of the third intracellular loop is replaced with phenylalanine (F).

In some embodiments, in the fusion polypeptide constructed based on the human DRD2 receptor, the third intracellular loop of the human DRD2 receptor is truncated and a circular permutated fluorescent protein is inserted at the truncated position.

In some preferred embodiments, in the fluorescent sensor constructed based on the human DRD2 receptor, the circular permutated fluorescent protein is connected to the third intracellular loop of the human DRD2 receptor through peptide linkers at the N-terminus and the C-terminus. In some preferred embodiments, the lengths of the peptide linkers of the circular permutated fluorescent protein are 2 amino acids at the N-terminus and 5 amino acids at the C-terminus, respectively. In some preferred embodiments, the peptide linkers of the circular permutated fluorescent protein are GG at the N-terminus and GGAAA at the C-terminus, respectively.

In some embodiments, the circular permutated fluorescent protein inserted into the human DRD2 receptor is cpEGFP. In some embodiments, the cpEGFP is cpEGFP from GCaMP6s. In other embodiments, the cpEGFP is cpEGFP from GCaMP6m or GECO1.2.

In some preferred embodiments, the amino acid sequence of the human DRD2 receptor is:

(SEQ ID NO: 5)
MDPLNLSWYDDDLERQNWSRPFNGSDGKADRPHYNYYATLLTLLIAVIV
FGNVLVCMAVSREKALQTTTNYLIVSLAVADLLVATLVMPWVVYLEVVG
EWKFSRIHCDIFVTLDVMMCTASILNLCAISIDRYTAVAMPMLYNTRYS
SKRRVTVMISIVWVLSFTISCPLLFGLNNADQNECIIANPAFVVYSSIV
SFYVPFIVTLLVYIKIYIVLRRRRKRVNTKRSSRAFRAHLRAPLKGNCT
HPEDMKLCTVIMKSNGSFPVNRRRVEAARRAQELEMEMLSSTSPPERTR
YSPIPPSHHQLTLPDPSHHGLHSTPDSPAKPEKNGHAKDHPKIAKIFEI
QTMPNGKTRTSLKTMSRRKLSQQKEKKATQMLAIVLGVFIICWLPFFIT
HILNIHCDCNIPPVLYSAFTWLGYVNSAVNPIIYTTFNIEFRKAFLKIL
HC;

wherein the underlined part is the third intracellular loop.

In some preferred embodiments, amino acids 253-357 of the third intracellular loop of the human DRD2 receptor are deleted, and a circular permutated fluorescent protein is inserted at the deleted position. In some preferred embodiments, amino acids 254-360 of the third intracellular loop of the human DRD2 receptor are deleted, and a circular permutated fluorescent protein is inserted at the deleted position.

In some embodiments, in the fusion polypeptide constructed based on the human DRD2 receptor, the third intracellular loop of the human DRD2 receptor is truncated and the circular permutated fluorescent protein is inserted at the truncated position.

In some preferred embodiments, in the fusion polypeptide constructed based on the human DRD2 receptor, the circular permutated fluorescent protein is connected to the third intracellular loop of the human DRD2 receptor through peptide linkers at the N-terminus and the C-terminus. In some preferred embodiments, the lengths of the peptide linkers of the circular permutated fluorescent protein are 5 amino acids at the N-terminus and 3 amino acids at the C-terminus, respectively. In some preferred embodiments, the peptide linkers of the circular permutated fluorescent protein are PVVSE at the N-terminus and ATR at the C-terminus, respectively.

In some embodiments, the circular permutated fluorescent protein inserted into the human DRD2 receptor is cpmApple. In some embodiments, the cpmApple is cpmApple from R-GECO1.

In some preferred embodiments, the amino acid sequence of the human DRD2 receptor is:

(SEQ ID NO: 5)
MDPLNLSWYDDDLERQNWSRPFNGSDGKADRPHYNYYATLLTLLIAVIV
FGNVLVCMAVSREKALQTTTNYLIVSLAVADLLVATLVMPWVVYLEVVG
EWKFSRIHCDIFVTLDVMMCTASILNLCAISIDRYTAVAMPMLYNTRYS
SKRRVTVMISIVWVLSFTISCPLLFGLNNADQNECIIANPAFVVYSSIV
SFYVPFIVTLLVYIKIYIVLRRRRKRVNTKRSSRAFRAHLRAPLKGNCT
HPEDMKLCTVIMKSNGSFPVNRRRVEAARRAQELEMEMLSSTSPPERTR
YSPIPPSHHQLTLPDPSHHGLHSTPDSPAKPEKNGHAKDHPKIAKIFEI
QTMPNGKTRTSLKTMSRRKLSQQKEKKATQMLAIVLGVFIICWLPFFIT
HILNIHCDCNIPPVLYSAFTWLGYVNSAVNPIIYTTFNIEFRKAFLKIL
HC;

wherein the underlined part is the third intracellular loop.

In some preferred embodiments, amino acids 223-349 of the third intracellular loop of the human DRD2 receptor are deleted, and a circular permutated fluorescent protein is inserted at the deleted position. In some preferred embodiments, amino acids 268-364 of the third intracellular loop of the human DRD2 receptor are deleted, and a circular permutated fluorescent protein is inserted at the deleted position. In some preferred embodiments, amino acids 224-365 of the third intracellular loop of the human DRD2 receptor are deleted, and a circular permutated fluorescent protein is inserted at the deleted position.

In some embodiments, in the fusion polypeptide constructed based on the human HTR2C receptor, the third intracellular loop of the human HTR2C receptor is truncated and the circular permutated fluorescent protein is inserted at the truncated position.

In some preferred embodiments, in the fusion polypeptide constructed based on the human HTR2C receptor, circular permutated fluorescent protein is connected to the third intracellular loop of the human HTR2C receptor through peptide linkers at the N-terminus and the C-terminus. In some preferred embodiments, the lengths of the peptide linkers of the circular permutated fluorescent protein are 5 amino acids at the N-terminus and 3 amino acids at the C-terminus, respectively. In some preferred embodiments, the peptide linkers of the circular permutated fluorescent protein are PVVSE at the N-terminus and ATR at the C-terminus, respectively.

In some embodiments, the circular permutated fluorescent protein inserted into the human HTR2C receptor is cpmApple. In some embodiments, the cpmApple is cpmApple from R-GECO1.

In some preferred embodiments, the amino acid sequence of the human HTR2C receptor is:

(SEQ ID NO: 4)
MDPLNLSWYDDDLERQNWSRPFNGSDGKADRPHYNYYATLLTLLIAVIVF
GNVLVCMAVSREKALQTTTNYLIVSLAVADLLVATLVMPWVVYLEVVGEW
KFSRIHCDIFVTLDVMIVICTASILNLCAISIDRYTAVAMPMLYNTRYSS
KRRVTVMISIVWVLSFTISCPLLFGLNNADQNECIIANPAFVVYSSIVSF
YVPFIVTLLVYIKIYIVLRRRRKRVNTKRSSRAFRAHLRAPLKGNCTHPE
DMKLCTVIMKSNGSFPVNRRRVEAARRAQELEMEMLSSTSPPERTRYSPI
PPSHHQLTLPDPSHHGLHSTPDSPAKPEKNGHAKDHPKIAKIFEIQTMPN
GKTRTSLKTMSRRKLSQQKEKKATQMLAIVLGVFIICWLPFFITHILNIH
CDCNIPPVLYSAFTWLGYVNSAVNPIIYTTFNIEFRKAFLKILHC;

wherein the underlined part is the third intracellular loop.

In some preferred embodiments, amino acids 241-306 of the third intracellular loop of the human HTR2C receptor are deleted, and a circular permutated fluorescent protein is inserted at the deleted position. In some preferred embodiments, amino acids 240-309 of the third intracellular loop of the human HTR2C receptor are deleted, and a circular permutated fluorescent protein is inserted at the deleted position.

In some embodiments, in the fusion polypeptides constructed based on the G protein-coupled receptor, the fusion polypeptide further comprises a Gα peptide segment connected to the C-terminus of the G protein-coupled receptor. The Gα peptide segment may be preferably connected to the last amino acid at the C-terminus of the GPCR. The Gα peptide segment may be the 20 amino acids at the C-terminus of any G proteins. In some preferred embodiments, the specific sequence of the Gα peptide segment is: VFAAVKDTILQLNLKEYNLV (Gαq20, SEQ ID NO: 6). In other preferred embodiments, the specific sequence of the Gα peptide segment is: VFNDCRDIIQRMHLRQYELL (Gαs20, SEQ ID NO: 7). In other preferred embodiments, the specific sequence of the Gα peptide segment is: VFDAVTDVIIKNNLKDCGLF (Gαi20, SEQ ID NO: 8).

In a preferred embodiment, in the fusion polypeptides constructed based on the human acetylcholine receptor M3R subtype, a Gα peptide segment is connected to the C-terminus of the human acetylcholine receptor M3R subtype. The Gα peptide segment may be preferably connected to the last amino acid at the C-terminus of the human acetylcholine receptor M3R subtype. The Gα peptide segment may be the 20 amino acids at the C-terminus of any G proteins. In some preferred embodiments, the specific sequence of the Gα peptide segment is: VFAAVKDTILQLNLKEYNLV (Gαq20, SEQ ID NO: 6). In other preferred embodiments, the specific sequence of the Gα peptide segment is: VFNDCRDIIQRMHLRQYELL (Gαs20, SEQ ID NO: 7). In other preferred embodiments, the specific sequence of the Gα peptide segment is: VFDAVTDVIIKNNLKDCGLF (Gαi20, SEQ ID NO: 8).

In some embodiments, in the fusion polypeptides constructed based on the G protein-coupled receptor, the modification further includes inserting a luciferase at the C-terminus of the G protein-coupled receptor, such that the light emitted by a luciferase-catalyzed chemical reaction can excite the circular permutated fluorescent protein.

In some embodiments, the peak wavelength of the light emitted by the luciferase-catalyzed chemical reaction is close to the wavelength of the exciting light of the circular permutated fluorescent protein inserted in the fusion polypeptide.

In some embodiments, the luciferase is Nanoluc.

In other embodiments, the luciferase is Fluc (firefly luciferase) or Rluc (renilla luciferase).

In some embodiments, in the fusion polypeptides constructed based on the human HTR2C receptor, the luciferase is inserted into the C-terminus of the fusion polypeptide. The luciferase is inserted into the C-terminus of the fusion polypeptide through peptide linkers at the N-terminus and the C-terminus of the luciferase, and the peptide linkers at the N-terminus and the C-terminus of the luciferase both are GSG.

In some embodiments, the luciferase is inserted between amino acid positions 582 and 583 of the fluorescent sensor GRAB-5-HT2.0, and the two ends of the luciferase are connected to the fluorescent sensor GRAB-5-HT2.0 through peptide linkers at the N-terminus and the C-terminus, wherein the peptide linkers at the N-terminus and the C-terminus of the luciferase both are GSG. The fluorescent sensor GRAB-5-HT2.0 is a fluorescent sensor obtained by deleting the amino acid residues at the positions 15-68 of the third intracellular loop of the human HTR2C receptor, and inserting cpEGFP (preferably cpEGFP from GCaMP6s) at the deleted position, wherein the N-terminus of cpEGFP is connected to the human HTR2C receptor through the N-terminal peptide linker NG, and the C-terminus of cpEGFP is connected to the human HTR2C receptor through the C-terminal peptide linker GFAAA. The amino acid sequence of the human HTR2C receptor is

(SEQ ID NO: 4)
MVNLRNAVHSFLVHLIGLLVWQCDISVSPVAAIVTDIFNTSDGGRFKFP
DGVQNWPALSIVIIIIMTIGGNILVIMAVSMEKKLHNATNYFLMSLAIA
DMLVGLLVMPLSLLAILYDYVWPLPRYLCPVWISLDVLFSTASIMHLCA
ISLDRYVAIRNPIEHSRFNSRTKAIMKIAIVWAISIGVSVPIPVIGLRD
EEKVFVNNTTCVLNDPNFVLIGSFVAFFIPLTIMVITYCLTIYVLRRQA
LMLLHGHTEEPPGLSLDFLKCCKRNTAEEENSANPNQDQNARRRKKKER
RPRGTMQAINNERKASKVLGIVFFVFLIMWCPFFITNILSVLCEKSCNQ
KLMEKLLNVFVWIGYVCSGINPLVYTLFNKIYRRAFSNYLRCNYKVEKK
PPVRQIPRVAATALSGRELNVNIYRHTNEPVIEKASDNEPGIEMQVENL
ELPVNPSSVVSERISSV;

wherein the underlined part is the third intracellular loop.

In a preferred embodiment, the third intracellular loop along with the circular permutated fluorescent protein inserted therein of a first sensor constructed based on a first G protein-coupled receptor was used to replace the third intracellular loop of a second G protein-coupled receptor, to obtain a sensor constructed based on the second G protein-coupled receptor.

The GRAB sensor constructed by the above method can be expressed on the cell membrane, and bind to the specific ligand of the second G protein-coupled receptor, thereby causing detectable change in the fluorescence intensity of the sensor. The GRAB sensor constructed by the above method can be used to qualitatively detect the binding and concentration change of the specific ligand of the second G protein-coupled receptor, or to quantitatively analyze the concentration of the specific ligand of the second G protein-coupled receptor.

In a preferred embodiment, the first G protein-coupled receptor is a human β2 adrenergic receptor, and the amino acid sequence of the human β2 adrenergic receptor is:

(SEQ ID NO: 1)
MGQPGNGSAFLLAPNRSHAPDHDVTQQRDEVWVVGMGIVMSLIVLAIVFG
NVLVITAIAKFERLQTVTNYFITSLACADLVMGLAVVPFGAAHILMKMWT
FGNFWCEFWTSIDVLCVTASIETLCVIAVDRYFAITSPFKYQSLLTKNKA
RVIILMVWIVSGLTSFLPIQMHWYRATHQEAINCYANETCCDFFTNQAYA
IASSIVSFYVPLVIMVFVYSRVFQEAKRQLQKIDKSEGRFHVQNLSQVEQ
DGRTGHGLRRSSKFCLKEHKALKTLGIIMGTFTLCWLPFFIVNIVHVIQD
NLIRKEVYILLNWIGYVNSGFNPLIYCRSPDFRIAFQELLCLRRSSLKAY
GNGYSSNGNTGEQSGYHVEQEKENKLLCEDLPGTEDFVGHQGTVPSDNID
SQGRNCSTNDSLL,

wherein the underlined part is the third intracellular loop.

In some embodiments, the circular permutated fluorescent protein is inserted into the human β2 adrenergic receptor between amino acid position 240 and amino acid position 241. In some embodiments, the circular permutated fluorescent protein is inserted into the human β2 adrenergic receptor between amino acid position 250 and amino acid position 251.

In some embodiments, the circular permutated fluorescent protein is connected to the third intracellular loop of the human β2 adrenergic receptor through peptide linkers at the N-terminus and the C-terminus, wherein the peptide linkers of the circular permutated fluorescent protein are GG at the N-terminus and GGAAA at the C-terminus, respectively, or the peptide linkers of the circular permutated fluorescent protein are GG at the N-terminus and SPSVA at the C-terminus, respectively, or the peptide linkers of the circular permutated fluorescent protein are GG at the N-terminus and APSVA at the C-terminus, respectively.

In some embodiments, circular permutated fluorescent protein inserted into the human β2 adrenergic receptor is cpEGFP. In some embodiments, the cpEGFP is cpEGFP from GCaMP6s. In other embodiments, the cpEGFP is cpEGFP from GCaMP6m or GECO1.2.

In a more preferred embodiment, the second G protein-coupled receptor is a human acetylcholine receptor M3R subtype. In some embodiments, the specific sequence is:

(SEQ ID NO: 3)
MTLHNNSTTSPLFPNISSSWIHSPSDAGLPPGTVTHFGSYNVSRAAGNFS
SPDGTTDDPLGGHTVWQVVFIAFLTGILALVTIIGNILVIVSFKVNKQLK
TVNNYFLLSLACADLIIGVISMNLFTTYIIMNRWALGNLACDLWLAIDYV
ASNASVMNLLVISFDRYFSITRPLTYRAKRTTKRAGVMIGLAWVISFVLW
APAILFWQYFVGKRTVPPGECFIQFLSEPTITFGTAIAAFYMPVTIMTIL
YWRIYKETEKRTKELAGLQASGTEAETENFVHPTGSSRSCSSYELQQQSM
KRSNRRKYGRCHFWFTTKSWKPSSEQMDQDHSSSDSWNNNDAAASLENSA
SSDEEDIGSETRAIYSIVLKLPGHSTILNSTKLPSSDNLQVPEEELGMVD
LERKADKLQAQKSVDDGGSFPKSFSKLPIQLESAVDTAKTSDVNSSVGKS
TATLPLSFKEATLAKRFALKTRSQITKRKRMSLVKEKKAAQTLSAILLAF
IITWTPYNIMVLVNTFCDSCIPKTFWNLGYWLCYINSTVNPVCYALCNKT
FRTTFKMLLLCQCDKKKRRKQQYQQRQSVIFHKRAPEQAL;

wherein the underlined sequence is the third intracellular loop and is replaced.

In other preferred embodiments, the first G protein-coupled receptor is a human HTR2C receptor, and the amino acid sequence of the human HTR2C receptor is:

(SEQ ID NO: 4)
MVNLRNAVHSFLVHLIGLLVWQCDISVSPVAAIVTDIFNTSDGGRFKFPD
GVQNWPALSIVIIIIMTIGGNILVIMAVSMEKKLHNATNYFLMSLAIADM
LVGLLVMPLSLLAILYDYVWPLPRYLCPVWISLDVLFSTASIMHLCAISL
DRYVAIRNPIEHSRFNSRTKAIMKIAIVWAISIGVSVPIPVIGLRDEEKV
FVNNTTCVLNDPNFVLIGSFVAFFIPLTIMVITYCLTIYVLRRQALMLLH
GHTEEPPGLSLDFLKCCKRNTAEEENSANPNQDQNARRRKKKERRPRGTM
QAINNERKASKVLGIVFFVFLIMWCPFFITNILSVLCEKSCNQKLMEKLL
NVFVWIGYVCSGINPLVYTLENKIYRRAFSNYLRCNYKVEKKPPVRQIPR
VAATALSGRELNVNIYRHTNEPVIEKASDNEPGIEMQVENLELPVNPSSV
VSERISSV;

wherein he underlined part is the third intracellular loop.

In some preferred embodiments, amino acids 16-55 of the third intracellular loop of the human HTR2C receptor are deleted, and a circular permutated fluorescent protein is inserted at the deleted position. In some preferred embodiments, amino acids 11-60 of the third intracellular loop of the human HTR2C receptor are deleted, and a circular permutated fluorescent protein is inserted at the deleted position. In some preferred embodiments, amino acids 16-70 of the third intracellular loop of the human HTR2C receptor are deleted, and a circular permutated fluorescent protein is inserted at the deleted position. In other preferred embodiments, amino acids 15-68 of the third intracellular loop of the human HTR2C receptor are deleted, and a circular permutated fluorescent protein is inserted at the deleted position.

In some more preferred embodiments, amino acids 15-68 of the third intracellular loop of the human HTR2C receptor are deleted, and a circular permutated fluorescent protein is inserted at the deleted position, and the leucine (L) at position 13 of the third intracellular loop is replaced with phenylalanine (F).

In some preferred embodiments, in the sensor constructed based on the human HTR2C receptor, the circular permutated fluorescent protein is connected to the third intracellular loop of the human HTR2C receptor through peptide linkers at the N-terminus and the C-terminus, wherein the peptide linkers of the circular permutated fluorescent protein are GG at the N-terminus and GGAAA at the C-terminus, respectively, or the peptide linkers of the circular permutated fluorescent protein are NG at the N-terminus and GFAAA at the C-terminus, respectively.

In some embodiments, the circular permutated fluorescent protein inserted into the human HTR2C receptor is cpEGFP. In some embodiments, the cpEGFP is cpEGFP from GCaMP6s. In other embodiments, the cpEGFP is cpEGFP from GCaMP6m or GECO1.2.

In a more preferred embodiment, the second G protein-coupled receptor is a human HTR2B receptor or a human HTR6 receptor.

In some embodiments, the amino acid sequence of the human HTR2B receptor is:

(SEQ ID NO: 9)
MALSYRVSELQSTIPEHILQSTFVHVISSNWSGLQTESIPEEMKQIVEEQ
GNKLHWAALLILMVIIPTIGGNTLVILAVSLEKKLQYATNYFLMSLAVAD
LLVGLFVMPIALLTIMFEAMWPLPLVLCPAWLFLDVLFSTASIMHLCAIS
VDRYIAIKKPIQANQYNSRATAFIKITVVWLISIGIAIPVPIKGIETDVD
NPNNITCVLTKERFGDFMLFGSLAAFFTPLAIMIVTYFLTIHALQKKAYL
VKNKPPQRLTWLTVSTVFQRDETPCSSPEKVAMLDGSRKDKALPNSGDET
LMRRTSTIGKKSVQTISNEQRASKVLGIVFFLFLLMWCPFFITNITLVLC
DSCNQTTLQMLLEIFVWIGYVSSGVNPLVYTLFNKTFRDAFGRYITCNYR
ATKSVKTLRKRSSKIYFRNPMAENSKFFKKHGIRNGINPAMYQSPMRLRS
STIQSSSIILLDTLLLTENEGDKTEERVSYV;

wherein the underlined part is the third intracellular loop.

In some embodiments, the amino acid sequence of the human HTR6 receptor is:

(SEQ ID NO: 10)
MVPEPGPTANSTPAWGAGPPSAPGGSGWVAAALCVVIALTAAANSLLIAL
ICTQPALRNTSNFFLVSLFTSDLMVGLVVMPPAMLNALYGRWVLARGLCL
LWTAFDVMCCSASILNLCLISLDRYLLILSPLRYKLRMTPLRALALVLGA
WSLAALASFLPLLLGWHELGHARPPVPGQCRLLASLPFVLVASGLTFFLP
SGAICFTYCRILLAARKQAVQVASLTTGMASQASETLQVPRTPRPGVESA
DSRRLATKHSRKALKASLTLGILLGMFFVTWLPFFVANIVQAVCDCISPG
LFDVLTWLGYCNSTMNPIIYPLFMRDFKRALGRFLPCPRCPRERQASLAS
PSLRTSHSGPRPGLSLQQVLPLPLPPDSDSDSDAGSGGSSGLRLTAQLLL
PGEATQDPPLPTRAAAAVNFFNIDPAEPELRPHPLGIPTN;

wherein the underlined part is the third intracellular loop.

Although the following examples describe the present disclosure with different neurotransmitters as examples, those skilled in the art should understand that based on the common seven transmembrane region structure of G protein-coupled receptors, the fluorescent sensor of the present disclosure can be used for other ligands of G protein-coupled receptors, such as hormones, metabolic molecules or nutritional molecules, in addition to neurotransmitters.

### BRIEF DESCRIPTION OF DRAWINGS

The foregoing and other aspects of the present disclosure are now further explained by the detailed description of and the accompanying drawings the present disclosure. It is understood that, the embodiments in the drawings are currently preferred embodiments to illustrate the invention, however, the invention is not limited to the specific embodiments disclosed.

FIG. 1 shows a typical response of GRAB-EPI 0.1 to saturation concentration ISO (2 μM). After adding ISO, the receptor undergoes a conformational change, resulting in a rapid increase in the fluorescence signal, with an average amplitude of 6% ΔF/F₀. After washing away the ISO with physiological solution, the conformation of the receptor recovers to the inactive state, and the corresponding fluorescence value from cell also returns to the baseline. The bottom panels are schematic diagrams of the fluorescence intensity of a single cell expressed in pseudo color before and after adding ISO. As shown, the fluorescence value on the cell membrane has a significant reversible change before and after adding ISO.

FIG. 2 shows the results of using different circular permutated fluorescent proteins to construct GRAB-EPI sensors. The sensors constructed with the circular permutated EGFP can be folded and transported to cell membrane correctly. The images in the bottom panels were taken with an Olympus IX81 inverted fluorescence microscope.

FIG. 3 shows the results obtained by changing the insertion site of the fluorescent protein in the third intracellular loop of the β2 adrenergic receptor. The sensor with a signal change of about 15% ΔF/F₀ shows a sensitive, rapid, and reversible change upon the ligand.

FIG. 4 shows the construction of GRAB-ACh sensor by inserting the short ICL3 of β2AR into M₁₋₅R. a: Sequence alignment of β2AR and M1-5R, wherein the region between TM5 and TM6 is shown, and the border of insertion is indicated by a black dotted line. b: Fluorescence response of M₁₋₅R-β2R ICL3-cpEGFP chimera to ACh (100 μM), wherein only sensor derived from M3R showed detectable increase in fluorescence. The data was collected by TECAN fluorescence analyzer (n=6-10 wells/chimera, >100 cells/well). M₁R, ΔF/F₀−2.11±1.58%; M₂R, ΔF/F 2.09±1.19%; M₃R, ΔF/F₀ 22.03±0.86%; M₄R, ΔF/F₀ 2.16±1.63%; M₅R, ΔF/F₀−0.49±0.16%.

FIG. 5 shows the construction of the GRAB-ACh sensor for acetylcholine. a: Principle of the GRAB-ACh sensor. b: The typical membrane localization modes of GRAB-ACh sensors based on different muscarinic receptors in HEK293T cells. The M₃R-based sensor, named GRAB-ACh 1.0, shows a good membrane localization. c & d: Optimization of GRAB-ACh 1.0: cpEGFPs containing peptide linkers (N-terminal 2 amino acids, C-terminal 5 amino acids) with random mutation were screened and the single mutation with the best effect (c) was further combined (d), to generate a sensor named GRAB-ACh 2.0, with ΔF/F₀ close to 100%. Each data point is an average response of 2-10 cells. e-g: the responses of GRAB-ACh 1.0 & 2.0 in HEK293T cells. The pseudocolor images show their peak response to perfusion of 100 μM ACh (e), f shows the quantitative value of the experiment of e, and g shows the data of GRAB-ACh 1.0 & 2.0 groups (GRAB-ACh 1.0: ΔF/F₀ 24.62±1.51%, n=19 cells; GRAB-ACh 2.0: ΔF/F₀ 90.12±1.74%, n=29 cells; Z=−5.79, p<0.001). h-j: Comparison of GRAB-ACh 2.0 with FRET sensor-based muscarinic receptors. Under 100 μM ACh perfusion, GRAB-ACh 2.0 shows a significantly improved signal compared with FRET sensor in terms of peak response (ΔF/F₀ 94.0±3.0% vs. 6.6±0.4% of FRET sensor, i) and signal-to-noise ratio (724±9 vs. 8.3±1.1, j) (n=10 cells per group). g shows the Mann-Whitney rank sum nonparametric test, *, p <0.05; **, p<0.01; ***; p<0.001; n.s., no significance. All scale bars are 10 μm.

FIG. 6 shows the results of screening the optimal length of the peptide linker between fluorescent protein and GPCR. Among them, the ON sensor with the highest signal change has a peptide linker length 2-5, and the best OFF sensor has a peptide linker length 1-1. The number under each column in the diagram represents the length of the peptide linker at the N-terminus and the length of the peptide linker at the C-terminus, for example, 1-3 means that there is 1 amino acid at the N-terminus and 3 amino acids at the C-terminus.

FIG. 7 shows the optimization of GRAB-ACh 1.0 by random mutation in the peptide linker. a: each residue of the peptide linkers, 2 amino acid residues in the N-terminus and 5 amino acid residues in the C-terminus of cpEGFP was randomly mutated to 20 possible amino acids individually (left panel). 373 variants on these 7 residues were tested individually and their ΔF/F₀ responses to ACh (100 μM) in HEK293T cells were quantified (right panel). The top four mutations with the best performance of each residue were chosen for the second round screening. b: The sequence information and the ΔF/F₀ response of each of the 23 candidates in the second round screening, in which, ΔF/F₀ of GRAB-ACh 2.0 is close to 0.9.

FIG. 8 shows that the muscarinic receptor-based FRET sensor has poor response to ACh. a: The FRET sensor based on M₁R was constructed as previously reported (Markovic, D., et al. FRET-based detection of M1 muscarinic acetylcholine receptor activation by orthosteric and allosteric agonists. PloS one 7, e29946 (2012)), wherein CFP was inserted between K361 and K362 of the ICL3, YFP was fused to its C-terminus. The chimeric protein exhibits poor membrane localization. b: ACh (100 μM) induces a slight fluorescence decrease in the YFP channel and a fluorescence increase in the CFP channel (average results of n=10 cells). c: After ACh perfusion, the FRET ratio (CFP/YFP) of the ACh sensor showed a moderate increase.

FIG. 9 shows the spectral properties of the GRAB sensor and its pH sensitivity. The GRAB sensor constructed based on green fluorescent protein has excitation and emission peaks similar to GFP, near 490 nm and 520 nm, respectively, meanwhile, its fluorescence intensity depends on the pH of the solution.

FIG. 10 shows the properties of various GRAB sensors with fluorescent proteins connected to the C-terminus of GPCRs. For different ligands (acetylcholine, dopamine, histamine, isoproterenol, serotonin, and Oxtocin), the binding of ligand with the corresponding GPCR always results in change of fluorescence intensity.

FIG. 11 shows the specificity of the fluorescence signal produced by GRAB sensor after ligand activation. When a specific blocker of the receptor is added, the agonist (the same concentration) cannot lead to the change in fluorescence signal because it cannot bind to the receptor.

FIG. 12 shows that mutations in the ligand-binding domain of the GPCR can significantly affect the performance of the sensor. A: After mutation is introduced to the ligand-binding domain of β2 adrenergic receptor, the agonist ISO cannot cause the fluorescence signal to rise. B: After reducing the affinity of the acetylcholine receptor to the ligand by mutation, the acetylcholine fluorescent sensor shows a reduced affinity for acetylcholine.

FIG. 13 shows the ligand dose-dependent change of fluorescence signal from GRAB sensor. A: The GRAB-EPI 1.0 sensor shows enhanced fluorescence signal for different concentrations of agonist ISO, similar to the endogenous β2 adrenergic receptor. B: The GRAB-ACh 1.0 sensor shows varied fluorescence intensity for different concentrations of acetylcholine, similar to the endogenous M3 acetylcholine receptor.

FIG. 14 shows that GRAB-ACh 2.0 exhibits subsecond dynamics and micromolar sensitivity for the detection of ACh. a: Diagram of the rapid perfusion system, in which a glass pipette filled with ACh and red rhodamine-6G dye is placed near the GRAB-ACh 2.0 expressing cells, and the white line indicates the scanning. b: Scanning on ACh and Tio. Perfusion of ACh or Tio causes the increase or decrease of fluorescence of GRAB-ACh 2.0 with a time constant of 185 ms and 696 ms, respectively. c: Data set in b, the average on time constant is 279.4±32.6 ms, n=18, and off time constant is 762.3±74.9 ms, n=11. d & e: The dose-dependent response of GRAB-ACh 2.0 to ACh. For GRAB-ACh 2.0 (˜0.7 μM, n=4), pEC₅₀=−6.12±0.11 M, which is very close to the Kd (0.5-2 μM) of WT-M3R (Jakubik, J., Bacáková, L., El-Fakahany, E. E. & Tucek, S. Positive cooperativity of acetylcholine and other agonists with allosteric ligands on muscarinic acetylcholine receptors. Molecular pharmacology 52, 172-179 (1997)). AF-DX384, an M3R antagonist, completely blocked the increase of fluorescence. The data in d is in μM, represents average response from 3 experiments conducted in the same HEK293T cell.

FIG. 15 shows that the coupling of GRAB sensors with G protein-mediated signaling pathways has a significant decrease. After the cells were treated with calcium dye, perfusion was performed using different concentrations of acetylcholine. The difference in calcium signal between cells expressing GRAB-ACh 1.0 sensor and that expressing endogenous M3 acetylcholine receptor was compared. The response curves of calcium signal versus ligand concentration (bottom panel) show that the degree of coupling of calcium signal in cells expressing GRAB sensor is reduced by about 5 times.

FIG. 16 shows that the activation of G protein-mediated downstream pathways can be reduced by connecting a Gα peptide segment to the end of the GRAB sensor to compete for endogenous G protein binding, wherein the peptide segment at the C-terminus of Gα can stabilize GPCR in the activated state without downstream signal transduction. A: Fluorescence image of the GRAB-ACh 2.0-Gq20 sensor, showing good membrane expression. B: GRAB-ACh 2.0-Gq20 exhibits increased fluorescence signal upon the addition of saturated acetylcholine, with a signal change of about 70% ΔF/F₀. C: The change of calcium signal of cells expressing different sensors under different concentrations of neurotransmitters obtained by calcium imaging method. By calculating the Kd value, it can be seen that the sensor with the Gα peptide segment has a significant decrease in G protein-mediated downstream pathway.

FIG. 17 shows the detection of the coupling of the fluorescent sensor and the endocytosis signaling pathway, wherein the fluorescent sensor is constructed based on the principle of GPCR receptor endocytosis. A: The principle of endocytosis sensor. B: The sensor pHluorin-β2AR constructed based on β2 adrenergic receptors shows obvious activation of the endocytosis signaling pathway, that is, the decrease of the cell fluorescence signal.

FIG. 18 shows that GRAB sensor has a greatly reduced coupling efficiency to arrestin-mediated endocytosis signal pathway. B: For the GRAB-EPI 1.0 sensor, after treating the cells with a saturated concentration of agonist ISO for 30 minutes, the fluorescence level on the cell membrane did not change with time. C: By comparing the endocytosis signal coupling efficiency of GRAB-EPI 1.0 sensor and endogenous β2 adrenergic receptor, it can be found that GRAB sensor almost completely eliminated the coupling of endocytosis signal pathway, thus truly reflecting the ligand concentration dynamic change.

FIG. 19 is fluorescence images of GRAB sensors in cultured neurons. A: Imaging of GRAB-EPI 1.0 in cortical neurons. B: Fluorescence imaging of GRAB-ACh 1.0 in cortical neurons (left panel) and a partially enlarged view (right panel).

FIG. 20 shows the response of GRAB sensors in cultured neurons. A: The GRAB-ACh 1.0 sensor shows ligand-specific fluorescence signal increase in cultured cortical neurons. B: GRAB-EPI 1.0 sensor and GRAB-ACh 1.0 sensor show does-dependent fluorescence response in neurons.

FIG. 21 shows the response specificity of GRAB sensor to specific neurotransmitter. A & B: GRAB-ACh 1.0 only produces a reproducible and reversible specific response to epinephrine (Epi) and its analogs (ISO), and such response disappears after the addition of the inhibitor ICI. C & D: The GRAB-ACh 1.0 sensor only produces a reproducible specific response to acetylcholine, but no fluorescence response to other major neurotransmitters.

FIG. 22 shows that the GRAB-ACh 1.0 sensor specifically detects endogenous acetylcholine release in the olfactory system of Drosophila. After the administration of isoamyl acetate odor, the optical signal of the sensor at the antennal nerve lobe showed a rapid rise, and the changes was dose-dependent (top panel). In addition, the rise of the fluorescent signal showed olfactory bulb specificity. In olfactory bulbs that received projections of olfactory receptor neurons sensing isoamyl acetate, such as DM2, the fluorescent signal increased, while in olfactory bulbs that did not receive projections of such neurons, such as DA1, there was no change in the fluorescence signal (bottom panel).

FIG. 23 shows the effect of overexpression of GRAB sensors on calcium signal of cells by using red calcium indicator RGECO. A: In Drosophila expressing RGECO alone and Drosophila co-expressing both RGECO and GRAB-ACh 1.0 sensor, the increases of the calcium signal in the DM2 olfactory bulb of the antennal nerve lobes triggered by odor molecules are similar. B: Statistical results from multiple samples.

FIG. 24 shows the performance of GRAB-ACh sensor on acute mouse hippocampus brain slices. A: Fluorescence images of GRAB-ACh sensor in hippocampal neurons under a two-photon microscope. From left to right: images of neurons stained with the red dye Alexa 594, images of neurons transfected with GRAB-ACh, and the merged images of the previous two images. GRAB sensors are evenly distributed on the cell membrane of the neuron, and visible in the axons of neurons and other structures. B: GRAB-ACh-expressing cells showed increased fluorescence induced by specific acetylcholine compared to non-expressing cells. C: The cells expressing GRAB-ACh sensor respond to the M-type receptor agonist acetylcholine, Oxo-M, but not to the nicotine and physiological solution (ACSF, artificial cerebrospinal fluid).

FIG. 25 shows the selection of human norepinephrine receptors for construction of fluorescent sensors. a: The chemical structure of norepinephrine NE and adrenaline Epi. b: The expression of three different norepinephrine receptors ADRA1D, ADRB3, ADRA2A with green fluorescent protein pHluorin at the N-terminus in mammalian HEK293T cells. The arrows in ADRA1D and ADRB3 indicate cells with poor membrane expression, and the arrows in ADRA2A indicate cells with better membrane expression. Scale=50 μm.

FIG. 26 shows the development and optimization of norepinephrine fluorescent sensors. a: Schematic diagram showing truncation of the third intracellular loop ICL3 of the ADRA2A receptor and insertion of the circular permutated fluorescent protein cpEGFP. b: GRAB-NE1.0 with fluorescence signal change in response to NE was obtained in the first round of screening. c: After carefully screening the insertion site in the second round, GRAB-NE2.0 with higher fluorescence brightness and greater change in response to NE was obtained. d: In the drug perfusion experiment, with the treatment of 100 μM NE, the NE1.0 and 2.0 versions have a change of more than 100% and 200% in the fluorescence signal, respectively. The change is reversible, and the fluorescence intensity returns to the initial value after the drug is washed away. e: Pseudocolor images of GRAB-NE1.0 and 2.0. Scale=10 μm.

FIG. 27 shows the further optimization of GRAB-NE2.0 peptide linkers. a: Schematic diagram of the screening library of the truncated peptide linkers. b: The truncation of the peptide linkers did not produce a sensor with higher fluorescence intensity or greater change in fluorescence signal than GRAB-NE2.0. c: Schematic diagram of the amino acid mutation library for the peptide linkers. d: After the third round of screening for the peptide linkers, GRAB-NE2.1 with higher fluorescence intensity and greater change in fluorescent signal in response to NE was obtained, in which the third amino acid glycine of the peptide linker was mutated to threonine.

FIG. 28 shows the characterization of the GRAB-NE sensors and the development of GRAB-NE2.2. a: Drug specificity analysis of GRAB-NE2.0. The sensor only exhibits a fluorescent signal change to the neurotransmitters NE and Epi, while no response to the saturated concentration of the β-type receptor specific activator ISO and other neurotransmitters. Addition of the alpha receptor specific blocker Yohimbine (2 μM) and 5204A mutation in the ligand binding region of the ADRA2A receptor can inhibit ligand-induced NE sensor signal changes. b: Sequential perfusion of 10 nM to 100 μM of NE shows the ligand concentration-dependent curve of GRAB-NE2.0. The changes can also be completely inhibited by the addition of 1 μM of blocker Yohimbine. c: GRAB-NE2.2 was obtained by the introduction of T373K mutation. The dose-dependent curve of GRAB-NE2.2 is shifted to the left compared to GRAB-NE2.1, and the affinity for ligand NE is increased by 10 times. d: Expression and membrane localization of GRAB-NE2.1, GRAB-NE2.1 S204A, GRAB-NE2.2 in HEK293T cells. e: The optimized and improved NE2.1 and NE2.2 versions of GRAB-NE have higher fluorescence intensity and fluorescence response signal compared to GRAB-NE2.0. f: GRAB-NE2.2 has similar dose-dependent curves for ligands NE and Epi, and the affinity to both ligands is improved. Scale bar, 10 μm.

FIG. 29 shows that the GRBA-NE2.2 sensor has fast reaction kinetics. a: Schematic diagram showing release of free NE and NPEC group from NPEC-caged-NE under UV light activation. b: Using 405 nm laser to stimulate photolysis in the white area around GRAB-NE2.2, a 20% change in fluorescence signal of GRAB-NE2.2 with 100 μM NPEC-NE can be observed. In the presence of the 10 μM blocker Yohimbine, the above change in fluorescence signal was inhibited. c: Fluorescence signal change of GRAB-NE2.2 with photolysis reaction of 100 μM NPEC-NE and addition of 10 μM Yohimbine. Amplification of 2000 ms around the photolysis time point can fit and obtain a rate constant of 104 ms corresponding to the increase of GRAB-NE2.2 sensor fluorescence signal to the photolysis reaction.

FIG. 30 is a depiction of the uncoupling of GRAB-NE sensor and downstream G protein signal. a & b: Schematic diagrams of the uncoupling of Gαi protein and GPCR by the insertion of green fluorescent protein in the third intracellular loop of NE receptor protein ADRA2A. c & d: The co-transfection of GRAB-NE2.0 and PTX did not change the concentration-dependent curve of NE2.0 to ligand (unit: μM). e: Adding GTPγS under the treatment of digitonin (a saponin for producing holes in the cell membrane to allow externally added drugs, especially small molecules (GTPγS, etc.) with poor lipid solubility to cross the cell membrane and enter the cell) to inhibit the activation cycle of Gα protein also did not change the concentration-dependent curve of GRAB-NE2.0 to NE. f. In the experiment of downstream TGFα release caused by GRAB-NE2.0, the receptor protein ADRA2A and TPA (which can directly activate intracellular PLC (downstream of GPCR), and can be used as a positive control in TGF-α assay to check whether the system works normally) under the treatment of 100 nM NE, it was found that the activation intensity of the downstream signal of GRAB-NE2.0 is only ⅓ of that of the receptor protein.

FIG. 31 shows that GRAB-NE2.1 in cultured neurons has optical signal changes to specific neurotransmitters. a & b: The co-transfection of GRAB-NE2.1 and PSD95-mcherry showed that GRAB-NE is evenly distributed on the neuron membrane, slightly aggregated in the cell body part (1), but is well distributed on the dendritic membrane (such as arrows 1, 2). There is also clear distribution on the dendritic spines co-located with PSD95 (such as triangles 1, 2). c: During the perfusion with 100 μM NE, there was about 200% change in fluorescence signal on the cell membrane and dendritic spines. Similar to that in cells, due to poor membrane expression, the cell body had a response of about 60%. d: Pseudocolor images of neurons transfected with GRAB-NE2.1 during drug perfusion and after drug elution. e: Comparison of fluorescent response signals of cell body, cell membrane, and dendritic spines. f & g: The dependence curve of GRAB-NE2.1 neuronal cell body to different concentrations of NE, with drug perfusion from 10 nM to 100 μM, and the ligand affinity is 790 nM. Scale bar, 10 μm.

FIG. 32 shows that the neurotransmitter fluorescent sensor GRAB-NE2.1 in cultured rat cardiomyocytes has optical signal changes to specific neurotransmitter. a: Expression and membrane localization of GRAB-NE2.1 in rat cardiomyocytes. b: With 100 μM NE perfusion, GRAB-NE2.1 has a fluorescence signal change greater than 300% in cardiomyocytes, and the reaction was reversible. c: Pseudocolor images of GRAB-NE2.1 response in cardiomyocytes. d & e: The reaction of the sensor in cardiomyocytes is also ligand-dependent. The sequential perfusion of 1 nM to 100 μM of NE can obtain the ligand concentration-dependent curve of this sensor, with an affinity of 500 nM. 1 μM blocker Yohimbine can inhibit this reaction (unit: μM). Scale bar, 50 μM.

FIG. 33 shows that the GRAB-5-HT2.1 sensor exhibits a ligand-dependent fluorescence response in HEK293T cells, with a Kd value of about 131 nM, similar to the affinity of the HTR2C receptor under physiological conditions.

FIG. 34 shows: A: The GRAB-5-HT2.1 sensor only produces a specific response to serotonin, but not other major neurotransmitters such as Gly, Epi, and Ach. B: Serotonin and HTR2C-specific agonist CP809 can cause the fluorescence signal change of the GRAB-5-HT2.1 sensor, while the HTR2B-specific agonist BWT23C83 and HTR1B-specific agonist CGS12066B cannot cause the change. HTR2C-specific antagonist RS102221 can antagonize the increase in the fluorescence signal of GRAB-5-HT2.1 sensor caused by serotonin, while HTR2B-specific antagonist SB204741 cannot antagonize the increase in signal.

FIG. 35 shows the responses of a series of serotonin fluorescent sensors constructed based on different HTR receptors after the addition of a saturated concentration of serotonin.

FIG. 36 shows that the GRAB-5-HT2.0 sensor specifically detects endogenous serotonin release in the olfactory system of Drosophila. After odor (isoamyl acetate, banana flavor) stimulation, the optical signal of the sensor showed a rapid rise.

FIG. 37 shows the signal change of the sensor GRAB-GDA3.0 constructed based on DRD2 under the treatment of saturation concentration dopamine.

FIG. 38 shows the pharmacological characterization of GRAB-GDA3.0 in HEK293T cells. GRAB-GDA3.0 can only be activated by dopamine and hDRD2-specific agonist quinpirole, and is blocked by hDRD2-specific antagonist Haloperidol.

FIG. 39 shows the signal from GRAB-GDA3.0 (shown as GDA in the figure) after odor-stimulated in MB. A: Schematic diagram of 2-PT imaging in Drosophila after odor stimulation. GRAB-GDA3.0 is expressed in dopaminergic neurons (DAN), driven by TH-GAL4. Mushroom bodies (MB) can receive dopaminergic signal. MB β'lobe is outlined with a dotted line. The scale bar is 25 μm. B: GDA located on the cell membrane of DAN can report the release of dopamine in the synaptic space. C1-C3: Pseudocolor images of GRAB-GDA3.0 in β'lobe after IA (1% isoamyl acetate, 5 seconds) stimulation. The scale bar is 25 μm. D: The average time of 3 trials of the GRAB-GDA3.0 signal in β'lobe after IA stimulation in Drosophila.

FIG. 40 shows that the GRAB-GDA3.0 (shown as GDA in the figure) signal stimulated by odor in MB is dopamine specific. A-C: The IA-stimulated GDA signal in β'lobe can be blocked by hDRD2-specific antagonist halo (10 μM haloperidol). Pseudocolor images in Drosophila before and after halo application. Scale bar is 25 μm (panel A); average time of three trials in the same fruit fly before and after halo application (panel B); statistical results showing significant inhibition of GDA by halo (panel C). Error bars indicate SEM (n=6). D-F: The IA-stimulated GDA signal in MB β'lobe cannot be blocked by the octopamine receptor antagonist epinastine (10 μM). Pseudocolor images in Drosophila before and after application of epinastine, scale bar 25 μm (panel D); average time of three trials in the same fruit fly before and after application of epinastine (panel E); statistical results showing that epinastine has no inhibitory effect on GDA (panel F). Error bars indicate SEM (n=6). G-J: When DAT-RNAi is expressed in DAN and driven by TH-GAL4, the attenuation of GDA signal is τ. DAT is located in the presynaptic membrane of DAN, and releases DA from the gap circulation (panel G). Average time in a WT Drosophila and a DAT-deficient Drosophila. The fitting result of the attenuation curve is shown in the panel H; the error bars represent SEM (n=6). Pseudocolor images after odor stimulation in the WT fruit fly and the DAT-deficient fruit fly are shown. The scale bar is 25 μm (panel J).

FIG. 41 shows the construction of a cpmApple-based dopamine fluorescent sensor. A: The ligand-induced response (ΔF/F₀) of the variants in the constructed library. Perfusion was performed to test the performance of 92 variants, 16 of which did not show fluorescence, 56 did not show ligand-induced response, 16 showed on response, and 5 showed off response. The dashed rectangle indicates the candidate with the highest ON or OFF responses. 222-349/267-364 indicate the insertion sites of cpmApple in HTR2C. B: the left panel is the images of the two selected candidates, and the right panel is the corresponding response curve. The scale bar is 20 μm. The result is shown as the mean±SEM. For one curve, n=6 cells, and for the other curve, n=5 cells. C: Left panel is ligand-induced response (ΔF/F₀) of the variants in peptide linker random mutation library. The figure shows only the response characteristics of the variant. The dashed rectangular box indicates the candidate with the largest response. The middle panel shows the imaging characteristics of the best candidates of the fine-tuning library. The right panel is the corresponding response curve. The scale bar is 20 μm. The results are shown as the average±SEM. For one curve n=5 cells, and for the other curve, n=6 cells. D: The right panel is a ligand-induced response (ΔF/F₀) and the relative brightness of the variants in peptide linker random mutation library. The library is a mixture of five individual libraries, wherein each individual library is a random mutation library of one amino acid. The red dot indicates the characteristics of the starting template, which is the best candidate selected from the fine-tuning library. The black dots indicate the characteristics of the variants of the random mutation library of the peptide linker. In the left panel, X indicates the position of the randomly mutated amino acids of the peptide linker, and the amino acids of the peptide linker are individually and randomly mutated one by one.

FIG. 42 shows the construction of cpmApple-based serotonin fluorescent sensor. A: The ligand-induced response (ΔF/F₀) of the variants in the library constructed by the cpRFP insertion strategy and the fine-tuning strategy. The dashed rectangle indicates the candidate with the highest on and off responses. 240-306/239-309 indicate the insertion sites of cpmApple into HTR2C. B: The left panel is the imaging characteristics of the two selected candidates, and the right picture is the corresponding response curve. The scale bar is 20 μm. The result is shown as the mean±SEM. For one curve, n=8 cells, and for the other curve, n=6 cells. C: The right panel is a ligand-induced response (ΔF/F₀) and the relative brightness of the variants in peptide linker random mutation library. The library is a mixture of five individual libraries, wherein each individual library is a random mutation library of one amino acid. The red dot indicates the characteristics of the starting template, which is the best candidate selected from the fine-tuning library. The black dots indicate the characteristics of the variants of the random mutation library of the peptide linker. In the left panel, X indicates the position of the randomly mutated amino acids of the peptide linker, and the amino acids of the peptide linker are individually and randomly mutated one by one.

FIG. 43 is the signal changes of the serotonin fluorescent sensor based on bioluminescence resonance energy transfer, wherein R is the ratio of the signal intensity of the 535 nm channel to the signal intensity of the 450 nm channel; dR is ΔR, i.e., the change value of R; the 535 nm channel indicates the emission wavelength of the GRAB sensor, and the 450 nm channel is the emission wavelength of Nanoluc, and the ratio of the two is used as a measure of energy resonance transfer.

FIG. 44 shows that a specific receptor blocker (Tio) can block the response of the acetylcholine sensor GRAB-ACh 1.0 to the ligand acetylcholine.

FIG. 45 shows the optimization of the fluorescent sensor constructed based on the acetylcholine M3R receptor. a & b: Randomly select one site from the 7 sites at the N-terminal and one site from the 8 sites at the C-terminal of ICL3, respectively, delete the fragment between these two sites and insert cpEGFP. c: Select candidates from Opera Phenix screening results and confirm with confocal perfusion. d: Perfusion results of some mutants.

FIG. 46 shows the optimization of the peptide linkers between cpEGFP and the M3R receptor, wherein when the first amino acid at the C-terminus is histidine, the sensor has a better performance.

FIG. 47 shows GRAB-ACh4.0 obtained through optimization of the peptide linkers.

FIG. 48 shows the perfusion results of GRAB-ACh4.0.

FIG. 49 shows that the affinity of GRAB-ACh4.0 to ligand acetylcholine is not significantly different from the wild-type M3R receptor in the literatures.

FIG. 50 shows that GRAB-ACh4.0 can and can only be activated by ACh to cause a change in fluorescence intensity.

FIG. 51 shows that GRAB-ACh4.0 does not activate the downstream Gq-mediated signaling pathway.

FIG. 52 shows the experimental results of drug screening using cell lines expressing GRAB-5HT1.0 sensor.

FIG. 53 shows that connecting different Gα peptide segments at the C-terminus of the acetylcholine sensor can decrease the coupling ability of the sensor to the downstream G protein signaling pathway.

### DETAILED DESCRIPTION

The term used in this application has the same meaning as in the prior art. In order to clearly indicate the meaning of the terms used, the specific meanings of some terms in this application are given below. When the definition herein conflicts with the conventional meaning of the term, the definition herein shall prevail.

The “G protein-coupled receptor (GPCR)” described herein belongs to a large protein family of transmembrane receptors, which sense molecules outside the cell, activate intracellular signal transduction pathways and ultimately activate cellular responses. Ligands that bind and activate these receptors include photosensitive compounds, odors, pheromones, hormones, and neurotransmitters, and vary in size from small molecules to peptides to large proteins. GPCRs involve many diseases and are the target of about half of all current drugs.

G protein-coupled receptor (GPCR) is a class of seven transmembrane proteins expressed on the plasma membrane of the cell. The GPCR protein is composed of seven α-helix structures across the plasma membrane as the main body, the N-terminus and 3 loops outside the cell, and the C-terminus and 3 loops inside the cell. In the study of G protein-coupled receptors, the analysis of crystal structure helps scientists understand the specific mechanism by which ligands trigger downstream pathways in cells. By stabilizing the receptors with G peptide segment, the Masashi Miyano group for the first time analyzed the crystal structure of the classic GPCR, the photoreceptor rhodopsin in vision (Palczewski, K. et al. Crystal Structure of Rhodopsin: A G Protein-Coupled Receptor. Science (New York, N.Y.) 289, 739-745 (2000)), and in the structural comparison of the activated state and inactive state, they found that GPCR undergoes a series of conformational changes after ligand binding, in which the most obvious change is the outward extension of the fifth and sixth transmembrane regions, thereby exposing a structural hole to facilitate the entry of the carbon end of the G protein. Subsequently, through a variety of methods to stabilize the crystal structure of GPCRs, especially the application of the single-chain antibody nanobody that can stabilize the receptor in the activated state, the Brian kobilka group successfully resolved the crystal structure of the β-adrenoceptor around 2012 (Rasmussen, S. G. F. et al. Crystal structure of the human beta2 adrenergic G-protein-coupled receptor. Nature 450, 383-387 (2007); Rasmussen, S. G. F. et al. Crystal structure of the human beta2 adrenergic G-protein-coupled receptor. Nature 450, 383-3; Cherezov, V. et al. High-Resolution Crystal Structure of an Engineered Human beta G protein-coupled receptor. Science 318, 1258-1265 (2007)). Similar to rhodopsin, the activation of adrenergic receptors also accompanied by obvious molecular conformational changes, among them, the fifth and sixth transmembrane regions experienced the most changes. In order to further confirm that this specific conformational change is a conservative activation mode common to most GPCRs, the crystal structure of the M-type acetylcholine receptor was analyzed (Kruse, A. C. et al. Structure and dynamics of the M3 muscarinic acetylcholine receptor. Nature 482, 552-556 (2012)), opioid (Huang, W. et al. Structural insights into micro-opioid receptor activation. Nature 524, 315-321 (2015)) and the results show that it has a similar conformational change pattern. Therefore, it is speculated that this activation pattern may be common to most GPCRs. According to the crystal structure analysis of GPCR, GPCR itself can be regarded as a specific ligand sensor that evolves naturally, and its reaction is a conservative conformational change to mediate the activation of downstream pathways.

All the sites with conformational changes revealed by the existing GPCR conformational studies can theoretically be used as the insertion positions of signal molecules. In one embodiment, the insertion position of the signal molecule is selected to be near the fifth and sixth transmembrane regions with the largest GPCR conformation changes. In a specific embodiment, the insertion position is selected as the third intracellular loop; and in another embodiment, the insertion position is selected as the C-terminal peptide with a large conformational change.

In this context, G protein-coupled receptor (GPCR) includes both naturally occurring forms, as well as the variants forms formed by replacing, inserting, or deleting one or more amino acids in the naturally occurring forms, which retains the function of the naturally occurring forms. The GPCR can exist independently or as part of a larger molecular structure (such as a fusion protein).

Based on sequence homology and functional similarity, GPCRs can be divided into at least 5 categories: class A rhodopsin-like, class B secretin-like, class C metabolic/pheromones, class D fungal pheromones, and class E cAMP receptor.

Class A rhodopsin-like eceptors include: amine receptors: acetylcholine, alpha adrenergic receptors, beta adrenergic receptors, dopamine, histamine, serotonin, octopamine, and trace amines; peptide receptors: angiotensin, bombesin, bradykinin, C5a anaphylatoxin, Fmet-leu-phe, APJ-like substance, interleukin-8, chemokine receptor (C-C chemokine, C-X-C chemokine, B0NZ0 receptor (CXC6R), C-X3-C chemokine and XC chemokine), CCK receptor, endothelin receptor, melanocortin receptor, neuropeptide Y receptor, neurotensin receptor, opioid receptor, somatostatin receptor, tachykinin receptor (substance P (NK1), substance K (NK2), neuromodulin K (NK3), tachykinin-like 1 and tachykinin-like 2), vasopressin-Like receptors (vasopressin, oxytocin, and Conopressin), galanin-like receptors (galanin, allatostatin, and GPCR 54), protease-activated receptors (e.g., thrombin), orexin & neuropeptide FF, urotensin II receptor, adrenomedullin (G10D) receptor, GPR37/endothelin B-like receptor, chemokine receptor-like receptor and neuromodulin U receptor; hormone protein receptor: follicle-stimulating hormone, luteinizing hormone-chorionic gonadotropin, thyrotropin and gonadotropin; (Rhod) opsin receptor; olfactory receptor; prostaglandin receptors: prostaglandin, prostacyclin and Thromboxane; nucleotide-like receptors: adenosine and purine receptors; cannabis receptors; platelet activating factor receptors; gonadotropin-releasing hormone receptors; thyrotropin-releasing hormone & secretagogue receptors: thyrotropin-releasing hormone, growth hormone secretagogue and growth hormone secretagogue-like; melatonin receptor; viral receptor; soluble sphingolipid (Lysosphingolipid) & LPA (EDG) receptor; leukotriene M receptor: leukotriene B4 Receptor BLT1 and leukotriene M receptor BLT2; and class A orphan/other receptors: platelet ADP & KI01 receptor, SREB, Mas proto-oncogene, RDC1, ORPH, LGR-like (hormone receptor), GPR, GPR45-like, Cysteinyl leukotrienes, Mas-related receptors (MRGs) and GP40-like receptors.

GPCR class B (family of secretin receptors) includes polypeptide hormone receptors (calcitonin, adrenocorticotropic hormone releasing factor, enterostatin, glucagon, glucagon-like peptide-1, -2, growth hormone-releasing hormone, parathyroid hormone, PACAP, secretin, vasoactive intestinal peptide, diuretic hormone, EMR1, aracotoxin receptor (Latrophilin)), molecules thought to mediate cell-cell interactions in the plasma membrane (brain Specific angiogenesis inhibitors (BAI)) and a group of Drosophila proteins (Methuselah-like proteins) that regulate stress response and longevity.

Class C metabolic glutamate/pheromone receptors include metabolic glutamate, group I metabolic glutamate, group II metabolic glutamate, group III metabolic glutamate, other metabolic glutamate, extracellular calcium sensing, putative pheromone receptor, GABA-B receptor (GABA-B receptor is composed of two subunits (B1, B2) and is a dimeric protein) and orphan GPRC5 receptor.

GPCRs involve various physiological processes, including vision, smell, behavior and mood regulation, immune system activity and inflammation regulation, autonomic nervous system transmission, cell density sensing, and many others. It is known that the inactivated G protein binds to the receptor in its inactivated state. After recognizing the ligand, the receptor or its subunits undergoes a conformational change and thus mechanically activate the G protein, which is then released from the receptor. Now the receptor can activate another G protein, or switch back to its inactive state. It is believed that the receptor molecule exists in a conformational balance between active and inactive biophysical states. Ligand binding to the receptor can shift the balance to the active receptor state.

G protein-coupled receptors can be used in the present disclosure include, but are not limited to, β2 adrenergic receptor (ADRB2), α2A adrenergic receptor (ADRA2A), acetylcholine receptor M3R subtype (M3 type muscarinic acetylcholine receptor, CHRM3), dopamine D2 receptor (DRD2), serotonin 2C receptor (HTR2C), serotonin 2B receptor (HTR2B), serotonin receptor 6 (HTR6), which are well known to those skilled in the art The sequence can be obtained through various ways, such as a publicly available database.

Those skilled in the art can easily determine the N-terminus, transmembrane region, intracellular loop and C-terminus of G protein-coupled receptors, for example, based on the similarity of its amino acid sequence with the transmembrane region of known G protein-coupled receptors. Various bioinformatics methods can be used to determine the location and structure of the transmembrane region in proteins. For example, BLAST programs or CLUSTAL W programs can be used to perform alignments and amino acid sequence comparisons routinely in the art. Based on the comparison with G protein-coupled receptors known to contain transmembrane regions, those skilled in the art can predict the location and structure of the transmembrane regions of other GPCRs. There are many programs that can be used to predict the location and structure of transmembrane regions in proteins. For example, one or a combination of the following programs can be used: TMpred, which predicts transmembrane protein fragments; TopPred, which predicts the topology of membrane proteins; PREDATOR, which predicts secondary structures from single and multiple sequences; TMAP, which predicts the transmembrane region of a protein from multiple aligned sequences; and ALOM2, which predicts a transmembrane region from a single sequence. According to standard nomenclature, the numbering of transmembrane regions and intracellular loops is relative to the N-terminus of GPCRs.

As used herein, the term “signal molecule” refers to any molecule (such as a polypeptide or protein) that can respond to a conformational change and convert the conformational change into a detectable signal (such as an optical signal or a chemical signal). In this context, the signaling molecule may exist independently or as part of a larger molecular structure (such as a fusion protein) to perform the function of its signaling molecule. In one embodiment, the signaling molecule is a molecule that emits light directly or indirectly in response to a conformational change. For example, the signal molecule may be a fluorescent protein or a luciferase, especially a circular permutated fluorescent protein (circular permutated FP, cpFP) or a circular permutated luciferase.

In a specific embodiment, the signaling molecule is a circular permutated fluorescent protein. Circular permutated fluorescent proteins are well-known to those skilled in the art, and means a protein obtained by connecting the nitrogen terminus (N-terminus) and carbon terminus (C-terminus) of the original fluorescent protein, and cleaving the resulting protein at any position to crease to form a new carbon and nitrogen terminus, thereby forming a fluorescent protein. The fluorescent protein itself has its own chromophore center composed of three amino acids, and the chemical reaction that occurs there determines the spectral properties and fluorescence intensity of the fluorescent protein. Through end changes, the chromophore of the circular permutated fluorescent protein is relatively close to the newly formed end. When it is connected to the target protein, the conformational change of the target protein will involve the end of the circular permutated fluorescent protein, resulting in a change in the surrounding environment of the chromophore, so that the fluorescent intensity of the fluorescent protein increases or decreases, thereby converting the conformational change of the target protein into the change of its fluorescence intensity, which can be detected in real time by optical imaging methods. The circular permutated fluorescent protein was originally derived from green fluorescent protein, and its amino acid sequence is very homologous to GFP. Roger Tsien first designed and applied the circular permutated green fluorescent protein in the process of developing genetically encoded calcium indicators (Baird, G. S., Zacharias, D. a. & Tsien, R. Y. Circular permutation and receptor insertion within green fluorescent proteins. Proceedings of the National Academy of Sciences of the United States of America 96, 11241-11246 (1999)) A variety of circular permutated fluorescent proteins have been constructed for the construction of sensors. They are highly sensitive to protein conformational changes, and can be used to characterize the conformational changes through changes in fluorescence. The cpFP useful in the present disclosure includes circular permutated enhanced green fluorescent protein (circular permutated EGFP, cpEGFP) and circular permutated red fluorescent protein (circular permutated RFP, cpRFP). cpEGFP can be cpEGFP from GCaMP6s or GCaMP6m (Chen, T.-W. et al. Ultrasensitive fluorescent proteins for imaging neuronal activity. Nature 499, 295-300 (2013)), or cpEGFP from GECO1.2 (Zhao, Y. et al. An Expanded Palette of Genetically Encoded Ca2+ Indicators. Science 333, 1888-1891 (2011)). The cpRFP may be cpmApple, such as cpmApple from R-GECO1 (Yongxin Zhao, et al, An Expanded Palette of Genetically Encoded Ca2+ indicators, Science, 2011). Their sequence can be obtained from NCBI database or addgene database. Those skilled in the art should understand that in the present disclosure, any other circular permutated fluorescent protein may also be used, including but not limited to, circular permutated green fluorescent protein, red fluorescent protein, infrared fluorescent protein, yellow fluorescent protein, blue Fluorescent proteins, such as circular permutated green fluorescent protein (cpGFP), circular permutated superfolder GFP, circular permutated mApple (cpmApple), circular permutated mCherry (cpmCherry), circular permutated mKate (cpmKate), circular permutated enhanced green fluorescent protein (cpEGFP), circular permutated Venus (cpVenus), circular permutated Citrin (cpCitrine), circular permutated enhanced yellow fluorescent protein (cpEYFP), and circular permutated infrared fluorescent protein (cp infrared fluorescent protein, cpiRFP, see Dania M Shcherbakova, et al, Near-infrared fluorescent proteins for multicolor in vivo imaging, Nature methods, 2013; Pandey N, et al, Tolerance of a Knotted Near-Infrared fluorescent protein to random circular permutation, Biochemistry, 2016), but not limited to the above cpEGFP and cpmApple. The excitation wavelength of cpiRFP is longer, so the fluorescent has better tissue penetration and is less affected by tissue autofluorescence.

In some embodiments of the invention, the circular permutated fluorescent protein cpEGFP from GCaMP6s is used, the specific sequence of which is:

(SEQ ID NO: 11)
NVYIKADKQKNGIKANFHIRHNIEDGGVQLAYHYQQNTPIGDGPVLLPDN
HYLSVQSKLSKDPNEKRDHMVLLEFVTAAGITLGMDELYKGGTGGSMVRK
GEELFTGVVPILVELDGDVNGHKFSVSGEGEGDATYGKLTLKFICTTGKL
PVPWPTLVTTLTYGVQCFSRYPDHMKQHDFFKSAMPEGYIQERTIFFKDD
GNYKTRAEVKFEGDTLVNRIELKGIDFKEDGNILGHKLEYN.

In some embodiments of the invention, the circular permutated fluorescent protein cpmApple is used, the specific sequence of which is:

(SEQ ID NO: 12)
PVVSERMYPEDGALKSEIKKGLRLKDGGHYAAEVKTTYKAKKPVQLPGAY
IVDIKLDIVSHNEDYTIVEQCERAEGRHSTGGMDELYKGGTGGSLVSKGE
EDNMAIIKEFMRFKVHMEGSVNGHEFEIEGEGEGRPYEAFQTAKLKVTKG
GPLPFAWDILSPQFMYGSKAYIKHPADIPDYFKLSFPEGFRWERVMNFED
GGIIHVNQDSSLQDGVFIYKVKLRGTNFPPDGPVMQKKTMGWEATR.

In another specific embodiment, the signal molecule is a circular permutated luciferase (cp luciferase). Using a similar principle, changes in the conformation of the receptor involve changes in the folding of luciferase, thereby changing the activity of its catalytic substrate.

For a specific G protein-coupled receptor, it is easy to experimentally determine the appropriate available circular permutated fluorescent protein. For example, whether the inserted circular permutated fluorescent protein is suitable can be determined by detecting whether the GRAB sensor can be correctly folded after inserting the circular permutated fluorescent protein and whether the fluorescence signal intensity can be changed after the GRAB sensor binds to its ligand.

In one embodiment, the sensor based on G protein-coupled receptor (ie, GRAB sensor) of the present disclosure can be expressed on the cell membrane. Methods for detecting whether the sensor can be expressed on the cell membrane are well known to those skilled in the art. For example, the sensor can be expressed in cells (such as HEK293T cells) and analyzed by the morphology of the cell expressing the fluorescent protein on the membrane, wherein the proteins expressed on the cell membrane will form a thin circle on the outermost periphery of the cell. By comparing the fluorescence channel with the bright field channel, one can obtain the cell contour for analyzing. Sensors that fail to properly be located on the cell membrane are often clustered in the cell, and show a cluster signal within the cell under the microscope. It can also be quantitatively measured by expressing another known cell membrane localized protein and calculating the co-localization of the fluorescent sensor signal with the protein.

In one embodiment, the sensor constructed based on the G protein-coupled receptor of the present disclosure can bind to the specific ligand of the G protein-coupled receptor, thereby causing detectable changes in the fluorescence intensity of the sensor. The detection method is known to those skilled in the art. For example, the sensor can be brought into contact with the specific ligand of the G protein-coupled receptor, and then the cells expressing the fluorescent sensor can be subjected to fluorescence imaging. Continuous photographing and recording are performed before and after the ligand is added, and whether the fluorescent sensor has a fluorescent response to the specific ligand is detected by analyzing changes in fluorescence intensity recorded before and after the ligand is added.

The term “detectable signal” as used herein refers to a signal value or a change in the signal value that can be detected by an appropriate detection means such as a light reaction or a chemical reaction, and the signal value or the change of the signal value is sufficient to be displayed by appropriate detection means. For the fluorescent signal (optical signal), the detectable signal means that the absolute value of the change in fluorescence intensity of GRAB sensor after ligand binding ΔF/F₀ is greater than or equal to 5%, greater than or equal to 10%, greater than or equal to 15%, greater than or equal to 20%, greater than or equal to 30%, greater than or equal to 40%, greater than or equal to 50%, greater than or equal to 60%, greater than or equal to 70%, greater than or equal to 80%, greater than or equal to 90%, greater than or equal to 100%, greater than or equal to 2 times, greater than or equal to 3 times, greater than or equal to 4 times, greater than or equal to 5 times or more changes. The change may be an increase in fluorescence intensity or a decrease in fluorescence intensity. The greater the change in fluorescence intensity, the better the properties of the sensor for intracellular detection.

The “change in fluorescence intensity of GRAB sensor after ligand binding ΔF/F₀” described herein refers to the relative change of the fluorescence intensity of GRAB sensor after ligand binding relative to that before ligand binding, where F₀ refers to the average fluorescence value of the GRAB sensor before ligand binding, and ΔF refers to the difference between the average fluorescence value F of the GRAB sensor after ligand binding and the average fluorescence value of the GRB sensor before ligand binding (ΔF=F−/F₀). In the present disclosure, ΔF may also be referred to as dF.

As used herein, when it is described that GPCR is connected to a signal molecule, the two can be connected in any suitable positional relationship, provided that the conformational change of GPCR can be converted into a detectable signal. For example, a signaling molecule (such as a circular permutated fluorescent protein or luciferase) can be connected to the intracellular region of GPCRs. In a specific embodiment, the signal molecule is connected to the C-terminus of GPCR, or inserted into the intracellular loop of GPCR, for example, inserted into the first intracellular loop, second intracellular loop or third intracellular loop of GPCR, especially into the third intracellular loop. Herein, the signal molecule and the GPCR may be directly connected, or indirectly connected via a linker sequence. In particular, whether directly or indirectly linking, one or more amino acids may be deleted at the linking position in the GPCR and/or signaling molecule.

The “ligand” or “specific ligand” of the G protein-coupled receptor described herein are used interchangeably and refer to a molecule capable of binding and activating (or inhibiting) the G protein-coupled receptor, including photosensitive compounds, odors, pheromones, hormones and neurotransmitters. The binding of G protein-coupled receptors to their ligands is highly specific, meaning that a ligand only binds to a specific receptor, and α receptor only bind to a specific ligand structure. The specificity of the binding of a G protein-coupled receptor to the ligand thereof means that the binding affinity of the G protein-coupled receptor to the ligand thereof is significantly higher than that to one or more other molecules. “Significantly” in the expression “significantly higher” may mean statistically significant. The ligands of different G protein-coupled receptors or the G protein-coupled receptors of different ligands are well known to those skilled in the art.

The ligand described in the present disclosure may be a natural ligand or a synthetic ligand. Natural ligands refer to molecules that naturally exist in the body and bind to G protein-coupled receptors in the body. Synthetic ligands refer to molecules that do not naturally exist in the body or bind to G protein-coupled receptors in the body. A synthetic ligand may be an analog of a natural ligand, may be an agonist or antagonist of G protein-coupled receptors, and may be used as a potential drug for activating or inhibiting G protein-coupled receptors.

In in some embodiments of the present disclosure, in the GRAB sensor, the third intracellular loop between the fifth transmembrane region and the sixth transmembrane region of the G protein-coupled receptor is truncated and a circular permutated fluorescent protein is inserted at the truncated position.

The “truncated” means that part of the sequence is deleted. “Truncated and a circular permutated fluorescent protein is inserted at the truncated position” refers to replacing the deleted sequence with a circular permutated fluorescent protein.

In in some embodiments of the present disclosure, the two ends of the circular permutated fluorescent protein are respectively connected to the third intracellular loop of the G protein-coupled receptor via a peptide linker.

As used herein, the term “peptide linker” or “linker peptide” can be used interchangeably, and refers to a short peptide that connects a G protein-coupled receptor (e.g., in a third intracellular loop) and a signal molecule (e.g., a circular permutated fluorescent protein). In the present disclosure, since the circular permutated fluorescent protein is inserted into the third intracellular loop of the G protein-coupled receptor, the “linker peptide” described herein includes an N-terminal linker peptide located at the N-terminus of the circular permutated fluorescent protein and the C-terminal linker peptide located at the C-terminus of the circular permutated fluorescent protein. In the present disclosure, the linker peptide serves to help the fusion protein to fold correctly, and at the same time serves as a bridge between the change in the conformation of the transfer receptor and the change in the brightness of the fluorescent protein. Therefore, the linker peptide used should have such roles. The selection of the linker peptide can be achieved by determining whether the GRAB sensor can be folded correctly and whether the binding of GRAB sensor to its ligand can cause the change of fluorescence signal intensity using various methods well known in the art. When the circular permutated fluorescent protein is inserted into the third intracellular loop of the G protein-coupled receptor, the N-terminus of the circular permutated fluorescent protein can be connected to the third intracellular loop through a N-terminal peptide linker, and the C-terminus of the circular permutated fluorescent protein can be connected to the third intracellular loop through a C-terminus peptide linker. In the present disclosure, it is allowed to express the peptide linker segment used in the sensor in the form of “N-terminal peptide linker-C-terminal peptide linker”.

In the present disclosure, the linker peptide may include or consist of flexible amino acids. The “flexible amino acid” is usually an amino acid with a smaller side chain and does not affect the conformation of the fusion protein. The flexible amino acid in the present disclosure may include glycine and alanine.

In the present disclosure, the amino acids constituting the linker peptide include but are not limited to flexible amino acids, and may also include other amino acids. Those skilled in the art can verify whether linker peptides composed of different amino acids are feasible through appropriate means.

In some embodiments of the present disclosure, the specific ligand is a neurotransmitter, including but not limited to epinephrine, norepinephrine, acetylcholine, serotonin, and/or dopamine. The G protein-coupled receptor is a G protein-coupled receptor capable of specifically binding to a neurotransmitter, such as but not limited to a G protein-coupled receptor specifically binding to epinephrine, norepinephrine, acetylcholine, serotonin, and/or dopamine.

There are two main types of adrenaline receptors, one is alpha receptors (such as human ADRA2A receptors), which have similar affinity for epinephrine and for norepinephrine. The other class is β receptors (such as human β2 adrenergic receptors), which have an affinity for epinephrine about 10-100 times higher than that of for norepinephrine. In the body, peripheral cardiovascular and other systems have functions mainly mediated by epinephrine, and the brain has functions mainly mediated by norepinephrine.

In one embodiment of the present disclosure, the circular permutated fluorescent protein and GPCR are constructed as a fusion protein. When a ligand molecule is combined with the GPCR, the conformation of the GPCR changes accordingly, thereby affecting the environment around the chromophore of the fluorescent protein, resulting in changes in fluorescence intensity, which can be detected in real time by optical imaging methods. Therefore, the fluorescence intensity changes of circular permutated fluorescent proteins can be used to indicate the binding of ligands (e.g., exogenous neurotransmitters) to GPCRs, thereby indicating changes in ligand concentration. In the present disclosure, the sensor is named GRAB sensor, which is the abbreviation of GPCR Activation Based Sensor. Since most known neurotransmitters have corresponding specific GPCRs, the fusion protein constructed by the circular permutated fluorescent protein of the present disclosure and GPCR can be used as a sensor for detecting neurotransmitters; in addition, the present disclosure sensors can also be used to detect other GPCR ligands.

In some embodiments of the present disclosure, the fluorescent sensor further coupled with a Gα peptide segment at the C-terminus can successfully compete for binding to endogenous G proteins, thereby significantly reducing the coupling of G protein signaling pathways, so that the GRAB sensor expressed in the cell will not cause obvious disturbance of the cell signaling system.

The “Gα peptide segment” used herein refers to the sequence of 20 amino acids at the C-terminus of the G protein, and belongs to the α subunit of the G protein. The a subunit of G protein includes three major categories: αs, αi, and αq. The “Gαq, Gαs, Gαi” and “Gq, Gs, Gi” described herein are used interchangeably. The Gα peptide segment may be the sequence of 20 amino acids at the C-terminus of any G protein. In some preferred embodiments, the Gα peptide segment may have the following sequence: VFAAVKDTILQLNLKEYNLV (Gαq20, SEQ ID NO:6). In other preferred embodiments, the Gα peptide segment may have the following sequence: VFNDCRDIIQRMHLRQYELL (Gαs20, SEQ ID NO:7). In other preferred embodiments, the Gα peptide segment may have the following sequence: VFDAVTDVIIKNNLKDCGLF (Gαi20, SEQ ID NO:8).

In some embodiments of the present disclosure, a luciferase is further inserted at the C-terminus of the fluorescent sensor. When a ligand binds to the GRAB sensor, the structure of the receptor will change. Such structural change will change the spatial distance and relative position of the luciferase located at the C-terminus and the circular permutated fluorescent protein located at the third intracellular loop, thus change the efficiency of resonance energy transfer between them, and in turn change and the fluorescent signal of the fluorescent protein. Therefore, the fluorescent sensor can be imaged without external excitation light.

The term “luciferase” as used herein refers to an enzyme capable of oxidizing luciferin (a naturally occurring fluorophore) to emit light energy. Those skilled in the art are familiar with a variety of different luciferase and luciferin/luciferase systems. Luciferase useful in the present disclosure includes but is not limited to Nanoluc, firefly luciferase (FLuc), Renilla luciferase (RLuc).

Luciferases useful in the present disclosure refer to those luciferases whose wavelength of light emitted when catalyzing the substrate luciferin is close to the excitation wavelength of the circular permutated fluorescent protein in the GRAB sensor of the invention. For different circular permutated fluorescent proteins with different excitation wavelengths, different luciferases can be used. For example, the excitation wavelength of cpEGFP in the present disclosure is 488 nm, so when cpEGFP is used in the GRAB sensor, useful luciferase includes Renilla luciferase, which uses coelenterazine as a substrate and emits light at 480 nm; and Gaussia luciferase, which uses coelenterazine as a substrate and emits light at 470 nm.

Without being limited to any theory, by analyzing the crystal structure of GPCR before and after the activation process, it is speculated that the conformational change of GPCR may be divided into two steps. The first step is the conformational change of the receptor transmembrane region (such as the fifth and sixth transmembrane regions) caused by ligand binding, and the second step is the opening of the intracellular loop caused by the conformational change of the transmembrane region of the receptor, thereby exposing the binding region of the G protein. For different receptors, their specific ligands binding causes different degrees of conformational changes in the transmembrane domain (Kruse, A. C. et al. Activation and allosteric modulation of a muscarinic acetylcholine receptor. Nature 504, 101-106 (2013); Wacker, D. et al. Structural features for functional selectivity at serotonin receptors. Science (New York, N.Y.) 340, 615-619 (2013)). However, since there are only a few types of endogenous G proteins, the conformational change of the intracellular loop is likely to have high homology (Rasmussen, S. G. F. et al. Crystal structure of the β2 adrenergic receptor-Gs protein complex. Nature 477, 549-555 (2011); Kruse, A. C. et al. Structure and dynamics of the M3 muscarinic acetylcholine receptor. Nature 482, 552-556 (2012); Huang, W. et al. Structural insights into micro-opioid receptor activation. Nature 524, 315-321 (2015)). Therefore, in the present disclosure, based on the conservativeness of the conformational changes of the intracellular loop, it is possible to replace the corresponding intracellular loops of other receptors with the intracellular loops of GPCR receptors that have been able to successfully induce changes in the brightness of fluorescent proteins, while keeping the region of the receptor that binds to the foreign ligand unchanged. By constructing chimeric receptors, on the one hand, the good coupling between the receptor and the fluorescent protein can be used to expand the sensor to sense different neurotransmitters. On the other hand, due to the different degree of conformational changes caused by the binding of different receptors and ligands, it can improve the signal-to-noise ratio of the sensor as a natural screening library. Therefore, the present disclosure also provides a method for constructing GRAB sensors, including replacing the third intracellular loop of the second G protein-coupled receptor with the whole third intracellular loop along with the circular permutated fluorescent protein inserted therein from the first G protein-coupled receptor, to obtain a sensor constructed based on the second G protein-coupled receptor.

As described above, those skilled in the art can easily determine the N-terminus, transmembrane region, intracellular loop, and C-terminus of G protein-coupled receptors.

In some embodiments, the first G protein-coupled receptor and the second G protein-coupled receptor may bind the same specific ligand or different specific ligands.

The present disclosure also relates to a polynucleotide encoding the GRAB sensor of the present disclosure, an expression vector containing the polynucleotide, and a host cell containing the polynucleotide or the expression vector.

The term “expression vector” refers to an expression vector capable of expressing a protein of interest in a suitable host cell, and is a gene construct that contains an operably linked basic regulatory element enable expression of the inserted gene. Preferably, a recombinant vector is constructed to carry the polynucleotide encoding the GRAB sensor of the present disclosure or a fragment thereof. The recombinant vector can be transformed or transfected into host cells.

The expression vector of the present disclosure can also be obtained by linking (inserting) the polynucleotide of the present disclosure to an appropriate vector. There is no particular restriction on the vector into which the gene of the present disclosure will be inserted, as long as it can replicate in the host. For example, plasmid vectors, phage vectors, viral vectors, etc. can be used. Specifically, commercial expression vectors, such as pDisplay vectors, available from Invitrogen may be used. In addition, animal viruses such as retroviruses, adenoviruses and vaccinia viruses and insect viruses such as baculoviruses can be used. The plasmids useful in the present disclosure are not limited to the examples indicated.

In order to operably link the polynucleotide of the present disclosure to a vector, in addition to the promoter and the polynucleotide of the present disclosure, the vector of the present disclosure may also include cis elements such as enhancers, splicing signals, Poly A addition signal, selectable marker, and ribosome binding sequence.

The constructed vector can be transformed (or transfected) into the host cell. Any method can be used for conversion, and generally, the transformation methods include: CaCl₂ precipitation method; electroporation method; calcium phosphate precipitation method; protoplast fusion method; silicon carbide fiber-mediated transformation method; Agrobacterium-mediated transformation method; PEG-mediated transformation method; dextran sulfate, lipofectamine and drying/inhibition transformation method, etc. By the above-mentioned vector and transfection using the vector, the polynucleotide vector encoding the GRAB sensor of the present disclosure can be introduced into the host cell.

The host cell used in the present disclosure is not particularly limited as long as it can express the GRAB sensors. In a preferred embodiment, the host cell is HEK293T. In other preferred embodiments, the host cell is a neuronal cell.

The invention also provides a method for detecting whether a specific ligand of the G protein-coupled receptor exists in a test sample or test tissue using GRAB fluorescent sensors, a method for qualitatively detecting the concentration change of the specific ligand of the G protein-coupled receptor in the test sample or the test tissue using GRAB fluorescent sensors, a method for quantitatively detecting the concentration change of the specific ligand of the G protein-coupled receptor in the test sample or the test tissue using GRAB fluorescent sensors, a drug screening method, and a method for detecting the distribution of specific ligands of G protein-coupled receptors in animals. In these detection methods, it is necessary to measure the change of the fluorescence signal intensity, thereby obtaining the detection result or the screening result.

It should be understood that the detection method of the present disclosure is used to determine whether the ligand or agonist is present and whether the ligand or agonist concentration is changed by detecting the change in the fluorescence intensity of the fluorescent sensor. The change in fluorescence intensity may be an increase or decrease in fluorescence intensity. According to the disclosure of the present disclosure, those skilled in the art known how to determine whether the ligand or agonist is present or whether the ligand or agonist concentration has changed according to the increase or decrease in fluorescence intensity. For example, if the obtained fluorescent sensor is an ON sensor, that is, a sensor whose fluorescence signal is enhanced after adding the ligand thereof, then during the detection process, when the fluorescence intensity increases, it can be judged that a ligand or an agonist is present, or the concentration of the ligand or the agonist increases; when the fluorescence intensity does not change, it can be judged that there is no ligand or agonist, or the concentration of the ligand or agonist does not change; when the fluorescence intensity decreases, it can be judged that there is no ligand or agonist, or the concentration of the ligand or the agonist decreases. If the obtained fluorescent sensor is an OFF sensor, that is, a sensor whose fluorescence signal is weakened after adding the ligand thereof, then during the detection process, when the fluorescence intensity decreases, it can be judged that a ligand or an agonist is present, or the concentration of the ligand or the agonist increases; when the fluorescence intensity does not change, it can be judged that there is no ligand or agonist, or the concentration of the ligand or agonist does not change; when the fluorescence intensity increases, it can be judged that there is no ligand or agonist, or the concentration of the ligand or the agonist decreases.

The “change in fluorescence signal intensity” in the present disclosure may refer to the change in fluorescence signal intensity ΔF/F₀ greater than or equal to 5%, greater than or equal to 10%, greater than or equal to 15%, greater than or equal to 20%, greater than or equal to 30%, greater than or equal to 40%, greater than or equal to 50%, greater than or equal to 60%, greater than or equal to 70%, greater than or equal to 80%, greater than or equal to 90%, greater than or equal to 100%, greater than or equal to 2 times, greater than or equal to 3 times, greater than or equal to 4 times, greater than or equal to 5 times or even greater change. The change may be an increase in fluorescence intensity or a decrease in fluorescence intensity.

The “change in fluorescence signal intensity ΔF/F₀” described in the present disclosure may refer to the relative change of the fluorescence intensity after the change relative to that before the change, where F₀ refers to the average fluorescence value before the change, and ΔF refers to the difference between the average fluorescence value F after the change and the average fluorescence value F₀ before the change (ΔF=F−/F₀).

The “test sample” described herein may include samples in vitro of living organisms, including but not limited to cell cultures or extracts thereof; biopsy materials or extracts thereof obtained from mammals; and blood, saliva, urine, Feces, semen, tears or other body fluids or extracts thereof. The detection of the test sample can be performed in vitro.

The “test tissue” described herein may include any tissue in a living body, including but not limited to heart tissue, brain tissue, and the like. The detection of the tissue to be tested may be performed in vivo.

In the present disclosure, the human muscarinic acetylcholine receptor M3R subtype is also referred to as human acetylcholine receptor M3R subtype, M3R receptor, M3R type receptor, M3R or M₃R, CHRM3, chrm3, and the like.

In the present disclosure, serotonin is also called 5-hydroxytryptamine.

The methods of any one of the technical solutions of the present disclosure can be performed in vitro or in vivo.

The methods of any one of the technical solutions of the present disclosure may be non-therapeutic.

It should be understood that the fluorescent sensor of the present disclosure may be a GPCR with inserted circular permutated fluorescent protein at different positions of the third intracellular loop. The two ends of the inserted fluorescent sensor may be connected to the third intracellular loop of GPCR through different peptide linkers, and the circular permutated fluorescent protein used may be various circular permutated fluorescent proteins. Therefore, in the present disclosure, different circular permutated fluorescent proteins, different insertion positions at the third intracellular loop, and different peptide linker may be combined with each other, and the resulting various combinations of solutions are within the scope of the present disclosure.

In addition, it should be understood that in the present disclosure, when referring to a numerical value or range, the term “about” means within 20%, within 10%, or within 5% of the given numerical value or range.

Abbreviations used in the present disclosure include:
GPCR G protein-coupled receptor
EGFP Enhanced green fluorescent protein
GFP Green fluorescent protein
YFP Yellow fluorescent protein
RFP Red fluorescent protein
CFP Blue fluorescent protein
cp Circular permutated (may followed by the abbreviation of fluorescent protein, for example, cpEGFP is circular permutated enhanced green fluorescent protein)
Epi Epinephrine
NE Norepinephrine
ISO Isoproterenol
Ach Acetylcholine
ICI or ICI118,551 β2-adrenoceptor specific blocker
Tio Tiotropium bromide
AF-DX384 or AF-DX Muscarinic acetylcholine receptor antagonist
5-HT Serotonin
GABA γ-Aminobutyric Acid
DA Dopamine
Gly Glycine
Glu Glutamate
ACSF Artificial cerebrospinal fluid
PTX Pertussis toxin
DAN Dopaminergic neurons
MB Mushroom body

### EXAMPLES

### Example 1: Materials and Methods

### 1. GRAB Sensor Construction, Mutation Screening and Cloning

In the present disclosure, the molecular cloning method of Gibson assembly (Gibson, D. G. et al. Enzymatic assembly of DNA molecules up to several hundred kilobases. Nature methods 6, 343-345 (2009)) is used, which uses sequence complementation to achieve the recombination of homologous fragments. An about 30 bases-homologous complementary sequence, which was designed on the primers, was used to achieve efficient splicing between sequences. All correctly recombined clones were confirmed by sequencing at the equipment center of the School of Life Sciences, Peking University.

The pDisplay vector from Invitrogen was used as the expression vector for GRAB sensors. The GPCR genes thereof were partially amplified from the full-length human cDNA (hORFeome database 8.1). It was first cloned to a pDisplay vector with att sequence by Gateway cloning method, and then a specific circular permutated fluorescent protein was inserted into a specific position of the receptor by Gibson assembly method. The different fluorescent proteins used in the optimization of the GRAB sensor were amplified in their corresponding fusion proteins, of which G-GECO (see Yongxin Zhao, et al, An Expanded Palette of Genetically Encoded Ca2+ indicators, Science, 2011) was provided by Professor Robert Campbell, ASAP1 (see Francois St-Pierre, et al, High-fidelity optical reporting of neuronal electrical activity with an ultrafast fluorescent voltage sensor, Nature Neuroscience, 2014) was provided by Professor Michael Lin, and GCaMP6 (Chen T W, et al, Ultrasensitive fluorescent peoteins for imaging neuronal activity, nature, 2013) was obtained from GCaMP5 mutation by our laboratory according to the literatures. During the mutation screening of the sensor, the method of introducing mutations was used to introduce random base combinations into specific primers to construct a site-directed mutation library.

GPCR gene sequences are available from the NCBI database and the Addgene database at the following URL:

NCBI:https://www.ncbi.nlm.nih.gov/

Addgene: https://www.addgene.org/

The method of constructing a chimeric sensor comprises amplifying a fragment of a specific receptor that does not contain the third intracellular loop and a fragment of the third intracellular loop of the GRAB sensor by PCR amplification, and then performing the sequence splicing by Gibson assembly method to construct chimeric sensor. For different GPCRs, the sequence prediction of the third intracellular loop thereof was performed based on the UNIPROT database.

The sensor constructed based on receptor endocytosis was constructed by pDisplay vector. Specifically, the pHluorin gene was connected to the N-terminus of the GPCR gene by Gibson assembly method, and a short peptide segment of 3 amino acids (GGA) was used to connect them to ensure the correct folding of the molecule. To further enhance the coupling of the GPCR to the endocytosis pathway, the last 29 amino acids (343-371 amino acids) of the human AVPR2 gene were fused at the C-terminus of the GPCR. This portion has been shown to have a high affinity for β-arrestin, and thus can enhance the coupling of GPCR to the endocytosis signaling pathway.

GRAB sensor plasmid for transgenic Drosophila was constructed by cloning a full-length GRAB sensor into a Drosophila expression vector pUAST vector, which contains a UAS sequence that can be regulated by the transcription factor Gal4. After a large amount of GRAB sensor transgenic Drosophila vector was extracted, it was injected into the Drosophila embryo and the screening of the transgenic Drosophila was performed by Fungene Biotechnology Company.

### 2. Cell Culture and Transfection

The screening and optimization of GRAB sensors were performed in the HEK293T cell line. HEK293T cells were cultured in DMEM (Gibco) medium containing 10% FBS (North TZ-Biotech Develop, Co. Ltd) in incubator containing 5% carbon dioxide at 37° C. According to the growth status and density of the cells, the cells were passaged every two days and a quarter of the cells were remained at each passage for cell culture. For plasmid transfection and imaging experiments on HEK293T cells, firstly round cover slides were placed in the wells of a 24-well plate, and then the cells after trypsin treatment were spread evenly on the slides. In order to ensure the uniformity of cells, the cells in the wells were mixed by horizontal shaking after the cells were seeded. Plasmid transfection was performed 8-12 hours after the cells were set into the plate, ensuring that the cells were tightly attached to the bottom of the slide and stretched. For HEK293T cells, PEI-mediated plasmid transfection method was used. Specifically, DNA and PEI were mixed in DMEM solution at the ratio of DNA:PEI=1:4, and the resulting solution was added into the cell medium in the well to be transfected after standing at room temperature for 15 minutes. After 4 hours of transfection, the cell medium was exchanged with DMEM medium containing 5% FBS, and PEI was washed away to ensure that the cells were in good condition. The expression and imaging of the GRAB sensor was performed about 36 hours after transfection.

Newborn Sprague-Dawley rats were used for the primary culture of rat neurons. After the skin of rat was cleaned with alcohol, the head was dissected with surgical instruments, the brain was removed out and the vascular membrane on the surface of the cortex was carefully removed. The cortical tissue was cut into pieces and placed in a 0.25% trypsin solution, and digested for 10 minutes in an incubator at 37° C. After the digestion, the digestion was terminated with DMEM medium containing 5% FBS, and the cells were further isolated by slowly pipetting up and down ten times with a pipette. After standing for 5 minutes, the pellet containing the tissue debris was discarded and the upper layer solution was collected and centrifuged at 1,000 rpm for 5 minutes in a centrifuge. Thereafter, the supernatant was discarded and the neurons were resuspended with the Neurobasal+B27 medium which was used for culturing the neurons. The cell density was calculated using a cell counting chamber. After that, the cells were diluted at a density of 0.5-1×10⁶ cells/ml and plated on the slides coated with poly-lysine (purchased from Sigma). Primary neurons were cultured in the Neurobasal+B27 medium, and a half of the medium was exchanged every two days. Primary cultured neurons were transfected 6-8 days after dissection using calcium phosphate transfection method. 1.5 hours after the transfection, the medium was observed under a microscope to see whether small and uniform calcium phosphate precipitates were produced, and the medium was exchanged with a HBS solution of pH 6.8. After HBS washing, neurons were re-cultured in Neurobasal+B27 medium, and imaging experiments were performed after 48 hours.

### 3. Fluorescence Imaging and Drug Perfusion

After DNA encoding specific GRAB sensor was introduced into cells by transfection, fluorescence imaging combined with perfusion experiment was. The imaging capture of HEK293T cells was performed using an Olympus IX81 inverted microscope, 40×NA: 1.35 oil lens, 475/28 excitation light filter, and 515LP emission light filter. The optical signal was collected by a Zyla sCMOS DG-152V-Cle FI camera (Andor), and Lambda DG-4 from Sutter Instuments company was used as a fluorescent light source. The exposure time was set to less than 50 ms and the acquisition frequency was 5 seconds. The entire imaging system was overall controlled by micromanager software.

The images of neurons were captured by an inverted Nikon laser scanning confocal microscope, which based on inverted Ti-E microscope and AlSi spectral detection confocal system. A 40× NA:1.35 oil lens and a 488 laser were used for imaging capture. The microscope body, PMT, and image acquisition and processing system of the laser scanning confocal microscope were controlled by NIS element software.

The detection of the response of the GRAB sensor to the ligand (ΔF/F₀) was performed using a drug perfusion method. The cells were placed in a standard physiological solution, and the formula thereof is:

| | | |
|---|---|---|
| NaCl | 150 | mM |
| KCl | 4 | mM |
| MgCl₂ | 2 | mM |
| CaCl₂ | 2 | mM |
| HEPES | 10 | mM |
| Glucose | 10 | mM |

The pH of the physiological solution was adjusted to about 7.4, and the small molecule drug was diluted with a part of the physiological solution to formulate solutions of respective concentrations of the small molecule ligand. Among the small molecule drugs, isoproterenol (ISO), IC1118551, and AF-DX were purchased from Sigma, acetylcholine was purchased from Solarbio, and Tiotropium Bromide was purchased from Dexinjia company. Unless explicitly specified, the concentration of isoproterenol (ISO) for perfusion was 2 μM, and the concentration of acetylcholine for perfusion was 100 μM.

The perfusion system was equipped on a microscope, including a solution-introduced system made of syringe for injection, a multi-way valve, an imaging workbench and a suction pump. During the perfusion process, the imaging workbench was placed above the objective lens of the inverted microscope, and then the slide on which cell plated were placed in the workbench. Perfusion of different drugs were performed by controlling the switches of different pipes. In order to control the level of the solution in the workbench, the perfusion speed was set to one drop or so per second. After each drug treatment, the cells were washed with a physiological solution for more than five minutes to ensure that there were no residual drugs that will affect subsequent experiments. After the end of each experiment, the perfusion pipe and the workbench were cleaned with 75% ethanol three times to ensure that the residual drugs and impurities were fully washed away.

For the detection of GRAB sensor kinetics, a local drug delivery system was used for the experiment. The experiment was performed using an Olympus upright microscope BX51, wherein a 40×NA: 0.80 water lens was used for imaging, and a 710M camera (DVC) was used for image acquisition. The drug delivery system was controlled by ROE-200 (from Sutter instruments company), and the position of the drug delivery needle was controlled by the MPS-1 operating lever. For the dynamic characterization experiment of the sensor, the imaging was processed using a frequency of 50 HZ (with a resolution of 768×484 pixels) and a surface element partition of 2×2.

### 4. Detection of Sensor Performance Using a Fluorescence Microplate Reader

For GRAB sensors, curves corresponding to different neurotransmitter concentrations were measured using a fluorescence microplate reader (Safire2 Full Spectrum Scanner from TECAN). Cells were plated evenly in a 96-well plate coated with polylysine. Transfection was performed using PEI method. Before measuring the fluorescence signal, the cell medium was exchanged to the physiological solution to eliminate the interference of the medium on the fluorescence signal acquisition. Using 480 nm as the excitation wavelength and 520 nm as the emission wavelength, the fluorescence values before and after adding specific drugs were collected, respectively. In the experiment, a small amount of drug solution at a concentration of 100× of the final concentration was added to avoid changes in the fluorescence signal caused by changes in the level of the solution. When different sensors are detected and screened, experiments were repeated in 6 wells for each sensor, and the average was taken to reduce the fluctuation in fluorescence value due to noise.

### 5. Two-Photon Imaging of Living Drosophila

Drosophila were kept in a 25-degree incubator with a standard medium. After the UAS-GRAB transgenic Drosophila was crossed with the GH146-Gal4 strain, the Drosophila with a strong fluorescent signal were selected for odor treatment experiment. The adult Drosophila to be tested were transferred to a new culture tube within 0-2 days after emergence, and left at room temperature for 8-12 days. Before the imaging, the Drosophila were first fixed in a small dish, and then the square skull part near the eye was surgically removed to expose part of the brain. The adipose tissue and air sacs near the antennal lobes to be imaged were surgically removed to prevent interference with the fluorescent signal. In order to further reduce the degradation of image quality caused by the movement of Drosophila during the imaging, surgical forceps were used to cut the muscles underneath its halter. Throughout the dissection and imaging procedures, the brain of Drosophila was kept in a pre-chilled physiological solution, and the formula thereof is as follows:

| | | |
|---|---|---|
| NaCl | 108 | mM |
| KCl | 5 | mM |
| HEPES | 5 | mM |
| Trehalose | 5 | mM |
| Sucrose | 5 | mM |
| NaHCO₃ | 26 | mM |
| NaH₂PO₄ | 1 | mM |
| CaCl₂ | 2 | mM |
| MgCl₂ | 2 | mM |

Drosophila imaging was performed using an Olympus two-photon microscope, including Olympus BX61 WI microscope, 25×NA: 1.05 water lens, and Ti:Sapphire laser mode-locked laser for two-photon excitation. For GRAB sensor imaging experiments, the wavelength of the emission light was set to 950 nm in order to successfully excite the fluorescent protein to produce fluorescence. The odor molecule isoamyl acetate (IA) used for stimulating Drosophila was purchased from Sigma, and was diluted in mineral oil at a ratio of 1:100. In the experiment, the isoamyl acetate was further diluted 5-40 times by mixing with air. The air mixed with odor molecules was provided at a position about 1 cm away from the tentacles of the Drosophila through a cavity about 1 cm wide on the experimental table. The odor molecule of different concentrations was tested by controlling the airflow. Each imaging cycle was 17.8 seconds, of which 5 to 10 seconds was the time for providing specific odor molecules. After the imaging experiment for odor function, the imaging area was scanned layer by layer with high resolution setting to obtain the distribution information of the olfactory bulb, which was later identified based on the information of olfactory bulb distribution at the antennal lobe reported in the literature.

### 6. Image Data Processing

Fluorescence imaging data was processed using ImageJ software. For the performance of GRAB sensors in HEK293T cells and neurons, the fluorescence of entire cell body was selected as the data processing region. For living Drosophila imaging, fluorescence images on the same Z axis were analyzed by ImageJ software. The change in the fluorescence signal was indicated by its relative difference. The signal of background area without the expression of the sensor was first subtracted from the fluorescence signal, so as to obtain the real expression of the intensity of the fluorescent protein. Then, by calculating the fluorescence value F after adding the drug and the average fluorescence value F0 before adding, the relative fluorescence change ΔF/F=(F−F₀)/F₀ was obtained as the fluorescence response of the sensor to a specific drug. The change of ΔF/F₀ with time was further plotted by Origin 8.6 software.

### 7. Statistical Testing

The data shown in the figures are mean±standard error of the mean.

Unless explicitly indicated, the materials and methods described in this example were applicable to the following Examples 2-7. In other examples, when not explicitly mentioned, the materials and methods described in this example were used, unless they conflict with the materials and methods described in these examples.

### Example 2 Construction of Adrenaline-Specific and Acetylcholine-Specific Fluorescent Sensors

1. Cellular Membrane Localization of Fusion Polypeptide Constructed Based on the (32 Adrenergic Receptor with Fluorescent Protein Inserting into the Third Intracellular Loop

The adrenaline-specific fluorescent sensor was constructed based on human β2 adrenergic receptor ((Rasmussen, S. G. F. et al. Crystal structure of the β2 adrenergic receptor-Gs protein complex. Nature 477, 549-555 (2011); Cherezov, V. et al. High-Resolution Crystal Structure of an Engineered Human beta G protein-coupled receptor. Science 318, 1258-1265 (2007)). The fluorescent protein was the circular permutated fluorescent protein cpEGFP used in G-GECO.

For the sequence of human β2 adrenergic receptor, refer to NCBI gene ID: 154, and the link is https://www.ncbi.nlm.nih.gov/gene/154. The specific amino acid sequence of human β2 adrenergic receptor is:

(SEQ ID NO: 1)
MGQPGNGSAFLLAPNRSHAPDHDVTQQRDEVWVVGMGIVMSLIVLAIVFG
NVLVITAIAKFERLQTVTNYFITSLACADLVMGLAVVPFGAAHILMKMWT
FGNFWCEFWTSIDVLCVTASIETLCVIAVDRYFAITSPFKYQSLLTKNKA
RVIILMVWIVSGLTSFLPIQMHWYRATHQEAINCYANETCCDFFTNQAYA
IASSIVSFYVPLVIMVFVYSRVFQEAKRQLQKIDKSEGRFHVQNLSQVEQ
DGRTGHGLRRSSKFCLKEHKALKTLGIIMGTFTLCWLPFFIVNIVHVIQD
NLIRKEVYILLNWIGYVNSGFNPLIYCRSPDFRIAFQELLCLRRSSLKAY
GNGYSSNGNTGEQSGYHVEQEKENKLLCEDLPGTEDFVGHQGTVPSDNID
SQGRNCSTNDSLL.

Wherein the underlined part is the third intracellular loop.

The cpEGFP used in this example is cpEGFP from GCaMP6s, and the specific sequence thereof is:

(SEQ ID NO: 11)
NVYIKADKQKNGIKANFHIRHNIEDGGVQLAYHYQQNTPIGDGPVLLPDN
HYLSVQSKLSKDPNEKRDHMVLLEFVTAAGITLGMDELYKGGTGGSMVSK
GEELFTGVVPILVELDGDVNGHKFSVSGEGEGDATYGKLTLKFICTTGKL
PVPWPTLVTTLTYGVQCFSRYPDHMKQHDFFKSAMPEGYIQERTIFFKDD
GNYKTRAEVKFEGDTLVNRIELKGIDFKEDGNILGHKLEYN.

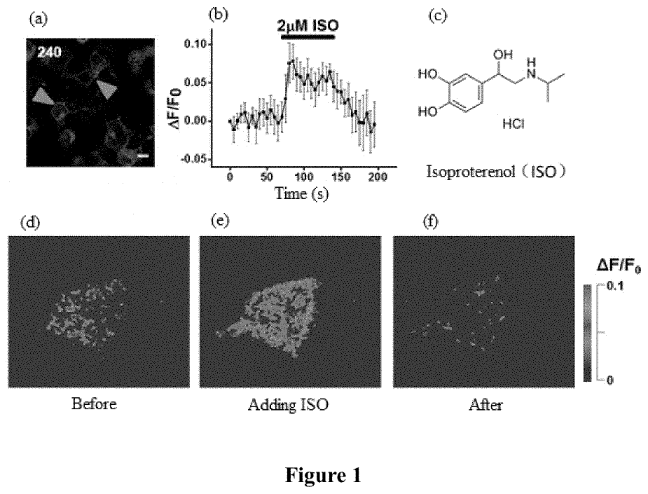

An expression vector expressing a fusion polypeptide in which the circular permutated fluorescent protein was inserted into the third intracellular loop of the human β2 adrenergic receptor was constructed, and then transfected into the HEK293T cells. First, the fluorescence intensity and membrane distribution of the fluorescence was observed to determine whether the GPCR was fused with circular permutated fluorescent proteins and the fusion protein can be efficiently localized on the cell membrane. The result was shown in FIG. 1a. The fusion polypeptide in which the circular permutated fluorescent protein was inserted into the third intracellular loop (amino acid position 240) of the human β2 adrenergic receptor showed good fluorescence intensity and cell membrane distribution.

### 2. The Sensor is Capable of Responding to Neurotransmitter and Generating Change in Optical Signal.

For detecting the fusion protein, a solution containing β2 adrenaline-specific agonist isoproterenol ISO was used to perfuse the HEK293T cells transfected with the fusion protein. Change in fluorescence intensity before and after the addition of the agonist was observed. The results show that the sensor generated a small but reversible fluorescence enhancement after adding 2 μM ISO, with an average change of about 6% (ΔF/F₀) (FIG. 1b-f). This fluorescent sensor is named GRAB-EPI 0.1, which is capable of detecting epinephrine and its analogs.

3. Different cpEGFP Circular Permutated Fluorescent Proteins have Similar Indication Function.

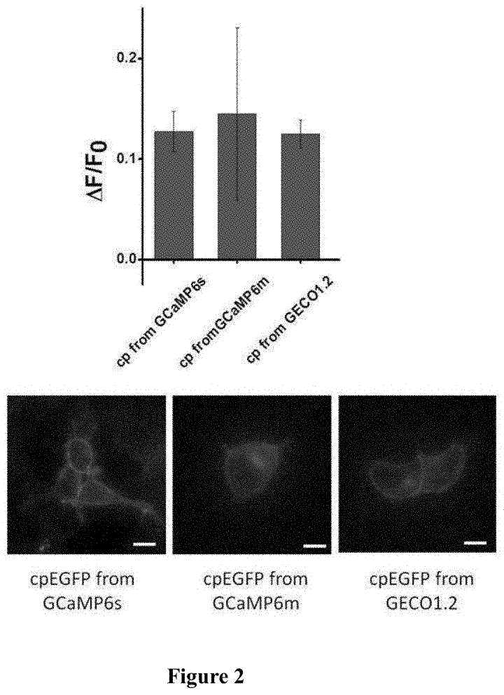

Different circular permutated fluorescent protein cpEGFP from GCaMP6s, GCaMP6m and GECO1.2 were cloned respectively (Chen, T.-W. et al. Ultrasensitive fluorescent proteins for imaging neuronal activity. Nature 499, 295-300 (2013)(GCaMP6s, GCaMP6m); Zhao, Y. et al. An Expanded Palette of Genetically Encoded Ca2+ Indicators. Science 333, 1888-1891 (2011) (GECO1.2). Their sequences are available from the NCBI database or the Addgene database. GECO1.2 is one version of G-GECO, and GCaMP6s/f/m are three different sub-versions of GCaMP6. These cpEGFPs were inserted into the human β2 adrenergic receptor after amino acid position 250. After construction, the fusion protein expression vectors were transfected into HEK293T cells. As shown in FIG. 2, the fluorescent sensors constructed with different cpEGFPs can be successfully folded and transported to the cell membrane, and show similar changes in fluorescence intensity when treated with the same concentration (2 μM) of agonist ISO.

### 4. Optimization of the Insertion Site of the Fluorescent Protein in the Third Intracellular Loop of the Human Adrenaline Receptor

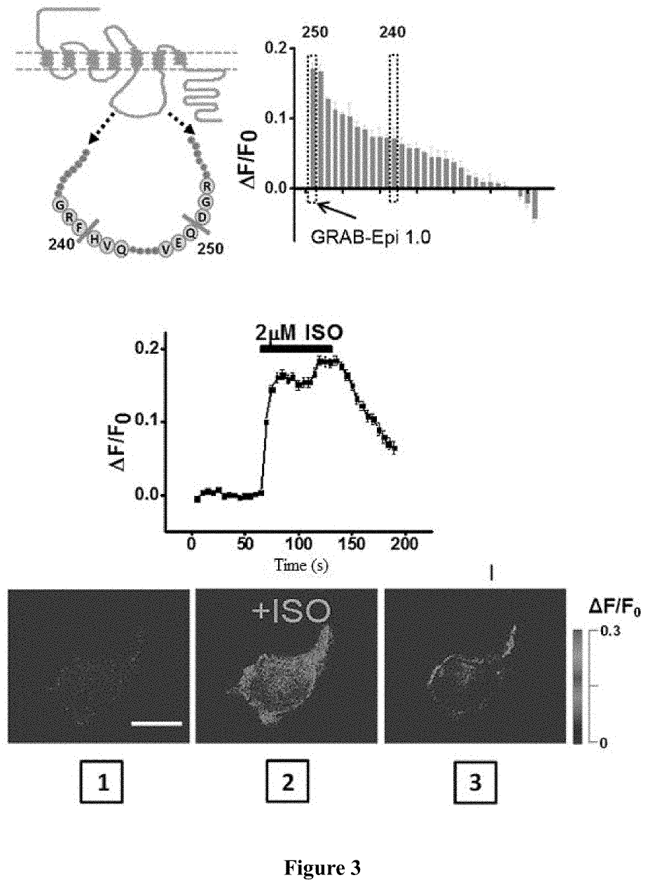

Circular permutated cpEGFP from GCaMP6s was inserted into the human β2 adrenergic receptor at different insertion sites in the third intracellular loop. The obtained fusion proteins were cloned and expressed in HEK293T cells. Perfusion was performed with the same concentration (2 μM) of agonist ISO, and the fluorescence changes before and after the addition of agonist were observed by fluorescence imaging. As shown in FIG. 3, the sensor constructed by inserting the fluorescent protein into the receptor at amino acid position 250 has a more significant increase in fluorescence intensity, which can achieve a 15% ΔF/F₀ fluorescence enhancement when treated with the same concentration (2 μM) of ISO. This sensor is named GRAB-EPI 1.0.

The amino acid sequence of GRAB-EPI 1.0 is as follows:

(SEQ ID NO: 13)
METDTLLLWVLLLWVPGSTGDYPYDVPDYAGAQPARSGQPGNGSAFLLAP
NGSHAPDHDVTQERDEVWVVGMGIVMSLIVLAIVFGNVLVITAIAKFERL
QTVTNYFITSLACADLVMGLAVVPFGAAHILMKMWTEGNEWCEFWTSIDV
LCVTASIETLCVIAVDRYFAITSPFKYQSLLTKNKARVIILMVWIVSGLT
SFLPIQMHWYRATHQEAINCYANETCCDFFTNQAYAIASSIVSFYVPLVE
VIVEVYSRVFQEAKRQLQKIDKSEGRFHVQNLSQVEQGGNVYIKADKQKN
GIKANFHIRHNIEDGGVQLAYHYQQNTPIGDGPVLLPDNHYLSVQSKLSK
DPNEKRDHMVLLEFVTAAGITLGMDELYKGGTGGSMVSKGEELFTGVVPI
LVELDGDVNGHKFSVSGEGEGDATYGKLTLKFICTTGKLPVPWPTLVTTL
TYGVQCFSRYPDHMKQHDFFKSAMPEGYIQERTIFFKDDGNYKTRAEVKF
EGDTLVNRIELKGIDFKEDGNILGHKLEYNGGAAADGRTGHGLRRSSKFC
LKEHKALKTLGIEVIGTFTLCWLPFFIVNIVHVIQDNLIRKEVYILLNWI
GYVNSGFNPLIYCRSPDFRIAFQELLCLRRSSLKAYGNGYSSNGNTGEQS
GYHVEQEKENKLLCEDLPGTEDFVGHQGTVPSDNIDSQGRNCSTNDSLL.

### 5. Construction of an Acetylcholine Fluorescent Sensor by Constructing a Chimeric Receptor

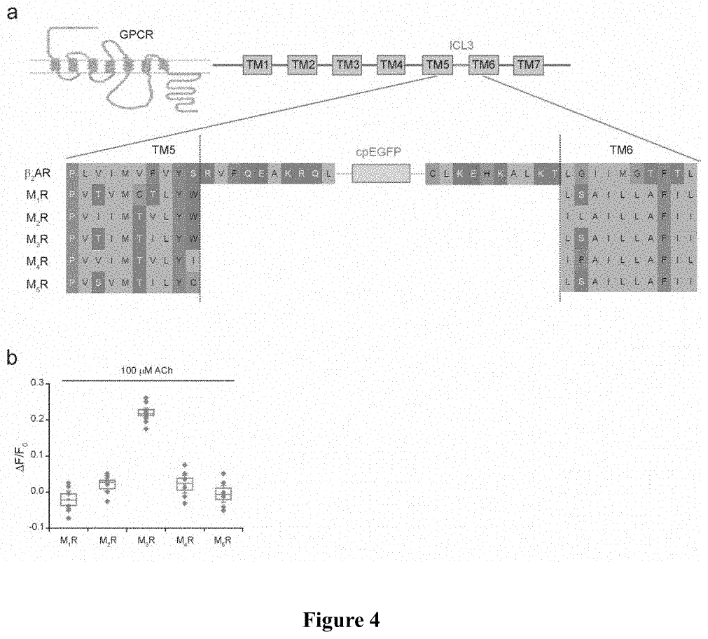

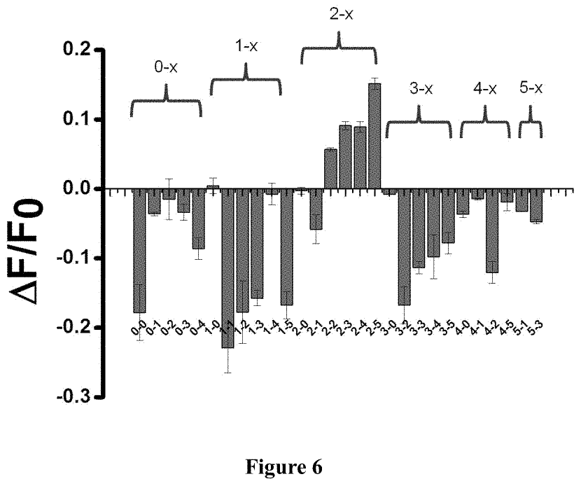

Based on the successfully constructed epinephrine fluorescent sensor GRAB-EPI 1.0, the third intracellular loop thereof was intercepted along with the inserted circular permutated fluorescent protein (FIG. 4) and replaced the corresponding third intracellular loop (ICL3) of the human muscarinic acetylcholine receptor (M₁₋₅R), so as to allow conformation-sensitive cpEGFP to be inserted into all five isoforms of the human muscarinic acetylcholine receptor (M₁₋₅R) (FIG. 6a) to construct chimeric fluorescent sensors M₁₋₅R-β₂R ICL3-cpEGFP of the corresponding neurotransmitter.

Wherein:

for the sequence of M₁R, refer to NCBI gene ID 1128;

for the sequence of M₂R, refer to NCBI gene ID 1129;

for the sequence of M₃R, refer to NCBI gene ID 1131;

for the sequence of M₄R, refer to NCBI gene ID 1132; and

for the sequence of M₅R, refer to NCBI gene ID 1133.

The specific sequence is:

RVFQEAKRQLQKIDKSEGRFHVQNLSQVEQGGNVYIKADKQKNGIKANFH
IRHNIEDGGVQLAYHYQQNTPIGDGPVLLPDNHYLSVQSKLSKDPNEKRD
HMVLLEFVTAAGITLGMDELYKGGTGGSMVSKGEELFTGVVPILVELDGD
VNGHKFSVSGEGEGDATYGKLTLKFICTTGKLPVPWPTLVTTLTYGVQCF
SRYPDHMKQHDFFKSAMPEGYIQERTIFFKDDGNYKTRAEVKFEGDTLVN
RIELKGIDFKEDGNILGHKLEYNGGAAADGRTGHGLRRSSKFCLKEHKAL
KT.

Wherein the underlined part is the inserted fluorescent protein sequence.

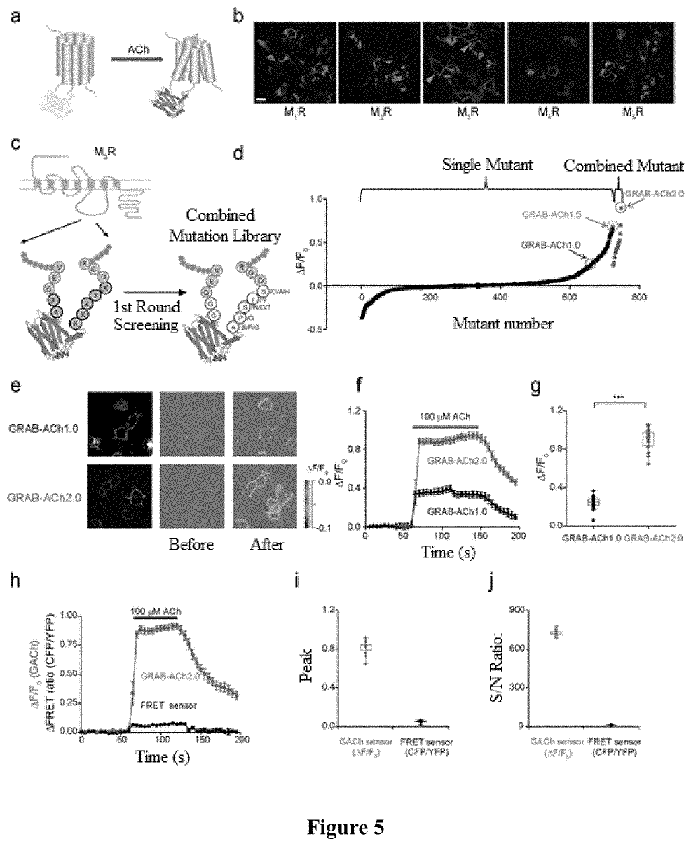

To test whether M₁₋₅R-β₂R ICL3-cpEGFP chimeras were able to detect acetylcholine ACh, they were expressed in HEK293T cells. M₃R-β₂R ICL3-cpEGFP showed good membrane expression (FIG. 5b, as indicated by the arrow) and showed an increase in fluorescence response (ΔF/F₀) (˜30%) when cells were perfused with ACh (100 μM) (FIG. 4b). These results indicate that M3R-derived sensor can detect ACh, and is named GRAB-ACh 1.0.

The specific sequence of the M3R receptor is as follows:

(SEQ ID NO: 3)
MTLHNNSTTSPLFPNISSSWIHSPSDAGLPPGTVTHFGSYNVSRAAGNFS
SPDGTTDDPLGGHTVWQVVFIAFLTGILALVTIIGNILVIVSFKVNKQLK
TVNNYFLLSLACADLIIGVISMNLFTTYIIMNRWALGNLACDLWLAIDYV
ASNASVMNLLVISFDRYFSITRPLTYRAKRTTKRAGVMIGLAWVISFVLW
APAILFWQYFVGKRTVPPGECFIQFLSEPTITFGTAIAAFYMPVTIMTIL
YWRIYKETEKRTKELAGLQASGTEAETENFVHPTGSSRSCSSYELQQQSM
KRSNRRKYGRCHFWFTTKSWKPSSEQMDQDHSSSDSWNNNDAAASLENSA
SSDEEDIGSETRAIYSIVLKLPGHSTILNSTKLPSSDNLQVPEEELGMVD
LERKADKLQAQKSVDDGGSFPKSFSKLPIQLESAVDTAKTSDVNSSVGKS
TATLPLSFKEATLAKRFALKTRSQITKRKRMSLVKEKKAAQTLSAILLAF
IITWTPYNIMVLVNTFCDSCIPKTFWNLGYWLCYINSTVNPVCYALCNKT
FRTTFKMLLLCQCDKKKRRKQQYQQRQSVIFHKRAPEQAL.

Wherein the underlined part (amino acids 253-491) is the third intracellular loop (ICL3) defined by referring to the uniprot database.

The sequence of the constructed GRAB-ACh1.0 is as follows:

(SEQ ID NO: 15)
METDTLLLWVLLLWVPGSTGDTSLYKKVGTMTLHNNSTTSPLFPNISSSW
IHSPSDAGLPPGTVTHFGSYNVSRAAGNFSSPDGTTDDPLGGHTVWQVVF
IAFLTGILALVTIIGNILVIVSFKVNKQLKTVNNYFLLSLACADLIIGVI
SMNLFTTYIIMNRWALGNLACDLWLAIDYVASNASVMNLLVISFDRYFSI
TRPLTYRAKRTTKRAGVMIGLAWVISFVLWAPAILFWQYFVGKRTVPPGE
CFIQFLSEPTITFGTAIAAFYMPVTIMTILYWRVFQEAKRQLQKIDKSEG
RFHVQNLSQVEQGGNVYIKADKQKNGIKANFHIRHNIEDGGVQLAYHYQQ
NTPIGDGPVLLPDNHYLSVQSKLSKDPNEKRDHMVLLEFVTAAGITLGMD
ELYKGGTGGSMVSKGEELFTGVVPILVELDGDVNGHKFSVSGEGEGDATY
GKLTLKFICTTGKLPVPWPTLVTTLTYGVQCFSRYPDHMKQHDFFKSAMP
EGYIQERTIFFKDDGNYKTRAEVKFEGDTLVNRIELKGIDFKEDGNILGH
KLEYNGGAAADGRTGHGLRRSSKFCLKEHKALKTLSAILLAFIITWTPYN
IMVLVNTFCDSCIPKTFWNLGYWLCYINSTVNPVCYALCNKTFRTTFKML
LLCQCDKKKRRKQQYQQRQSVIFHKRAPEQAL.

### 6. Optimization of Peptide Linker Between the Circular Permutated Fluorescent Protein and GPCR

There is a peptide linker between the circular permutated fluorescent protein cpEGFP and the third intracellular loop of the receptor. The peptide linker was an artificial peptide segment consisting of a few amino acids, which can help the fusion protein to fold correctly on the one hand, and on the other hand acts as a bridge between change in the conformation of the receptor and change in the brightness of the fluorescent protein. According to the design principle of the circular permutated fluorescent protein, the asparagine at position 145 of the original fluorescent protein was replaced with the peptide linker, which has close interaction with the chromophore (Baird, G S, Zacharias, Da & Tsien, R Y Circular permutation and receptor insertion within green fluorescent proteins. Proceedings of the National Academy of Sciences of the United States of America 96, 11241-11246 (1999)).

In the foregoing steps, in the construction of the adrenaline fluorescent sensor, flexible amino acids (glycine, alanine) were used in a short peptide linker to help the fusion protein to fold correctly. The length of the short peptide linker was 2 amino acids GG at the N-terminus and 5 amino acids GGAAA at the C-terminus. The peptide linker was cut from GRAB-EPI 1.0 together with ICL3 and transplanted into an acetylcholine fluorescent sensor.

Based on GRAB-ACh 1.0, this peptide linker was optimized. First, based on a flexible amino acid (glycine, alanine), the peptide segment was screened for optimal length. The specific strategy was to change the length of the peptide linkers at the N-terminus and C-terminus from 0 to 5 amino acids, respectively, and to randomly combine the N-terminus with C-terminus to obtain all possible permutations. Fusion protein expression vectors containing various arrangements were expressed in HEK293T cells and subjected to perfusion experiments. The results were shown in FIG. 6. The number of amino acids at the N-terminus has an important effect on deciding the increase or decrease of the fluorescence of the sensor after adding the ligand. The number of amino acids at the C-terminus affects the specific signal changes of the sensor. Specifically, the sensors can be divided into two types according to the fluorescence change after adding the ligand: one was the ON sensor with enhanced fluorescence signal after adding the ligand, and the other was the OFF sensor with decreased fluorescence after adding the ligand. According to the coupling principle of the fluorescent sensor and GPCR, it was speculated that the chromophore of the fluorescent protein in the ON sensor was partially exposed before the ligand was added, so it was quenched by water molecules and thus had lower fluorescence intensity before the ligand was added. However, after the conformation of the receptor is changed due to the addition of ligand, the movement of the intracellular loop will induce the refolding of the fluorescent protein, which gives the chromophore better protection, thereby enhancing the fluorescence emission intensity of the sensor. Accordingly, the mechanism of the OFF sensor may be reversed. In the screening results, the ON sensors were found only in sensors of which the length of the peptide linkers at the N-terminus was 2 amino acids, while OFF sensors were found in sensors of which the length of the peptide linkers at the N-terminus was 1, 3, 4, and 5 amino acids. As for the signal change of the sensor, in the ON sensor, the longer the amino acids of the peptide linker at the C-terminus, the higher the signal change; while in the OFF sensor, the shorter the amino acids of the peptide linker at the C-terminus, the higher the signal change. Combining the screening results of the peptide linkers at the N-terminus and the C-terminus, the optimum ON sensor was determined as a combination of the peptide linker length of 2-5 (i.e., GG at the N-terminus and GGAAA at the C-terminus), and the optimum OFF sensor was determined as a combination of the peptide determined length of 1-1 (i.e., G at the N-terminus and G at the C-terminus).

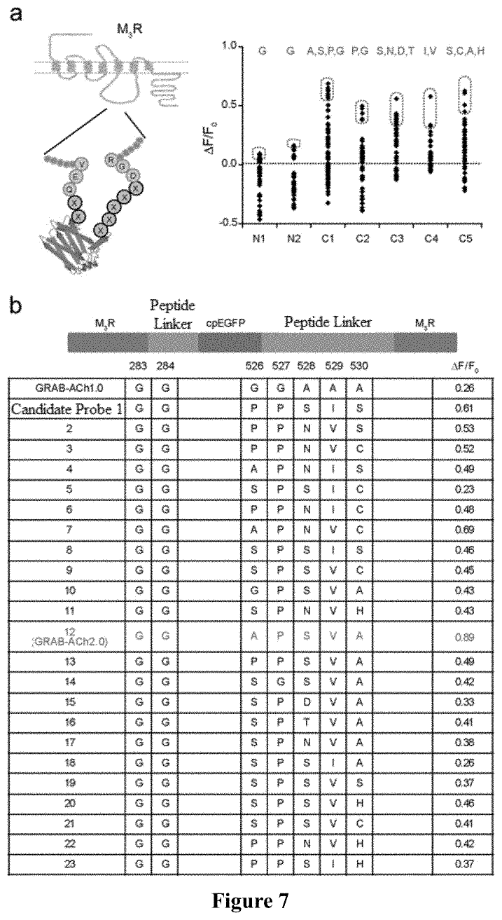

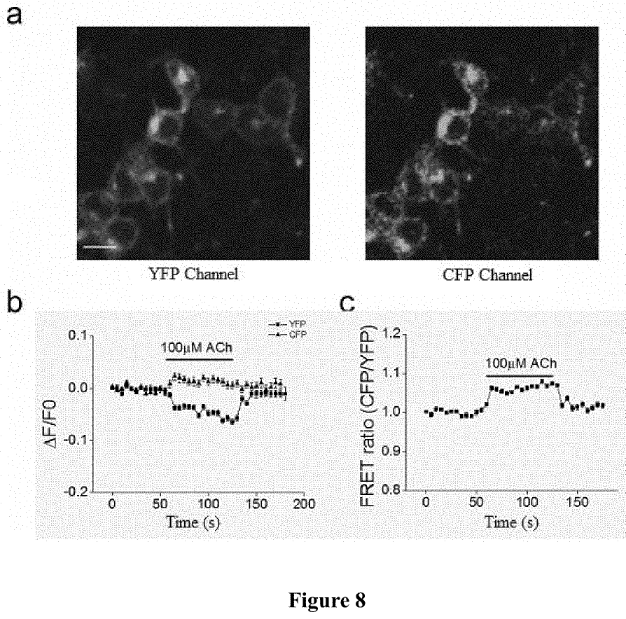

Next, the 2-5 combination of the length of the peptide linker was fixed, and then the amino acids of the sensor sequence were changed to screen for sensor with a greater signal change. Site-directed mutagenesis was used to generate a library of 723 random point mutations on the 2- and 5-amino acid peptide linkers at the N- and C-terminus of cpEGFP in GRAB-ACh 1.0 (FIGS. 5c and 7). These mutants were then expressed in HEK293T cells, respectively, and candidates were screened for the one with greater response (ΔF/F₀) to ACh perfusion. The variant identified as having the optimum ΔF/F₀ (˜70%) was screened out and named GRAB-ACh 1.5 (the peptide linker sequence was GG at N-terminus and SPSVA at C-terminus) (FIG. 5d). A second round of site-directed mutagenesis and screening was then performed, using a combination of the optimal peptide linker residues (FIGS. 5c and 7). After screening 23 variants, the variant with the largest increase in ΔF/F₀ during ACh perfusion was identified and named GRAB-ACh 2.0 (FIGS. 5c and 7), which used GG-APSVA as the peptide linkers. Further analysis showed that GRAB-ACh 2.0 retains excellent expression and membrane localization characteristics (FIG. 5e), and its kinetic range has been expanded (by 2.5 times) when compared to GRAB-ACh 1.0 (FIGS. 5f, g); and its peak signal response was increased when compared to the FRET-based sensor (GRAB-ACh 2.0: ΔF/F₀=94.0±3.0%, and the FRET-based sensor: ΔRatioFRET=6.6±0.4%, and GRAB-ACh 2.0 was ˜20 times higher); and its signal-to-noise ratio (SNR) was increased (˜60 times) when compared to the traditional FRET-based ACh sensors (Markovic, D., et al. FRET-based detection of M1 muscarinic acetylcholine receptor activation by orthosteric and allosteric agonists. PloS one 7, e29946 (2012)) (FIGS. 5i-j and FIG. 8).

Wherein the amino acid sequence of GRAB-ACh2.0 is as follows:

(SEQ ID NO: 16)
METDTLLLWVLLLWVPGSTGDTSLYKKVGTMTLHNNSTTSPLFPNISSSW
IHSPSDAGLPPGTVTHFGSYNVSRAAGNFSSPDGTTDDPLGGHTVWQVVF
IAFLTGILALVTIIGNILVIVSFKVNKQLKTVNNYFLLSLACADLIIGVI
SMNLFTTYIIMNRWALGNLACDLWLAIDYVASNASVMNLLVISFDRYFSI
TRPLTYRAKRTTKRAGVMIGLAWVISFVLWAPAILFWQYFVGKRTVPPGE
CFIQFLSEPTITFGTAIAAFYMPVTIMTILYWRVFQEAKRQLQKIDKSEG
RFHVQNLSQVEQGGNVYIKADKQKNGIKANFHIRHNIEDGGVQLAYHYQQ
NTPIGDGPVLLPDNHYLSVQSKLSKDPNEKRDHMVLLEFVTAAGITLGMD
ELYKGGTGGSMVRKGEELFTGVVPILVELDGDVNGHKFSVSGEGEGDATY
GKLTLKFICTTGKLPVPWPTLVTTLTYGVQCFSRYPDHMKQHDFFKSAMP
EGYIQERTIFFKDDGNYKTRAEVKFEGDTLVNRIELKGIDFKEDGNILGH
KLEYNAPSVADGRTGHGLRRSSKFCLKEHKALKTLSAILLAFITTWTPYN
IMVLVNTFCDSCIPKTFWNLGYWLCYINSTVNPVCYALCNKTFRTTFKML
LLCQCDKKKRRKQQYQQRQSVIFHKRAPEQAL.

The entire ICL3 of GRAB-ACh 1.5 and the entire ICL3 of GRAB-ACh 2.0 were inserted into GRAB-EPI 1.0 including the cpEGFP and peptide linkers therein, to replace the third intracellular loop and obtain GRAB-EPI 1.1 and GRAB-EPI 2.0. The expression vectors were then transfected into HEK293T cells. Then the drug perfusion experiments were conducted. GRAB-EPI 1.1 showed increase of the sensor fluorescence by about 60% after adding the agonist ISO (2 μM), and GRAB-EPI 2.0 showed increase of the sensor fluorescence by about 70% after adding the agonist ISO (2 μM).

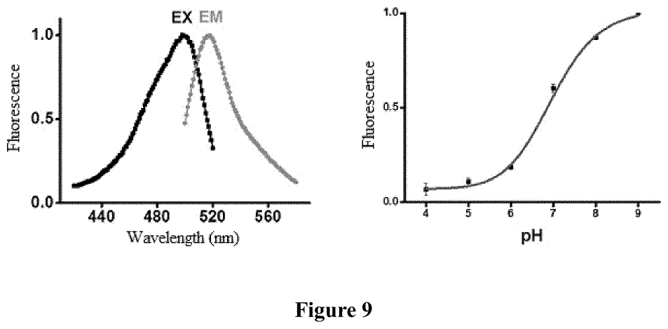

The spectral properties and pH sensitivity of fluorescent sensors were measured using HEK293T cells expressing GRAB-EPI 1.0. The main excitation peak was near 490 nm and the emission peak was near 520 nm. With the Triton treatment to achieve cell membrane permeable, different pH values of the external solution lead to a change in the brightness of the fluorescent protein with a pKa of about 7.0 (FIG. 9).

Wherein the sequence of GRAB-EPI 2.0 is as follows:

(SEQ ID NO: 14)
METDTLLLWVLLLWVPGSTGDYPYDVPDYAGAQPARSGQPGNGSAFLLAP
NGSHAPDHDVTQERDEVWVVGMGIVMSLIVLAIVFGNVLVITAIAKFERL
QTVTNYFITSLACADLVMGLAVVPFGAAHILMKMWTFGNFWCEFWTSIDV
LCVTASIETLCVIAVDRYFAITSPFKYQSLLTKNKARVIILMVWIVSGLT
SFLPIQMHWYRATHQEAINCYANETCCDFFTNQAYAIASSIVSFYVPLVE
VIVFVYSRVFQEAKRQLQKIDKSEGRFHVQNLSQVEQGGNVYIKADKQKN
GIKANFHIRHNIEDGGVQLAYHYQQNTPIGDGPVLLPDNHYLSVQSKLSK
DPNEKRDHMVLLEFVTAAGITLGMDELYKGGTGGSMVRKGEELFTGVVPI
LVELDGDVNGHKFSVSGEGEGDATYGKLTLKFICTTGKLPVPWPTLVTTL
TYGVQCFSRYPDHMKQHDFFKSAMPEGYIQERTIFFKDDGNYKTRAEVKF
EGDTLVNRIELKGIDFKEDGNILGHKLEYNAPSVADGRTGHGLRRSSKFC
LKEHKALKTLGIEVIGTFTLCWLPFFIVNIVHVIQDNLIRKEVYILLNWI
GYVNSGFNPLIYCRSPDFRIAFQELLCLRRSSLKAYGNGYSSNGNTGEQS
GYHVEQEKENKLLCEDLPGTEDFVGHQGTVPSDNIDSQGRNCSTNDSLL.

7. Cellular Membrane Localization of Fusion Polypeptides with Insertion of Fluorescent Protein at the C-Terminus of G Protein-Coupled Receptor and the Ability Thereof to Generate Optical Signal Change

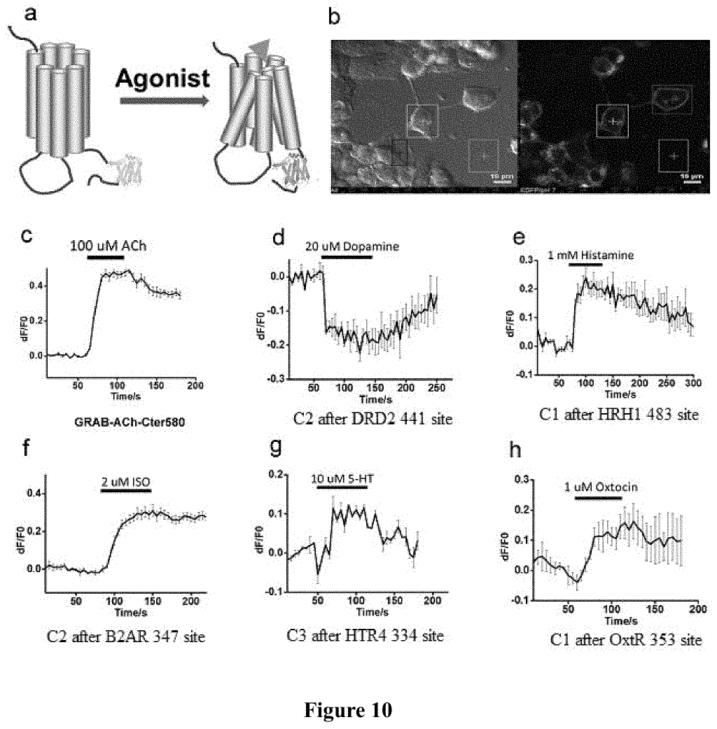

Based on the previously constructed acetylcholine fluorescent sensor GRAB-ACh2.0, the portion of the polypeptide linkers and cpEGFP in the third intracellular loop was inserted after the position 580 of the C-terminus sequence of the human muscarinic acetylcholine receptor M3R protein, and Gq20 peptide segment and TS and ER export sequences were connected to the end of the M3R protein to obtain a fusion peptide acetylcholine sensor GRAB-ACh-Cter580 with a fluorescent protein fused at the C-terminus. After transfecting the fusion polypeptide into the HEK293T cells, the results show that it has good fluorescence intensity and membrane fluorescence distribution (see FIGS. 10a and b), indicating that the fusion polypeptide can be effectively localized to the cell membrane and shows increased fluorescence response (ΔF/F₀) (˜40%) when perfused with ACh (100 μM) (FIG. 10c).

Next, the method of constructing a chimeric receptor was used to construct fusion polypeptide sensors with a fluorescent protein at the C-terminus of different G protein-coupled receptors. The three sites, K567, R568 and R569, at the C-terminus of GRAB-ACh-Cter580 were selected as the cutting sites and labeled as C1, C2 and C3, respectively. After cutting, the multiple C-terminus sequences containing fluorescent protein were fused to HRH1 (SEQ ID NO: 27), OxtR (SEQ ID NO: 29), DRD2, B2AR, and HTR4 (SEQ ID NO: 28) at specific positions, respectively. Changes in signal generated by these fusion polypeptides in perfusion experiments with corresponding receptor agonists were observed.

Wherein the amino acid sequence of HRH1 is as follows:

(SEQ ID NO: 27)
MSLPNSSCLLEDKMCEGNKTTMASPQLMPLVVVLSTICLVTVGLNLLVLY
AVRSERKLHTVGNLYIVSLSVADLIVGAVVMPMNILYLLMSKWSLGRPLC
LFWLSMDYVASTASIFSVFILCIDRYRSVQQPLRYLKYRTKTRASATILG
AWFLSFLWVIPILGWNHFMQQTSVRREDKCETDFYDVTWFKVMTAIINFY
LPTLLMLWFYAKIYKAVRQHCQHRELINRSLPSFSEIKLRPENPKGDAKK
PGKESPWEVLKRKPKDAGGGSVLKSPSQTPKEMKSPVVFSQEDDREVDKL
YCFPLDIEHMQAAAEGSSRDYVAVNRSHGQLKTDEQGLNTHGASEISEDQ
MLGDSQSFSRTDSDTTTETAPGKGKLRSGSNTGLDYIKFTWKRLRSHSRQ
YVSGLHMNRERKAAKQLGFIMAAFILCWIPYFIFFMVIAFCKNCCNEHLH
MFTIWLGYINSTLNPLIYPLCNENFKKTFKRILHIRS.

The amino acid sequence of HTR4 is as follows:

(SEQ ID NO: 28)
MDKLDANVSSEEGFGSVEKVVLLTFLSTVILMAILGNLLVMVAVCWDRQL
RKIKTNYFIVSLAFADLLVSVLVMPFGAIELVQDIWIYGEVFCLVRTSLD
VLLTTASIFHLCCISLDRYYAICCQPLVYRNKMTPLRIALMLGGCWVIPT
FISFLPIMQGWNNIGIIDLIEKRKFNQNSNSTYCVFMVNKPYAITCSVVA
FYIPFLLMVLAYYRIYVTAKEHAHQIQMLQRAGASSESRPQSADQHSTHR
MRTETKAAKTLCIIIVIGCFCLCWAPFFVTNIVDPFIDYTVPGQVWTAFL
WLGYINSGLNPFLYAFLNKSFRRAFLIILCCDDERYRRPSILGQTVPCST
TTINGSTHVLRDAVECGGQWESQCHPPATSPLVAAQPSDT.

The amino acid sequence of OxtR is as follows:

(SEQ ID NO: 29)
MEGALAANWSAEAANASAAPPGAEGNRTAGPPRRNEALARVEVAVLCLIL
LLALSGNACVLLALRTTRQKHSRLEFFMKHLSIADLVVAVFQVLPQLLWD
ITERFYGPDLLCRLVKYLQVVGMFASTYLLLLMSLDRCLAICQPLRSLRR
RTDRLAVLATWLGCLVASAPQVHIFSLREVADGVEDCWAVFIQPWGPKAY
ITWITLAVYIVPVIVLAACYGLISFKIWQNLRLKTAAAAAAEAPEGAAAG
DGGRVALARVSSVKLISKAKIRTVKMTFIIVLAFIVCWTPFFFVQMWSVW
DANAPKEASAFIIVMLLASLNSCCNPWIYMLFTGHLFHELVQRFLCCSAS
YLKGRRLGETSASKKSNSSSFVLSHRSSSQRSCSQPSTA.

Results: GRAB-ACh-Cter580, starting from C1 site, was inserted at the position after the site 483 L of HRH1 to obtain a GRAB-His-Cter580 sensor, which has a 20% change in fluorescence signal responding to 1 mM Histamine. GRAB-ACh-Cter580, starting from C1 site, was inserted at the position after the site 353 K of OxtR to obtain a GRAB-Oxt-Cter580 sensor, which has a 10% change in fluorescence signal responding to 1 μM Oxytocin. GRAB-ACh-Cter580, starting from C2 site, was inserted at the position after the site 441 L of DRD2 to obtain GRAB-DA-Cter580 sensor, which has 20% change (decrease) in fluorescence signal responding to 20 μM Dopamine. GRAB-ACh-Cter580, starting from C2 site, was inserted at the position after the site 347S of B2AR to obtain a GRAB-Epi-Cter580 sensor, which has 30% change in fluorescence signal responding to 2 μM ISO. GRAB-ACh-Cter580, starting from C2 site, was inserted at the position after the site 334Y of HTR4 to obtain GRAB-5HT-Cter580 sensor, which has 10% change in fluorescence signal responding to 10 μM 5HT (FIGS. 10d-h). The above results indicate that the proteins constructed by fusing the signal molecule at the C-terminus of the G protein-coupled receptor also give a good detectable effect.

The amino acid sequence of GRAB-ACh-Cter580 is:

(SEQ ID NO: 17)
METDTLLLWVLLLWVPGSTGDTSLYKKVGTTLHNNSTTSPLFPNISSSWI
HSPSDAGLPPGTVTHFGSYNVSRAAGNFSSPDGTTDDPLGGHTVWQVVFI
AFLTGILALVTIIGNILVIVSFKVNKQLKTVNNYFLLSLACADLIIGVIS
MNLFTTYIIMNRWALGNLACDLWLAIDYVASNASVMNLLVISFDRYFSIT
RPLTYRAKRTTKRAGVMIGLAWVISFVLWAPAILFWQYFVGKRTVPPGEC
FIQFLSEPTITFGTAIAAFYMPVTIMTILYWRVFQEAKRQLQKIDKSEGR
FHVQNLSQVEQDGRTGHGLRRSSKFCLKEHKALKTLSAILLAFIITWTPY
NIMVLVNTFCDSCIPKTFWNLGYWLCYINSTVNPVCYALCNKTFRTTFKM
LLLCQCDKKKRRKQQYQQRQSVIGGNVYIKADKQKNGIKANFHIRHNIED
GGVQLAYHYQQNTPIGDGPVLLPDNHYLSVQSKLSKDPNEKRDHMVLLEF
VTAAGITLGMDELYKGGTGGSMVRKGEELFTGVVPILVELDGDVNGHKFS
VSGEGEGDATYGKLTLKFICTTGKLPVPWPTLVTTLTYGVQCFSRYPDHM
KQHDFFKSAMPEGYIQERTIFFKDDGNYKTRAEVKFEGDTLVNRIELKGI
DFKEDGNILGHKLEYNGGAAAFHKRAPEQALVFAAVKDTILQLNLKEYNL
VKSRITSEGEYIPLDQIDINVNSFCYENEVALT.

The amino acid sequence of GRAB-DA-Cter580 is as follows:

(SEQ ID NO: 18)
METDTLLLWVLLLWVPGSTGDTSLYKKVGTDPLNLSWYDDDLERQNWSRP
FNGSDGKADRPHYNYYATLLTLLIAVIVFGNVLVCMAVSREKALQTTTNY
LIVSLAVADLLVATLVMPWVVYLEVVGEWKFSRIHCDIFVTLDVMMCTAS
ILNLCAISIDRYTAVAMPMLYNTRYSSKRRVTVMISIVWVLSFTISCPLL
FGLNNADQNECIIANPAFVVYSSIVSFYVPFIVTLLVYIKIYIVLRRRRK
RVNTKRSSRAFRAHLRAPLKGNCTHPEDMKLCTVIMKSNGSFPVNRRRVE
AARRAQELEMEMLSSTSPPERTRYSPIPPSHHQLTLPDPSHHGLHSTPDS
PAKPEKNGHAKDHPKIAKIFEIQTMPNGKTRTSLKTMSRRKLSQQKEKKA
TQMLAIVLGVFIICWLPFFITHILNIHCDCNIPPVLYSAFTWLGYVNSAV
NPIIYTTFNIEFRKAFLKILRRKQQYQQRQSVIGGNVYIKADKQKNGIKA
NFHIRHNIEDGGVQLAYHYQQNTPIGDGPVLLPDNHYLSVQSKLSKDPNE
KRDHMVLLEFVTAAGITLGMDELYKGGTGGSMVRKGEELFTGVVPILVEL
DGDVNGHKFSVSGEGEGDATYGKLTLKFICTTGKLPVPWPTLVTTLTYGV
QCFSRYPDHMKQHDFFKSAMPEGYIQERTIFFKDDGNYKTRAEVKFEGDT
LVNRIELKGIDFKEDGNILGHKLEYNGGAAAFHKRAPEQALVFAAVKDTI
LQLNLKEYNLVKSRITSEGEYIPLDQIDINVNSFCYENEVALT.

The amino acid sequence of GRAB-His-Cter580 is as follows:

(SEQ ID NO: 19)
METDTLLLWVLLLWVPGSTGDTSLYKKVGTSLPNSSCLLEDKMCEGNKTT
MASPQLMPLVVVLSTICLVTVGLNLLVLYAVRSERKLHTVGNLYIVSLSV
ADLIVGAVVMPMNILYLLMSKWSLGRPLCLFWLSMDYVASTASIFSVFIL
CIDRYRSVQQPLRYLKYRTKTRASATILGAWFLSFLWVIPILGWNHFMQQ
TSVRREDKCETDFYDVTWFKVMTAIINFYLPTLLMLWFYAKIYKAVRQHC
QHRELINRSLPSFSEIKLRPENPKGDAKKPGKESPWEVLKRKPKDAGGGS
VLKSPSQTPKEMKSPVVFSQEDDREVDKLYCFPLDIEHMQAAAEGSSRDY
VAVNRSHGQLKTDEQGLNTHGASEISEDQMLGDSQSFSRTDSDTTTETAP
GKGKLRSGSNTGLDYIKFTWKRLRSHSRQYVSGLHMNRERKAAKQLGFIM
AAFILCWIPYFIFFMVIAFCKNCCNEHLHMFTIWLGYINSTLNPLIYPLC
NENFKKTFKRILKRRKQQYQQRQSVIGGNVYIKADKQKNGIKANFHIRHN
IEDGGVQLAYHYQQNTPIGDGPVLLPDNHYLSVQSKLSKDPNEKRDHMVL
LEFVTAAGITLGMDELYKGGTGGSMVRKGEELFTGVVPILVELDGDVNGH
KFSVSGEGEGDATYGKLTLKFICTTGKLPVPWPTLVTTLTYGVQCFSRYP
DHMKQHDFFKSAMPEGYIQERTIFFKDDGNYKTRAEVKFEGDTLVNRIEL
KGIDFKEDGNILGHKLEYNGGAAAFHKRAPEQALVFAAVKDTILQLNLKE
YNLVKSRITSEGEYIPLDQIDINVNSFCYENEVALT.

The amino acid sequence of GRAB-5HT-Cter580 is:

(SEQ ID NO: 20)
METDTLLLWVLLLWVPGSTGDTSLYKKVGTDKLDANVSSNEGFRSVEKVV
LLTFLAVVILMAILGNLLVMVAVCRDRQLRKIKTNYFIVSLAFADLLVSV
LVMPFGAIELVQDIWAYGEMFCLVRTSLDVLLTTASIFHLCCISLDRYYA
ICCQPLVYRNKMTPLRIALMLGGCWVLPMFISFLPIMQGWNNIGIVDVIE
KRKFSHNSNSTWCVFMVNKPYAITCSVVAFYIPFLLMVLAYYRIYVTAKE
HAQQIQMLQRAGATSESRPQPADQHSTHRMRTETKAAKTLCVIMGCFCFC
WAPFFVTNIVDPFIDYTVPEQVWTAFLWLGYINSGLNPFLYAFLNKSFRR
AFLIILCCDDERYRKQQYQQRQSVIGGNVYIKADKQKNGIKANFHIRHNI
EDGGVQLAYHYQQNTPIGDGPVLLPDNHYLSVQSKLSKDPNEKRDHMVLL
EFVTAAGITLGMDELYKGGTGGSMVRKGEELFTGVVPILVELDGDVNGHK
FSVSGEGEGDATYGKLTLKFICTTGKLPVPWPTLVTTLTYGVQCFSRYPD
HMKQHDFFKSAMPEGYIQERTIFFKDDGNYKTRAEVKFEGDTLVNRIELK
GIDFKEDGNILGHKLEYNGGAAAFHKRAPEQALVFAAVKDTILQLNLKEY
NLVKSRITSEGEYIPLDQIDINVNSFCYENEVALT.

The amino acid sequence of GRAB-Oxt-Cter580 is as follows:

(SEQ ID NO: 21)
METDTLLLWVLLLWVPGSTGDTSLYKKVGTEGALAANWSAEAANASAAPP
GAEGNRTAGPPRRNEALARVEVAVLCLILLLALSGNACVLLALRTTRQKH
SRLFFFMKHLSIADLVVAVFQVLPQLLWDITFRFYGPDLLCRLVKYLQVV
GMFASTYLLLLMSLDRCLAICQPLRSLRRRTDRLAVLATWLGCLVASAPQ
VHIFSLREVADGVFDCWAVFIQPWGPKAYITWITLAVYIVPVIVLAACYG
LISFKIWQNLRLKTAAAAAAEAPEGAAAGDGGRVALARVSSVKLISKAKI
RTVKMTFIIVLAFIVCWTPFFFVQMWSVWDANAPKEASAFIIVMLLASLN
SCCNPWIYMLFTGHLFHELVQRFLCCSASYLKKRRKQQYQQRQSVIGGNV
YIKADKQKNGIKANFHIRHNIEDGGVQLAYHYQQNTPIGDGPVLLPDNHY
LSVQSKLSKDPNEKRDHMVLLEFVTAAGITLGMDELYKGGTGGSMVRKGE
ELFTGVVPILVELDGDVNGHKFSVSGEGEGDATYGKLTLKFICTTGKLPV
PWPTLVTTLTYGVQCFSRYPDHMKQHDFFKSAMPEGYIQERTIFFKDDGN
YKTRAEVKFEGDTLVNRIELKGIDFKEDGNILGHKLEYNGGAAAFHKRAP
EQALVFAAVKDTILQLNLKEYNLVKSRITSEGEYIPLDQIDINVNSFCYE
NEVALT.

The amino acid sequence of GRAB-Epi-Cter580 is as follows:

(SEQ ID NO: 22)
METDTLLLWVLLLWVPGSTGDTSLYKKVGTGQPGNGSAFLLAPNGSHAPD
HDVTQERDEVWVVGMGIVMSLIVLAIVFGNVLVITAIAKFERLQTVTNYF
ITSLACADLVMGLAVVPFGAAHILMKMWTFGNFWCEFWTSIDVLCVTASI
ETLCVIAVDRYFAITSPFKYQSLLTKNKARVIILMVWIVSGLTSFLPIQM
HWYRATHQEAINCYANETCCDFFTNQAYAIASSIVSFYVPLVEVIVFVYS
RVFQEAKRQLQKIDKSEGRFHVQNLSQVEQDGRTGHGLRRSSKFCLKEHK
ALKTLGIEVIGTFTLCWLPFFIVNIVHVIQDNLIRKEVYILLNWIGYVNS
GFNPLIYCRSPDFRIAFQELLCLRRSSRRKQQYQQRQSVIGGNVYIKADK
QKNGIKANFHIRHNIEDGGVQLAYHYQQNTPIGDGPVLLPDNHYLSVQSK
LSKDPNEKRDHMVLLEFVTAAGITLGMDELYKGGTGGSMVRKGEELFTGV
VPILVELDGDVNGHKFSVSGEGEGDATYGKLTLKFICTTGKLPVPWPTLV
TTLTYGVQCFSRYPDHMKQHDFFKSAMPEGYIQERTIFFKDDGNYKTRAE
VKFEGDTLVNRIELKGIDFKEDGNILGHKLEYNGGAAAFHKRAPEQALVF
AAVKDTILQLNLKEYNLVKSRITSEGEYIPLDQIDINVNSFCYENEVAL
T.

### Example 3: GRAB Sensors have Specific Optical Signal Change Caused by Change in Receptor Conformation Resulting from Ligand Binding

In order to determine whether the fluorescence change of the sensor was specific from receptor activation, the HEB293T cells expressing GRAB-EPI 1.0 and GRAB-ACh 1.0 in Example 2 were treated with the receptor-specific blocking agents, respectively. Whether the fluorescence signal changes caused by the ligand binding can be eliminated in the presence of the blocking agents was observed. For single cell, the experiment was first performed with the corresponding ligand. After an increase in the signal was observed, the ligand was washed away completely, and the cell was perfused with a mixed solution of the blocking agent and the ligand. The selected blocking agent is: ICI, for the β2 adrenergic receptor (Rasmussen, S. G. F. et al. Crystal structure of the human beta2 adrenergic G-protein-coupled receptor. Nature 450, 383-387 (2007)), and Tiotropium Bromide (Tio), for the M3 acetylcholine receptor (Wood, M. D. et al. Functional comparison of muscarinic partial agonists at muscarinic receptor subtypes hM1, hM2, hM3, hM4 and hM5 using microphysiometry. British journal of pharmacology 126, 1620-1624 (1999)). It was found that, the agonist (ISO) or ligand (Ach) cannot cause an increase in fluorescence intensity in the presence of the blocking agent (FIG. 11), which reveals that the blocking agent specifically blocked the interaction between receptor fluorescent sensor and the agonist (ISO) or ligand (Ach), so that the receptor fluorescent sensor cannot be activated, resulting in no conformational change and therefore no fluorescence intensity change.

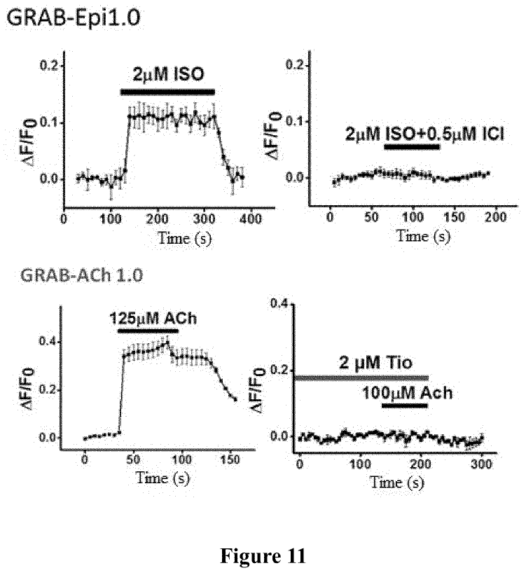

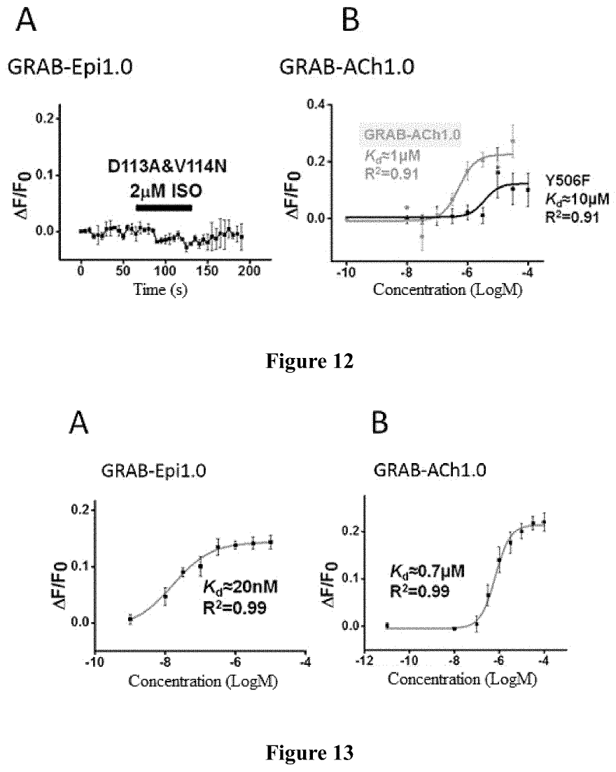

Mutation experiments were performed on the binding site of the receptor and ligand to further verify that the fluorescence change of the sensor is specific from receptor activation. For the β2 adrenergic receptor, amino acid positions 113 and 114 were mutated, which were in the binding sites of the receptor and the ligand in GRAB-EPI 1.0. HEK293T cells expressing the mutated sensors were observed for the fluorescence response to the agonist ISO. These two mutations have been shown to significantly reduce the affinity between the receptor and ligand (Del Carmine, R. et al. Mutations inducing divergent shifts of constitutive activity reveal different modes of binding among catecholamine analogues to the beta-adrenergic receptor. British journal of pharmacology 135, 1715-1722 (2002)). Cells expressing mutated sensors did not show any increase in fluorescence intensity when responding to the agonist ISO. For the M3 type acetylcholine receptor, the amino acid position 506 in the ligand binding region of GRAB-ACh 1.0 was mutated (Y506F), and the HEK293T cells expressing this mutated sensor were observed for the fluorescence response to the ligand (acetylcholine). This mutation was known to reduce the ligand binding capacity by about ten times (Wood, M. D. et al. Functional comparison of muscarinic partial agonists at muscarinic receptor subtypes hM1, hM2, hM3, hM4 and hM5 using microphysiometry. British journal of pharmacology 126, 1620-1624 (1999)). In the cells expressing this mutated acetylcholine sensor, it was observed that the affinity of acetylcholine to the sensor decreased by about 10 times and the Kd value decreased from 1 μM to 10 μM (FIG. 12).

The above experiments show that the fluorescent signal changes of GRAB sensors after adding ligand indeed resulted from the conformational change after the receptor was activated. This conformational change involves a change in the microenvironment of the circular permutated fluorescent proteins, resulting in an increase in fluorescence.

### Example 4: Optical Response of GRAB Sensor is Ligand Concentration-Dependent

Experiments were performed at different concentrations of ligand on the epinephrine sensor GRAB-EPI 1.0 and the acetylcholine sensor GRAB-ACh 1.0 (FIG. 13) using HEK293T cells expressing GRAB-EPI 1.0 and GRAB-ACh 1.0, respectively. It was found that the sensors showed a concentration-dependent change in fluorescence signal in a wide range of neurotransmitter concentrations, and the curves thereof are in line with the Hill distribution. By calculating the Kd values of the curves and comparing them with the Kd value of the receptor for the ligand in the literature (Wacker, D. et al. Structural features for functional selectivity at serotonin receptors. Science (New York, N.Y.) 340, 615-619 (2013); Gainetdinov, R. R., Premont, R. T., Bohn, L. M., Lefkowitz, R. J. & Caron, M. G. Desensitization of G protein-coupled receptors and neuronal functions. Annual review of neuroscience 27, 107-144 (2004)), it was found that the GRAB sensors did not change the affinity of the receptor for specific ligands. The ligand concentration-dependent response curves show that the GRAB sensors can sensitively and quantitatively detect neurotransmitters of different concentrations under physiological conditions.

These experiments show that the GRAB neurotransmitter sensors constructed based on the receptors can not only qualitatively report the binding and concentration change of neurotransmitters, but also quantitatively analyze the absolute concentration of neurotransmitters in specific regions.

### Example 5: Sub-Second Kinetics and Micromolar Sensitivity of GRAB-ACh Sensor Detection

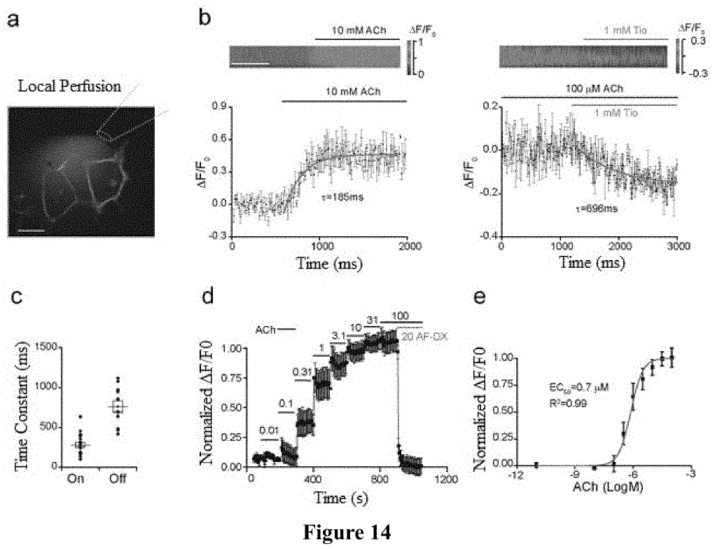

High-speed line scanning ((˜2,000 Hz/line) confocal imaging was performed on the membrane surface fluorescence signals of HEK293T cells expressing GRAB-ACh 2.0 that were under a rapid local perfusion system for agonists or antagonists (FIGS. 14a-b). The local ACh perfusion caused a rapid increase in the fluorescence intensity of GRAB-ACh 2.0 expressing cells, with a time constant of 280±32 ms when fitted with a single exponential function (FIGS. 14b-c, left panel). The local perfusion of tiotropium (Tio) and muscarinic antagonist (AF-DX384) (Casarosa, P., et al. Preclinical evaluation of long-acting muscarinic antagonists: comparison of tiotropium and investigational drugs. The Journal of pharmacology and experimental therapeutics 330, 660-668 (2009)) reduced the fluorescence signal in GRAB-ACh 2.0 expressing cells perfused with 100 μM ACh, with a smaller time constant of 762±75 ms (FIGS. 14b-c, right panel).

In order to determine the sensitivity of the sensor, the fluorescence intensity of HEK293T cells expressing GRAB-ACh 2.0 was perfused with a solution containing different concentrations of ACh (FIG. 14d) and the signal was measured. Increasing the ACh concentration from 10 nM to 100 μM gradually increased the fluorescence intensity of GRAB-ACh 2.0-expressing cells in a concentration-dependent manner, with an EC₅₀ of ˜0.7 μM when fitted with the Boltzmann's equation (FIG. 14e), which is equivalent to wild-type M3R (WT-M₃R) (Jakubik, J., Bacakova, L., El-Fakahany, E. E. & Tucek, S. Positive cooperativity of acetylcholine and other agonists with allosteric ligands on muscarinic acetylcholine receptors. Molecular pharmacology 52, 172-179 (1997)).

### Example 6: Decoupling of GRAB Sensors from Downstream Signaling Pathways

This example is to verify whether the overexpression of a fluorescent sensor based on the important signal molecule GPCR in a cell would cause unnecessary signaling pathway activation. To answer this question, two known major signaling pathways of GPCR, G protein-mediated signaling pathway and the arrestin-mediated endocytosis signaling pathway (Gainetdinov, R. R., Premont, R. T., Bohn, L. M., Lefkowitz, R. J. & Caron, M. G. Desensitization of G protein-coupled receptors and neuronal functions. Annual review of neuroscience 27, 107-144 (2004)) were tested respectively, to observe the coupling ability of the GRAB sensor to the downstream pathway of GPCR.

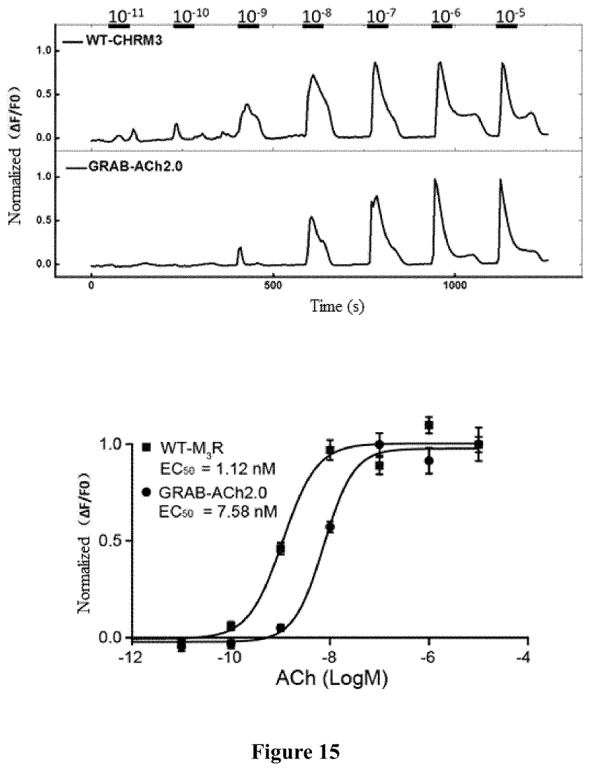

For the G protein-mediated signaling pathway, the downstream calcium signal was detected by calcium imaging to characterize the sensitivity of the GRAB-ACh 1.0 sensor constructed in Example 2 to the G protein-mediated signaling pathway. Since the GRAB sensor was in the green light spectrum, the red calcium dye Ca1590 was used in HEK293T cells expressing GRAB-ACh 1.0 and treated with different concentrations of acetylcholine. The Kd value was calculated by obtaining the response curve of calcium signal and ligand concentration, and then the sensitivities were compared to determine whether there was significant difference. The experimental results (FIG. 15) show that compared to the endogenous M3 type acetylcholine receptor (shown as WT-CHRM3 or WT-M₃R in FIG. 15, that is, the natural M3 type acetylcholine receptor without cpEGFP insertion compared to GRAB-ACh 1.0), the GRAB-ACh 1.0 sensor has reduced sensitivity to activate the G protein-mediated signaling pathway, and its Kd value decreased by about 5 times.

Next, the method of using Gα protein fragment to replace G protein was used, which was commonly used in GPCR crystal structure analysis. The Gα protein fragment was 20 amino acids at the C-terminus of the G protein. In the crystal structure, the C-terminus of the Gα protein inserts into the activated GPCR intracellular loop and plays an important role in stabilizing the GPCR in the activated state (Palczewski, K. et al. Crystal Structure of Rhodopsin: A G Protein-Coupled Receptor. Science (New York, N.Y.) 289, 739-745 (2000)). Because the C-terminus peptide of the Gα protein can replace G protein to stabilize the GPCR in the activated state and would not induce downstream signal by itself, it was speculated that artificially coupling the C-terminus peptide of the Gα protein to the end of the C-terminus of the GRAB sensor might lead to the C-terminus peptide of the Gα protein competing with the endogenous G protein for the position in the intracellular loop of the GRAB sensor, thereby reducing the activation of downstream signal mediated by G protein.

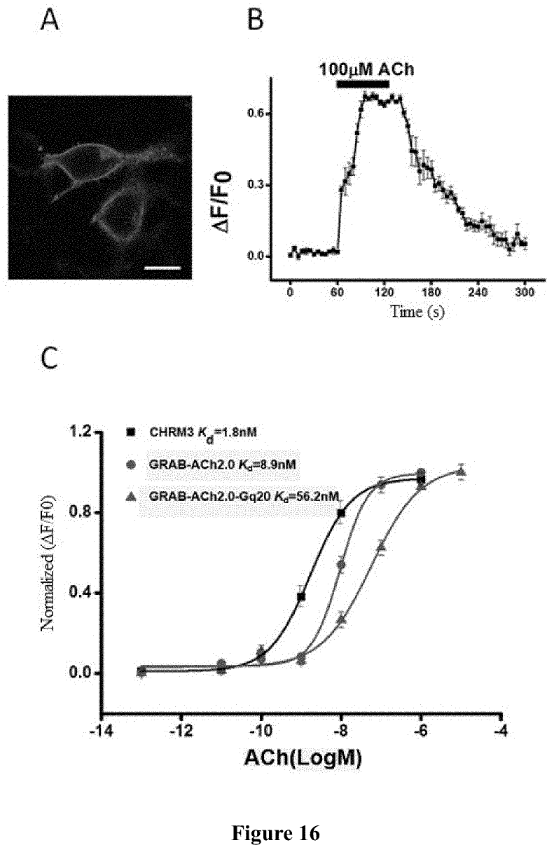

Taking the acetylcholine sensor as an example, peptide of 20 amino acids at the C-terminus of the Gαq protein (the specific sequence: VFAAVKDTILQLNLKEYNLV) was connected to the last amino acid at the C-terminus of the GRAB-ACh 2.0 sensor constructed in Example 2. The change in fluorescence induced by acetylcholine was measured, and also, calcium imaging was used to detect whether the signal transduction of the downstream G protein pathway was reduced. The sensor with the Gα protein peptide was named GRAB-ACh 2.0-Gq20, wherein Gq20 indicated that it was connected with the 20 amino acids at the C-terminus of the Gαq protein. From the results (FIG. 16), it was found that GRAB-ACh 2.0-Gq20 still has good cell membrane localization and fluorescence intensity, and a significant increase in fluorescence signal after the treatment of the ligand acetylcholine, with a signal change of 70% ΔF/F₀ in average, which was slightly lower than GRAB-ACh 2.0 (90%). Using the calcium imaging method described above, response curves of calcium signal were obtained corresponding to different concentrations of acetylcholine, which show a significant decrease in calcium signal coupling. The calcium signal coupling ability of GRAB-ACh 2.0-Gq20 was reduced by about 10 times compared with the sensor without the Gα peptide fragment, and reduced by 50 times compared with the endogenous M3R type receptor (i.e., CHRM3). This result indicates that after the Gα peptide fragment is fused at the end of the GRAB sensor, it successfully competes with the endogenous G protein, thereby significantly reducing the coupling of the sensor to the G protein signaling pathway, so that preventing the GRAB sensor from causing significant disturbances in the cell signal system when expressed in the cells.

In addition, on the basis of the GRAB-Ach 2.0 sensor constructed in Example 2, the 20 amino acids fragments derived from C-terminus of different Gα proteins (that is, Gq20: VFAAVKDTILQLNLKEYNLV (SEQ ID NO: 6), Gs20: VFNDCRDIIQRMHLRQYELL (SEQ ID NO: 7), and Gi20: VFDAVTDVIIKNNLKDCGLF (SEQ ID NO: 8)) were connected to the last amino acid at the C-terminus of the sensor, respectively. The coupling ability to the downstream G protein signaling pathway of these acetylcholine sensors was detected by changes of the calcium signal at different acetylcholine concentrations (see FIG. 53 for results, where chrm3 was the unmodified wild-type acetylcholine receptor M3R). It can be seen from FIG. 53 that, the coupling ability of these acetylcholine sensors to the downstream G protein signaling pathway was reduced due to the competition of the Gα protein fragment and the endogenous G protein.

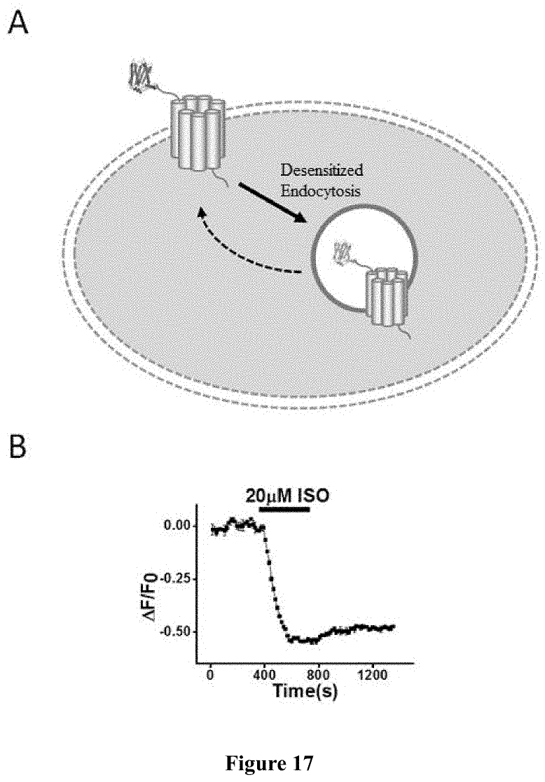

In addition to the downstream pathway mediated by G protein, another important downstream pathway of GPCR was the signaling pathway related to receptor-mediated endocytosis by proteins such as arrestin. In order to stably detect the dynamic changes of extracellular neurotransmitters, the ideal sensor should not be regulated by the endocytosis system, so that it can truly reflect the concentration changes of the exogenous neurotransmitters. To address this problem, it was first detected whether the GRAB sensor would couple with the arrestin signaling pathway and cause the endocytosis of the receptor sensor. It was further speculated that, if the GRAB sensor can be coupled to the endocytosis signaling pathway and cause receptor endocytosis, a decrease in the fluorescent signal on the cell membrane would be observed. If the fluorescence signal on the cell membrane does not show obvious change under long-term (more than five minutes) ligand treatment, the coupling between the sensor and the endocytosis signaling pathway may be damaged. First, a fluorescent sensor based on receptor endocytosis (GPCR fused with pH-sensitive fluorescent protein) was constructed (see Example 1 for the specific construction methods). The pHluorin gene was connected to the N-terminus of the natural β₂ adrenergic receptor (β2AR) gene by the Gibson assembly method, and a short peptide segment of 3 amino acids (GGA) was used to connect them, and the last 29 amino acids (343-371 amino acids) of the human AVPR2 were fused at the end of the C-terminus to obtain pHluorin-β₂AR. It was proved that the adrenergic receptor can activate the endocytosis signaling pathway, which was specifically manifested in that the fluorescence intensity of pHluorin-β₂AR showed a significant decrease after a period of time after the addition of the agonist ISO (FIG. 17).

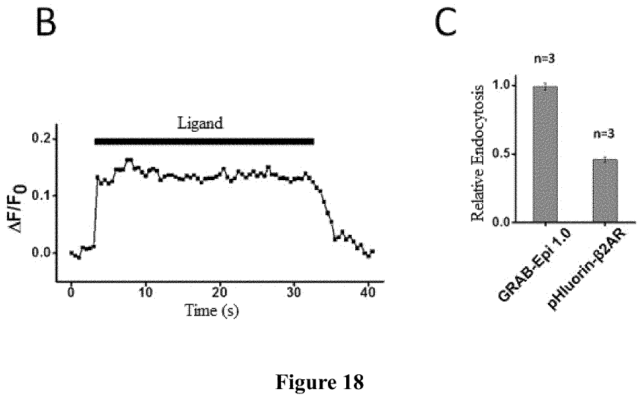

Since the adrenergic receptor can stably activate the endocytosis signaling pathway, a long-term ligand treatment experiment was performed on the epinephrine sensor GRAB-EPI 1.0 constructed. The fluorescence intensity of the cell membrane was observed to determine whether there was a significant decrease in fluorescence with the increase time of the agonist ISO treatment. The fluorescence value curve of the sensor during 30 minutes agonist ISO treatment was obtained, and it was found that the fluorescence value of GRAB-EPI 1.0 did not change significantly with time and maintained a similar fluorescence intensity for a long period during the agonist treatment. The agonist was washed away after 30 minutes, and the fluorescence signal returned to the basal level, indicating that the change in fluorescence was a reversible response to receptor activation (FIG. 18). Based thereon, it can be considered that the coupling efficiency of the GRAB sensor and arrestin-mediated endocytosis signaling pathway was greatly reduced, which may be because the fluorescent protein in the GRAB sensor occupied the binding site of proteins such as arrestin, making it difficult to couple with this signaling pathway. For the sensor itself, the reduced signal coupling can better ensure that the change in fluorescence truly reflects the change in the concentration of the exogenous ligand.

### Example 7: Application of Fluorescent Sensor for Neurotransmitter

### 1. The Fluorescent Sensor Responds to the Specific Neurotransmitter in Cultured Neurons.

The epinephrine sensor GRAB-EPI 1.0 and acetylcholine sensor GRAB-ACh 1.0 were expressed in cultured neurons, respectively, and their expression in this system and their response to specific neurotransmitters were observed.

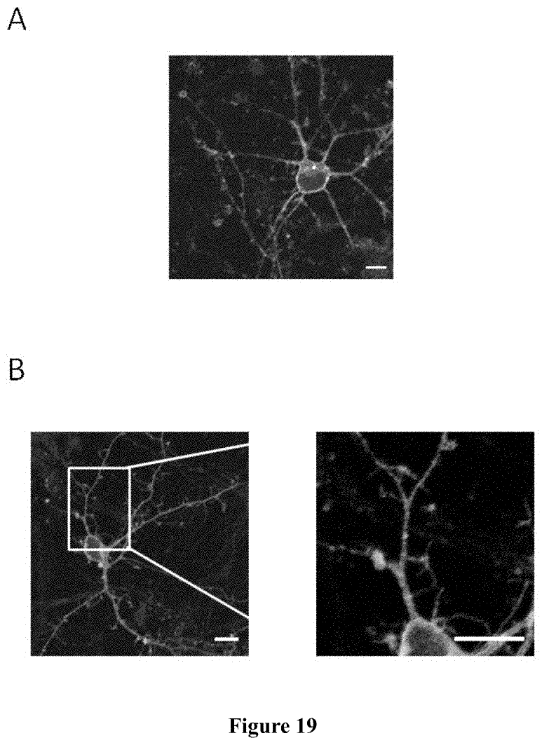

The primary cultured rat cortical neurons were transfected with the expression vectors by calcium phosphate, and the neurons were imaged after about 48 hours. Neurons expressing neurotransmitter fluorescent sensors have normal morphology and nice axonal and dendritic network. The fluorescent sensor constructed based on the receptor was uniformly expressed on the cell membrane of the neuron, and the expression of the sensor can also be clearly seen on different structures of the neuron, such as the synaptic spine (FIG. 19).

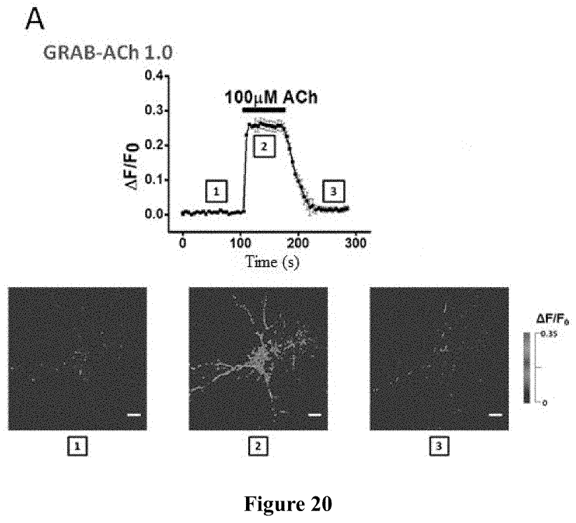

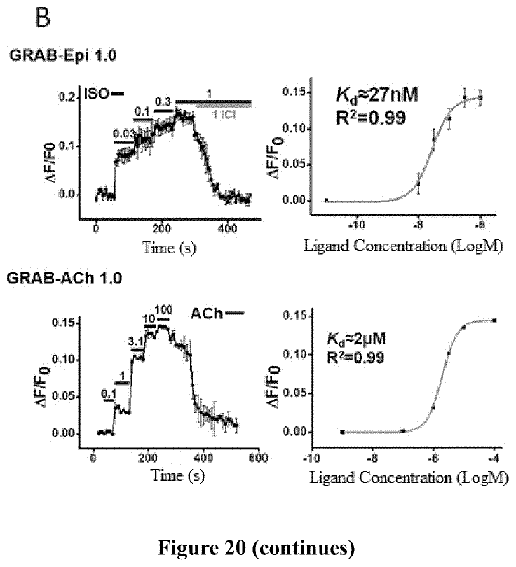

The optical response of neurons expressing the neurotransmitter sensor was observed by perfusion with a neurotransmitter solution. The neurotransmitter-specific optical signals were recorded, which were fast and stable, and have good repeatability in different neurons. Furthermore, a specific receptor blocking agent (such as Tio) was used to stabilize the receptor in an inactive state. In this case, the neurotransmitter cannot induce the activation of the receptor, and the corresponding optical signal would not change (FIG. 44). In order to prove that the neurotransmitter sensors expressed in neurons still response sensitively to different concentrations of neurotransmitters, ligand concentration-dependent curves of the optical signal of the sensors were measured, as shown in FIG. 20, which were in line with Hill equation in cultured cell lines, and the Kd values were similar to those reported in the literature (Wood, M. D. et al. Functional comparison of muscarinic partial agonists at muscarinic receptor subtypes hM1, hM2, hM3, hM4 and hM5 using microphysiometry. British journal of pharmacology 126, 1620-1624 (1999); Hoffmann, C., Leitz, M. R., Oberdorf-Maass, S., Lohse, M. J. & Klotz, K. N. Comparative pharmacology of human adrenergic receptor subtypes—Characterization of stably transfected receptors in CHO cells. Naunyn-Schmiedeberg's archives of pharmacology 369, 151-159 (2004)).

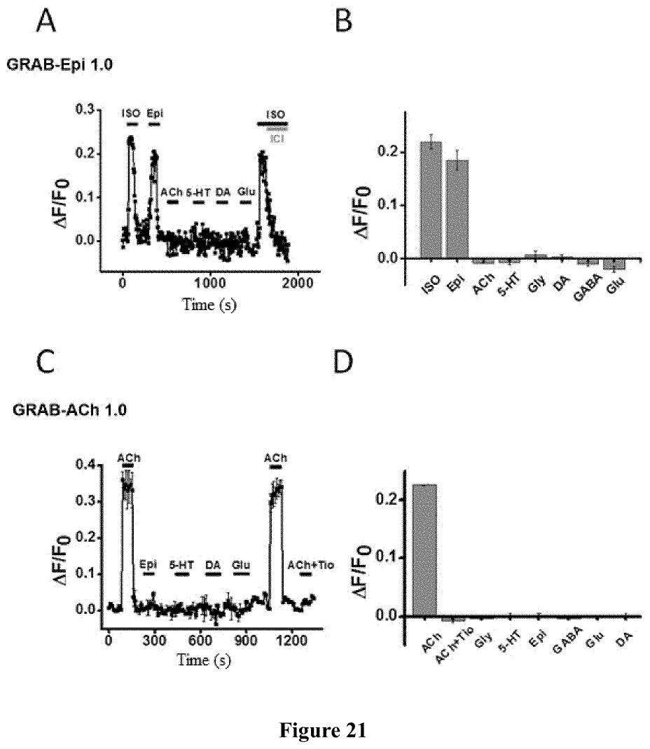

Neurons expressing the same neurotransmitter sensor were treated sequentially with different neurotransmitters at saturated concentration. It was found that only the corresponding neurotransmitter can induce a reproducible optical signal response by the sensor, while other neurotransmitters cannot induce any change in optical signal even at high concentrations (FIG. 21). The results demonstrate that the fluorescent sensors can specifically detect the corresponding neurotransmitters without being affected by change in the concentration of other neurotransmitters.

### 2. Detection of the Release of Acetylcholine in the Olfactory System of Drosophila Using Two-Photon Imaging

In the central nervous system of Drosophila, acetylcholine is used as the main excitatory neurotransmitter to participate in signal transduction. In its olfactory system, after activating by odor molecules, neurons with olfactory receptors transmit sensory information to secondary olfactory neurons, i.e. antennal lobe, by releasing acetylcholine (Ng, M. et al. Transmission of olfactory information between three populations of neurons in the antennal lobe of the fly. Neuron 36, 463-474 (2002)). The classical calcium imaging method is used to observe the transmission of olfactory information by expressing calcium indicators in the antennal lobe. However, as the second messenger in the cell, the calcium signal itself lacks specificity and cannot reflect the specific neurotransmitter that plays a role in information transmission (Wang, J. W., Wong, A. M., Flores, J., Vosshall, L. B. & Axel, R. Two-photon calcium imaging reveals an odor-evoked map of activity in the fly brain. Cell 112, 271-282 (2003)). In this example, the acetylcholine sensor encoded by gene was developed and specifically expressed at the antennal lobe, that is, on the opposite side of the synapses of the olfactory receptor neurons, to detect the acetylcholine released by the olfactory receptor neurons after receiving olfactory information. A transgenic Drosophila UAS-GRAB-ACh 1.0 was constructed by transferring GRAB-ACh 1.0 into Drosophila through Drosophila embryo injection combined with genetic screening. After the transgenic Drosophila was crossed with the GH146-Gal4 strain (Ruta, V. et al. A dimorphic pheromone circuit in Drosophila from sensory input to descending output. Nature 468, 686-690 (2010)), the GRAB-ACh 1.0 sensor was specifically expressed at the antennal lobe, and the release of endogenous acetylcholine in the synaptic network after odor stimulation was observed by two-photon imaging.

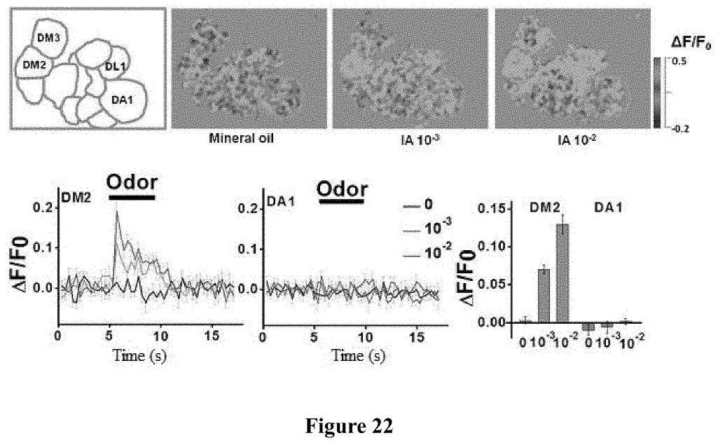

It was observed that after the odor treatment with isoamyl acetate (IA), a specific increase of fluorescence occurred at specific sites in the antennal lobe. In order to verify that this response was the acetylcholine release induced specifically by odor, different concentrations of odor molecule were given. It was observed that, there was no change in the fluorescence signals when the concentration of the odor molecule was zero; and the fluorescence response showed a concentration-dependent manner when the concentration of the odor molecule gradually increased (FIG. 22, top panels), suggesting that the response was indeed a release of neurotransmitter caused by odor molecule.

The antennal lobe can be divided into different regions corresponding to different olfactory bulbs. Because of receiving projections from different olfactory neurons, different olfactory bulbs have specific activation patterns for specific olfactory odor molecules. It was found that isoamyl acetate could specifically induce the change in the fluorescence signal at the olfactory bulbs DM2, DM3, and DL1, with the highest change at DM2, which was consistent with the results obtained using calcium imaging in the previous reports. Correspondingly, isoamyl acetate failed to induce the enhancement of optical signal at the olfactory bulb DA1, which was consistent with previous reports that DA1 mainly received the information from sex hormone odor molecules (Wang, J. W., Wong, A. M., Flores, J., Vosshall, L. B. & Axel, R. Two-photon calcium imaging reveals an odor-evoked map of activity in the fly brain. Cell 112, 271-282 (2003); Couto, A., Alenius, M. & Dickson, B. J. Molecular, anatomical, and functional organization of the Drosophila olfactory system. Current Biology 15, 1535-1547 (2005)) (FIG. 22, lower panels).

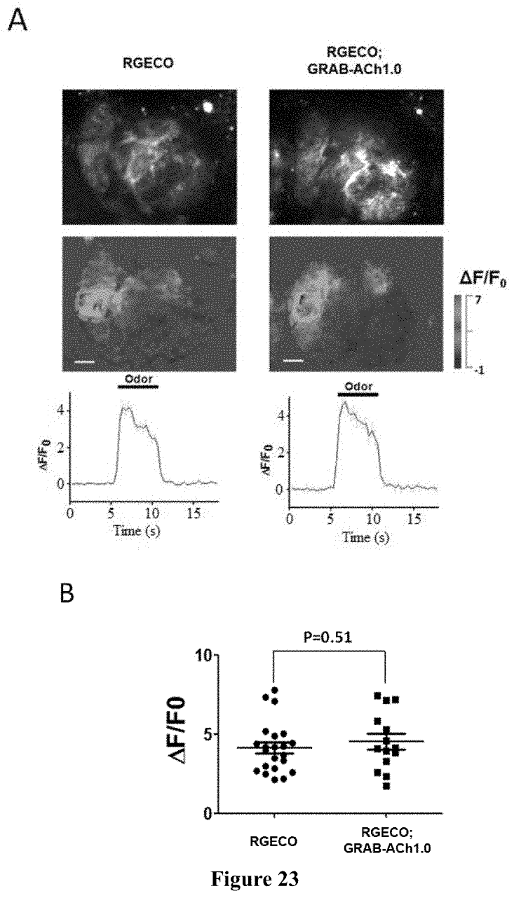

In order to test whether the GRAB-ACh 1.0 sensor overexpressed at the antennal lobe was coupled to the endogenous G protein signaling pathway and thus affect the calcium signal of the cell, the red calcium indicator RGECO (Yongxin Zhao, et al, An Expanded Palette of Genetically Encoded Ca2+ indicators, Science, 2011) was used to directly measure the calcium signal at the antennal lobe. When stimulated with the isoamyl acetate odor, Drosophila expressing GRAB-ACh1.0 sensor show no significant difference in calcium signal compared to Drosophila expressing only RGECO (FIG. 23), which implies that GRAB sensors that are overexpressed in vivo do not cause observable disturbance in calcium signal.

### 3. Application of Acetylcholine Sensor in Mice Brain Slices by Virus Expression and Two-Photon Imaging

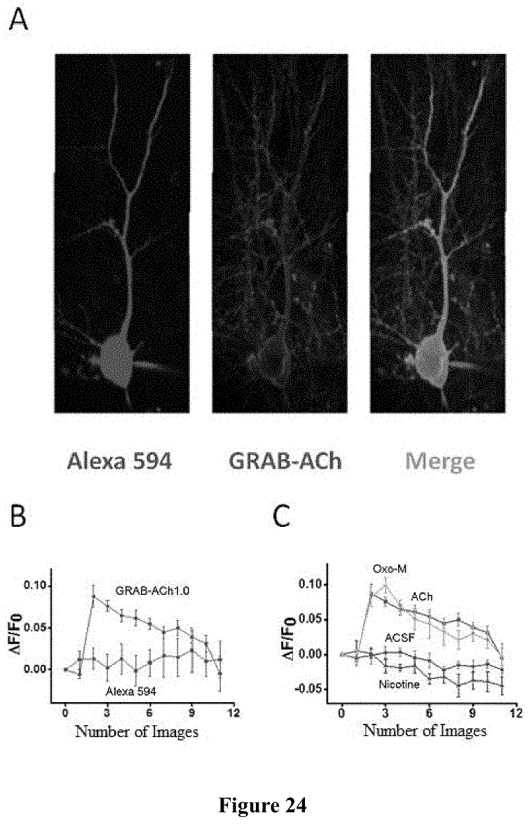

Since GRAB sensors have demonstrated good ability to detect specific neurotransmitters in cultured neurons and living Drosophila, it was further hoped that the fluorescent sensors can be expressed in mammalian brains to detect dynamic changes of neurotransmitters in more complex neural networks. The DNA encoding GRAB-ACh 1.0 sensor was packed in lentivirus and expressed in the hippocampal neurons of mice, and its fluorescence performance was detected by local perfusion of acetylcholine. The GRAB sensor also showed a stable response in the brain slices and showed a rapid increase in fluorescence after adding acetylcholine, with an average amplitude of about 10%-15%. Because the GRAB-ACh 1.0 acetylcholine sensor was constructed based on the M3 receptor, an obvious fluorescence enhancement can also be induced by the M3 receptor-specific agonist Oxo-M, while the N-type acetylcholine agonist nicotine cannot induce changes in the fluorescence signal, which reveals the specificity of GRAB sensor signal (FIG. 24).

### Example 8: Construction of Specific Fluorescent Sensors for Epinephrine and/or Norepinephrine Using Human ADRA2A Receptor

### 1. Materials and Methods

### GRAB Sensor Construction, Mutation Screening and Cloning

In the present disclosure, the molecular cloning method of Gibson assembly (Gibson, D. G. et al. Enzymatic assembly of DNA molecules up to several hundred kilobases. Nature methods 6, 343-345 (2009)) is used, which uses sequence complementation to achieve the recombination of homologous fragments. An about 30 bases-homologous complementary sequence, which was designed on the primers, was used to achieve efficient splicing between sequences. All correctly recombined clones were confirmed by sequencing at the equipment center of the School of Life Sciences, Peking University.

The pDisplay vector from Invitrogen was used as the expression vector for GRAB sensors. The GPCR genes thereof were partially amplified from the full-length human cDNA (hORFeome database 8.1). It was first cloned to a pDisplay vector with att sequence by Gateway cloning method, and then a specific circular permutated fluorescent protein was inserted into a specific position of the receptor by Gibson assembly method. During the mutation screening of the sensor, the method of introducing mutations was to introduce random base combinations into specific primers to construct a site-directed mutation library. The remaining cloning and construction methods are similar.

### Cell Culture and Transfection

HEK293T cells were cultured in DMEM (DPF whole medium) containing 10% FBS and 1% PS in a 10 cm petri dish, with an incubator temperature of 37° C. and 5% CO₂. Exchanging or passaging was performed according to cell growth status. When exchanging the medium, the original medium was removed and 15 mL of the fresh medium was added. Passaging was performed when the cell confluence reached more than 80%. The original medium was removed, and 2 mL 0.01 M PBS was used to wash the cells twice to remove residual magnesium ion and serum. 0.5 mL, 0.25% trypsin-EDTA was added to the cells for digestion for 1 min at 37° C. The reaction was terminated with 2 mL of medium. The cells were gently pipetted until they completely detached from the bottom of the dish and dispersed. Then 2 mL of medium was added and pipetted evenly. Approximately 1 mL of the cell suspension was removed and placed in a new 10 cm Petri dish, and 14 mL of medium was added, gently shaken, and returned to the incubator.

For the screening experiments, cells need to be transferred into a 24-well plate (for perfusion experiment) or a 96-well plate (for opera phenix). First, the cells were digested with trypsin according to the above-mentioned passage method, and 4 mL of medium was added to form a uniform cell suspension. An appropriate volume of the cell suspension was taken and seeded at a density of 50% to the 24-well or 96-well plate with round glass slides for imaging. Approximately 500 μL of medium was added to each well of the 24-well plate, and approximately 100 μL of medium was added to each well of the 96-well plate, mixed well, and placed in an incubator for culture.

The cells were transfected 8-12 h after adherence. DNA and PEI were mixed in DMEM at a ratio of 1:3, and incubated at room temperature for 15-20 minutes. The mixture was added into the cell culture medium of the well to be transfected and mixed well. Approximately 800 ng of DNA was transfected into each well of the 24-well plate and 300 ng into each well of the 96-well plate. Exchanging of the medium was performed 4 h after transfection, and the fluorescence was observed 24 h after transfection.

Newborn Sprague-Dawley rats were used for the primary culture of rat neurons. After the skin was washed with alcohol, the head of newborn rat was dissected with surgical instruments, and the vascular membrane on the surface of the cortex was carefully removed after removing the brain. The cortical tissue was cut into pieces and placed in a 0.25% trypsin solution, and digested for 10 minutes in an incubator at 37° C. The digestion was terminated with a DMEM medium containing 5% FBS, and the cells were further isolated by slowly pipetting up and down ten times with a pipette. After standing for 5 minutes, the pellet containing the tissue debris was discarded and the upper layer solution was collected and centrifuged at 1000 rpm for 5 minutes in a centrifuge. Thereafter, the supernatant was discarded and the neurons were resuspended with the Neurobasal+B27 medium used for culturing the neurons. The density was calculated using a cell counting chamber. After that, the cells were diluted at a density of 0.5-1×10⁶ cells/ml and plated on the slides coated with poly-lysine (Sigma). Primary neurons were cultured in the Neurobasal+B27 medium, and a half of the medium was exchanged every two days. Primary cultured neurons were transfected 6-8 days after dissection using calcium phosphate transfection method. 1.5 hours after the transfection, whether there was a small and uniform calcium phosphate precipitate was observed through a microscope, and the solution was exchanged with a HBS solution of pH 6.8. After HBS washing, neurons were re-cultured in Neurobasal+B27 medium, and imaging experiments were performed after 48 hours.

### Fluorescence Imaging and Drug Perfusion

The perfusion system was set on a microscope, and including a solution-introduced system, an imaging cell, and a suction pump. During the perfusion process, the imaging cell was placed above the objective lens of the inverted microscope, and the slide with cultured cells was placed in the cell. The perfusion experiments of different drugs were performed by controlling the switches of the different pipes of the solution-introduced system. The perfusion speed was set to one drop or so per second. In order to ensure the stability of the focal plane, the liquid level was kept constant at all times, and the flow rate of each solution was adjusted accordingly. The aspiration speed was adjusted to maintain the liquid level in the cell over the slide.

The region of interest (ROI) and background area were selected manually prior to the perfusion experiment. The exposure time was set to less than 50 ms and the acquisition frequency was 5 seconds. The solution used for the perfusion was a physiological solution 4k (pH 7.3-7.4), which was used to formulate the drug to the desired concentration. The running time of the program was set to no more than 5 min. After using 4k to balance and stabilize the cells for about 60-90 s, the cells were perfused with drug for 60 s, and then washed with 4k. The excitation light for green fluorescent protein was 488 nm, and for red fluorescent protein was 568 nm. The laser intensity was adjusted according to the working state of the laser and the expression level of the cells.

After the end of the program, the time-fluorescence intensity data sheet was exported. The background was subtracted from the ROI to get the corresponding fluorescence value Ft, and the average value of the fluorescence value before adding the drug was used as the starting fluorescence F₀ to calculate

$$Δ F / {F}_{0} = \frac{( {F}_{t} - {F}_{0} )}{{F}_{0}} .$$

A curve of the relationship between the ratio value and time was plotted and the effect of the addition of the drug on the fluorescence intensity was analyzed.

The imaging experiments of neurons employed an inverted Nikon laser scanning confocal microscope, which based on an inverted Ti-E microscope and AlSi spectral detection confocal system. A 40× NA:1.35 oil lens and a 488 laser were used for imaging. The microscope body, PMT, and image acquisition and processing system of the laser scanning confocal microscope were controlled by NIS element software.

The detection of the response of the GRAB sensor to the ligand (ΔF/F₀) was performed by drug perfusion. The cells were maintained in a standard physiological solution, and the formula thereof is:

| | | |
|---|---|---|
| NaCl | 150 | mM |
| KCl | 4 | mM |
| MgCl₂ | 2 | mM |
| CaCl₂ | 2 | mM |
| HEPES | 10 | mM |
| Glucose | 10 | mM |

After the pH value of the physiological solution was adjusted to about 7.4, the small molecule drug was diluted with the physiological solution to formulate solutions of respective concentrations of the small molecule ligand.

### Opera Phenix™

Opera Phenix™ enables confocal imaging of 60 wells in the center of a 96-well plate at a time, using a 63× water lens. Before the experiment, the cell medium was exchanged with 100 μL of the physiological solution, and the plate was placed on a sample holder and put into the instrument. The appropriate imaging focal plane, excitation wavelength and laser intensity, imaging wells and the imaging field of each well were set. After running the program, all selected areas will be automatically imaged by the instrument. After the first imaging was completed, the 96-well plate was taken out, and the physiological solution in each well was exchanged with a physiological solution containing a drug of desired concentration, and imaging was performed again.

After the two images were completed, using the Harmony analysis software, red fluorescence from mCherry was employed to locate the membrane area of the cells in each field (the CAAX sequence in the RFP enables it to be located on the membrane), and the number of ROI was counted. The cpEGFP and mCherry fluorescence intensity ratio (GR ratio) in the ROI was calculated, and finally a report of the analysis results was exported. Comparing the change of GR ratio before and after adding drug, it can be determined whether the fluorescent sensor responds to the drug and the strength of the response.

There are two main differences between Opera Phenix™ experiment and perfusion experiment. First, Opera Phenix™ cannot automatically deduct background fluorescence due to the limitation of the data processing program of the software. Second, because the image acquisition was not dynamic and continuous, the plate must be taken out during the treatment, and thus the ROI of the two imaging, previous and subsequent acquisitions, may be different, and the confocal focal planes may also be different. So the absolute value of the cpEGFP fluorescence change may be caused by these measurement changes, and cannot be used as a standard for whether to respond to the drug. This was why RFP was introduced in the experiment. In the present disclosure, through rational design, the GFP and RFP expression ratio was constant, that is, the GR ratio of each cell may be approximately stable (but the absolute fluorescence intensity was not necessarily the same) when the external conditions were unchanged. RFP can reflect changes in ROI, focal plane and the like, but does not give fluorescence intensity change to the drug. Therefore, the GR ratio can be used to measure the response of the sensor. A sensor with a decreased GR ratio corresponding to a decrease in cpEGFP fluorescence intensity after treatment is considered an OFF sensor, and a sensor with an increased GR ratio corresponding to an increase in cpEGFP fluorescence intensity after treatment is considered an ON sensor.

### Explore the Neurotransmitter Response Kinetics by NPEC-Caged-NE

100 μM NPEC-NE was prepared in DMSO. The photo-uncaging experiment was performed on a Nikon laser scanning confocal microscope. The light stimulation was 80% of the 405 nm laser for 76 ms, and the stimulation area was 2×2 pixel rectangle (1 pixel=0.62 μm).

### Image Data Processing

Fluorescence imaging data was processed using ImageJ software. For the fluorescence performance of GRAB sensors in HEK293T cells and neurons, the entire cell body was selected as the data processing region. The change in the fluorescence signal was usually indicated by its relative intensity change. The background area without the expression of the sensor was first subtracted from the fluorescence signal, so as to obtain the true expression intensity of the fluorescent protein. Then, by calculating the fluorescence value F after adding the drug and the average fluorescence value F₀ before adding, the relative fluorescence change value ΔF/F=(F−F₀)/F₀ was obtained as the response of the fluorescence sensor to a specific drug. The change of ΔF/F₀ with time was further plotted in Origin 8.6 software. Pseudo-color images were made by Matlab.

### Statistical Analysis

In the present disclosure, the data shown in the figures are mean±standard error of the mean.

### 2. Selection of Human Norepinephrine Receptor Proteins for Construction of Fluorescent Sensors

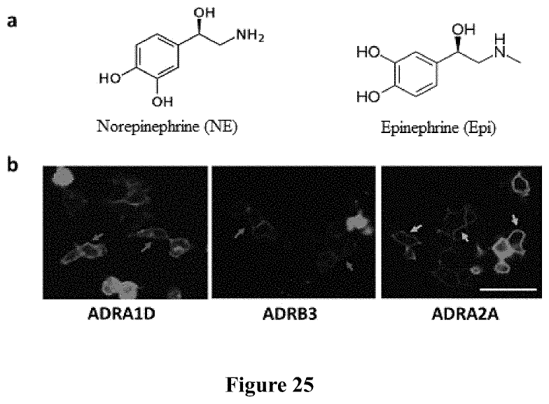

Three different subtypes of human derived norepinephrine receptor proteins were selected for constructing fusion proteins with the green fluorescent protein pHluorin, and the green fluorescence was observed under a 488 nm laser by a confocal microscope to detect the fusion protein expression on the cell membrane (FIG. 25b). Among them, the human ADRA2A receptor showed good membrane localization and high affinity for ligands. Therefore, the human ADRA2A receptor was selected as the basic unit for fluorescent sensor construction.

For the sequence of human ADRA2A, see NCBI gene ID: 150, and the amino acid sequence thereof is:

(SEQ ID NO: 2)
MFRQEQPLAEGSFAPMGSLQPDAGNASWNGTEAPGGGARATPYSLQVTLT
LVCLAGLLMLLTVFGNVLVIIAVFTSRALKAPQNLFLVSLASADILVATL
VIPFSLANEVMGYWYFGKAWCEIYLALDVLFCTSSIVHLCAISLDRYWSI
TQAIEYNLKRTPRRIKAIIITVWVISAVISFPPLISIEKKGGGGGPQPAE
PRCEINDQKWYVISSCIGSFFAPCLIMILVYVRIYQIAKRRTRVPPSRRG
PDAVAAPPGGTERRPNGLGPERSAGPGGAEAEPLPTQLNGAPGEPAPAGP
RDTDALDLEESSSSDHAERPPGPRRPERGPRGKGKARASQVKPGDSLPRR
GPGATGIGTPAAGPGEERVGAAKASRWRGRQNREKRFTFVLAVVIGVFVV
CWFPFFFTYTLTAVGCSVPRTLFKFFFWFGYCNSSLNPVIYTIFNHDFRR
AFKKILCRGDRKRIV.

Wherein the underlined part is the third intracellular loop, specifically amino acids positions 218-374, as defined by the uniprot database.

3. Obtaining of GRAB-NE1.0 with Optical Signal Changes Responding to High Concentrations of NE by Truncating the Third Intracellular Loop ICL3 of the Human ADRA2A Receptor and Inserting the Circular Permutated Fluorescent Protein cpEGFP.

There are 157 (referring to the uniprot database) amino acids in the third intracellular loop of the human ADRA2A receptor. ICL3 of the human ADRA2A receptor was truncated and cpEGFP was inserted. The cpEGFP used here was the same as that in Example 2, which was the circular permutated fluorescent protein cpEGFP from GCaMP6s, and the specific sequence is:

(SEQ ID NO: 11)
NVYIKADKQKNGIKANFHIRHNIEDGGVQLAYHYQQNTPIGDGPVLLPDN
HYLSVQSKLSKDPNEKRDHMVLLEFVTAAGITLGMDELYKGGTGGSMVRK
GEELFTGVVPILVELDGDVNGHKFSVSGEGEGDATYGKLTLKFICTTGKL
PVPWPTLVTTLTYGVQCFSRYPDHMKQHDFFKSAMPEGYIQERTIFFKDD
GNYKTRAEVKFEGDTLVNRIELKGIDFKEDGNILGHKLEYN.

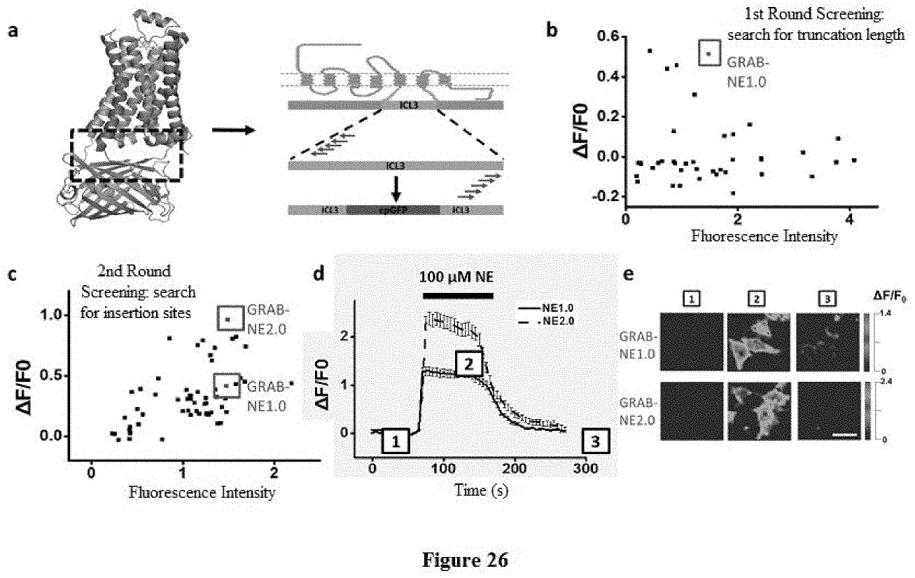

Using the truncation and insertion method, insertion sites were selected every 10 amino acids on ICL3 (157 amino acids), and a total of 14 insertion sites were designed. The sites were divided into two groups, a group close to the N-terminus and a group close to the C-terminus, with 7 sites in each group. Two insertion sites from the two groups were randomly selected for ICL3 truncation and cpEGFP insertion to obtain a truncation and insertion library with 49 possibilities (FIG. 26a). They were expressed in HEK293T cells, respectively. Using the high-content imaging system Opera Phenix, the fluorescence intensity on the cell membrane was detected with saturated concentration of agonist (norepinephrine NE, 100 μM) and without agonist. Red fluorescent protein localized on the membrane by CAAX modification was expressed under the same promoter, and the signal was detected as an internal reference. Through screening, the clone with high green fluorescence intensity (Fluorescent Intensity=GR ratio pre) before ligand addition and the largest change in fluorescence intensity (ΔF/F₀) after ligand addition was selected and named GRAB-NE1.0 (as shown in FIG. 26b). The sequencing results showed that the truncated insertion sites of this clone were positions 78 and 138 of ICL3 (i.e., segment 79-138 of ICL3 was cut out), that is, ICL3 was truncated 60 amino acids. The drug perfusion experiment was performed under a laser confocal microscope. With the treatment of 100 μM norepinephrine, the GRAB-NE1.0 sensor has a fluorescence intensity change of greater than 100%, and this change was reversible (as shown in FIGS. 26d, e).

In the present disclosure, when the truncated site was described, the numbers given correspond to the C-terminus amino acid, so the truncated position was numbered corresponding to the C-terminus amino acid. Unless otherwise indicated, this example, the following examples, and throughout the present disclosure were understood as such.

4. Obtaining of GRAB-NE2.0 with Higher Optical Signal Change Responding to NE by Finely Adjusting the Insertion Position of cpEGFP on the Truncated Human ADRA2A Receptor ICL3.

Around insertion sites 78 and 138 in ICL3, 11 candidates insertion sites were set respectively, that is, from position 73 to position 83 (insertion after any of the positions), and from position 133 to position 143 (insertion before any of the positions). The insertion sites were carefully adjusted, with a total of 121 possibilities. Similarly, they were expressed in HEK293T cells, respectively. Through screening (the drug was 100 μM NE norepinephrine), the clone with higher fluorescence intensity and the largest ΔF/F₀ was obtained and named GRAB-NE2.0 (as shown in FIG. 26c). The sequencing results show that the insertion sites of this clone were positions 78 and 143 of ICL3 (i.e., segment 79-143 of ICL3 was cut out). The drug perfusion experiment was performed under a laser confocal microscope. With the treatment of 100 μM norepinephrine, the GRAB-NE2.0 sensor has a fluorescence intensity change of greater than 200% (as shown in FIGS. 26d, e).

5. Obtaining of GRAB-NE2.1 with Higher Basal Fluorescence Intensity and Higher Optical Signal Change Responding to NE by Screening Peptide Linkers Between the Truncated Human ADRA2A Receptor ICL3 and cpEGFP.

After determining the optimal fluorescent protein insertion sites, the peptide linkers between the fluorescent protein and the receptor was optimized based on the GRAB-NE2.0 sensor.

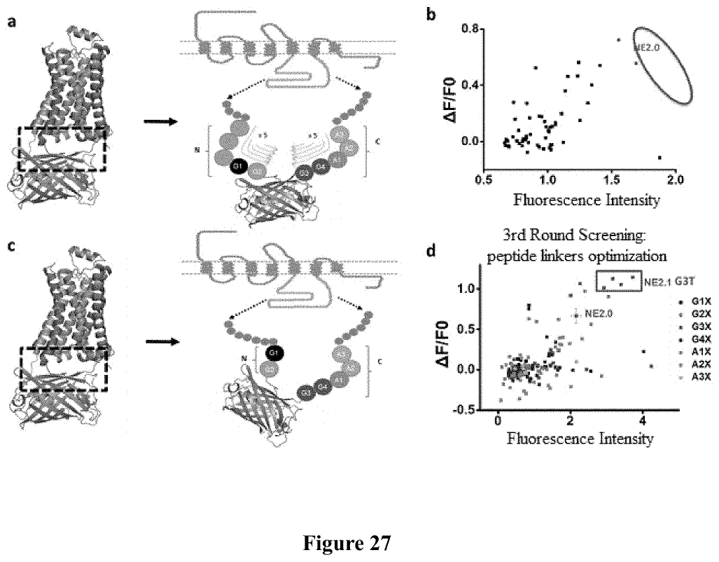

In the construction of the noradrenaline-specific fluorescent sensor based on the human ADRA2A receptor, a short peptide linker composed of flexible amino acids (glycine, alanine) was used to help the correct folding of the fusion protein. The lengths of the short peptide linker were 2 amino acids GG at the N-terminus and 5 amino acids GGAAA at the C-terminus. Based thereon, the linkers were screened for their length. The strategy was, changing the length of the linker on both sides of cpEGFP between 0-5 amino acids, truncating from the direction away from cpEGFP, and combining randomly the N-terminus with C-terminus to obtain all possible 25 permutations (as shown in FIG. 27a). They were expressed in HEK293T cells, respectively. It was found through high-throughput screening (using 100 μM NE) that, the change in the length of the peptide linker using GRAB-NE2.0 as a template did not improve the brightness and the response of the fluorescent sensor (as shown in FIG. 27b).

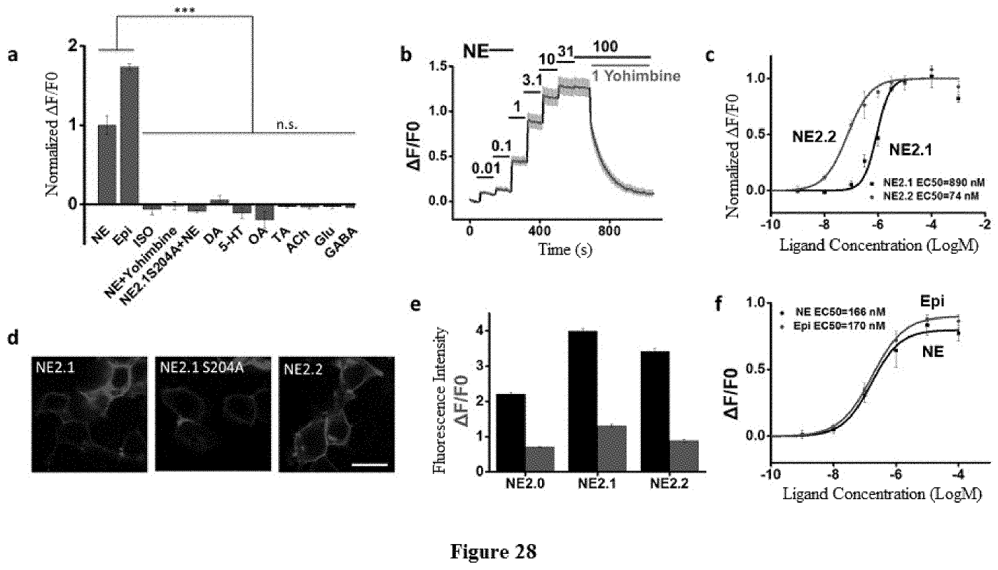

Based on the above experiments, the length of the peptide linker was fixed to the combination 2-5, and an attempt was made to change the type of amino acid to obtain a sensor with a greater signal change. Using random primer design method, NNB bases were used for coding at the amino acid site to be mutated to obtain the possibility of 20 different amino acids, and the probability of occurrence of termination codons was minimized. A total of 7 screening libraries for random mutations of amino acid sites were constructed, and each screening library has 20 possibilities (as shown in FIG. 27c). They were expressed in HEK293T cells, and through high-throughput screening, GRAB-NE2.1, which was brighter and more responsive than GRAB-NE2.0, was obtained. The sequence of the peptide linker of this new fluorescent sensor was GG-TGAAA, and the brightness and response thereof were about 1.5 times of GRAB-NE2.0 (as shown in FIGS. 27d and 28e).

The amino acid sequence of GRAB-NE2.1 is as follows:

(SEQ ID NO: 23)
METDTLLLWVLLLWVPGSTGDTSLYKKVGTTGGSLQPDAGNASWNGTEAP
GGGARATPYSLQVTLTLVCLAGLLMLLTVFGNVLVIIAVFTSRALKAPQN
LFLVSLASADILVATLVIPFSLANEVMGYWYEGKAWCEIYLALDVLECTS
SIVHLCAISLDRYWSITQAIEYNLKRTPRRIKAIIITVWVISAVISFPPL
ISIEKKGGGGGPQPAEPRCEINDQKWYVISSCIGSFFAPCLEVIILVYVR
IYQIAKRRTRVPPSRRGPDAVAAPPGGTERRPNGLGPERSAGPGGAEAEP
LPTQLNGAPGEPAPAGPRDTDALDLEEGGNVYIKADKQKNGIKANFHIRH
NIEDGGVQLAYHYQQNTPIGDGPVLLPDNHYLSVQSKLSKDPNEKRDHMV
LLEFVTAAGITLGMDELYKGGTGGSMVRKGEELFTGVVPILVELDGDVNG
HKFSVSGEGEGDATYGKLTLKFICTTGKLPVPWPTLVTTLTYGVQCFSRY
PDHMKQHDFFKSAMPEGYIQERTIFFKDDGNYKTRAEVKFEGDTLVNRIE
LKGIDFKEDGNILGHKLEYNTGAAARWRGRQNREKRFTFVLAVVIGVFVV
CWFPFFFTYTLTAVGCSVPRTLFKFFFWFGYCNSSLNPVIYTIENHDERR
AFKKILCRGDRKRIVL.

6. Norepinephrine GRAB Sensor has a Specific Optical Signal Change Caused by Change in Receptor Conformation Resulting from Ligand Binding, and the Specificity was Consistent with the Corresponding Receptor.

The cells expressing GRAB-NE2.1 were treated with ADRA2A receptor-specific blocker (Yohimbine, 2 μM), β2 adrenergic receptor-specific blocker (ICI118,551, 2 μM) and other neurotransmitters, respectively, and the changes in fluorescence signal under these conditions were observed. The results showed that, both norepinephrine NE and epinephrine Epi can activate GRAB-NE2.1, while the β-type receptor-specific activator ISO cannot, which was consistent with that the ADRA2A receptor can bind NE and Epi but not ISO. These results demonstrate that the neurotransmitter fluorescent sensor modified from GPCR retained the selectivity and specificity of endogenous GPCR for ligand. In addition, Yohimbine, a specific blocker of the α receptor, can inhibit the enhancement of fluorescent signal of the sensor caused by NE. Also, the mutation S204A in the ligand binding pocket of the ADRA2A receptor can disrupt the fluorescence signal change of GRAB-NE2.1 (as shown in FIGS. 28a and d). These results further demonstrate that the GRAB-NE sensor has specific fluorescence signal change responding to receptor activation. In addition, the cells transfected with GRAB-NE2.1 were treated with other common neurotransmitters respectively, none of them were able to significantly activate GRAB-NE2.1 to generate changes in fluorescent signal (as shown in FIG. 28a). This shows that the fluorescence change of the noradrenaline GRAB sensor was specific to receptor activation.

### 7. Noradrenaline GRAB Sensor has Ligand Concentration-Dependent Optical Response

HEK293T cells expressing the GRAB-NE2.1 sensor were treated with ligand of different concentrations from 1 nM to 1 mM (norepinephrine NE, at concentrations of 1 nM to 1 mM). It was found that, the sensor showed a concentration-dependent change of fluorescence signal in a wide range of neurotransmitter concentration, and the curve fitted to the Boltzmann distribution (as shown in FIG. 28b, c). The EC₅₀ calculated from the curve was 0.9 μM, which was in the same order of magnitude as the Kd value in the literature. It can be seen that the noradrenaline GRAB sensor did not change the affinity of the receptor for a specific ligand. Since the affinity of the receptor and ligand has evolved continuously, it can sensitively transmit neurotransmitter signals to downstream signals in the cell. Therefore, neurotransmitter fluorescent sensor with similar sensitivity to the wild-type receptor can sensitively and quantitatively detect the signal of different concentrations of neurotransmitter under physiological conditions.

The T373K mutation was introduced into the GRAB-NE2.1 sensor to obtain a GRAB-NE2.2 sensor with a 10-fold increase in ligand affinity (as shown in FIGS. 28c, d, and e). Although this sensor has lower basal fluorescence intensity and less fluorescence signal change than GRAB-NE2.1, the ligand affinity of about 100 nM made it more sensitive to detect the neurotransmitter signal. This sensor has an affinity for both norepinephrine NE and epinephrine Epi at the level of a few hundred nM (as shown in FIG. 28f). This indicates that mutations in the binding region of the GPCR and the ligand can regulate the binding affinity of the sensor, so as to obtain a fluorescent sensor with higher or lower ligand affinity. On the one hand, it can cover a wider detection range, and on the one hand, it can detect the release of neurotransmitter under the stimulation of a single action potential.

The amino acid sequence of GRAB-NE2.2 is as follows:

(SEQ ID NO: 24)
METDTLLLWVLLLWVPGSTGDTSLYKKVGTTGGSLQPDAGNASWNGTEAP
GGGARATPYSLQVTLTLVCLAGLLMLLTVFGNVLVIIAVFTSRALKAPQN
LFLVSLASADILVATLVIPFSLANEVMGYWYFGKAWCEIYLALDVLFCTS
SIVHLCAISLDRYWSITQAIEYNLKRTPRRIKAIIITVWVISAVISFPPL
ISIEKKGGGGGPQPAEPRCEINDQKWYVISSCIGSFFAPCLIMILVYVRI
YQIAKRRTRVPPSRRGPDAVAAPPGGTERRPNGLGPERSAGPGGAEAEPL
PTQLNGAPGEPAPAGPRDTDALDLEEGGNVYIKADKQKNGIKANFHIRHN
IEDGGVQLAYHYQQNTPIGDGPVLLPDNHYLSVQSKLSKDPNEKRDHMVL
LEFVTAAGITLGMDELYKGGTGGSMVRKGEELFTGVVPILVELDGDVNGH
KFSVSGEGEGDATYGKLTLKFICTTGKLPVPWPTLVTTLTYGVQCFSRYP
DHMKQHDFFKSAMPEGYIQERTIFFKDDGNYKTRAEVKFEGDTLVNRIEL
KGIDFKEDGNILGHKLEYNTGAAARWRGRQNREKRFKFVLAVVIGVFVVC
WFPFFFTYTLTAVGCSVPRTLFKFFFWFGYCNSSLNPVIYTIFNHDFRRA
FKKILCRGDRKRIVL.

### 8. The Rapid Kinetics of Noradrenaline GRAB Sensor Enabls Sub-Second Level Dynamic Detection

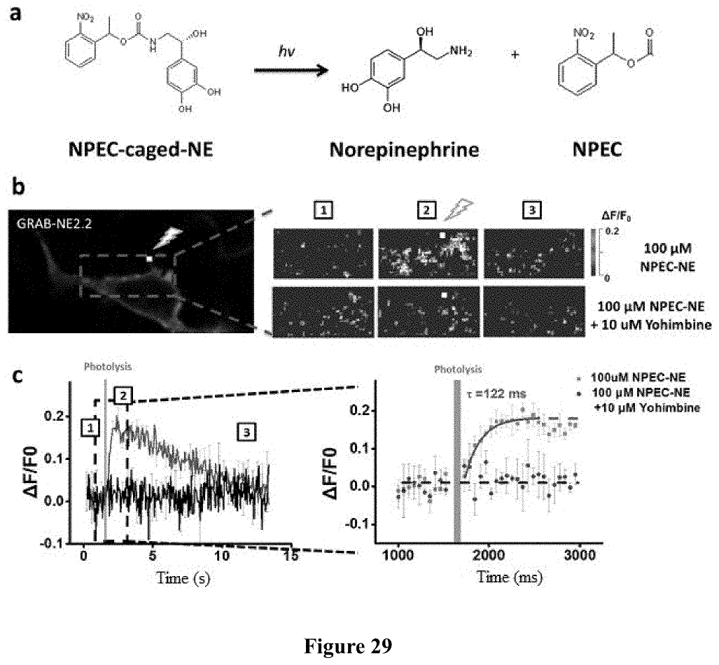

By caging neurotransmitters, neurotransmitters can be quickly activated and released in a certain area, and the signal of the sensor was fast scanned by a microscope to obtain the time constant for the change in fluorescence intensity after the sensor binds to the ligand. The experiment was performed in HEK293T cells using the neurotransmitter NPEC caged NE (i.e., NPEC-NE) (FIG. 29a), 405 nm laser for activating NPEC-caged NE, and GRAB-NE2.2. When uncaging was performed with a short-term high-energy 405 nm laser, it can be seen that the fluorescence signal after uncaging increased, which cannot be seen in the case of uncaging after the addition of the sensor-specific inhibitor Yohimbine and without adding the caged neurotransmitter NPEC-NE. No increase in fluorescence signal was observed after photolysis (FIG. 29b, c). This shows that, the increase in fluorescence signal after photolysis resulted from the detection of NE released specifically by photolysis by the GRAB-NE2.2 sensor. Using the single exponential growth equation to fit the increase of the fluorescence signal, a rate constant at which this fluorescence signal increases of about 100 ms can be obtained. This rate constant was enough to specifically capture the process of chemical synaptic signal transmission in complex neural networks.

9. Uncoupling of Noradrenaline GRAB Sensor from Downstream Signaling Pathway

G protein-mediated signaling pathways of norepinephrine receptors were tested, and the coupling ability of GRAB sensors to the downstream pathway of GPCR was observed to determine whether overexpression of the sensors in cells would cause unnecessary signaling pathway activation.

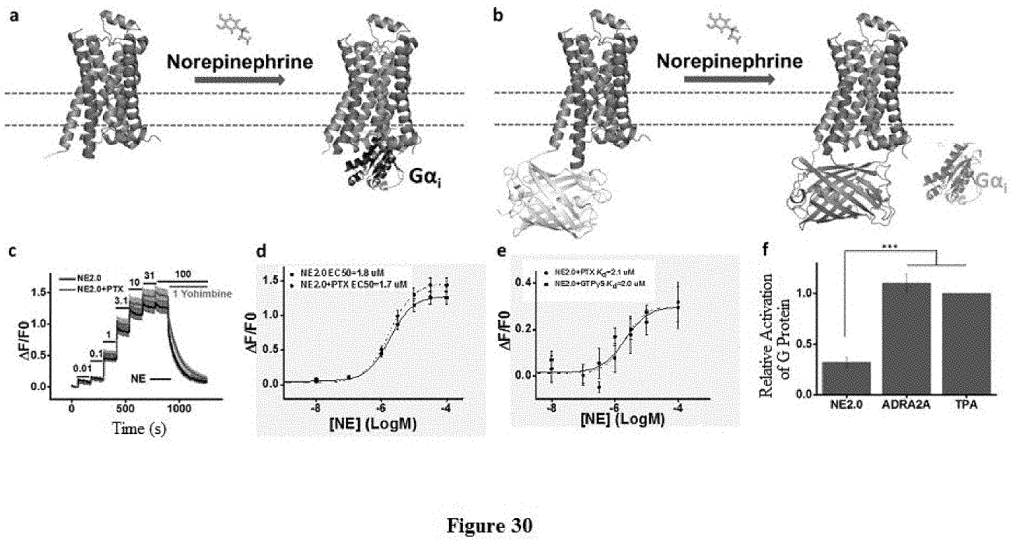

As to the G protein-dependent signaling pathway, the GRAB-NE receptor was a fluorescent sensor developed based on the ADRA2A receptor, and the ADRA2A receptor was endogenously coupled with Gαi protein to transduce inhibitory signal to downstream. By inhibiting the coupling of the Gαi protein and the sensor, the affinity of the fluorescent sensor to the ligand was detected to determine whether the fluorescent sensor needs the coupling of the downstream Gαi protein to maintain its activation state (as shown in FIG. 30a, b). The two methods were used to inhibit the coupling of Gαi protein, that is, co-expressing PTX pertussis toxin (PTX inhibits the activation of Gαi by catalyzing the ADP ribosylation of Gαi protein), and adding GTPγS (which can bind to Gαi protein to inhibit the dissociation of GTP, and thereby inhibiting the activation of G protein). It was found that neither of these two methods can change the affinity of the GRAB-NE2.0 fluorescent sensor for the ligand (FIGS. 30c-e). This indicates that the fluorescent sensor itself does not need the G protein coupling to stabilize its own activated conformation.

Whether the activation of the receptor causes the activation of the downstream G protein needs to be determined by the direct signal transmission intensity. The stable cell line for Gqi chimera was constructed using traditional resistance screening methods. A plasmid encoding the Gqi chimera protein and antibiotic resistance protein was transfected into the cells, and the encoding sequence was inserted into the genome of the cell through the homologous recombination sequence on the plasmid. The stable cell line was obtained through resistance screening. The role of the Gqi chimera protein is to convert the activation of the GPCR receptor (coupled to the Gi protein pathway) into the activation of the Gq pathway, so that the downstream detection methods of the Gq pathway (such as TGFα assay) can be used for Gi pathway detection. By the TGFα shedding assay, a common detection method for Gαq signaling pathway, the activation of Gαi-coupled signal was converted to the shedding signal of TGFα induced by Gαq, so that the strength of downstream G protein activation can be measured by the intensity of TGFα shedding. With the treatment of 10 μM NE, the TGFα signal caused by the GRAB-NE2.0 sensor was only ⅓ of the endogenous ADRA2A receptor (FIG. 30f). It can be concluded that the construction of this sensor indeed greatly reduced the coupling of this sensor to the downstream G protein, making the GRAB sensor free from causing obvious disturbance of the cell signaling system. This may be because the insertion of cpEGFP affects the position where GPCR was coupled to the G protein, so that the G protein coupling cannot be completed. Also, the insertion of cpEGFP mimics the structure change of GPCR after coupling to the G protein at certain way, which helps stabilizing GPCR in the activated state after binding to the ligand.

### 10. Noradrenaline GRAB Fluorescent Sensor has Optical Signal Changes for Specific Neurotransmitter in Cultured Neurons.

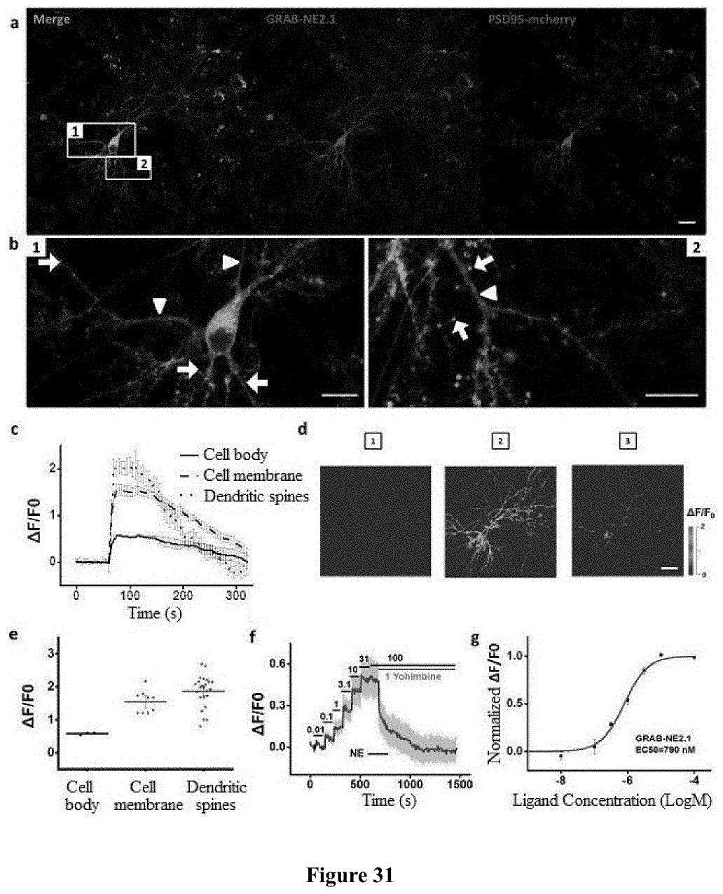

The neurotransmitter fluorescent sensor GRAB-NE2.1 constructed based on the ADRA2A receptor was expressed in cultured neurons, and the expression of the sensor and its response to specific neurotransmitter were observed. The primary cultured rat cortical neurons were transfected by calcium phosphate, and the neurons were imaged after about 48 hours. Neurons expressing GRAB-NE2.1 have normal morphology and nice axonal dendritic network. GRAB-NE2.1 was uniformly expressed on the cell membrane of neurons, except for a small amount of aggregation at the cell body. In different structures of neurons, the distribution of fluorescent sensors on dendritic spines can also be observed by co-transfecting PSD95-mcherry (FIGS. 31a, b).

The optical signal changes of neurons expressing neurotransmitter sensors were detected by perfusion with a neurotransmitter solution. The neurotransmitter-specific optical signals were recorded (FIGS. 31c, d). Due to the small amount of aggregation in the cell body, the change of optical signal was small. But the signals at the cell membrane and on the synapses were similar to those in HEK293T cells (FIG. 31e). Also, the signals were fast and stable, and have good repeatability in different neurons. The ligand concentration-dependent curve of the optical signal of the sensor was similar to that in the cultured cells and conformed to the Boltzmann equation, and its EC₅₀ value was similar to that reported in the literature (FIGS. 31f, g). The specific receptor blocker Yohimbine can inhibit the optical signal changes of the neurotransmitter sensors induced by ligand (FIG. 31f).

11. Epinephrine/Norepinephrine Fluorescent Sensors have Optical Signal Changes to Specific Neurotransmitters in Cultured Rat Cardiomyocytes.

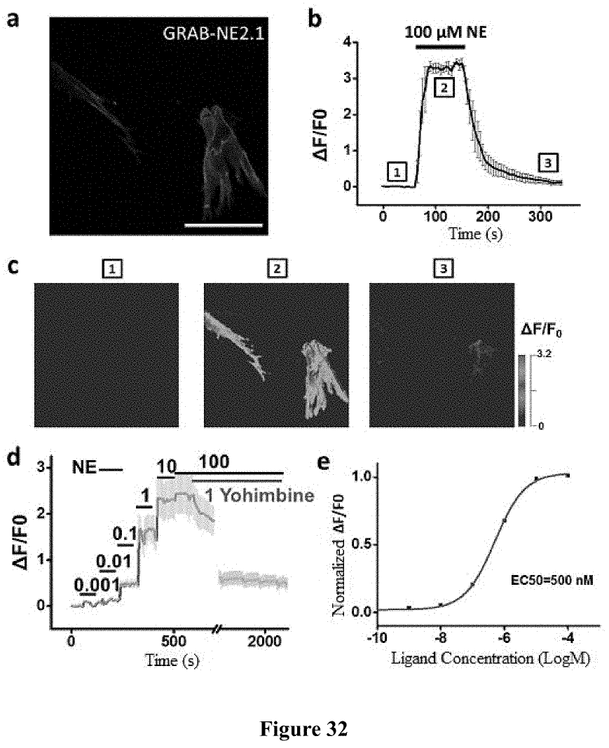

The GRAB-NE2.1 sensor was transfected into primary cultured rat cardiomyocytes by liposomes, and the optical signal changes for the ligand binding and affinity for ligand were detected by drug perfusion. The results showed that, the sensor has a good membrane localization in the cardiomyocytes and has an optical signal change of greater than 300% with the treatment of 100 μM saturated norepinephrine (FIGS. 32a, b and c). When treated with different concentrations of agonist (norepinephrine NE), the affinity of this sensor for the ligand in the cardiomyocytes was also similar to that previously determined, about 0.5 μM (FIGS. 32d, e).

### Example 9: Construction of Serotonin Fluorescent Sensor

Unless explicitly indicated, the materials and methods used in this example were the same as in Example 1.

Preliminary screening of different serotonin receptors reveals that human HTR1D and human HTR2C receptors have good expression and membrane localization after the insertion of fluorescent proteins. Then, taking human HTR2C as an example, the optimal insertion site of the fluorescent protein was screened.

### 1. Construction of a Serotonin-Specific Fluorescent Sensor Using Human HTR2C Receptor

A serotonin-specific fluorescent sensor was constructed using the human HTR2C receptor as a backbone, and a method of gradually determining the optimal insertion site of a fluorescent protein was adopted. For the third intracellular loop of the human HTR2C receptor, insertion site was set every 5 amino acids to insert a fluorescent protein, and the third intracellular loop was truncated at different amino acid positions for the insertion of fluorescent proteins, to obtain a sensor library. A fluorescent confocal microscope and a perfusion system were used to screen the sensor library. During the screening process, the sensor of which fluorescence intensity decreased after the adding of ligand was referred to as “OFF sensor”, and the sensor of which fluorescence intensity increased was referred to as “ON sensor”. After the first round of screening (the screening drug was serotonin 5HT with a concentration of 10 Mm), the two sensors with the highest response were selected for sequencing. It was found that, both sensors were truncated at the third intracellular loop of the human HTR2C receptor. One of the sensors was truncated at positions 15 to 55 of the third intracellular loop (ICL3) (that is, segment 16-55 of ICL3 was cut out, named 15N-55c), and the other sensor was truncated at positions 10 to 60 of ICL3 (that is, segment 11 to 60 of ICL3 was cut out, named 10N-60c).

Wherein, for the sequence of the human HTR2C receptor, see NCBI gene ID: 3358, isoform a, and the amino acid sequence thereof is:

(SEQ ID NO: 4)
MVNLRNAVHSFLVHLIGLLVWQCDISVSPVAAIVTDIFNTSDGGRFKFPD
GVQNWPALSIVIIIIMTIGGNILVIMAVSMEKKLHNATNYFLMSLAIADM
LVGLLVMPLSLLAILYDYVWPLPRYLCPVWISLDVLFSTASIMHLCAISL
DRYVAIRNPIEHSRFNSRTKAIMKIAIVWATSIGVSVPIPVIGLRDEEKV
FVNNTTCVLNDPNFVLIGSFVAFFIPLTIMVITYCLTIYVLRRQALMLLH
GHTEEPPGLSLDFLKCCKRNTAEEENSANPNQDQNARRRKKKERRPRGTM
QAINNERKASKVLGIVFFVFLIMWCPFFITNILSVLCEKSCNQKLMEKLL
NVFVWIGYVCSGINPLVYTLFNKIYRRAFSNYLRCNYKVEKKPPVRQIPR
VAATALSGRELNVNIYRHTNEPVIEKASDNEPGIEMQVENLELPVNPSSV
VSERISSV.

Wherein the underlined part (positions 236-311) is the third intracellular loop, as defined by the uniprot database.

Wherein the fluorescent protein used was the circular permutated cpEGFP, which was the same as in Example 2 and was the circular permutated fluorescent protein cpEGFP used in GCaMP6s, and the specific sequence thereof is:

(SEQ ID NO: 11)
NVYIKADKQKNGIKANFHIRHNIEDGGVQLAYHYQQNTPIGDGPVLLPDN
HYLSVQSKLSKDPNEKRDHMVLLEFVTAAGITLGMDELYKGGTGGSMVRK
GEELFTGVVPILVELDGDVNGHKFSVSGEGEGDATYGKLTLKFICTTGKL
PVPWPTLVTTLTYGVQCFSRYPDHMKQHDFFKSAMPEGYIQERTIFFKDD
GNYKTRAEVKFEGDTLVNRIELKGIDFKEDGNILGHKLEYN.

The sequence of the obtained sensor is as follows:

(SEQ ID NO: 25)
METDTLLLWVLLLWVPGSTGDTSLYKKVGTTGMVNLRNAVHSFLVHLIG
LLVWQCDISVSPVAAIVTDIFNTSDGGRFKFPDGVQNWPALSIVIIIIM
TIGGNILVIMAVSMEKKLHNATNYFLMSLAIADMLVGLLVMPLSLLAIL
YDYVWPLPRYLCPVWISLDVLFSTASIMHLCAISLDRYVAIRNPIEHSR
FNSRTKAIMKIAIVWAISIGVSVPIPVIGLRDEEKVFVNNTTCVLNDPN
FVLIGSFVAFFIPLTIMVITYCLTIYVLRRQALMLLNGNVYIKADEQKN
GIKAYFKIRHNIEGGGVQLAYHYQQNTPIGDGPVLLPDNHYLSVQSMLS
KDPNEKRDHMVLLEFVTAAGITLGMDELYKGGTGGSESMVSKGEELFTG
VVPIQVELDGDVNGHKFSVSGEGEGDATYGKLTLKFICTTGKLPVPWPT
LVTTLTYGVQCFSRYPDHMKQHDFFKSAMPEGYIQERTIFFKGDGNYKT
RAEVKFEGDTLVNRIELKGIDFKEDGNILGHKLEYNGFAAANNERKASK
VLGIVFFVFLIMWCPFFITNILSVLCEKSCNQKLMEKLLNVFVWIGYVC
SGINPLVYTLFNKIYRRAFSNYLRCNYKVEKKPPVRQIPRVAATALSGR
ELNVNIYRHTNEPVIEKASDNEPGIEMQVENLELPVNPSSVVSERISS
V.

### 2. Optimization of the Insertion Site of the Fluorescent Protein in the Third Intracellular Loop of the HTR2C Receptor

The screening strategy was to fix the first 10 or 15 amino acids at the N-terminus of the third intracellular loop, and systematically scan the insertion site of the fluorescent protein at the C-terminus of the intracellular loop (named 10_{N}-X_{C} or 15_{N}-X_{C}, that is, the amino acids 11-X or 16-X of ICL3 were truncated). Similarly, fix the amino acid after position 55 or 60 of the third intracellular loop, and scan the insertion site of the fluorescent protein at the N-terminus of the intracellular loop (named X_{N}-55_{C} or X_{N}-60_{C}, that is, the amino acids X+1-55 or X+1-60 of ICL3 were truncated). The specific screening method was to transfect human HTR2C receptors inserted with fluorescent protein at different positions into HEK293T cells, perfuse with serotonin, and measure ΔF/F₀. In the 10_{N}-X_{C} screening library, the combination 10_{N}-60_{C} showed the largest OFF response; and in the 15_{N}-X_{C} screening library, when the combination of the insertion sites of the fluorescent protein was 15_{N}-70_{C} (that is, positions 16-70 of ICL3 were truncated), the sensor showed an ON response of about 20%. Thereafter, the amino acid sequence after position 70 of the third intracellular loop of the human HTR2C receptor was fixed, and the insertion site of the fluorescent protein at the N-terminus of the third intracellular loop was scanned (named X_{N}-70_{C}), but no better sensor was found.

Then, the insertion site was more precisely screened. The insertion site on the left was determined to be the amino acids positions 13 to 17 of the third intracellular loop, and the insertion site on the right was determined to be the amino acids positions 66 to 74. These sites were combined to construct a sensor library and subjected to screening. The specific screening method was the same as above. By systematically screening the insertion site of the fluorescent protein in the human HTR2C receptor, a serotonin fluorescent sensor 14_{N}-68_{C} with 80% ON response was obtained (that is, positions 15-68 of ICL3 were truncated) and named GRAB-5-HT1.0.

### 3. Optimization of the Peptide Linker Between the Circular Permutated Fluorescent Protein and the HTR2C Receptor

In the initial peptide linkers, the peptide linker at the N-terminus was 2 amino acids in length and the sequence was GG; and the peptide linker at the C-terminus was 5 amino acids in length and the sequence was GGAAA. On the basis of GRAB-5-HT1.0, random mutations were introduced sequentially at each site of the peptide linker. Once the excellent sensor was screened, the amino acid at this position was fixed and the random mutation at the next site was continued. It was found that when the glycine G at the first position of the peptide linker at the N-terminus was mutated to asparagine N, the signal of the sensor increased by 3 times, from 80% to nearly 300%. Therefore, the first amino acid was fixed as asparagine. After completing the screening of all positions in the peptide linkers, the optimal serotonin sensor obtained has a fluorescence signal increase of about 350% responding to the saturated concentration of serotonin. The sensor was named GRAB-5-HT2.0, and the peptide linkers thereof were NG at the N-terminus and GFAAA at the C-terminus.

According to the screening results of the peptide linker, it was found that the change of the first amino acid of the peptide linker at the N-terminus has a greater impact on the performance of the sensor. Considering the interaction between amino acids, the three sites in front of the peptide linker at the N-terminus, positions 12, 13 and 14 of the third intracellular loop of the human HTR2C receptor, were screened using the same strategy. As a result, it was found that the changes in amino acids positions 12 and 14 have no effect on the performance of the sensor, while after mutating leucine L at position 13 to phenylalanine F, the signal of the sensor increased to nearly 500%, and this sensor was named GRAB-5-HT2.1.

4. Serotonin Sensors have Ligand Concentration-Dependent Optical Response

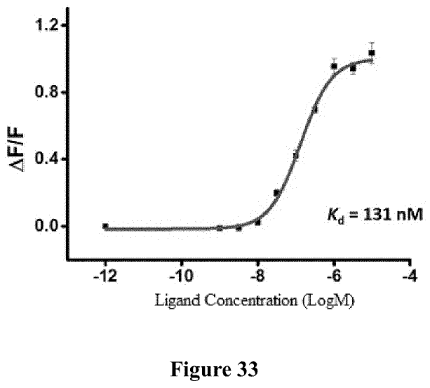

Using different concentrations of serotonin to activate the GRAB-5-HT2.1 sensor, it was found that the sensor showed a concentration-dependent increase in fluorescence signal over a wide range of serotonin concentrations (FIG. 33), and the curve conformed to Hill distribution. By calculating the Kd value of GRAB-5-HT2.1 and comparing it with the Kd value of HTR2C receptor reported in the literature for 5-HT, it was found that the modification of the human HTR2C receptor did not affect its affinity for the ligand. The reason may be that the binding site of 5-HT to the human HTR2C receptor was mainly located in the transmembrane region and extracellular region of the latter, and the third intracellular loop of the human HTR2C receptor was modified in the present disclosure. Ligand concentration-dependent response curves show that serotonin sensors can sensitively and quantitatively detect serotonin signal of different concentrations under physiological conditions.

5. Serotonin Sensors have Specific Ligand-Induced Optical Signal Changes

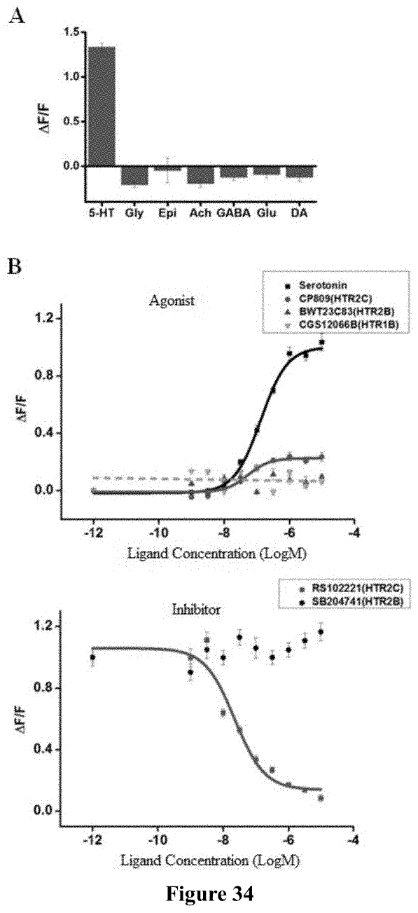

HEK293T cells expressing the serotonin sensor GRAB-5-HT2.1 were treated with different concentrations of neurotransmitters at saturated concentrations. It was found that only serotonin can induce a big change in the fluorescence signal of this sensor (FIG. 34A), while other neurotransmitters cannot induce a change in the optical signal of the GRAB-5-HT2.1 sensor even at high concentrations.

The addition of HTR2C receptor-specific agonist (CP809) to the GRAB-5-HT2.1 sensor can induce changes in fluorescent signal, while an HTR2B receptor-specific agonist (BWT23C83) and an HTR1B receptor-specific agonist (CGS12066B) cannot change the fluorescent signal. In addition, first adding serotonin induced an increase in the fluorescent signal, and then adding an HTR2C receptor-specific antagonist (RS102221) can inhibit the serotonin-induced increase in the fluorescence signal of the GRAB-5-HT2.1 sensor; while the addition of HTR2B receptor-specific antagonist (SB204741) failed to inhibit the serotonin-induced increase in the fluorescence signal of the sensor (FIG. 34B). These results indicate that the sensor constructed based on human HTR2C receptor has receptor subtype specificity.

### 6. Construction of a Series of Serotonin Fluorescent Sensors by the Method of Constructing a Chimeric Receptor

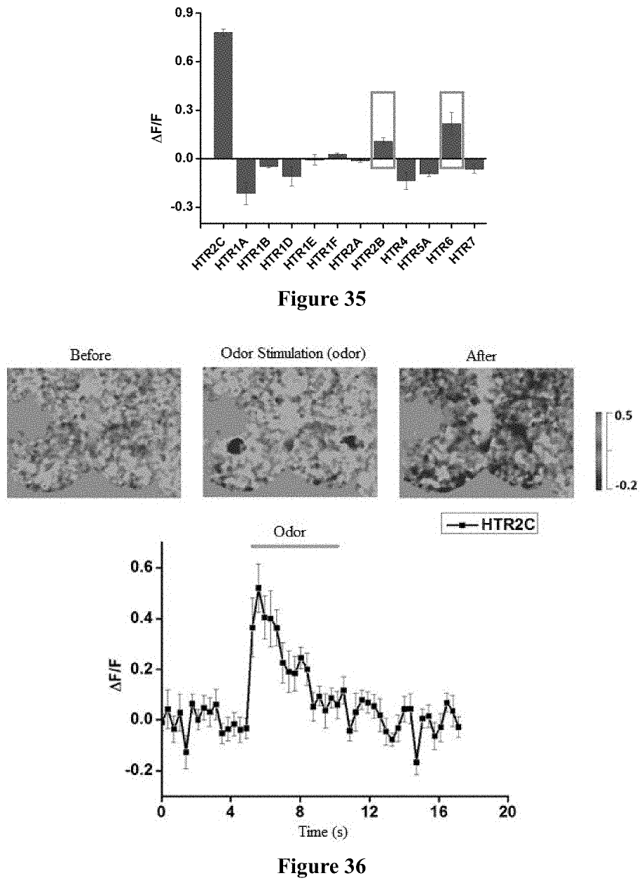

Based on the known structures of the human HTR1B and human HTR2B receptors, the binding sites to their ligand were analyzed, and it was found that none of these sites involved the third intracellular loop of the HTR receptors. Therefore, the method of constructing chimeric receptors can be used to construct fluorescent sensors based on other serotonin receptors. Sequence alignment was performed for different receptors of HTR, and their original third intracellular loops were replaced with the third intracellular loop of GRAB-5-HT2.1 constructed with the human HTR2C receptor. The sensors were treated with the saturated concentration of 5-HT, and the changes in fluorescence signal were observed. It was found that the sensors constructed with human HTR2B and human HTR6 receptors show good membrane localization, and at the same time, there is an increase in fluorescence signal after the addition of the saturated concentration of 5-HT (FIG. 35).

For the sequence of HTR2B, see NCBI gene ID: 3357, isoform 1. The specific sequence is:

(SEQ ID NO: 9)
MALSYRVSELQSTIPEHILQSTFVHVISSNWSGLQTESIPEEMKQIVEEQ
GNKLHWAALLILMVIIPTIGGNTLVILAVSLEKKLQYATNYFLMSLAVAD
LLVGLFVMPIALLTIMFEAMWPLPLVLCPAWLFLDVLFSTASIMHLCAIS
VDRYIAIKKPIQANQYNSRATAFIKITVVWLISIGIAIPVPIKGIETDVD
NPNNITCVLTKERFGDFMLFGSLAAFFTPLAIMIVTYFLTIHALQKKAYL
VKNKPPQRLTWLTVSTVFQRDETPCSSPEKVAMLDGSRKDKALPNSGDET
LMRRTSTIGKKSVQTISNEQRASKVLGIVFFLFLLMWCPFFITNITLVLC
DSCNQTTLQMLLEIFVWIGYVSSGVNPLVYTLFNKTFRDAFGRYITCNYR
ATKSVKTLRKRSSKIYFRNPMAENSKFFKKHGIRNGINPAMYQSPMRLRS
STIQSSSIILLDTLLLTENEGDKTEERVSYV.

Wherein the underlined part (positions 240-324) is the third intracellular loop, as defined by the uniprot database.

For the sequence of HTR6, see NCBI gene ID: 3362, isoform 1. The specific sequence is:

(SEQ ID NO: 10)
MVPEPGPTANSTPAWGAGPPSAPGGSGWVAAALCVVIALTAAANSLLIAL
ICTQPALRNTSNFFLVSLFTSDLMVGLVVMPPAMLNALYGRWVLARGLCL
LWTAFDVMCCSASILNLCLISLDRYLLILSPLRYKLRMTPLRALALVLGA
WSLAALASFLPLLLGWHELGHARPPVPGQCRLLASLPFVLVASGLTFFLP
SGAICFTYCRILLAARKQAVQVASLTTGMASQASETLQVPRTPRPGVESA
DSRRLATKHSRKALKASLTLGILLGMFFVTWLPFFVANIVQAVCDCISPG
LFDVLTWLGYCNSTMNPIIYPLFMRDFKRALGRFLPCPRCPRERQASLAS
PSLRTSHSGPRPGLSLQQVLPLPLPPDSDSDSDAGSGGSSGLRLTAQLLL
PGEATQDPPLPTRAAAAVNFFNIDPAEPELRPHPLGIPTN.

Wherein the underlined part (positions 209-265) is the third intracellular loop, as defined by the uniprot database.

### 7. Detection of the Release of Serotonin in the Central Nervous System of Drosophila by Two-Photon Imaging

The transgenic Drosophila UAS-GRAB-5-HT was constructed using GRAB-5-HT2.0. After crossing it with the Trh-Gal4 strain, the sensor was specifically expressed in serotonergic neurons. The neuronal activity of serotoninergic neurons induced by olfactory stimulation when isoamyl acetate was given was successfully detected using two-photon imaging (FIG. 36).

### 8. High-Throughput Drug Screening Using Cell Lines Expressing GRAB-5-HT1.0

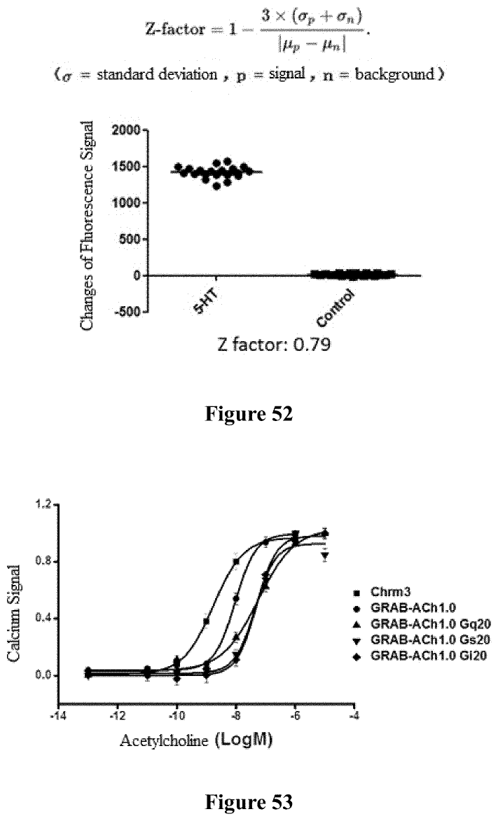

HEK293T stable cell line expressing the GRAB-5-HT1.0 sensor was constructed. Using a high-throughput drug screening platform to detect the fluorescence signal of cells expressing GRAB-5-HT1.0 sensor after adding serotonin and compare the signal with the control group (solvent group) (FIG. 52). The platform is based on computer-controlled robotic arms for experimental operations, using computer control for drug addition, precipitation, and the detection of fluorescence signal, so as to achieve better repeatability and stability. It can be seen from the figure that the detection method has good repeatability and sensitivity (represented by Z factor, which is a parameter that depicts whether the system is sufficiently sensitive and stable during the high-throughput screening process, and the formula thereof was shown in FIG. 52). In general, the Z factor of a system suitable for high-throughput screening needs to be greater than 0.4. This indicates that the method for constructing stable cell lines based on GRAB sensors has sufficient sensitivity and stability for high-throughput drug screening.

### Example 10: Construction of Dopamine Sensor

Unless explicitly indicated, the materials and methods used in this example were the same as in Example 1.

### 1. Construction of a Dopamine Sensor Encoding by DNA

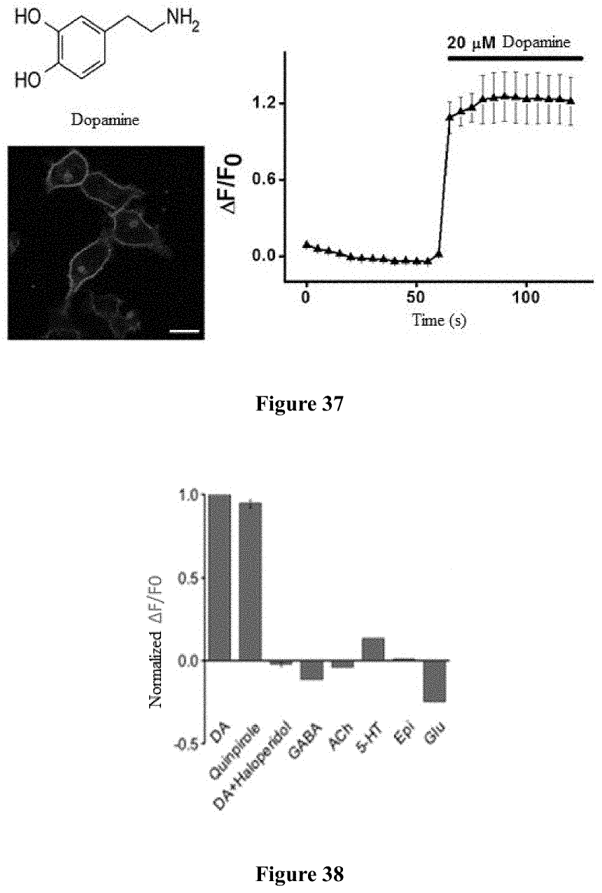

There are 5 subtypes of human dopamine receptors in the body, which are named DRD1-DRD5. For constructing a fluorescent sensor, the receptors were screened preliminarily by inserting a fluorescent protein at any position in the third intracellular loop of the receptor. A good candidate receptor, human DRD2 receptor, was identified for its expression and membrane localization. Subsequently, a strategy similar to the epinephrine sensor in Example 2 was adopted to determine the optimal insertion site of the fluorescent protein. Specifically, for the third intracellular loop of the human DRD2 receptor, the insertion sites were set every 15 amino acids. Human DRD2 receptors inserted with circular permutated fluorescent proteins at different positions were expressed in HEK293T cells, and drug perfusion experiments were conducted with dopamine. Finally, multiple fluorescent sensors sensitive to dopamine were identified. Among them, the fluorescent sensor with the largest signal change was one in which amino acids positions 253 to 357 of the human DRD2 receptor were truncated and a circular permutated fluorescent protein was inserted at the truncated position. After the positions sensitive to conformational change were determined, the surrounding amino acid sites thereof were further screened. The sites surrounding the amino acid position 252 at N-terminus and the sites surrounding the amino acid position 357 at the C-terminus were combined and different sensors were constructed. The sensors were expressed in HEK293T cells and subjected to drug perfusion experiments with dopamine. The optimal sensor was one in which amino acid positions 254 to 360 were truncated and a circular permutated fluorescent protein was inserted at the truncated position, which could achieve a signal change of 110% under the treatment of saturated concentration of dopamine (FIG. 37), and named GRAB-GDA3.0. The peptide linkers were GG at the N-terminus and GGAAA at the C-terminus.

Wherein, for the sequence of the human DRD2 receptor, see NCBI gene ID: 1813, isoform long. The specific amino acid sequence is:

(SEQ ID NO: 5)
MDPLNLSWYDDDLERQNWSRPFNGSDGKADRPHYNYYATLLTLLIAVIV
FGNVLVCMAVSREKALQTTTNYLIVSLAVADLLVATLVMPWVVYLEVVG
EWKFSRIHCDIFVTLDVMMCTASILNLCAISIDRYTAVAMPMLYNTRYS
SKRRVTVMISIVWVLSFTISCPLLFGLNNADQNECIIANPAFVVYSSIV
SFYVPFIVTLLVYIKIYIVLRRRRKRVNTKRSSRAFRAHLRAPLKGNCT
HPEDMKLCTVIMKSNGSFPVNRRRVEAARRAQELEMEMLSSTSPPERTR
YSPIPPSHHQLTLPDPSHHGLHSTPDSPAKPEKNGHAKDHPKIAKIFEI
QTMPNGKTRTSLKTMSRRKLSQQKEKKATQMLAIVLGVFIICWLPFFIT
HILNIHCDCNIPPVLYSAFTWLGYVNSAVNPIIYTTFNIEFRKAFLKIL
HC.

Wherein the underlined part is the third intracellular loop, specifically positions 214-373, as defined by the uniprot database.

Wherein the fluorescent protein used here was the circular permutated cpEGFP, which was the same as in Example 2 and was the circular permutated fluorescent protein cpEGFP used in GCaMP6s, and the specific sequence thereof is:

(SEQ ID NO: 11)
NVYIKADKQKNGIKANFHIRHNIEDGGVQLAYHYQQNTPIGDGPVLLPD
NHYLSVQSKLSKDPNEKRDHMVLLEFVTAAGITLGMDELYKGGTGGSMV
RKGEELFTGVVPILVELDGDVNGHKFSVSGEGEGDATYGKLTLKFICTT
GKLPVPWPTLVTTLTYGVQCFSRYPDHMKQHDFFKSAMPEGYIQERTIF
FKDDGNYKTRAEVKFEGDTLVNRIELKGIDFKEDGNILGHKLEYN.

Pharmacological studies show that GRAB-GDA3.0 can only be activated by DA, but not other neurotransmitters. In addition, it can also be activated or inhibited by DRD2-specific agonists (Dopamine) or antagonists (haloperidol), respectively (FIG. 38).

The sequence of the constructed GRAB-GDA3.0 is as follows:

(SEQ ID NO: 26)
METDTLLLWVLLLWVPGSTGDTSLYKKVGTMDPLNLSWYDDDLERQNWS
RPFNGSDGKADRPHYNYYATLLTLLIAVIVFGNVLVCMAVSREKALQTT
TNYLIVSLAVADLLVATLVMPWVVYLEVVGEWKFSRIHCDIFVTLDVMM
CTASILNLCAISIDRYTAVAMPMLYNTRYSSKRRVTVMISIVWVLSFTI
SCPLLFGLNNADQNECIIANPAFVVYSSIVSFYVPFIVTLLVYIKIYIV
LRRRRKRVNTKRSSRAFRAHLRAPLKGNCTHPEDMKLGGNVYIKADKQK
NGIKANFHIRHNIEDGGVQLAYHYQQNTPIGDGPVLLPDNHYLSVQSKL
SKDPNEKRDHMVLLEFVTAAGITLGMDELYKGGTGGSMVRKGEELFTGV
VPILVELDGDVNGHKFSVSGEGEGDATYGKLTLKFICTTGKLPVPWPTL
VTTLTYGVQCFSRYPDHMKQHDFFKSAMPEGYIQERTIFFKDDGNYKTR
AEVKFEGDTLVNRIELKGIDFKEDGNILGHKLEYNGGAAAMSRRKLSQQ
KEKKATQMLAIVLGVFIICWLPFFITHILNIHCDCNIPPVLYSAFTWLG
YVNSAVNPIIYTTFNIEFRKAFLKILHC.

### 2. Odor Stimulates GDA Signal in Mushroom Body (MB).

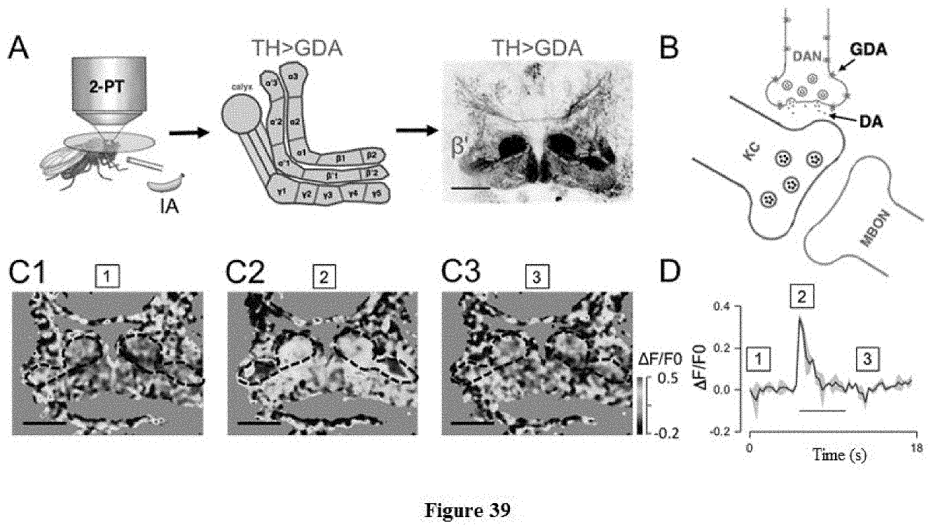

The transgenic Drosophila UAS-GRAB-GDA3.0 was constructed to express the GRAB-GDA3.0 sensor in specific cells, driven by the corresponding GAL4 strain. First, GRAB-GDA3.0 was expressed in dopaminergic neurons (DAN) in all Drosophila, and the response induced by odor (isoamyl acetate) was examined by in vivo two-photon imaging (FIG. 39A). GRAB-GDA3.0 expressed in the cell membrane of DAN was capable of reporting the release of dopamine (DA) via a sensor located at presynaptic site (FIG. 39B). A few seconds after the odor was released, a robust signal from GRAB-GDA3.0 was observed throughout the mushroom body (MB), especially (3′ lobe (FIGS. 39C and D).

### 3. The Odor-Stimulated GDA Signals in MB are Specific to DA.

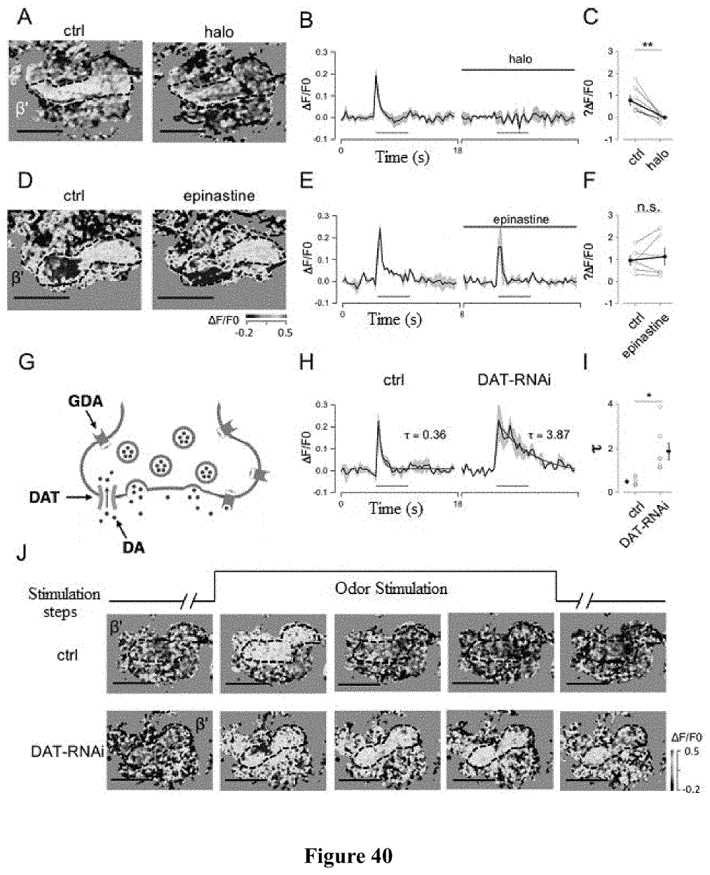

A pharmacological study was performed on GRAB-GDA3.0. Different drugs were incubated in the solution where Drosophila was imaged, and it was found that the GDA signal was completely blocked by the DRD2-specific antagonist halo (haloperidol) (FIGS. 40A-C), and as a control, it failed to be blocked by epinastine, an octopamine receptor-specific antagonist (FIGS. 40D-F). These results demonstrate that the GDA signal was specific to DA.

In addition, the specificity for GDA was confirmed by genetic research. The rate of GDA signal attenuation between normal Drosophila and Drosophila with reduced DAT expression was compared (this is the experiment of gene level research using DAT-RNAi described later). In DAN, the dopamine transporter (DAT) is located in the presynaptic membrane and releases DA intermittently and cyclically from the synapse. DAT-RNAi was used to inhibit DAT expression in DAN (FIG. 40G). Theoretically, the decay time of GDA signal in DAT-RNAi Drosophila should be longer than that of WT Drosophila. In fact, the duration of the odor-induced GDA signal in DAT-RNAi Drosophila (τ=1.85 s) was indeed longer than that of WT Drosophila (τ=0.48 s) (FIGS. 40H-J).

### Example 11: Construction of Dopamine and Serotonin Sensors with Red Fluorescent Protein

### 1. Materials and Methods

### Molecular Cloning

The GRAB sensor plasmid was constructed by pDisplay vector (Invitrogen) with an IgK leader sequence before the coding region and a termination codon before the transmembrane region. The cpmApple gene (a type of cpRFP, and RFP was the red fluorescent protein) was amplified from R-GECO1 (Yongxin Zhao, et al, An Expanded Palette of Genetically Encoded Ca2+ indicators, Science, 2011) (gift by Dr. Robert E. Campbell). The full-length human GPCR cDNA was amplified from hORFeome database 8.1. Gibson assembly was used for all molecular cloning, including site-directed mutations, and the primers used have a 30-base overlap. The correct clones were verified by Sanger sequencing.

The amino acid sequence of cpmApple:

(SEQ ID NO: 12)
PVVSERMYPEDGALKSEIKKGLRLKDGGHYAAEVKTTYKAKKPVQLPGA
YIVDIKLDIVSHNEDYTIVEQCERAEGRHSTGGMDELYKGGTGGSLVSK
GEEDNMAIIKEFMRFKVHMEGSVNGHEFEIEGEGEGRPYEAFQTAKLKV
TKGGPLPFAWDILSPQFMYGSKAYIKHPADIPDYFKLSFPEGFRWERVM
NFEDGGIIHVNQDSSLQDGVFIYKVKLRGTNFPPDGPVMQKKTMGWEAT
R.

### Cell Culture and Transfection

HEK293T cells were grown at 37° C. and 5% CO₂ using DMEM supplemented with 10% FBS and penicillin-streptomycin. Cells were placed on 12-mm coverslips in a 24-well plate. Cortical neurons were cultured as described below. Cortex tissue from P1 rat was dissected and digested with 0.25% Trypsin-EDTA (Gibco), and then seeded on poly-D-lysine-coated coverslips at a density of 0.5-1×10⁶ cells/ml. For transfection, HEK293T cells were transiently transfected by the PEI method at a ratio of 1 μg DNA:4 μg PEI. The medium was changed 4-6 h after transfection, and cells were imaged 24 h later. The cultured neurons were transfected using the calcium phosphate method. After 1.5 h, the precipitations were dissolved with 1×HBS (pH 6.8). Neurons were transfected after 7-9 d in vitro, and experiments were performed 48 h after transfection.

### Fluorescence Imaging and Perfusion of Cultured Cells

HEK293T cells and cultured cortical neurons were perfused with a standard extracellular Tyrode solution containing (in mM): 150 NaCl, 4 KCl, 2 MgCl₂, 2CaCl₂, 10 HEPES and 10 Glucose, pH 7.4. Coverslips were placed in a perfusion chamber and connected to a miniature manifold for perfusion. HEK293T cells and neurons were imaged using the Nikon confocal system.

### 2. Strategy for Constructing a Red Fluorescent GRAB Sensor

The circular permutated red fluorescent protein cpmApple was inserted into the third intracellular loop of the GPCR, thereby converting the conformational change of the ligand-induced GPCR into an optical signal. The process for constructing a red GRAB sensor is as follows: 1) find the optimal insertion site of cpmApple in the GPCR. CpmApple insertion sites were designed every 5 amino acids throughout the third intracellular loop, and cpmApple was inserted at different truncated positions. HEK293 cells expressing the sensors were subjected to perfusion experiment with saturated ligand to screen for the construct with the largest fluorescence response. 2) fine tune the insertion sites. After locating the possible insertion sites, a similar method as in the first step was used to fine tune the insertion sites one by one residue around the optimal response site. 3) optimize the sequences of the peptide linker at N-terminus and C-terminus of cpmApple. First, the N-terminus and C-terminus peptide linkers of cmpApple were optimized independently by repeated mutations and screening, and then the optimal N and C peptide linker sequences were combined for screening. During the screening process, mutants with both higher ΔF/F₀ and higher fluorescence brightness were selected.

### 3. Construction of a Red Fluorescent Dopamine Sensor

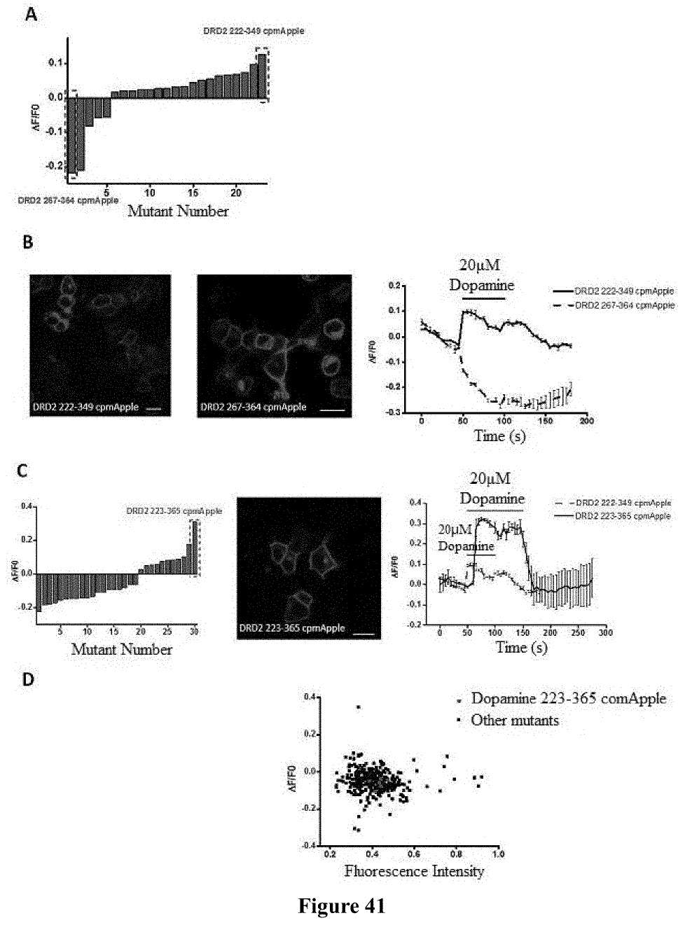

Followed the steps of the above strategy to find the optimal insertion site for cmpApple with the largest ligand-induced response and optimal membrane localization. The human dopamine receptor DRD2 (the same receptor as in Example 10) was selected to construct a sensor, and 92 variants in the constructed library were subjected to perfusion experiments. Among them, 16 have no fluorescence, 56 have no ligand-induced response, 15 showed on-response and 5 showed off-response. Herein, the on-response indicates that the fluorescence signal was enhanced when the cells were perfused with a buffer containing a saturated concentration of the ligand, and the off-response indicates that the fluorescence signal decreased when the cells were perfused with a buffer containing a saturated concentration of the ligand. The results of the on-response and off-response were shown in FIG. 41A. The optimal on-response candidate DRD 222-349 cmpApple (that is, the positions 223-349 of DRD were truncated and cmpApple was inserted at the truncated position) shows more than 13% on-response, and the optimal off-response candidate DRD 267-364 cmpApple (that is, the positions 268-364 of DRD were truncated and cmpApple was inserted at the truncated position) shows more than 22% off response. Imaging characteristics and response curves were shown in FIG. 41B. Herein, the number after DRD indicates the insertion site of cmpApple. Both candidates showed good membrane localization. Because on-response sensors usually have a better signal-to-noise ratio in imaging, on-response candidates were subjected to further optimization. After fine tuning the insertion site, the ligand-induced response increased to 32%. The left panel of FIG. 41C shows the strongest response. The optimal on-response candidate DRD 223-365 cmpApple (that is, the positions 224-365 of DRD were truncated and cmpApple was inserted at the truncated position) shows a 32% on-response (FIG. 41C, right panel). The sensor was localized well on the membrane (FIG. 41C, middle panel). Then, the third step was used to optimize the sequence of DRD 223-365 cmpApple peptide linkers. There were 5 amino acids peptide linker at the N-terminus and three amino acids peptide linker at the C-terminus of cmpApple. Random mutation was performed independently on each amino acid of the peptide linker one by one. Some variants show higher ΔF/F₀ and higher brightness, and the initial sequence of the peptide linker of one of the variants was PVVSE (N-terminus) and ATR (C-terminus) (FIG. 41D).

### 4. Construction of a Red Fluorescent Serotonin Sensor

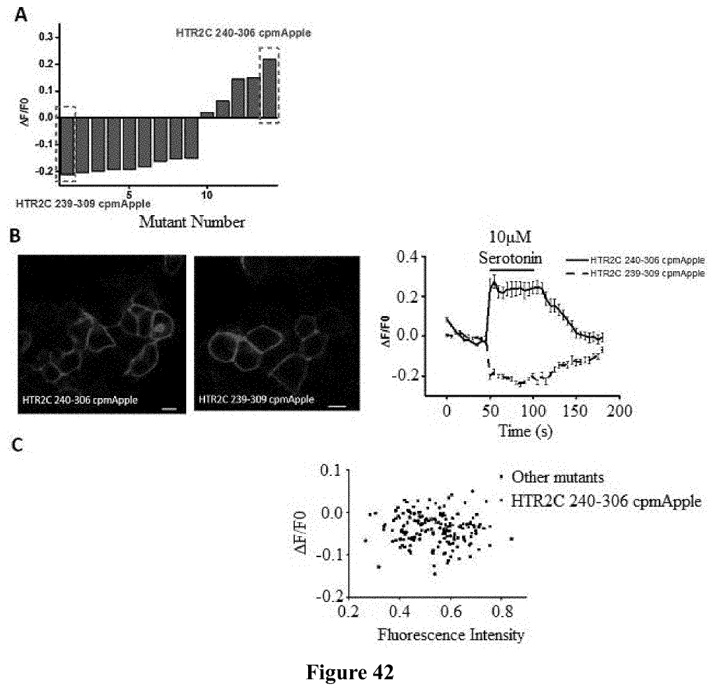

A red GRAB sensor for serotonin was constructed using a strategy similar to the red fluorescent dopamine sensor. The human serotonin receptor HTR2C (with the same sequence as in Example 9) was selected to construct the sensor. In the library constructed by the cpmApple insertion strategy and subsequent fine-tuning strategy, HTR2C 240-306 cpmApple (that is, the positions 241-306 of HTR2C were truncated and cmpApple was inserted at the truncated position) with 27% on-response and HTR2C 239-309 cpmApple (that is, the positions 240-309 of HTR2C were truncated and cmpApple was inserted at the truncated position) with 21% off-response were obtained (FIGS. 42A and 42B). Their peptide linkers were PVVSE (N-terminus) and ATR (C-terminus). Random mutation was performed on 5 amino acids of the peptide linker at the N-terminus of cpmApple, and some variants show higher ΔF/F₀ and higher brightness (FIG. 42C).

### Example 12: Construction of the Serotonin BRET Sensor

Bioluminescence is derived from chemical reactions. Compared with fluorescence, bioluminescence can be imaged without the excitation of an external light source, which avoids unfavorable effects such as tissue autofluorescence, phototoxicity, and photobleaching induced by external excitation light, and is particularly suitable for imaging of living animals, especially deep tissue imaging. Nanoluc is a luciferase with extremely high catalytic activity and luminous brightness. It uses furimazine (2-furanylmethyl-deoxycoelenterazine) as a substrate, and the peak value of the light emitted when catalyzing chemical reactions is 450 nm, which is similar to the excitation light of cpEGFP of 488 nm used by GRAB sensor of the present disclosure. According to the principle of resonance energy transfer of light, when the spatial distance and relative position of Nanoluc and the respective GRAB sensors of the present disclosure meet the requirements, energy transfer would occur.

Therefore, in this example, the light emitted by Nanoluc was used as the energy donor of the serotonin sensor, so that the fluorescent signal of the sensor was detected without external excitation light. This type of serotonin sensor can be imaged without external excitation light, therefore will be useful for studying the function of serotonin-related neural microcircuits in living animals.

Based on the structural changes of G protein-coupled receptor during ligand binding, the serotonin receptor HTR2C was selected for Nanoluc insertion. Based on GRAB-5-HT2.0 obtained in Example 9, Nanoluc was inserted at different positions in the C-terminus and expressed in HEK293T cells. After the sensor was expressed in the cells for 24 hours, furimazine was added. The fluorescence signal was detected using a microplate reader.

When the ligand serotonin (5-HT) binds to the receptor, the structure of the receptor changes, which will induce the change in the spatial distance and relative position of Nanoluc at the C-terminus and cpEGFP in the third intracellular loop, therefore changes the resonance energy transfer efficiency between the two, resulting in the change the fluorescence signal of cpEGFP. The sensor can be imaged without external excitation light, and the change in fluorescent signal reflects the binding process of serotonin to the receptor.

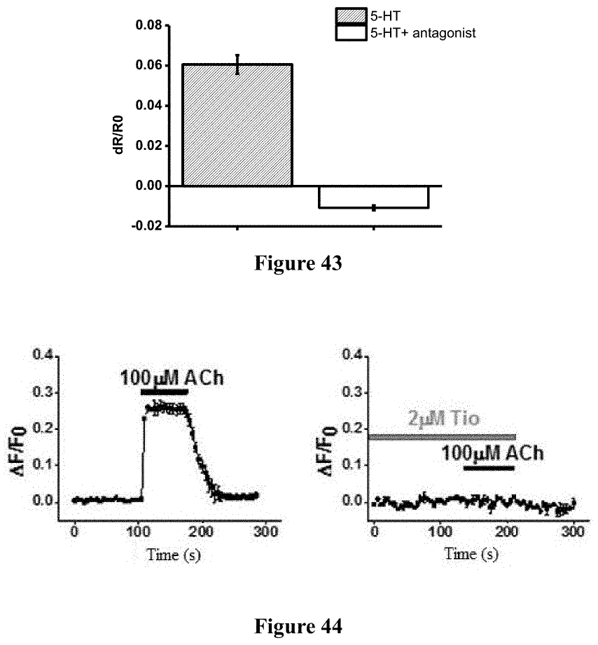

A sensor was obtained by optimizing the insertion position and peptide linkers. The sensor showed a 6% signal enhancement after 10 μM 5-HT treatment, and this signal change was suppressed by the HTR2C antagonist, as shown in FIG. 43. The specific insertion position of this sensor was between amino acids positions 582 and 583 of GRAB-5-HT2.0 (that is, between the amino acids positions 582 and 583 of the entire sensor after the insertion of fluorescent protein) obtained in Example 9. The peptide linkers at the N-terminus and C-terminus of Nanoluc were GSG.

The applications of the sensor could be: expressing the sensor in the brain region of a living animal by transgene or viral injection, adding furimazine, a substrate of Nanoluc, to the foods of the animal, providing the substrate to the animal by feeding. After a period of time, the changes in the serotonin signal in the brain of animals are observed using the bioluminescence imaging device.

### Example 13: Optimization and Screening of Acetylcholine Sensors

### 1. Materials and Methods

Same as in Example 1.

2. Acetylcholine Receptor and cpEGFP

Same as in Example 2, wherein the human acetylcholine receptor M3R subtype was also referred to as M3R receptor or CHRM3 in this example.

3. ICL3 Truncation and cpEGFP Insertion

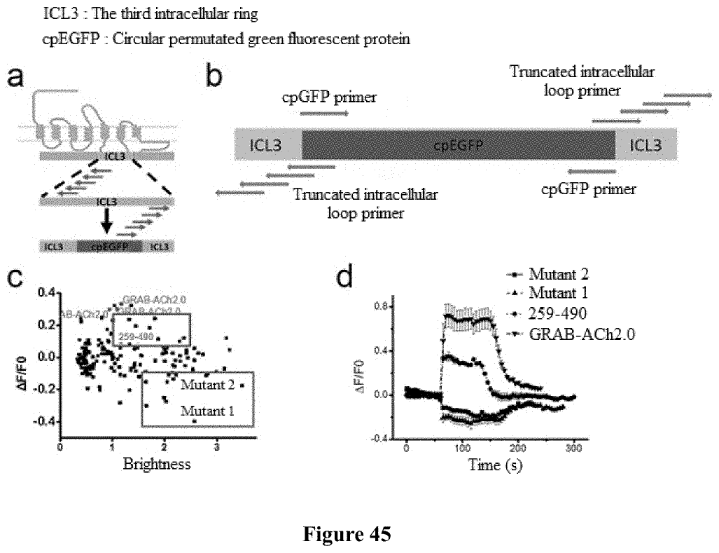

ICL3 was truncated between two random sites on the M3R receptor, and cpEGFP was inserted between the truncated positions (FIG. 45a). A library with a size of 7*8=56 was constructed by primer design (FIG. 45b). Since ICL3 was truncated randomly, in order to cover more possible combinations in the library, the screening scale was expanded to 200 clones (FIG. 45c). A clone with a signal enhancement of 30% was obtained using the high-throughput screening system, and its truncation sites on the M3R receptor were at positions 259 and 490 (FIG. 45d). In FIG. 45, the clone is shown as “490”.

4. Optimization of the Peptide Linkers Between cpEGFP and M3R Receptor

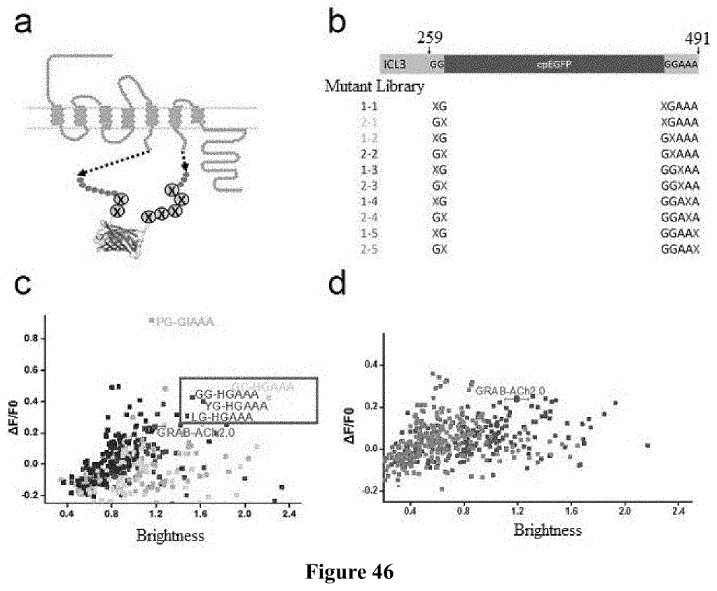

In order to systematically optimize the performance of the acetylcholine sensor (mainly the basic fluorescence intensity of the acetylcholine sensor and its response to saturated concentration of the ligand), based on the clone 259-490 obtained in the above steps, random mutations were performed on one amino acid of the peptide linker at the N-terminus and one amino acid of the peptide linker at the C-terminus (the original peptide linker was GG-GGAAA). The peptide linker at the N-terminus has 2 amino acid sites, and the peptide linker at the C-terminus has 5 amino acid sites, which were combined together to constitute a total of 2*5=10 libraries. Because each site, under random mutation, may be mutated into any one of the 20 amino acids in the human body, each library includes 20*20=400 possible combinations of amino acid residues (FIGS. 46a and b). The preliminary screening of a total of 4000 plasmids in these 10 libraries was performed using the Opera Phenix high-content screening platform. Since the Opera Phenix high-content screening platform can only screen 60 plasmids at a time, only 100 plasmids were chosen from each library fortest.

After screening 1000 plasmids from 10 libraries, it was found that when the first site of the peptide linker at the C-terminus between cpEGFP and M3R was histidine (His, H), the basal fluorescence intensity of the sensor was strong and the response to the saturated concentration of the ligand was also greater (FIG. 46c), that is, the peptide linker was GG-HGAAA. Therefore, the first site of the peptide linker at the C-terminus was fixed to H. Based thereon, random mutations were performed on the remaining 6 sites one by one (FIGS. 46c and d).

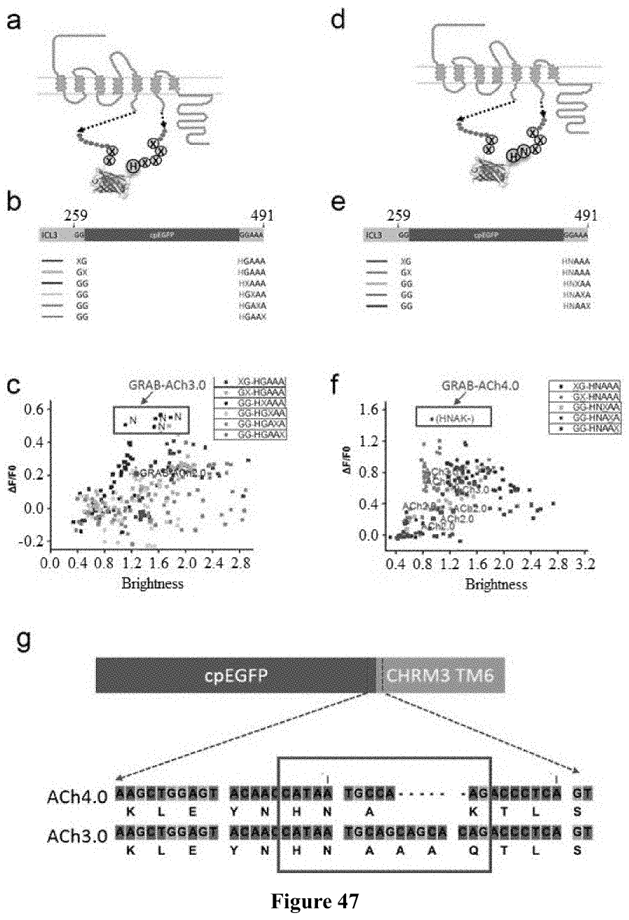

After fixing the first site of the peptide linker at the C-terminus to H, random mutations were performed on the remaining 6 sites one by one. It was found that, when the second site of the peptide linker at the C-terminus was mutated to N, the response of the sensor to acetylcholine almost doubled, and the basic fluorescence intensity also increased slightly (FIG. 47e). The sequence of the peptide linker was GG-HNAAA, and the sensor was named GRAB-ACh3.0.

The first and second sites of the peptide linker at the C-terminus were fixed to H and N, respectively, and based thereon, random mutations were performed on the remaining 5 sites one by one (FIGS. 47b and e). Through this round of random mutations, it was found that the loss of 6 base pairs doubled the response of the acetylcholine sensor (FIG. 47f). In the library with randomly mutation in the fourth site of the peptide linker at the C-terminus, 6 base pairs were deleted due to unexpected reasons, so the amino acid Q at position 491 of the M3R receptor was further truncated, and the fourth site of the peptide linker at the C-terminus was mutated to lysine (Lys, K) (FIG. 47g). This sensor was named GRAB-ACh4.0, in which, positions 260-491 of the M3R receptor were truncated and inserted with cpEGFP. The peptide linker between cpEGFP and M3R receptor was GG at the N-terminus and HNAK at the C-terminus.

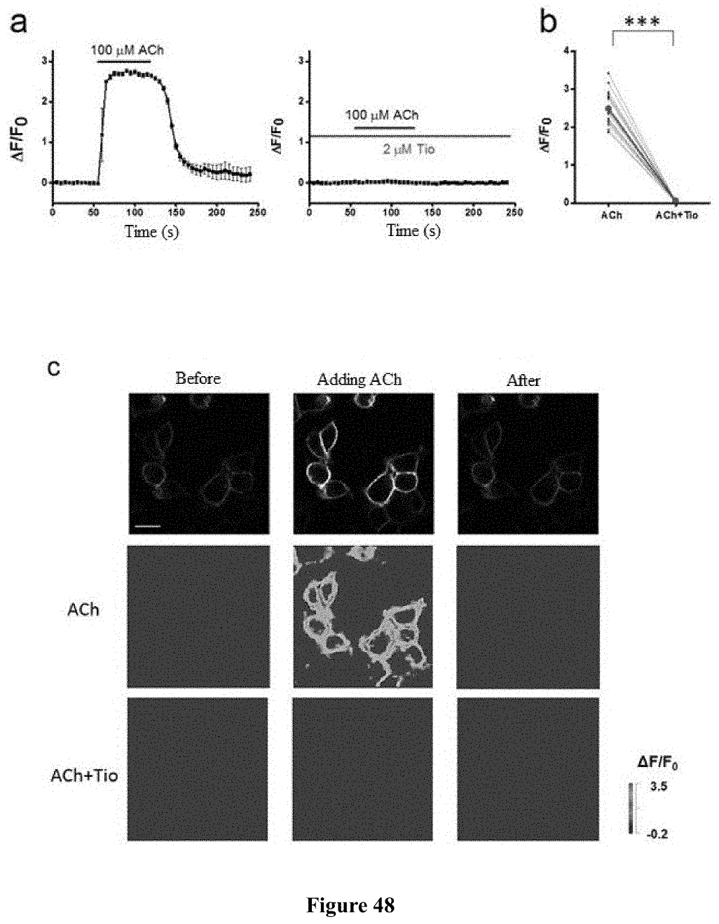

Perfusion experiments were performed to verify the performance of GRAB-ACh4.0. Under confocal microscope, when a saturated concentration of ACh was added, the response of the GRAB-ACh4.0 sensor expressed in a single cell could reach more than 250% (FIG. 48a); while in the presence of the antagonist Tiotropium bromide (Tio), the enhancement of the fluorescent signal of the sensor was almost completely inhibited, which indicated that the enhancement of the fluorescent signal of the sensor (that is, the response when ACh was added) was completely induced by ACh (FIG. 48a). The same perfusion experiment was performed on 18 cells, and their response after adding of Ach in the presence or absence of antagonists was recorded. It can be seen that the average response of these 18 cells when dosing exceeded 250%, the response of most cells was higher than 200%, and some even reached 350% (FIGS. 48b and c).

### 5. No Significant Difference in ACh Binding Capacity Between GRAB-ACh4.0 and Wild-Type M3R Receptor

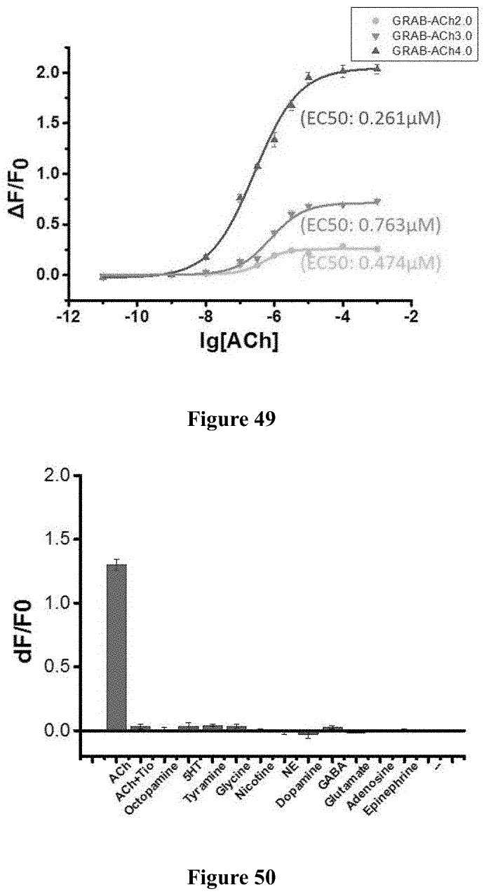

An important property of acetylcholine sensors is its binding capacity (Kd) to acetylcholine. The acetylcholine sensor can detect the concentration of acetylcholine in the body only if Kd is within the appropriate range. If Kd is too large or too small, the concentration of acetylcholine in the body may already be higher than the saturation concentration, or lower than the detection limit of the acetylcholine sensor, so the sensor cannot quantitatively detect the concentration of acetylcholine. The binding capacity of GRAB-ACh4.0 to acetylcholine was measured using Opera Phenix (FIG. 49). It can be seen that, the acetylcholine sensor GRAB-ACh4.0 has a Kd of 2.61h4.⁻⁷ and can detect acetylcholine at a concentration from 10⁻⁹ to 10⁻⁵ mol/L. The binding capacity of GRAB-ACh4.0 sensor to acetylcholine was close to that of the reported human-derived acetylcholine M3R receptor in the literature (Jakubik, J., Bacakova, L., El-Fakahany, E. E. & Tucek, S. Positive cooperativity of acetylcholine and other agonists with allosteric ligands on muscarinic acetylcholine receptors. Mol Pharmacol 52, 172-179 (1997)). So the GRAB-ACh4.0 sensor can be used to quantitatively measure the concentration of acetylcholine in the body.

### 6. GRAB-ACh4.0 has Strong Specificity to Ligand.

In order to verify the specificity of the acetylcholine sensor GRAB-ACh4.0, different neurotransmitters were added to HEK293T cells expressing GRAB-ACh4.0, and the response of the sensor was detected by Opera Phenix™. It can be seen that the GRAB-ACh4.0 sensor has high specificity in HEK293T cells—after adding the antagonist Tio, the response of the sensor was almost reduced to none, indicating that the change in the fluorescence intensity of GRAB-ACh4.0 was indeed induced by the binding of acetylcholine. When other neurotransmitters were added, the fluorescence intensity of GRAB-ACh4.0 also has little change, indicating that the GRAB-ACh4.0 sensor did not bind to other neurotransmitters (FIG. 50). In summary, GRAB-ACh4.0 can only be activated by Ach to produce change in fluorescence intensity.

7. GRAB-ACh4.0 does not Activate Downstream Gq-Mediated Signaling Pathways.

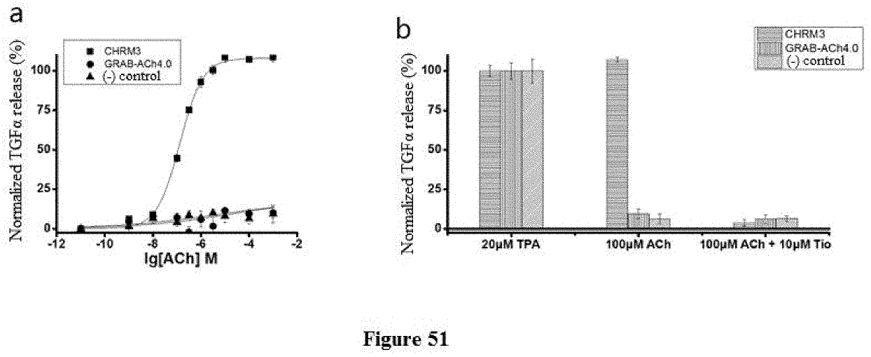

The coupling of GRAB-ACh4.0 and downstream Gαq was detected. First, three stable cell lines were constructed based on the Gαq cell line: M3R (marked as CHRM3 in FIG. 51), GRAB-ACh4.0, and blank (marked as Gq in FIG. 51) cell lines; then, the release of TGFα was used to characterize the extent to which the Gαq protein was activated. It can be seen that, only stable cell line expressing wild-type M3R activated the Gαq-mediated signal transduction pathway when applying gradient concentrations of ACh, while the coupling of GRAB-ACh4.0 to the G protein a subunit in the stable cell lines expressing GRAB-ACh4.0 was almost equivalent to that of the background cell line, suggesting that the GRAB-ACh4.0 sensor did not activate the Gαq-mediated signal transduction pathway (FIG. 51a). Tissue Plasminogen Activator (TPA) is a serum protease that can directly dissolve cell membranes, causing the release of TGF-α with alkaline phosphatase even when there is no signal from Gαq. Therefore, TPA was added to cells as a positive control. Response of the supernatant was the greatest after adding TPA, indicating that there is no problem with substrates and enzymes. After adding ACh, the activation of G protein only appeared in the cells expressing M3R, but not in the cells expressing GRAB-ACh4.0, indicating that the GRAB-ACh4.0 sensor will not couple to G protein and activate downstream signaling pathway, so as to disrupt the normal physiological functions of cells. The antagonist Tio could completely block the downstream signal induced by M3R, indicating that the activation of downstream G protein of M3R was indeed induced by the binding of ACh (FIG. 51b).

1. A fusion polypeptide comprising a G protein-coupled receptor (GPCR) part and a signal molecule part, wherein the G protein-coupled receptor is capable of specifically binding to ligand thereof, and the signal molecule is capable of directly or indirectly generating a detectable signal, such as an optical signal or a chemical signal, in response to the binding.

2. The fusion polypeptide according to claim 1, wherein the signal molecule is connected to an intracellular region of the G protein-coupled receptor; particularly, the signal molecule is connected to an intracellular loop or the C-terminus of the GPCR, for example the first intracellular loop, the second intracellular loop, the third intracellular loop or the C-terminus of the GPCR, preferably the third intracellular loop or C-terminus of the GPCR, and more preferably the third intracellular loop of the GPCR.

3. The fusion polypeptide according to claim 2, wherein the signal molecule is connected to the third intracellular loop or C-terminus of the GPCR, and the third intracellular loop or C-terminus is a truncated third intracellular loop or C-terminus, preferably, the third intracellular loop or the C-terminus is truncated 10-200 amino acids, such as 10, 20, 30, 40, 50, 60, 70, 80, 90, 100, 110, 120, 130, 140, 150, 160, 170, 180, 190, 200 amino acids, or a range between any two of the values thereof.

4. The fusion polypeptide according to claim 2, wherein the signal molecule is connected to the GPCR through a peptide linker, for example, the signal molecule is connected to the third intracellular loop of the GPCR through a linker peptide; preferably, the peptide linker comprises a flexible amino acid; more preferably, the flexible amino acid comprises glycine and/or alanine; even more preferably, the peptide linker consists of glycine and alanine; and most preferably, the peptide linker at the N-terminus of the signal molecule is GG, and/or the peptide linker at the C-terminus of the signal molecule is GGAAA.

5. The fusion polypeptide according to claim 1, wherein the detectable signal is an optical signal; preferably, the signal molecule is a fluorescent protein or luciferase; more preferably, the signal molecule is a circular permutated fluorescent protein or a circular permutated luciferase.

6. The fusion polypeptide according to claim 5, wherein the signal molecule is a circular permutated fluorescent protein,
for example, the circular permutated fluorescent protein is selected from the group consisting of circular permutated green fluorescent protein (cpGFP), circular permutated yellow fluorescent protein (cpYFP), circular permutated red fluorescent protein (cpRFP), circular permutated blue fluorescent protein (cpBFP), circular permutated enhanced green fluorescent protein (cpEGFP), circular permutated enhanced yellow fluorescence protein (cpEYFP) and circular permutated infrared fluorescent protein (cpiRFP);
for example, the circular permutated enhanced green fluorescent protein is from GCaMP6s, GCaMP6m or G-GECO;
for example, the circular permutated red fluorescent protein is selected from the group consisting of cpmApple, cpmCherry, cpmRuby2, cpmKate2, and cpFushionRed,
particularly, the cpmApple is from R-GECO1;
for example, the circular permutated yellow fluorescent protein is selected from circular permutated Venus (cpVenus) and circular permutated Citrin (cpCitrine).

7. The fusion polypeptide according to claim 1, wherein the GPCR is capable of specifically binding to ligand thereof, wherein the ligand is selected from the group consisting of a neurotransmitter, hormone, metabolic molecule, nutrition molecule, and an artificially synthesized small molecule or drug candidate capable of activating a specific receptor; and the GPCR is capable of specifically binding to the neurotransmitter, hormone, metabolic molecule, nutrition molecule, or the artificially synthesized small molecule or drug candidate;
for example, the neurotransmitter is epinephrine, norepinephrine, acetylcholine, serotonin and/or dopamine;
for example, the artificially synthesized small molecule or drug candidate capable of activating a specific receptor is isoproterenol (ISO);
for example, the G protein-coupled receptor is derived from human or mammalian G protein-coupled receptor;
for example, the fusion polypeptide is a fluorescent sensor for detecting epinephrine, and the GPCR is capable of specifically binding to epinephrine;
particularly, the GPCR capable of specifically binding to epinephrine is a human β2 adrenergic receptor, and the fusion polypeptide is a fluorescent sensor constructed based on the human β2 adrenergic receptor.

8. The fusion polypeptide according to claim 7, wherein the signal molecule is the circular permutated fluorescent protein and the circular permutated fluorescent protein is inserted into the third intracellular loop of the human β2 adrenergic receptor through peptide linkers at the N-terminus and the C-terminus;
preferably, the lengths of the peptide linkers are 1 or 2 amino acids at the N-terminus and/or 1, 2, 3, 4 or 5 amino acids at the C-terminus of the circular permutated fluorescent protein, respectively;
more preferably, the lengths of the peptide linkers are 2 amino acids at the N-terminus and 5 amino acids at the C-terminus of the circular permutated fluorescent protein, respectively; and
preferably, the peptide linkers are GG at the N-terminus and GGAAA at the C-terminus of the circular permutated fluorescent protein, respectively, or
the peptide linkers are GG at the N-terminus and SPSVA at the C-terminus of the circular permutated fluorescent protein, respectively, or
the peptide linkers are GG at the N-terminus and APSVA at the C-terminus of the circular permutated fluorescent protein, respectively;
or
more preferably, the lengths of the peptide linkers are 1 amino acid at the N-terminus and 1 amino acid at the C-terminus of the circular permutated fluorescent protein, respectively; particularly preferably, the peptide linkers are G at the N-terminus and G at the C-terminus of the circular permutated fluorescent protein, respectively;
further preferably, the circular permutated fluorescent protein inserted into the human β2 adrenergic receptor is cpEGFP; preferably, the cpEGFP is cpEGFP from GCaMP6s, GCaMP6m or GECO1.2,
particularly preferably, the amino acid sequence of the human β2 adrenergic receptor is:
(SEQ ID NO: 1)
MGQPGNGSAFLLAPNRSHAPDHDVTQQRDEVWVVGMGIVMSLIVLAIVF
GNVLVITAIAKFERLQTVTNYFITSLACADLVMGLAVVPFGAAHILMKM
WTFGNFWCEFWTSIDVLCVTASIETLCVIAVDRYFAITSPFKYQSLLTK
NKARVIILMVWIVSGLTSFLPIQMHWYRATHQEAINCYANETCCDFFTN
QAYAIASSIVSFYVPLVIMVFVYSRVFQEAKRQLQKIDKSEGRFHVQNL
SQVEQDGRTGHGLRRSSKFCLKEHKALKTLGIIMGTFTLCWLPFFIVNI
VHVIQDNLIRKEVYILLNWIGYVNSGFNPLIYCRSPDFRIAFQELLCLR
RSSLKAYGNGYSSNGNTGEQSGYHVEQEKENKLLCEDLPGTEDFVGHQG
TVPSDNIDSQGRNCSTNDSLL,
wherein the underlined part is the third intracellular loop;
preferably, the circular permutated fluorescent protein is inserted into the human β2 adrenergic receptor between amino acid position 240 and amino acid position 241, or between amino acid position 250 and amino acid position 251.

9. The fusion polypeptide according to claim 1, wherein the fusion polypeptide is a fluorescent sensor for detecting epinephrine and/or norepinephrine, and the GPCR is capable of specifically binding to adrenaline and/or norepinephrine;
preferably, the GPCR capable of specifically binding to adrenaline and/or norepinephrine is a human ADRA2A receptor, and the fusion polypeptide is a fluorescent sensor constructed based on the human ADRA2A receptor;
further preferably, the third intracellular loop of the human ADRA2A receptor is truncated and a circular permutated fluorescent protein is inserted at the truncated position;
further preferably, the circular permutated fluorescent protein is inserted into the third intracellular loop of the human ADRA2A receptor through peptide linkers at the N-terminus and the C-terminus, and the lengths of the peptide linkers are 2 amino acids at the N-terminus and 5 amino acids at the C-terminus of the circular permutated fluorescent protein, respectively;
preferably, the peptide linkers are GG at the N-terminus and GGAAA at the C-terminus of the circular permutated fluorescent protein, respectively, or the peptide linkers are GG at the N-terminus and TGAAA at the C-terminus of the circular permutated fluorescent protein, respectively;
further preferably, the circular permutated fluorescent protein inserted into the human ADRA2A receptor is cpEGFP; preferably, the cpEGFP is cpEGFP from GCaMP6s, GCaMP6m or GECO1.2;
more preferably, the amino acid sequence of the human ADRA2A receptor is:
(SEQ ID NO: 2)
MFRQEQPLAEGSFAPMGSLQPDAGNASWNGTEAPGGGARATPYSLQVTLT
LVCLAGLLMLLTVFGNVLVIIAVFTSRALKAPQNLFLVSLASADILVATL
VIPFSLANEVMGYWYFGKAWCEIYLALDVLFCTSSIVHLCAISLDRYWSI
TQAIEYNLKRTPRRIKAIIITVWVISAVISFPPLISIEKKGGGGGPQPAE
PRCEINDQKWYVISSCIGSFFAPCLIMILVYVRIYQIAKRRTRVPPSRRG
PDAVAAPPGGTERRPNGLGPERSAGPGGAEAEPLPTQLNGAPGEPAPAGP
RDTDALDLEESSSSDHAERPPGPRRPERGPRGKGKARASQVKPGDSLPRR
GPGATGIGTPAAGPGEERVGAAKASRWRGRQNREKRFTFVLAVVIGVFVV
CWFPFFFTYTLTAVGCSVPRTLFKFFFWFGYCNSSLNPVIYTIFNHDFRR
AFKKILCRGDRKRIV,
wherein the underlined part is the third intracellular loop;
preferably, amino acids 71-130 of the third intracellular loop of the human ADRA2A receptor are truncated, and the circular permutated fluorescent protein is inserted at the truncated positions; or amino acids 71-135 of the third intracellular loop of the human ADRA2A receptor are truncated, and the circular permutated fluorescent protein is inserted at the truncated positions.

10. The fusion polypeptide according to claim 1, wherein the fusion polypeptide is a fluorescent sensor constructed based on a G protein-coupled receptor for detecting acetylcholine, and the G protein-coupled receptor is capable of specifically binding to acetylcholine;
preferably, the GPCR capable of specifically binding to adrenaline is a human acetylcholine receptor M3R subtype, and the fluorescent protein constructed based on the G protein-coupled receptor is a fluorescent sensor constructed based on the human acetylcholine receptor M3R subtype;
further preferably, the third intracellular loop of the human acetylcholine receptor M3R subtype is truncated and a circular permutated fluorescent protein is inserted at the truncated positions;
further preferably, the circular permutated fluorescent protein is inserted into the third intracellular loop of the human acetylcholine receptor M3R subtype through peptide linkers at the N-terminus and the C-terminus; preferably, the lengths of the peptide linker are 2 amino acids at the N-terminus and 5 amino acids at the C-terminus of the circular permutated fluorescent protein, respectively;
preferably, the peptide linkers are GG at the N-terminus and GGAAA at the C-terminus of the circular permutated fluorescent protein, respectively, or the peptide linkers are GG at the N-terminus and HGAAA at the C-terminus of the circular permutated fluorescent protein, respectively, or the peptide linkers are GG at the N-terminus and HNAAA at the C-terminus of the circular permutated fluorescent protein, respectively, or the peptide linkers are GG at the N-terminus and HNAK at the C-terminus of the circular permutated fluorescent protein, respectively;
more preferably, the circular permutated fluorescent protein inserted into the human acetylcholine receptor M3R subtype is cpEGFP; preferably, the cpEGFP is cpEGFP from GCaMP6s, GCaMP6m, or GECO1.2;
more preferably, the amino acid sequence of the human acetylcholine receptor M3R subtype is:
(SEQ ID NO: 3)
MTLHNNSTTSPLFPNISSSWIHSPSDAGLPPGTVTHFGSYNVSRAAGNFS
SPDGTTDDPLGGHTVWQVVFIAFLTGILALVTIIGNILVIVSFKVNKQLK
TVNNYFLLSLACADLIIGVISMNLFTTYIIMNRWALGNLACDLWLAIDYV
ASNASVMNLLVISFDRYFSITRPLTYRAKRTTKRAGVMIGLAWVISFVLW
APAILFWQYFVGKRTVPPGECFIQFLSEPTITFGTAIAAFYMPVTIMTIL
YWRIYKETEKRTKELAGLQASGTEAETENFVHPTGSSRSCSSYELQQQSM
KRSNRRKYGRCHFWFTTKSWKPSSEQMDQDHSSSDSWNNNDAAASLENSA
SSDEEDIGSETRAIYSIVLKLPGHSTILNSTKLPSSDNLQVPEEELGMVD
LERKADKLQAQKSVDDGGSFPKSFSKLPIQLESAVDTAKTSDVNSSVGKS
TATLPLSFKEATLAKRFALKTRSQITKRKRMSLVKEKKAAQTLSAILLAF
IITWTPYNIMVLVNTFCDSCIPKTFWNLGYWLCYINSTVNPVCYALCNKT
FRTTFKMLLLCQCDKKKRRKQQYQQRQSVIFHKRAPEQAL,
wherein the underlined part is the third intracellular loop;
preferably, amino acids 260-490 of the third intracellular loop of the human acetylcholine receptor M3R subtype are truncated, and the circular permutated fluorescent protein is inserted at the truncated positions; or amino acids 260-491 of the third intracellular loop of the human acetylcholine receptor M3R subtype are truncated, and the circular permutated fluorescent protein is inserted at the truncated positions.

11. The fusion polypeptide according to claim 1, wherein the fusion polypeptide is a fluorescent sensor for detecting serotonin, and the GPCR is capable of specifically binding to serotonin;
preferably, the GPCR capable of specifically binding to serotonin is a human HTR2C receptor, and the fusion polypeptide is a fluorescent sensor constructed based on the human HTR2C receptor;
further preferably, the third intracellular loop of the human HTR2C receptor is truncated and a circular permutated fluorescent protein is inserted at the truncated position;
further preferably, the circular permutated fluorescent protein is connected to the third intracellular loop of the human HTR2C receptor through peptide linkers at the N-terminus and the C-terminus, and the lengths of the peptide linkers are 2 amino acids at the N-terminus and 5 amino acids at the C-terminus of the circular permutated fluorescent protein, respectively;
preferably, the peptide linkers are GG at the N-terminus and GGAAA at the C-terminus of the circular permutated fluorescent protein, respectively, or the peptide linkers are NG at the N-terminus and GFAAA at the C-terminus of the circular permutated fluorescent protein, respectively;
more preferably, the circular permutated fluorescent protein inserted into the human HTR2C receptor is cpEGFP; preferably, the cpEGFP is cpEGFP from GCaMP6s, GCaMP6m or GECO1.2;
particularly preferably, the amino acid sequence of the human HTR2C receptor is:
(SEQ ID NO: 4)
MVNLRNAVHSFLVHLIGLLVWQCDISVSPVAAIVTDIFNTSDGGRFKFPD
GVQNWPALSIVIIIIMTIGGNILVIMAVSMEKKLHNATNYFLMSLAIADM
LVGLLVMPLSLLAILYDYVWPLPRYLCPVWISLDVLFSTASIMHLCAISL
DRYVAIRNPIEHSRFNSRTKAIMKIAIVWAISIGVSVPIPVIGLRDEEKV
FVNNTTCVLNDPNFVLIGSFVAFFIPLTIMVITYCLTIYVLRRQALMLLH
GHTEEPPGLSLDFLKCCKRNTAEEENSANPNQDQNARRRKKKERRPRGTM
QAINNERKASKVLGIVFFVFLIMWCPFFITNILSVLCEKSCNQKLMEKLL
NVFVWIGYVCSGINPLVYTLFNKIYRRAFSNYLRCNYKVEKKPPVRQIPR
VAATALSGRELNVNIYRHTNEPVIEKASDNEPGIEMQVENLELPVNPSSV
VSERISSV,
wherein the underlined part is the third intracellular loop;
preferably, amino acids 16-55 of the third intracellular loop of the human HTR2C receptor are truncated, and a circular permutated fluorescent protein is inserted at the truncated positions; or amino acids 11-60 of the third intracellular loop of the human HTR2C receptor are truncated, and a circular permutated fluorescent protein is inserted at the truncated positions; or amino acids 16-70 of the third intracellular loop of the human HTR2C receptor are truncated, and a circular permutated fluorescent protein is inserted at the truncated positions; or amino acids 15-68 of the third intracellular loop of the human HTR2C receptor are truncated, and a circular permutated fluorescent protein is inserted at the truncated positions; or amino acids 15-68 of the third intracellular loop of the human HTR2C receptor are truncated, and a circular permutated fluorescent protein is inserted at the truncated position, and the leucine L at position 13 of the third intracellular loop is replaced with phenylalanine F.

12. The fusion polypeptide according to claim 11, wherein the circular permutated fluorescent protein is inserted into the third intracellular loop of the human HTR2C receptor through peptide linkers at the N-terminus and the C-terminus; preferably, the lengths of the peptide linkers are 5 amino acids at the N-terminus and 3 amino acids at the C-terminus of the circular permutated fluorescent protein, respectively; more preferably, the peptide linkers are PVVSE at the N-terminus and ATR at the C-terminus of the circular permutated fluorescent protein, respectively;
preferably, the circular permutated fluorescent protein inserted into the human HTR2C receptor is cpmApple; preferably, the cpmApple is cpmApple from R-GECO1;
further preferably, the amino acid sequence of the human HTR2C receptor is:
(SEQ ID NO: 4)
MDPLNLSWYDDDLERQNWSRPFNGSDGKADRPHYNYYATLLTLLIAVIV
FGNVLVCMAVSREKALQTTTNYLIVSLAVADLLVATLVMPWVVYLEVVG
EWKFSRIHCDIFVTLDVMMCTASILNLCAISIDRYTAVAMPMLYNTRYS
SKRRVTVMISIVWVLSFTISCPLLFGLNNADQNECIIANPAFVVYSSIV
SFYVPFIVTLLVYIKIYIVLRRRRKRVNTKRSSRAFRAHLRAPLKGNCT
HPEDMKLCTVIMKSNGSFPVNRRRVEAARRAQELEMEMLSSTSPPERTR
YSPIPPSHHQLTLPDPSHHGLHSTPDSPAKPEKNGHAKDHPKIAKIFEI
QTMPNGKTRTSLKTMSRRKLSQQKEKKATQMLAIVLGVFIICWLPFFIT
HILNIHCDCNIPPVLYSAFTWLGYVNSAVNPIIYTTFNIEFRKAFLKIL
HC,
wherein the underlined part is the third intracellular loop;
preferably, amino acids 241-306 of the third intracellular loop of the human HTR2C receptor are truncated, and the circular permutated fluorescent protein is inserted at the truncated positions; or amino acids 240-309 of the third intracellular loop of the human HTR2C receptor are truncated, and the circular permutated fluorescent protein is inserted at the truncated positions.

13. The fusion polypeptide according to claim 1, wherein the fusion polypeptide is a fluorescent sensor for detecting dopamine, and the GPCR is capable of specifically binding to dopamine;
preferably, the GPCR capable of specifically binding to dopamine is a human DRD2 receptor, and the fusion polypeptide is a fluorescent sensor constructed based on the human DRD2 receptor;
further preferably, the third intracellular loop of the human DRD2 receptor is truncated and a circular permutated fluorescent protein is inserted at the truncated position;
further preferably, the circular permutated fluorescent protein is inserted into the third intracellular loop of the human DRD2 receptor through peptide linkers at the N-terminus and the C-terminus; preferably, the lengths of the peptide linkers are 2 amino acids at the N-terminus and 5 amino acids at the C-terminus of the circular permutated fluorescent protein, respectively; further preferably, the peptide linkers are GG at the N-terminus and GGAAA at the C-terminus of the circular permutated fluorescent protein, respectively;
further preferably, the circular permutated fluorescent protein inserted into the human DRD2 receptor is cpEGFP; preferably, the cpEGFP is cpEGFP from GCaMP6s, GCaMP6m or GECO1.2;
particularly preferably, the amino acid sequence of the human DRD2 receptor is:
(SEQ ID NO: 5)
MDPLNLSWYDDDLERQNWSRPFNGSDGKADRPHYNYYATLLTLLIAVIV
FGNVLVCMAVSREKALQTTTNYLIVSLAVADLLVATLVMPWVVYLEVVG
EWKFSRIHCDIFVTLDVMMCTASILNLCAISIDRYTAVAMPMLYNTRYS
SKRRVTVMISIVWVLSFTISCPLLFGLNNADQNECIIANPAFVVYSSIV
SFYVPFIVTLLVYIKIYIVLRRRRKRVNTKRSSRAFRAHLRAPLKGNCT
HPEDMKLCTVIMKSNGSFPVNRRRVEAARRAQELEMEMLSSTSPPERTR
YSPIPPSHHQLTLPDPSHHGLHSTPDSPAKPEKNGHAKDHPKIAKIFEI
QTMPNGKTRTSLKTMSRRKLSQQKEKKATQMLAIVLGVFIICWLPFFIT
HILNIHCDCNIPPVLYSAFTWLGYVNSAVNPIIYTTFNIEFRKAFLKIL
HC,
wherein the underlined part is the third intracellular loop;
preferably, amino acids 253-357 of the third intracellular loop of the human DRD2 receptor are truncated, and the circular permutated fluorescent protein is inserted at the truncated positions; or amino acids 254-360 of the third intracellular loop of the human DRD2 receptor are truncated, and the circular permutated fluorescent protein is inserted at the truncated positions.

14. The fusion polypeptide according to claim 13, wherein the circular permutated fluorescent protein is inserted into the third intracellular loop of the human DRD2 receptor through peptide linkers at the N-terminus and the C-terminus; preferably, the lengths of the peptide linkers are 5 amino acids at the N-terminus and 3 amino acids at the C-terminus of the circular permutated fluorescent protein, respectively; more preferably, the peptide linkers are PVVSE at the N-terminus and ATR at the C-terminus of the circular permutated fluorescent protein, respectively;
preferably, the circular permutated fluorescent protein inserted into the human DRD2 receptor is cpmApple; preferably, the cpmApple is cpmApple from R-GECO1;
further preferably, the amino acid sequence of the human DRD2 receptor is:
(SEQ ID NO: 5)
MDPLNLSWYDDDLERQNWSRPFNGSDGKADRPHYNYYATLLTLLIAVIV
FGNVLVCMAVSREKALQTTTNYLIVSLAVADLLVATLVMPWVVYLEVVG
EWKFSRIHCDIFVTLDVMMCTASILNLCAISIDRYTAVAMPMLYNTRYS
SKRRVTVMISIVWVLSFTISCPLLFGLNNADQNECIIANPAFVVYSSIV
SFYVPFIVTLLVYIKIYIVLRRRRKRVNTKRSSRAFRAHLRAPLKGNCT
HPEDMKLCTVIMKSNGSFPVNRRRVEAARRAQELEMEMLSSTSPPERTR
YSPIPPSHHQLTLPDPSHHGLHSTPDSPAKPEKNGHAKDHPKIAKIFEI
QTMPNGKTRTSLKTMSRRKLSQQKEKKATQMLAIVLGVFIICWLPFFIT
HILNIHCDCNIPPVLYSAFTWLGYVNSAVNPIIYTTFNIEFRKAFLKIL
HC,
wherein the underlined part is the third intracellular loop;
preferably, amino acids 223-349 of the third intracellular loop of the human DRD2 receptor are truncated, and the circular permutated fluorescent protein is inserted at the truncated positions; or amino acids 268-364 of the third intracellular loop of the human DRD2 receptor are truncated, and the circular permutated fluorescent protein is inserted at the truncated positions; or amino acids 224-365 of the third intracellular loop of the human DRD2 receptor are truncated, and the circular permutated fluorescent protein is inserted at the truncated positions.

15. The fusion polypeptide according to claim 1, wherein the fusion polypeptide further comprises a Gα peptide segment connected to the C-terminus of the GPCR, for example, the Gα peptide segment is 20 amino acids at the C-terminus of the Gα protein; preferably, the Gα peptide segment is connected to the last amino acid at the C-terminus of the GPCR; more preferably, the sequence of the Gα peptide segment is selected from the group consisting of VFAAVKDTILQLNLKEYNLV (SEQ ID NO: 6), VFNDCRDIIQRMHLRQYELL (SEQ ID NO: 7) and VFDAVTDVIIKNNLKDCGLF (SEQ ID NO: 8).

16. The fusion polypeptide according to claim 1, wherein the fusion polypeptide further comprises a luciferase connected to the C-terminus of the GPCR, and the light emitted by the luciferase-catalyzed chemical reaction excites the circular permutated fluorescent protein; preferably, the luciferase is Nanoluc, Fluc (firefly luciferase) or Rluc (renilla luciferase);
for example, the fusion polypeptide is a fluorescent sensor constructed based on a human HTR2C receptor, the luciferase is inserted into the C-terminus of the fusion polypeptide, and the luciferase is inserted into the C-terminus of the fusion polypeptide through peptide linkers at the N-terminus and the C-terminus of the luciferase, and the peptide linkers at the N-terminus and the C-terminus of the luciferase both are GSG;
for example, the luciferase is inserted between amino acid positions 582 and 583 of the fluorescent sensor GRAB-5-HT2.0, and the luciferase are connected to the fluorescent sensor GRAB-5-HT2.0 through peptide linkers at the N-terminus and the C-terminus, wherein the peptide linkers at the N-terminus and the C-terminus of the luciferase both are GSG;
wherein fluorescent sensor GRAB-5-HT2.0 is a fluorescent sensor obtained by deleting the amino acid residues at the positions 15-68 of the third intracellular loop of the human HTR2C receptor, and inserting cpEGFP at the deleted position, wherein the N-terminus of cpEGFP is connected to the human HTR2C receptor through the N-terminal peptide linker NG, and the C-terminus of cpEGFP is connected to the human HTR2C receptor through the C-terminal peptide linker GFAAA;
wherein the amino acid sequence of the human HTR2C receptor is
(SEQ ID NO: 4)
MVNLRNAVHSFLVHLIGLLVWQCDISVSPVAAIVTDIFNTSDGGRFKFPD
GVQNWPALSIVIIIIMTIGGNILVIMAVSMEKKLHNATNYFLMSLAIADM
LVGLLVMPLSLLAILYDYVWPLPRYLCPVWISLDVLFSTASIMHLCAISL
DRYVAIRNPIEHSRFNSRTKAIMKIAIVWAISIGVSVPIPVIGLRDEEKV
FVNNTTCVLNDPNFVLIGSFVAFFIPLTIMVITYCLTIYVLRRQALMLLH
GHTEEPPGLSLDFLKCCKRNTAEEENSANPNQDQNARRRKKKERRPRGTM
QAINNERKASKVLGIVFFVFLIMWCPFFITNILSVLCEKSCNQKLMEKLL
NVFVWIGYVCSGINPLVYTLFNKIYRRAFSNYLRCNYKVEKKPPVRQIPR
VAATALSGRELNVNIYRHTNEPVIEKASDNEPGIEMQVENLELPVNPSSV
VSERISSV,
wherein the underlined part is the third intracellular loop;
preferably, the cpEGFP is cpEGFP from GCaMP6s.

17. A composition comprising:
a ligand recognition polypeptide comprising 1) the extracellular region of the G protein-coupled receptor (GPCR) in the fusion polypeptide of claim 1, and 2) a first protein interaction segment; and
a signal generating polypeptide comprising 1) a second protein interaction segment capable of specifically binding to the first protein interaction segment, and 2) the transmembrane and intracellular regions of the G protein-coupled receptor (GPCR) and the signal molecule in the fusion polypeptide of claim 1;
preferably, the extracellular region of the G protein-coupled receptor (GPCR) in the ligand recognition polypeptide and the transmembrane region and intracellular regions of the G protein-coupled receptor (GPCR) in the signal generating polypeptide are derived from different G protein-coupled receptors.

18. The composition according to claim 17, wherein the protein interaction segment is a leucine zipper domain; preferably, one of the protein interaction segments is BZip (RR), and the other protein interaction segment is AZip (EE).

19. The composition of claim 17, wherein the first and second protein interaction segments are selected from the group consisting of:
1) PSD95-Dlgl-zo-1 (PDZ) domain;
2) Streptavidin and streptavidin binding protein (SBP);
3) FTORP binding domain (FRB) and FK506 binding protein (FKBP) of mTOR;
4) Cyclophilin-Fas fusion protein (CyP-Fas) and FK506 binding protein (FKBP);
5) Calcineurin A (CNA) and FK506 binding protein (FKBP);
6) SNAP tags and Halo tags; and
7) PYL and ABI.

20.-25. (canceled)

26. A method for detecting a GPCR ligand in an object, comprising
exposing the object to the fusion polypeptide of claim 1, wherein the GPCR in the fusion polypeptide is capable of specifically binding to the ligand,
comparing the detectable signal caused by the exposure with one or more references containing a predetermined amount of the ligand, and analyzing the presence, content, or time and/or spatial change of the ligand in the analysis object;
for example, the one or more references containing a predetermined amount of the ligand include at least a reference not containing the ligand, and preferably also include at least one reference containing a non-zero amount of the ligand.

27. The method of claim 26, wherein the detectable signal is an optical signal; preferably the fusion polypeptide is a fluorescent sensor that responds to the specific binding of the GPCR to its ligand, wherein the specific binding causes a change in the fluorescent signal, such as a change in the intensity of the fluorescent signal, for example an increase or decrease in the intensity of the fluorescent signal.

28. The method according to claim 26, wherein the detection is performed in an ex vivo cell or in a living body; for example, the detection is to detect the distribution of the ligand in a living body; or for example, the ligand is selected from a neurotransmitter, hormone, metabolite and nutrient.

29. A method for identifying a substance targeting a GPCR, comprising
exposing the substance to the fusion polypeptide of claim 1, wherein the GPCR in the fusion polypeptide is capable of specifically binding to its ligand,
comparing the detectable signal caused by the exposure with one or more references containing a predetermined amount of the ligand, and analyzing the binding of the test substance to the GPCR, which indicates that the test substance is a candidate active substance targeting the GPCR;
for example, the one or more references containing a predetermined amount of the ligand include at least a reference not containing the ligand, and preferably also include at least one reference containing a non-zero amount of the ligand.

30. The method according to claim 29, wherein the detectable signal is an optical signal; preferably the fusion polypeptide is a fluorescent sensor that responds to the specific binding of the GPCR to its ligand, wherein the specific binding causes a change in the fluorescent signal, such as a change in the intensity of the fluorescent signal, for example an increase or decrease in the intensity of the fluorescent signal.

31.-32. (canceled)
